(19) United States
(12) Patent Application Publication
(10) Pub. No.: US 2022/0020934 A1
(43) Pub. Date: Jan. 20, 2022

(54) 5,6-DIPHENYL-5,6-DIHYDRO-DIBENZ[C,E][1,2]AZAPHOSPHORIN AND 6-PHENYL-6H-DIBENZO[C,E][1,2]THIAZIN-5,5-DIOXIDE DERIVATIVES AND SIMILAR COMPOUNDS AS ORGANIC ELECTROLUMINESCENT MATERIALS FOR OLEDS

(71) Applicant: Merck Patent GmbH, Darmstadt (DE)

(72) Inventors: Amir PARHAM, Frankfurt am Main (DE); Jens ENGELHART, Darmstadt (DE); Christian EICKHOFF, Mannheim (DE); Christian EHRENREICH, Darmstadt (DE); Jonas KROEBER, Frankfurt am Main (DE)

(21) Appl. No.: 17/291,991

(22) PCT Filed: Nov. 4, 2019
(86) PCT No.: PCT/EP2019/080039

§ 371 Date: May 6, 2021

(30) Foreign Application Priority Data
Nov. 6, 2018 (EP) 18204542.7

(51) Int. Cl. H01L 51/00; C07F 9/6584; C09K 11/06; C07D 417/04; C07D 279/02; C07D 513/04; C07D 291/08; C07D 285/36

(52) U.S. Cl. CPC H01L 51/0069; H01L 51/5012; C09K 11/06; H01L 51/0072; H01L 51/0074; H01L 51/0067; H01L 51/008; H01L 51/0052; H01L 51/0073; H01L 51/0054; C07D 417/04; C07D 279/02; C07D 513/04; C07D 291/08; C07D 285/36; H01L 51/0058; C07F 9/6584

(57) ABSTRACT

The present invention relates to 5,6-diphenyl-5,6-dihydrodibenz[c,e][1,2]azaphosphorin and 6-phenyl-6H-dibenzo[c,e][1,2]thiazine 5,5-dioxide derivatives and similar compounds of the formula (1) as organic electroluminescent materials for use in organic electroluminescent devices, for example in organic light-emitting diodes (OLEDs), where the symbols used are as follows: Z is the same or different at each instance and is PAr² or S(═O); E is the same or different at each instance and is O or S when the symbol Z to which this E binds is PAr², and O when the symbol Z to which this E binds is S(═O); L is selected from the group consisting of a single bond, NAr², O, S, S(═O)₂, P(═O)Ar², —X═X— and —C(═O)—NAr²; the rest of the symbols are defined in the claims. The present invention discloses synthesis examples of inventive compounds, productions of OLEDs containing these example compounds, and results for these electroluminescent devices.

The present invention relates to materials for use in electronic devices, especially in organic electroluminescent devices, and to electronic devices, especially organic electroluminescent devices comprising these materials.

Emitting materials used in organic electroluminescent devices (OLEDs) are frequently phosphorescent organometallic complexes. In general terms, there is still a need for improvement in OLEDs, especially also in OLEDs which exhibit triplet emission (phosphorescence), for example with regard to efficiency, operating voltage and lifetime. The properties of phosphorescent OLEDs are not just determined by the triplet emitters used. More particularly, the other materials used, such as matrix materials, are also of particular significance here.

It is an object of the present invention to provide compounds that are suitable for use in an OLED, especially as matrix material for phosphorescent emitters, but also as electron transport materials or hole blocker materials and, according to the substitution pattern, also as hole transport or hole injection or electron blocker materials. It is a further object of the present invention to provide further organic semiconductors for organic electroluminescent devices, in order thus to enable the person skilled in the art to have a greater possible choice of materials for the production of OLEDs.

It has been found that, surprisingly, this object is achieved by particular compounds described in detail hereinafter that are of good suitability for use in OLEDs. These OLEDs especially have a long lifetime, high efficiency and low operating voltage. The present invention therefore provides these compounds and electronic devices, especially organic electroluminescent devices, comprising such compounds.

WO 2011/137951 discloses cyclic phosphine oxide and sulfone derivatives in which the phosphorus or sulfur atom is bonded to a nitrogen atom that is part of a cyclic system, for example carbazole. WO 2012/121398 discloses phosphine oxide derivatives in which the phosphorus atom is bonded to a nitrogen atom, where the two further substituents on the phosphorus atom each form a ring with the two further substituents on the nitrogen atom. There is no disclosure of compounds according to the present invention.

The present invention provides a compound of formula (1)

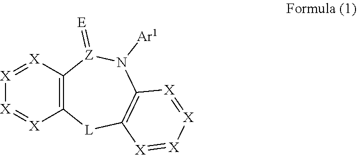

where the symbols used are as follows:
Z is the same or different at each instance and is PAr² or S(═O);
E is the same or different at each instance and is O or S when the symbol Z to which this E binds is PAr², and O when the symbol Z to which this E binds is S(═O);
L is selected from the group consisting of a single bond, NAr², O, S, S(═O)₂, P(═O)Ar², —X═X— or —C(═O)—NAr²—;
X is the same or different at each instance and is CR or N, where not more than two X groups per cycle are N; or two adjacent X are a group of the following formula (2), (3) or (4):

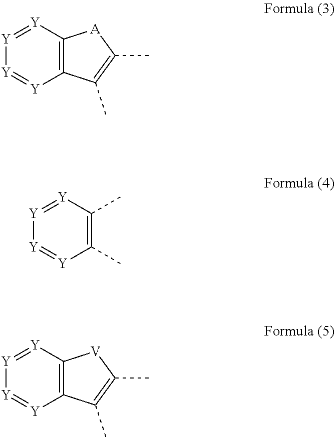

where the dotted bonds indicate the linkage of this group in the formula (1);
Y is the same or different at each instance and is CR or N, where not more than two Y groups per cycle are N;
Ar¹, Ar² is the same or different at each instance and is an aromatic or heteroaromatic ring system which has 5 to 40 aromatic ring atoms and may be substituted by one or more R radicals, where Ar¹ and Ar² are not joined to one another, with the proviso that the substituents R that bind to Ar¹ are not fluorine;
A is the same or different at each instance and is NAr², O, S or CR₂;
V is the same or different at each instance and is —NAr²—Z(=E)-;
R is the same or different at each instance and is H, D, F, Cl, Br, I, N(Ar³)₂, OAr³, SAr³, CN, NO₂, NAr³R¹, N(R¹)₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 40 aromatic ring atoms and may be substituted in each case by one or more R¹ radicals; at the same time, two R radicals together may also form an aliphatic or heteroaliphatic ring system;
Ar³ is the same or different at each instance and is an aromatic or heteroaromatic ring system which has 5 to 30 aromatic ring atoms and may be substituted by one or more R¹ radicals;
R¹ is the same or different at each instance and is H, D, F, C, Br, I, N(R²)₂, CN, NO₂, OR², SR², Si(R²)₃, B(OR²)₂, C(═O)R², P(═O)(R²)₂, S(═O)R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals, where one or more nonadjacent CH₂ groups may be replaced by Si(R²)₂, C═O, NR², O, S or CONR², or an aromatic or heteroaromatic ring system which has 5 to 40 aromatic ring atoms and may be substituted in each case by one or more R² radicals; at the same time, two or more R¹ radicals together may form an aliphatic ring system;
R² is the same or different at each instance and is H, D, F, CN or an aliphatic, aromatic or heteroaromatic organic radical, especially a hydrocarbyl radical, having 1 to 20 carbon atoms, in which one or more hydrogen atoms may also be replaced by F;

where the following compounds are excluded from the invention:

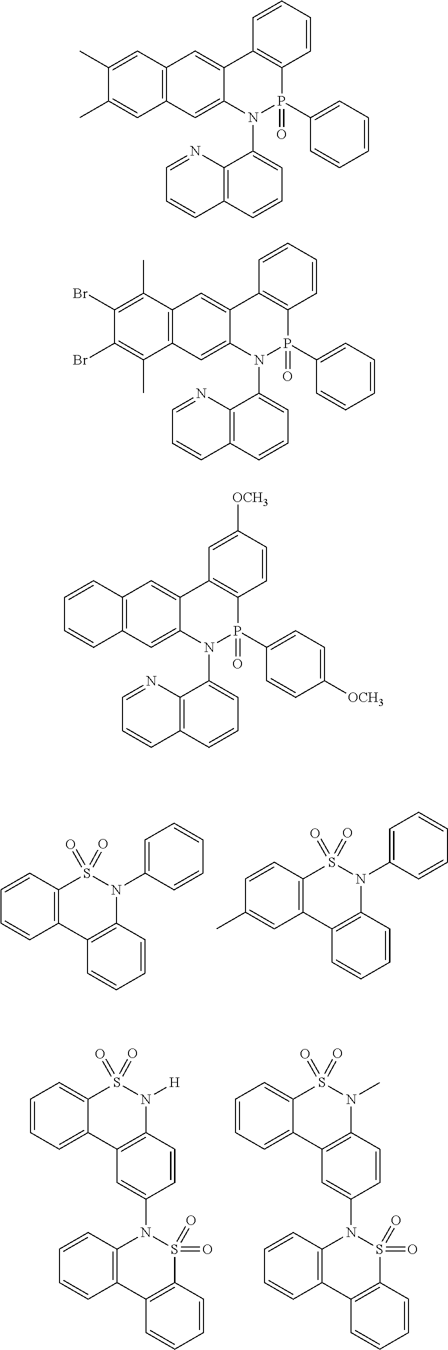

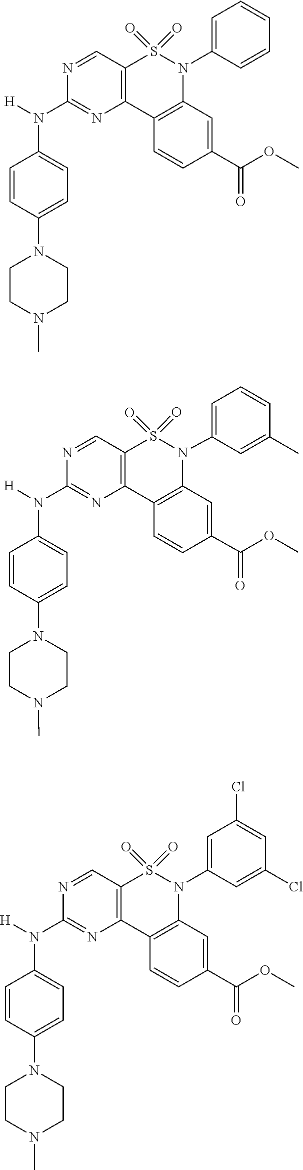 [SMILES: COC(=O)c1ccc2c(c1)N(c1ccccc1)S(=O)(=O)c1cnc(Nc3ccc(N4CCN(C)CC4)cc3)nc1-2 ; COC(=O)c1ccc2c(c1)N(c1cccc(C)c1)S(=O)(=O)c1cnc(Nc3ccc(N4CCN(C)CC4)cc3)nc1-2 ; COC(=O)c1ccc2c(c1)N(c1cc(Cl)cc(Cl)c1)S(=O)(=O)c1cnc(Nc3ccc(N4CCN(C)CC4)cc3)nc1-2]

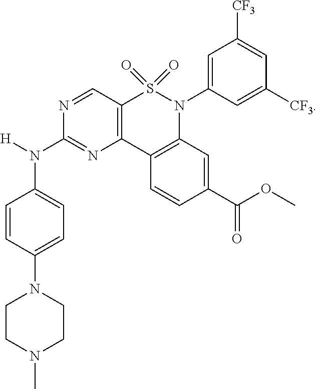 [SMILES: COC(=O)c1ccc2c(c1)N(c1cc(C(F)(F)F)cc(C(F)(F)F)c1)S(=O)(=O)c1cnc(Nc3ccc(N4CCN(C)CC4)cc3)nc1-2]

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. Here, an aryl group or heteroaryl group is understood to mean either a simple aromatic ring, i.e. benzene, or a simple heteroaromatic ring, for example pyridine, pyrimidine, thiophene, etc., or a condensed (fused) aryl or heteroaryl group, for example naphthalene, anthracene, phenanthrene, quinoline, isoquinoline, etc. Aromatic systems joined to one another by a single bond, for example biphenyl, by contrast, are not referred to as an aryl or heteroaryl group but as an aromatic ring system.

An aromatic ring system in the context of this invention contains 6 to 40 carbon atoms in the ring system. A heteroaromatic ring system in the context of this invention contains 2 to 40 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a nonaromatic unit, for example a carbon, nitrogen or oxygen atom. These shall likewise be understood to mean systems in which two or more aryl or heteroaryl groups are joined directly to one another, for example biphenyl, terphenyl, bipyridine or phenylpyridine. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferred aromatic or heteroaromatic ring systems are simple aryl or heteroaryl groups and groups in which two or more aryl or heteroaryl groups are joined directly to one another, for example biphenyl or bipyridine, and also fluorene or spirobifluorene.

In the context of the present invention, an aliphatic hydrocarbyl radical or an alkyl group or an alkenyl or alkynyl group which may contain 1 to 40 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic or heteroaromatic ring system which has 5-60 aromatic ring atoms and may also be substituted in each case by the abovementioned R² radicals or a hydrocarbyl radical and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from a combination of these systems.

The wording that two or more radicals together may form an aliphatic ring, in the context of the present description, should be understood to mean, inter alia, that the two radicals are joined to one another by a chemical bond with formal elimination of two hydrogen atoms. This is illustrated by the following scheme:

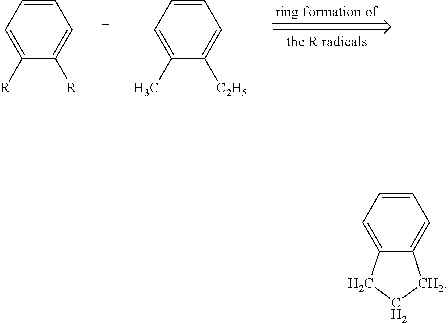

In addition, however, the abovementioned wording shall also be understood to mean that, if one of the two radicals is hydrogen, the second radical binds to the position to which the hydrogen atom was bonded, forming a ring. This shall be illustrated by the following scheme:

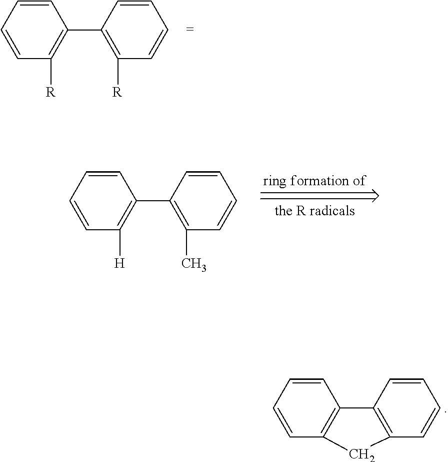

In one embodiment of the invention, the compound of the formula (1) does not contain any group of the formula (2), (3) or (4), such that the compound is one of the following formula (1-1):

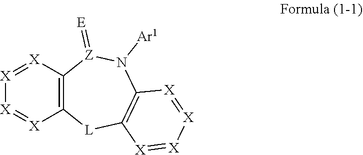

where X is the same or different at each instance and is CR or N, where not more than two X groups per cycle are N, and the further symbols have the definitions given above.

When the compound contains a group of the formula (2), (3) or (4), different isomers arise according to the position of the bond. For compounds of the formula (1) and the group of the formula (2), these are shown by way of example below by the formulae (5) to (16):

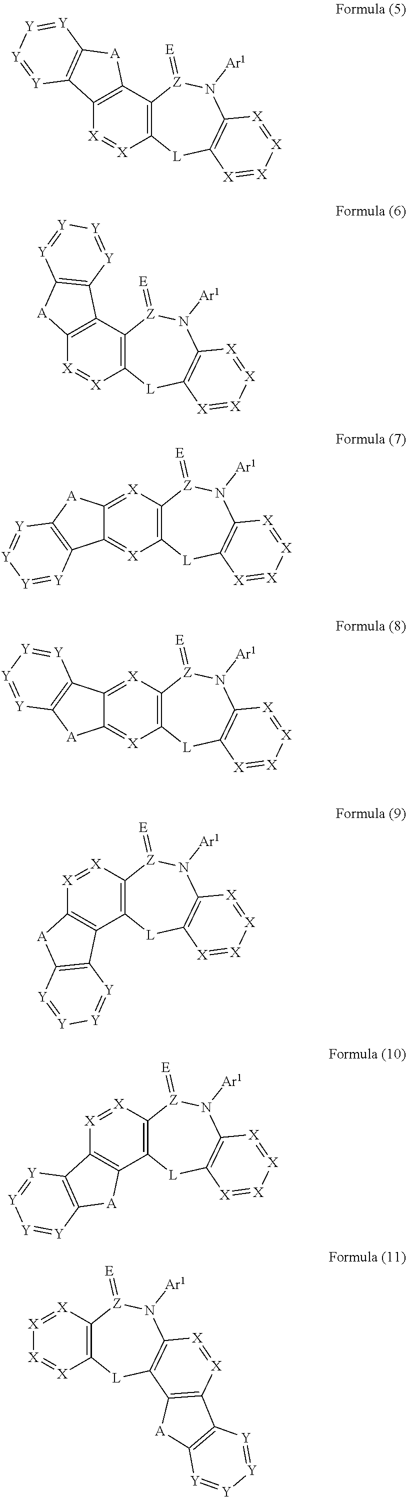

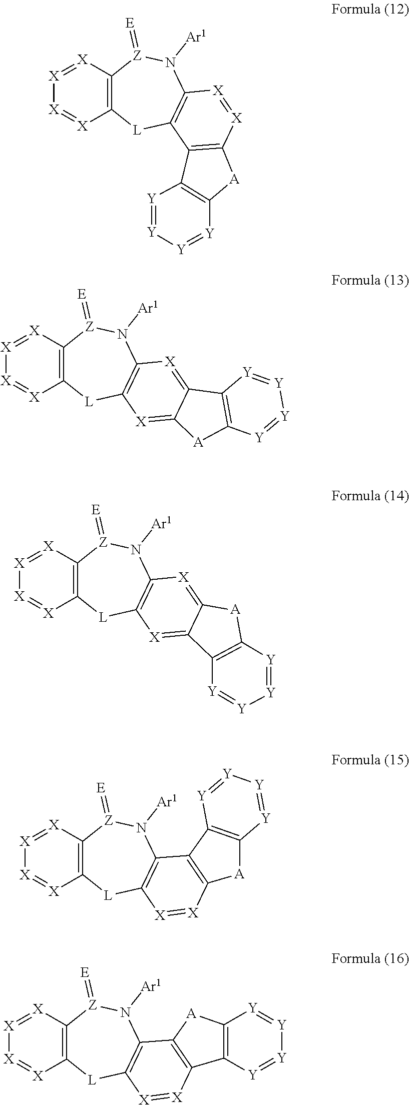

where X has the definitions given above and is preferably the same or different at each instance and is CR or N, where not more than two X groups per cycle are N, and the further symbols have the definitions given above.

Corresponding structures arise analogously for the fused-on groups of the formulae (3) and (4).

In a preferred embodiment of the invention, in the group of the formula (2), (3) or (4), not more than one symbol Y is N, and the other symbols Y are the same or different and are CR. More preferably, all symbols Y in the formulae (2), (3) and (4) are CR, such that the group is preferably respectively one of the formulae (2a), (3a) and (4a):

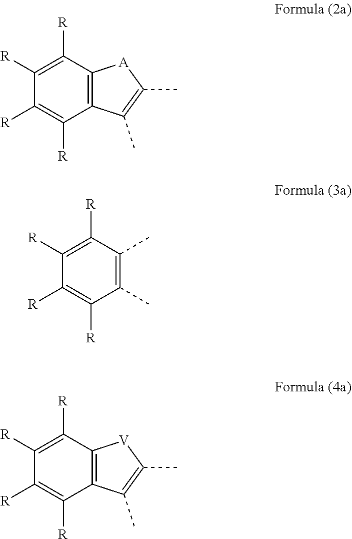

where the symbols used have the definitions given above.

In a preferred embodiment of the formula (2) or (2a) or of the formulae (5) to (16) and the formulae that follow, A is the same or different at each instance and is NAr².

In a preferred embodiment of the invention, the compound of the formula (1) contains no, one or two groups of the formula (2), (3) or (4) in total, with preferably not more than one group of the formula (2), (3) or (4) attached per cycle. More preferably, the compound of the formula (1) contains no or one group of the formula (2), (3) or (4). The remaining symbols X that are not a group of the formula (2), (3) or (4) are the same or different at each instance and are CR or N. Most preferably, the compound of the formula (1) does not contain any group of the formula (2), (3) or (4), and the symbols X are the same or different and are CR or N.

In a further preferred embodiment of the invention, not more than one symbol X per cycle in formula (1) is N, and more preferably not more than one symbol X in total is N. Most preferably, the symbols X that are not a group of the formula (2), (3) or (4) are the same or different at each instance and are CR. In a particularly preferred embodiment of the invention, the symbols Y are the same or different at each instance and are CR, such that the group is preferably one of the formula (2a), (3a) or (4a), and the symbols X that are not a group of the formula (2a), (3a) or (4a) are the same or different at each instance and are CR.

Preferred embodiments of the formula (1) are thus the structures of the following formulae (1a) and (5a) to (16a):

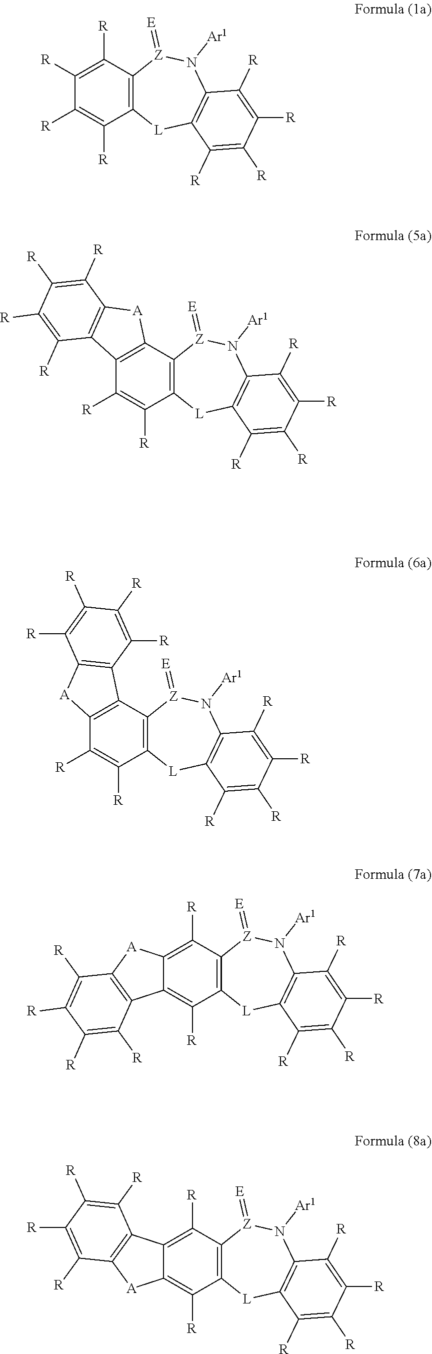

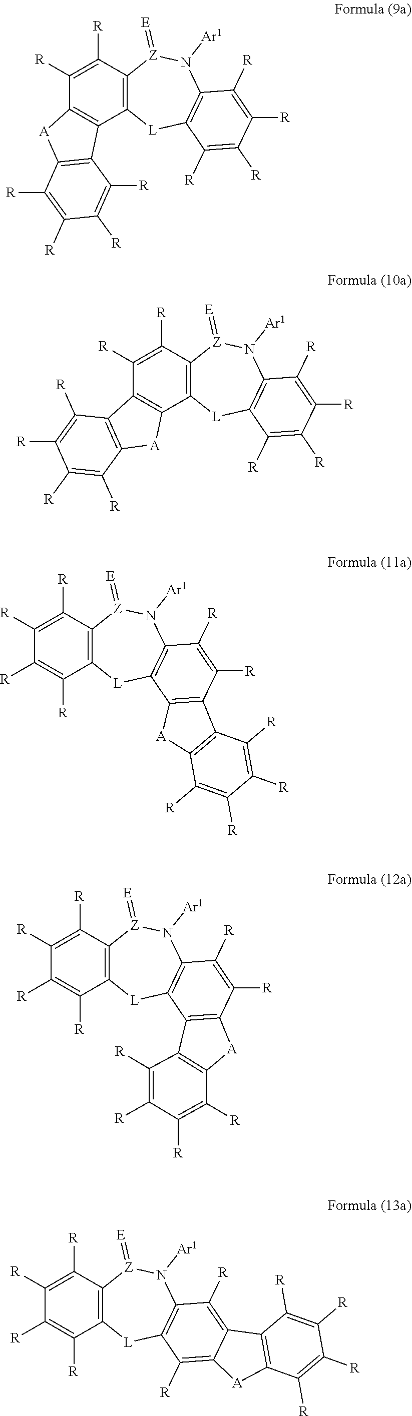

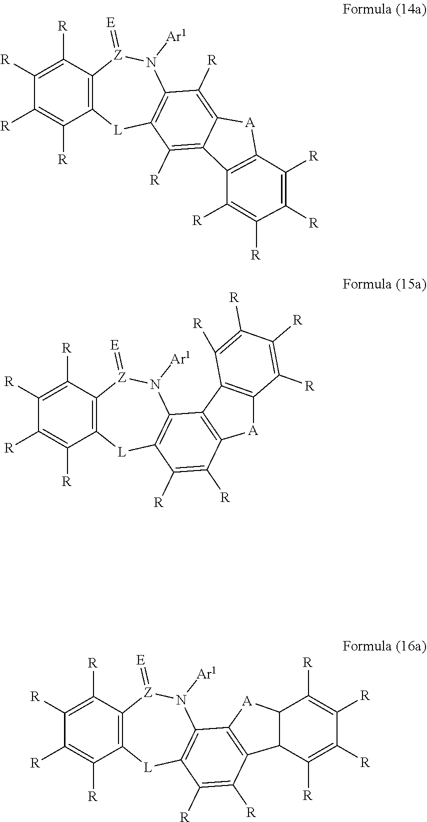

where the symbols used have the definitions given above. A here is preferably NAr².

In formula (1) and the preferred embodiments of the invention detailed above, E is preferably 0.

In one embodiment of the invention, L is a single bond, an R radical bonded adjacent to L is P(═O)R¹ or S(═O)₂R¹, and the other R radical bonded adjacent to L is N(Ar³)R¹, where these two R radicals together form a ring. This gives rise to the structures of the following formulae (17) and (18), and the preferred embodiments (17a) and (18a):

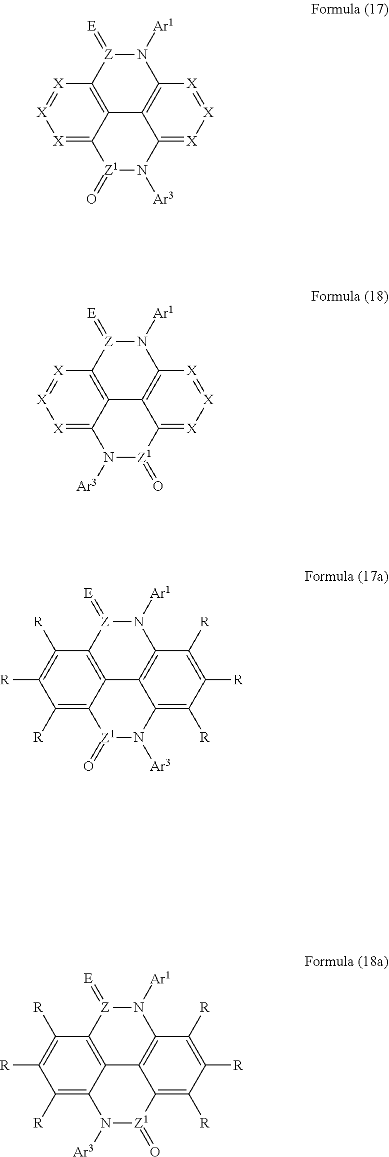

where the symbols used have the definitions given above and Z¹ is PR¹ or S(═O). E here is preferably O, and the R¹ radical that binds to Z¹ is preferably an aromatic or heteroaromatic ring system which has 6 to 30 aromatic ring atoms, preferably 6 to 13 aromatic ring atoms, and may be substituted by one or more R² radicals.

In a preferred embodiment of the invention, not more than three R radicals in total, more preferably not more than two R radicals and most preferably not more than one R radical in the compound of the formula (1) or in the preferred structures detailed above are/is a group other than hydrogen.

Particular preference is given to the structures of the following formulae (1 b) or (5b) to (16b):

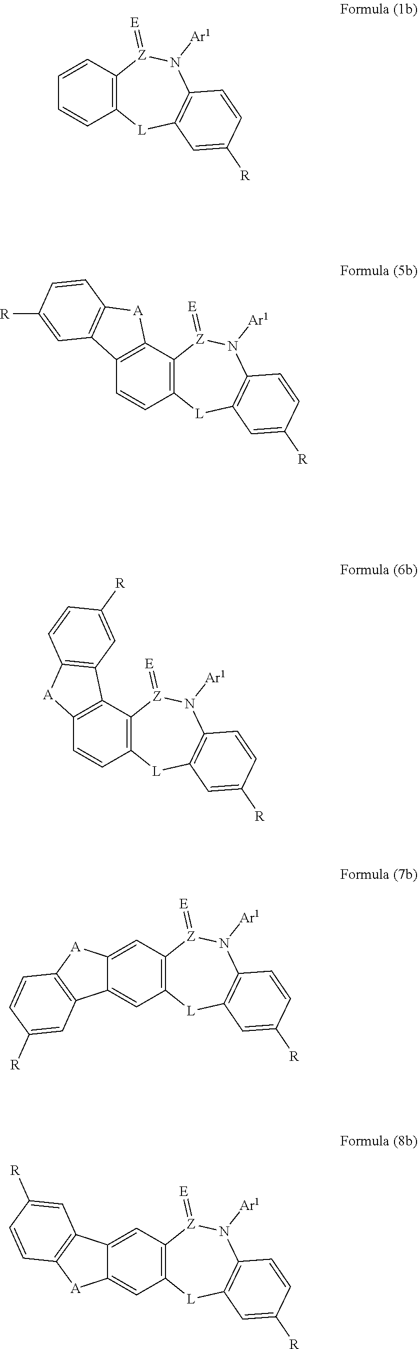

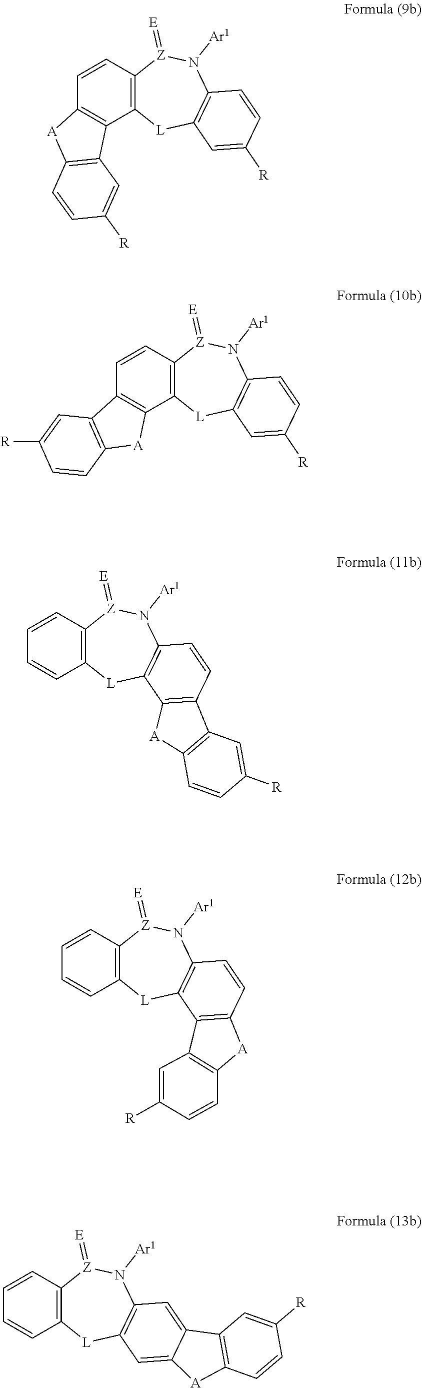

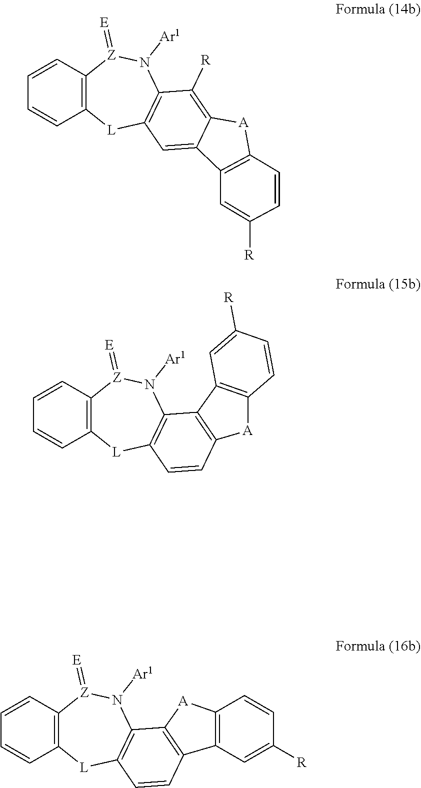

where the symbols used have the definitions given above.

In a preferred embodiment of the invention, Z is the same or different at each instance and is PAr².

In a further preferred embodiment of the invention, E is O.

In a further preferred embodiment of the invention, L is a single bond, NAr², O, S, S(═O)₂ or —CR═CR—, more preferably a single bond, NAr² or O and most preferably a single bond.

In a particularly preferred embodiment of the invention, Z is PAr², and E is O, and L is a single bond. Particular preference is thus given to the compounds of the following formulae (1c) and (5c) to (16c):

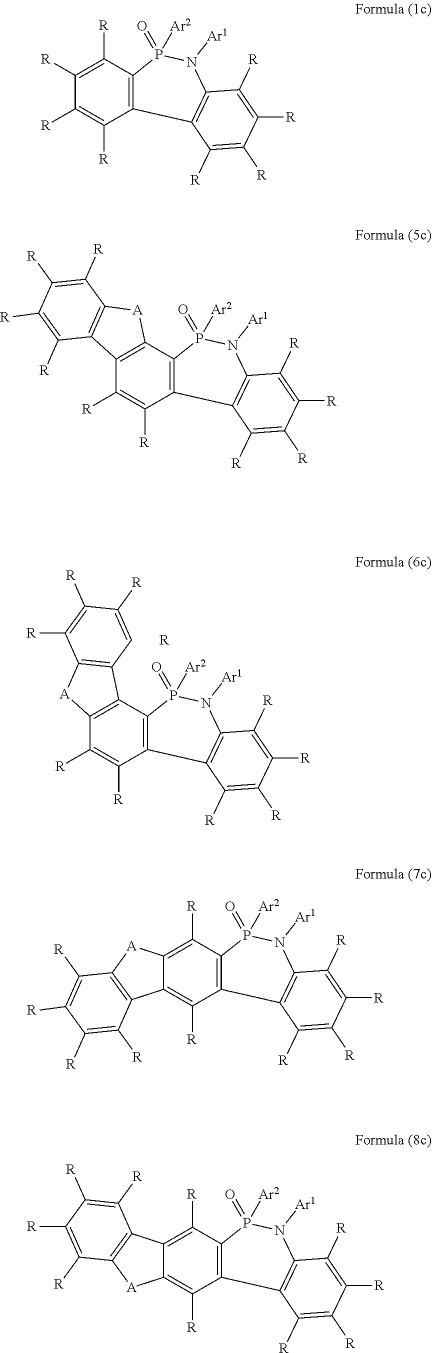

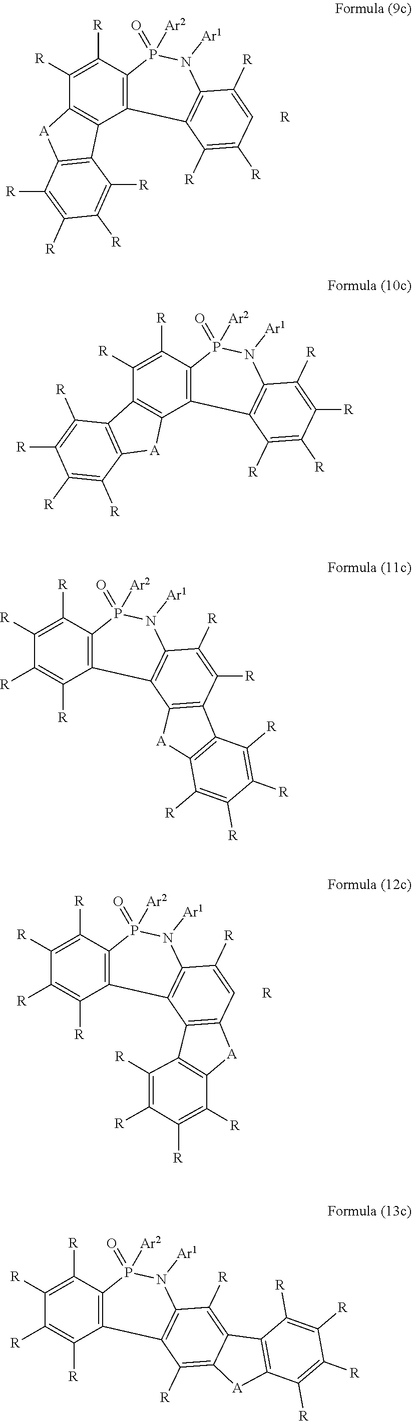

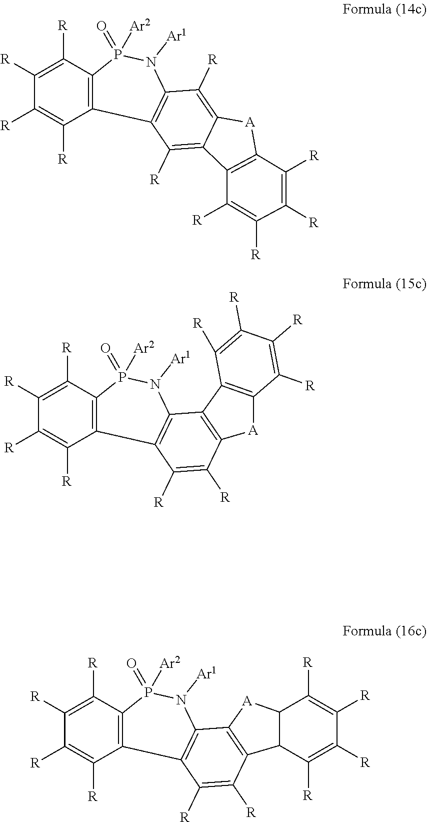

where the symbols used have the definitions given above. A here is preferably NAr². The R radicals are preferably bonded in the positions analogously to the structures of the formulae (1 b) and (5b) to (16b).

There follows a description of preferred substituents Ar¹, Ar², R, Ar³, R¹ and R². In a particularly preferred embodiment of the invention, the preferences specified above for X, Y, Z, E and L and the preferences specified hereinafter for Ar¹, Ar², R, Ar³, R¹ and R² occur simultaneously and are applicable to the structures of the formula (1) and to all preferred embodiments detailed above.

In a preferred embodiment of the invention, Ar¹ and Ar² are the same or different at each instance and are an aromatic or heteroaromatic ring system which has 6 to 30 aromatic ring atoms and may be substituted by one or more R radicals, where the R radicals in the case of Ar¹ are not fluorine. More preferably, Ar¹ and Ar² are the same or different at each instance and are an aromatic or heteroaromatic ring system which has 6 to 24 aromatic ring atoms, especially 6 to 12 aromatic ring atoms, and may be substituted by one or more, preferably nonaromatic R radicals, where the R radicals in the case of Ar¹ are not fluorine. When Ar is a heteroaryl group, especially triazine, pyrimidine, quinazoline or carbazole, preference may also be given to aromatic or heteroaromatic substituents R on this heteroaryl group. It may further be preferable when Ar¹ or Ar² is substituted by an N(Ar³)₂ group, such that the substituent Ar¹ or Ar² constitutes a triarylamine or triheteroarylamine group overall.

Suitable aromatic or heteroaromatic ring systems Ar¹ or Ar² are the same or different at each instance and are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R radicals, preferably nonaromatic R radicals.

When Ar is a heteroaryl group, especially triazine, pyrimidine, quinazoline or carbazole, preference may also be given to aromatic or heteroaromatic R radicals on this heteroaryl group.

Ar¹ and Ar² here are preferably the same or different at each instance and are selected from the groups of the following formulae Ar-1 to Ar-76:

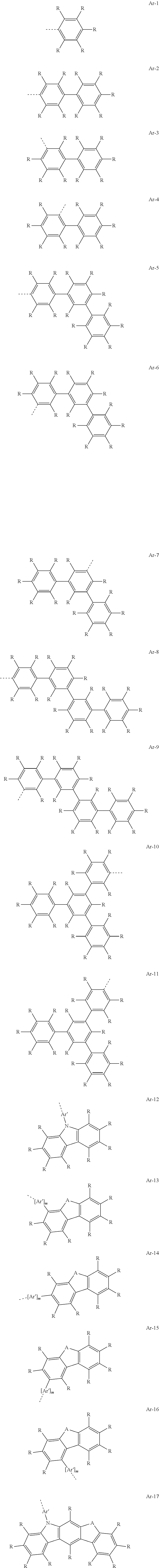

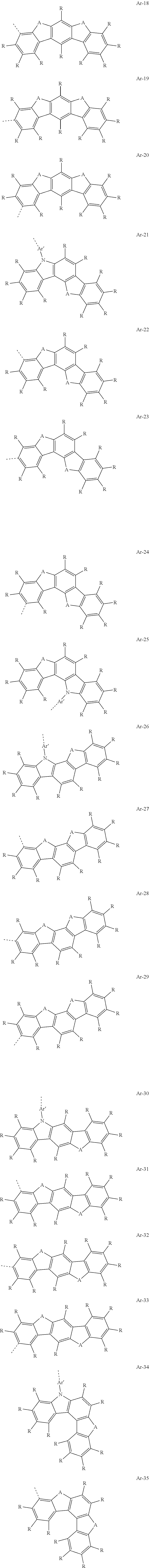

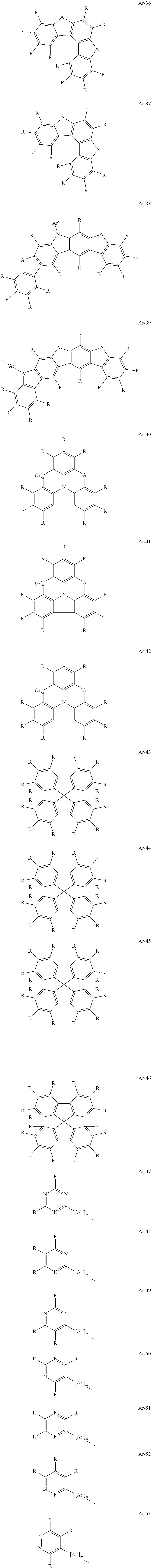

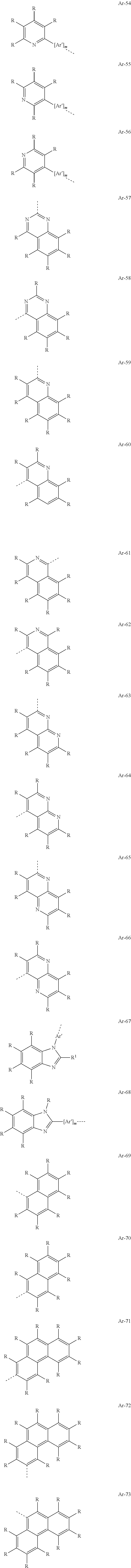

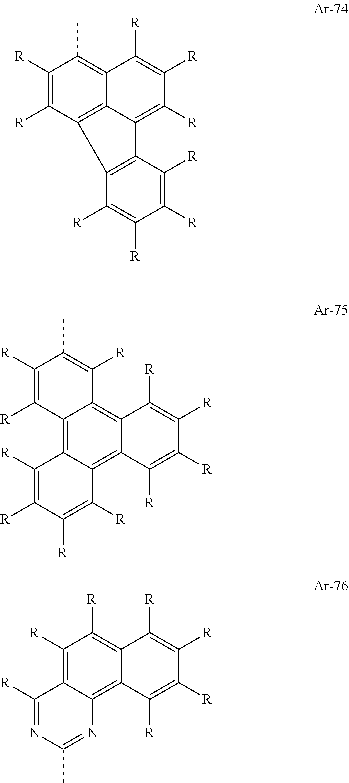

where R has the definitions given above and is not fluorine in the case of substituents on Ar¹, the dotted bond in the case of Ar¹ represents the bond to the nitrogen atom and in the case of Ar² the bond to the phosphorus or nitrogen atom, and in addition:
Ar′ is the same or different at each instance and is a divalent aromatic or heteroaromatic ring system which has 6 to 18 aromatic ring atoms, preferably 6 to 13 aromatic ring atoms, and may be substituted in each case by one or more R radicals;
A is the same or different at each instance and is CR₂, NR, O or S;
n is 0 or 1, where n=0 means that no A group is bonded at this position and R radicals are bonded to the corresponding carbon atoms instead;
m is 0 or 1, where m=0 means that the Ar′ group is absent and that the corresponding aromatic or heteroaromatic group is bonded directly to the nitrogen atom or the phosphorus atom.

In a preferred embodiment of the invention, R is the same or different at each instance and is selected from the group consisting of H, D, F, N(Ar³)₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic ring system. More preferably, R is the same or different at each instance and is selected from the group consisting of H, N(Ar³)₂, a straight-chain alkyl group having 1 to 6 carbon atoms, especially having 1, 2, 3 or 4 carbon atoms, or a branched or cyclic alkyl group having 3 to 6 carbon atoms, where the alkyl group in each case may be substituted by one or more R¹ radicals, but is preferably unsubstituted, or an aromatic or heteroaromatic ring system which has 6 to 24 aromatic ring atoms and may be substituted in each case by one or more R¹ radicals, preferably nonaromatic R¹ radicals. Most preferably, R is the same or different at each instance and is selected from the group consisting of H or an aromatic or heteroaromatic ring system which has 6 to 24 aromatic ring atoms, more preferably 6 to 13 aromatic ring atoms, and may be substituted in each case by one or more R¹ radicals, preferably nonaromatic R¹ radicals. It may additionally be preferable when R is a triaryl- or -heteroarylamine group which may be substituted by one or more R¹ radicals. This group is one embodiment of an aromatic or heteroaromatic ring system, in which case two or more aryl or heteroaryl groups are joined to one another by a nitrogen atom. When R is a triaryl- or -heteroarylamine group, this group preferably has 18 to 30 aromatic ring atoms and may be substituted by one or more R¹ radicals, preferably nonaromatic R¹ radicals.

In a further preferred embodiment of the invention, Ar³ is an aromatic or heteroaromatic ring system which has 6 to 24 aromatic ring atoms, more preferably 6 to 13 aromatic ring atoms, and may be substituted by one or more, preferably nonaromatic R¹ radicals.

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R¹ radicals together may form an aliphatic ring system. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms, preferably 6 to 13 aromatic ring atoms, and may be substituted in each case by one or more R² radicals, but is preferably unsubstituted.

In a further preferred embodiment of the invention, R² is the same or different at each instance and is H, F, an alkyl group having 1 to 4 carbon atoms or an aryl group having 6 to 10 carbon atoms, which may be substituted by an alkyl group having 1 to 4 carbon atoms, but is preferably unsubstituted.

Suitable aromatic or heteroaromatic ring systems R or Ar³ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals. When R or Ar³ is a heteroaryl group, especially triazine, pyrimidine, quinazoline or carbazole, preference may also be given to aromatic or heteroaromatic R¹ radicals on this heteroaryl group.

The R groups here, when they are an aromatic or heteroaromatic ring system, or Ar³ are preferably selected from the groups of the following formulae R-1 to R-76:

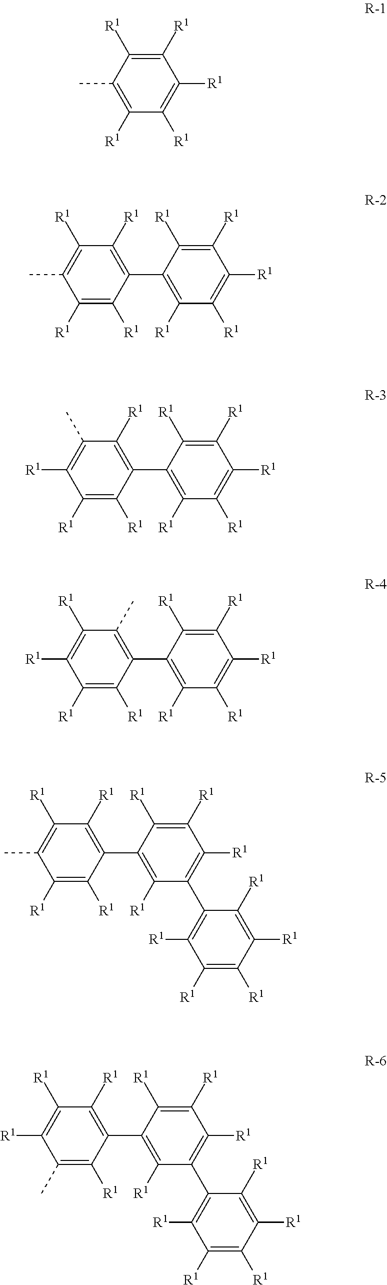

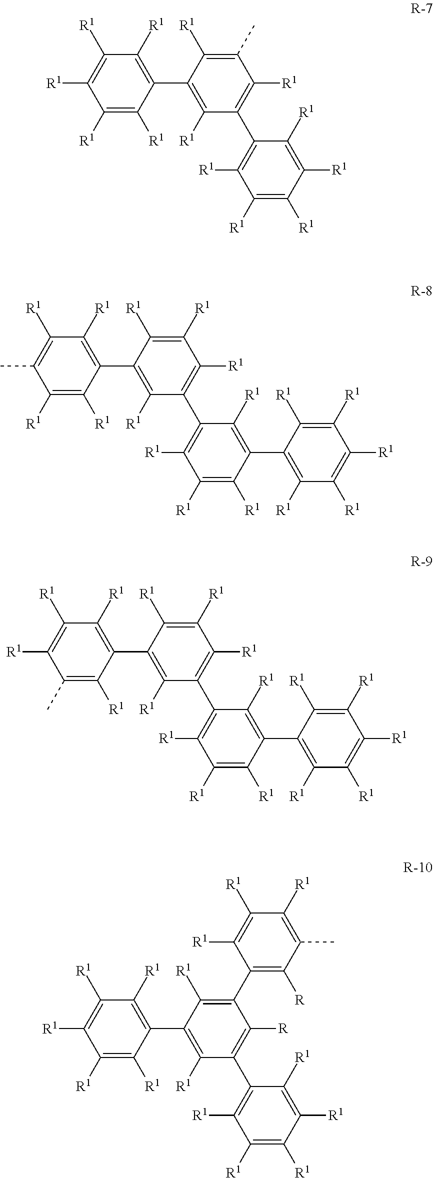

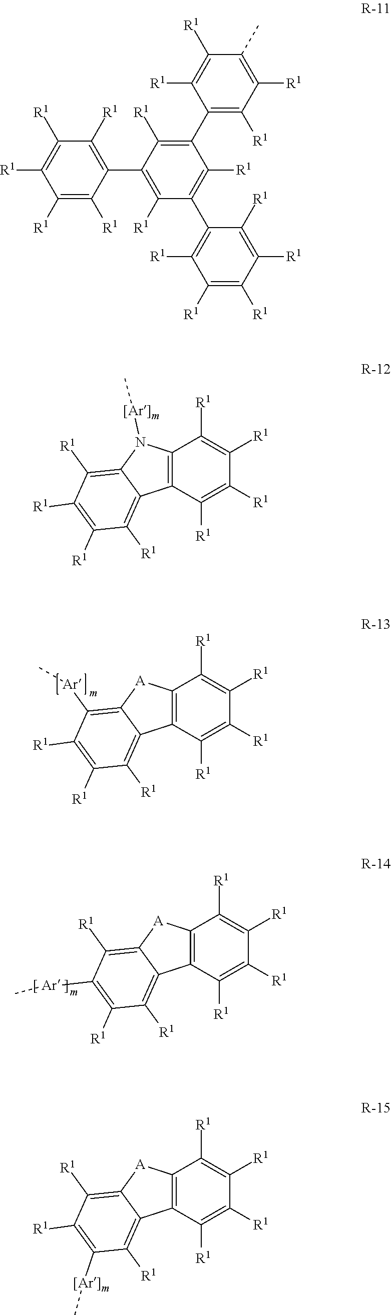

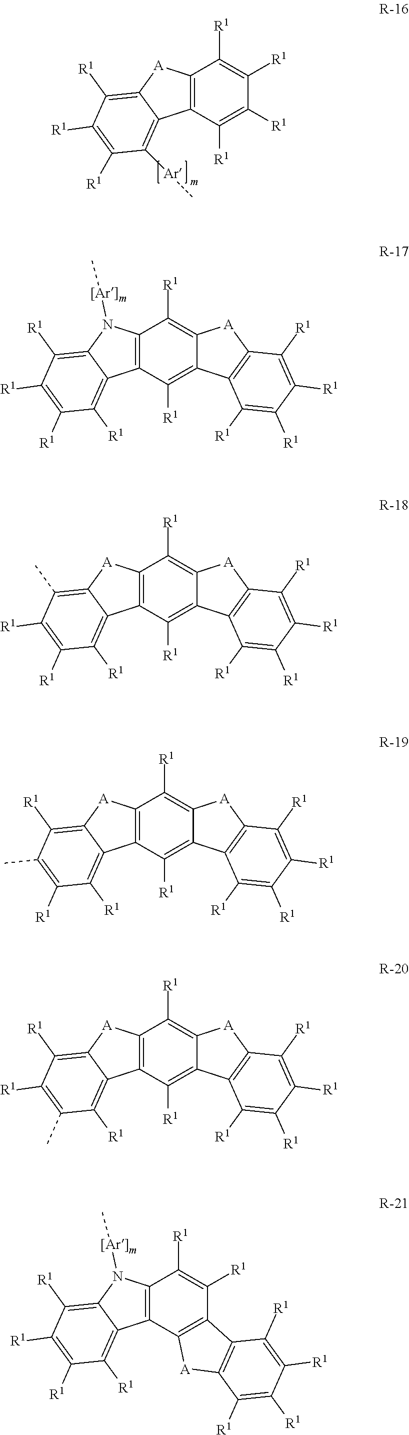

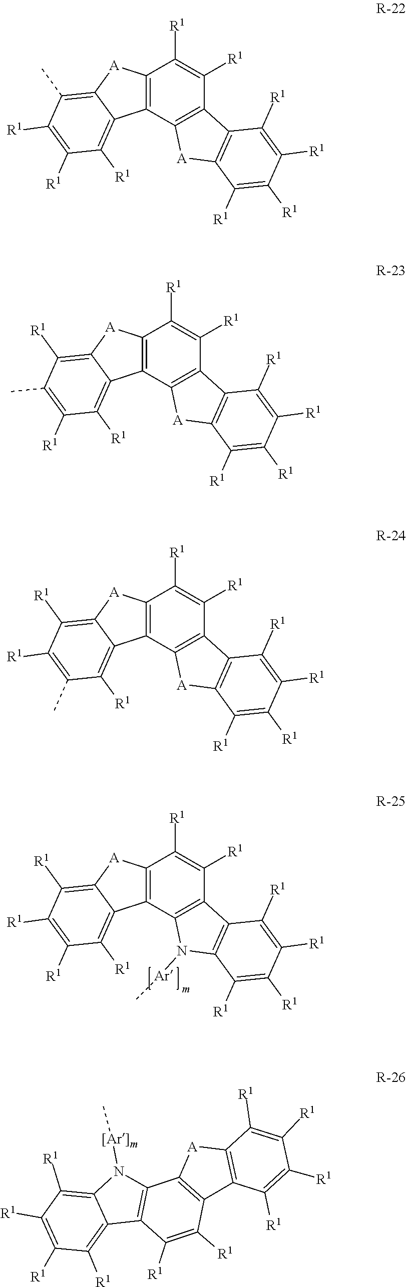

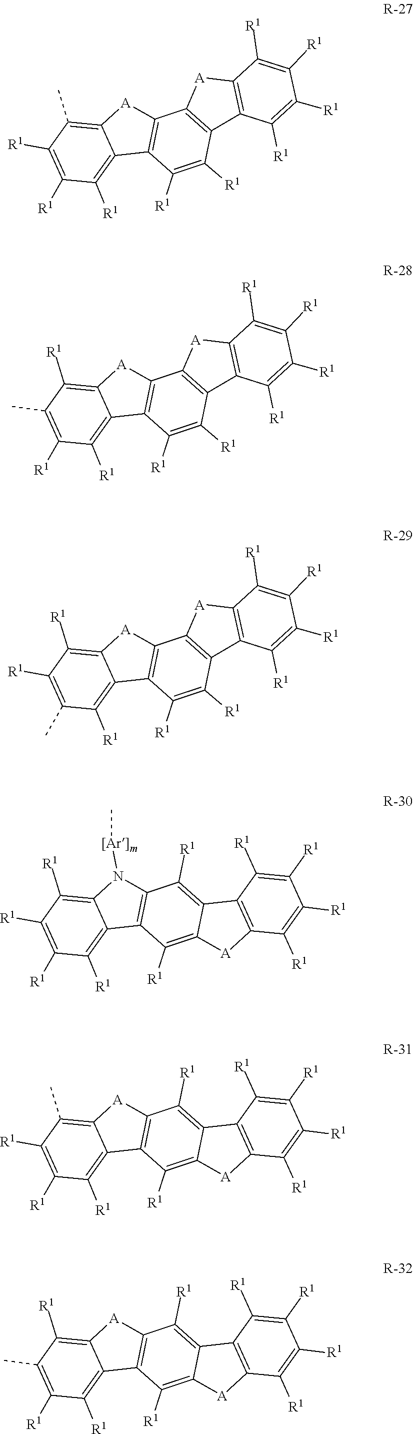

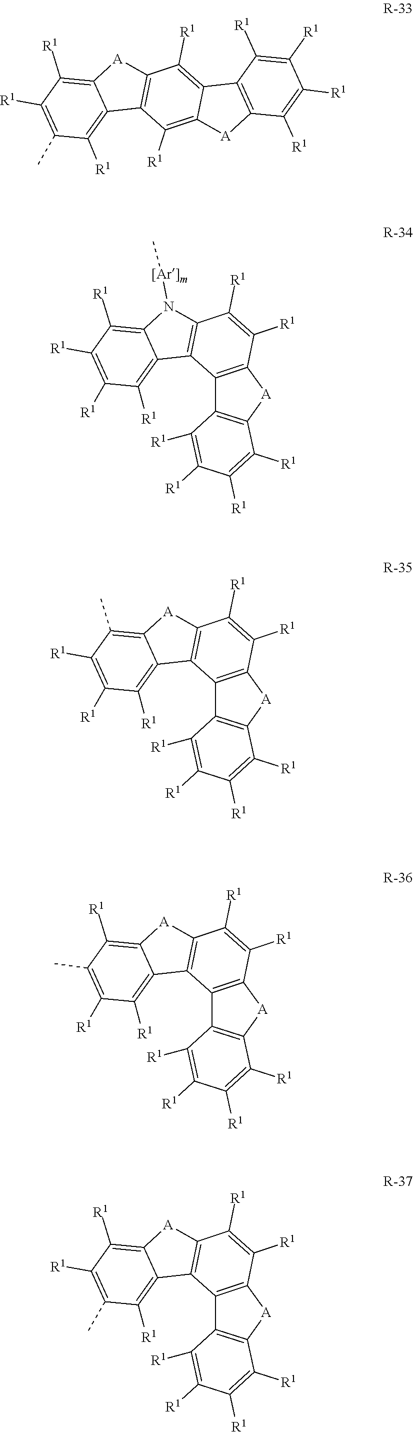

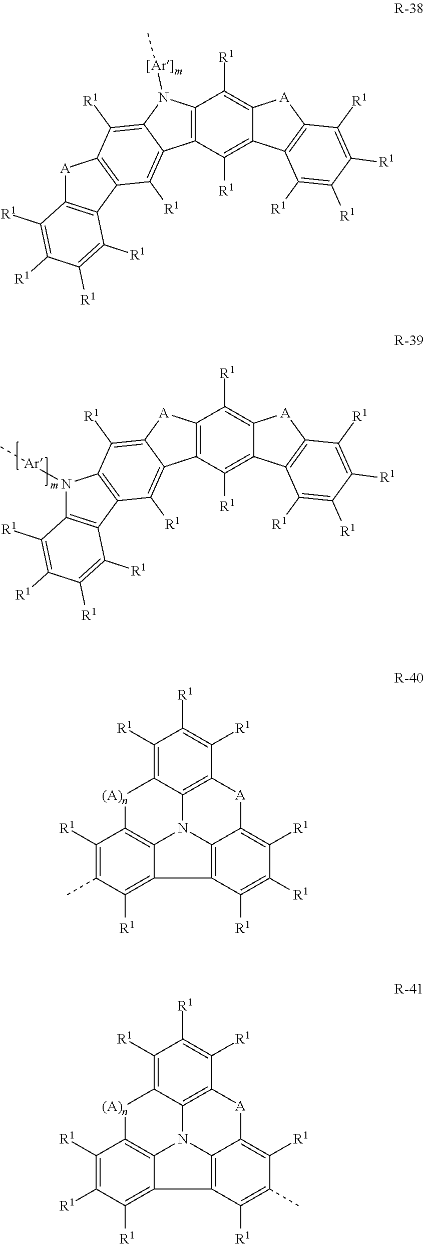

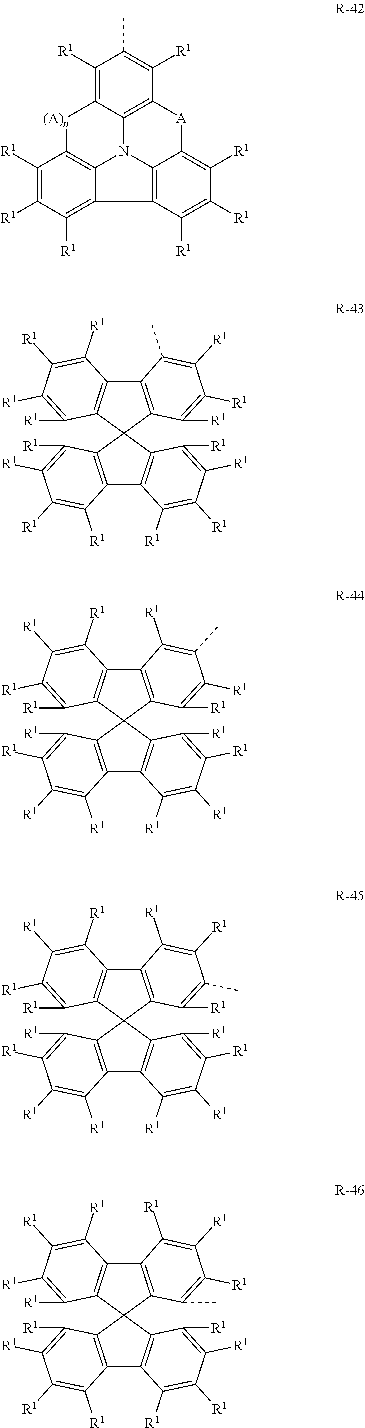

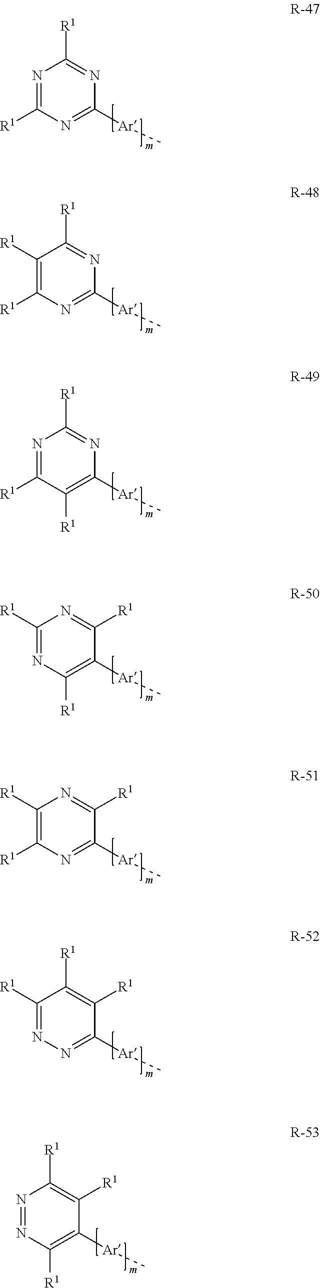

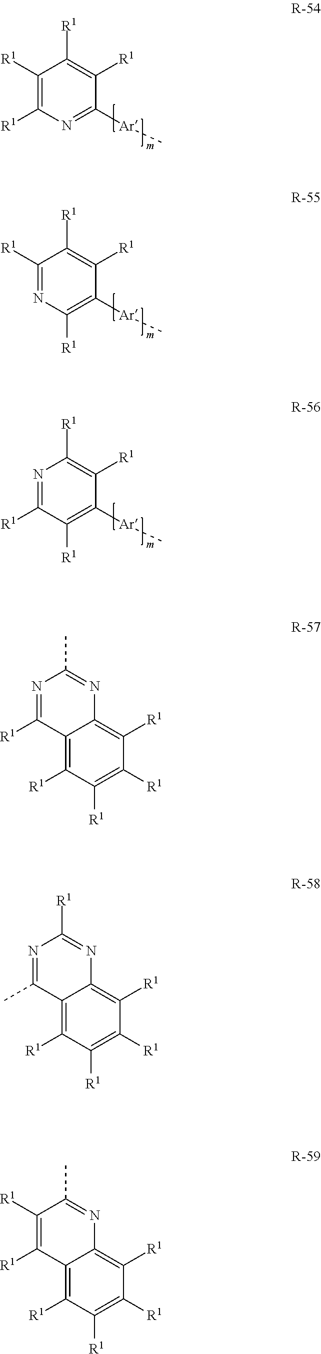

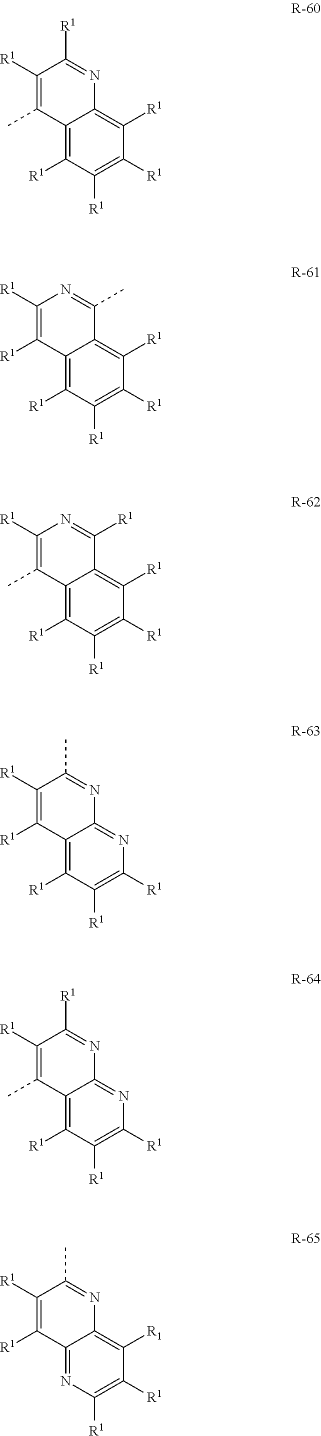

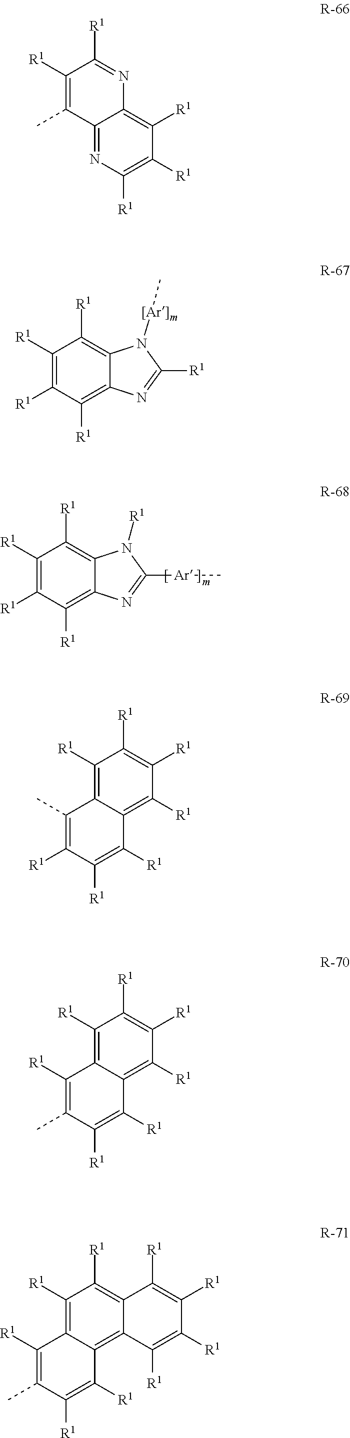

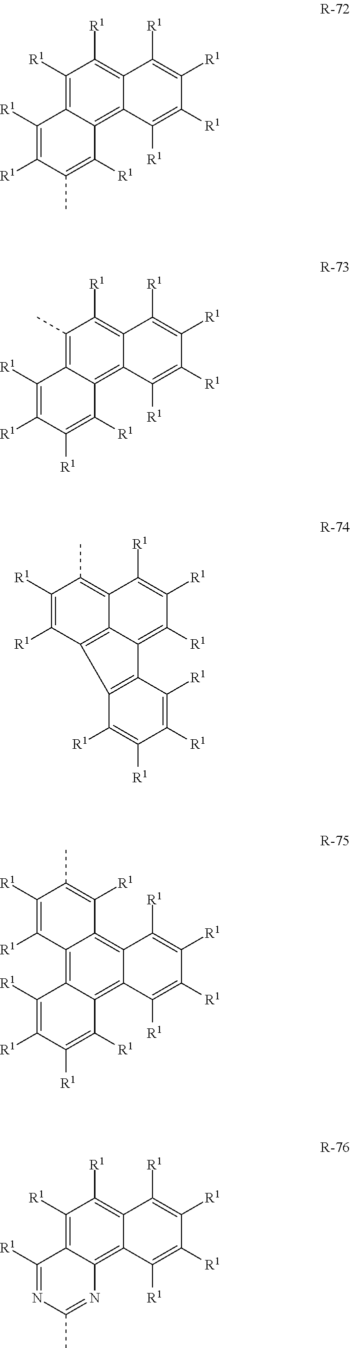

where R¹ is as defined above, the dotted bond represents the linkage of this group, and in addition:
Ar′ is the same or different at each instance and is a bivalent aromatic or heteroaromatic ring system which has 6 to 18 aromatic ring atoms and may be substituted in each case by one or more R¹ radicals;
A is the same or different at each instance and is C(R¹)₂, NR¹, 0 or S;
n is 0 or 1, where n=0 means that no A group is bonded at this position and R¹ radicals are bonded to the corresponding carbon atoms instead;
m is 0 or 1, where m=0 means that the Ar³ group is absent and that the corresponding aromatic or heteroaromatic group is bonded directly to a carbon atom of the base skeleton in formula (1) or in the preferred embodiments, or to Ar¹ or Ar², or to the nitrogen atom in the N(Ar³)₂ group; with the proviso that m=1 for the structures (R-12), (R-17), (R-21), (R-25), (R-26), (R-30), (R-34), (R-38) and (R-39) when these groups are embodiments of Ar³.

When the abovementioned Ar-1 to Ar-76 groups in the case of Ar¹ or Ar² or R-1 to R-76 groups in the case of R or Ar³ have two or more A groups, possible options for these include all combinations from the definition of A. Preferred embodiments in that case are those in which one A group is NR or NR¹ and the other A group is C(R)₂ or C(R¹)₂ or in which both A groups are NR or NR¹ or in which both A groups are O. In a particularly preferred embodiment of the invention, in Ar¹, Ar², R or Ar³ groups having two or more A groups, at least one A group is C(R)₂ or C(R¹)₂ or is NR or NR¹.

When A in formula (2) or formula (2a) or in the formulae Ar-1 to Ar-76 or in the formulae R-1 to R-76 is NR or NR¹, the substituent R or R¹ bonded to the nitrogen atom is preferably an aromatic or heteroaromatic ring system which has 5 to 24 aromatic ring atoms and may also be substituted by one or more R¹ or R² radicals. In a particularly preferred embodiment, this R or R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R¹ or R² radicals. Particular preference is given to phenyl, biphenyl, terphenyl and quaterphenyl having bonding patterns as listed above for Ar-1 to Ar-11 or R-1 to R-11, where these structures may be substituted by one or more R¹ or R² radicals, but are preferably unsubstituted.

When A in formula (2) or formula (2a) or in the formulae Ar-1 to Ar-76 or in the formulae R-1 to R-76 is C(R)₂ or C(R¹)₂, the substituents R or R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system which has 5 to 24 aromatic ring atoms and may also be substituted by one or more R¹ or R² radicals. Most preferably, R or R¹ is a methyl group or a phenyl group. In this case, the R or R¹ radicals together may also form a ring system, which leads to a spiro system.

Further suitable Ar¹, Ar², R or Ar′ groups are groups of the formula —Ar⁴—N(Ar⁵)(Ar⁶) where Ar⁴, Ar⁵ and Ar⁶ are the same or different at each instance and are an aromatic or heteroaromatic ring system which has 5 to 24 aromatic ring atoms and may be substituted in each case by one or more R¹ radicals. Ar¹ or Ar² results in such a group when the Ar¹ or Ar² group is substituted by an N(Ar³)₂ group. The total number of aromatic ring atoms in Ar⁴, Ar⁵ and Ar⁶ here is not more than 60 and preferably not more than 40.

In this case, Ar⁴ and Ar⁵ may also be bonded to one another and/or Ar⁵ and Ar⁶ to one another via a group selected from C(R¹)₂, NR¹, O or S. Preferably, Ar⁴ and Ar⁵ are joined to one another and Ar⁵ and Ar⁶ to one another in the respective ortho position to the bond to the nitrogen atom. In a further embodiment of the invention, none of the Ar⁴, Ar⁵ and Ar⁶ groups are bonded to one another.

Preferably, Ar⁴ is an aromatic or heteroaromatic ring system which has 6 to 24 aromatic ring atoms, especially 6 to 12 aromatic ring atoms, and may be substituted in each case by one or more R¹ radicals. More preferably, Ar⁴ is selected from the group consisting of ortho-, meta- or para-phenylene or ortho-, meta- or para-biphenyl, each of which may be substituted by one or more R¹ radicals, but are preferably unsubstituted. Most preferably, Ar⁴ is an unsubstituted phenylene group.

Preferably, Ar⁵ and Ar⁶ are the same or different at each instance and are an aromatic or heteroaromatic ring system which has 6 to 24 aromatic ring atoms and may be substituted in each case by one or more R¹ radicals. Particularly preferred Ar⁵ and Ar⁶ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene, triphenylene or combinations of two, three or four of these groups, each of which may be substituted by one or more R¹ radicals. More preferably, Ar⁵ and Ar⁶ are the same or different at each instance and are an aromatic ring system which has 6 to 24 aromatic ring atoms and may be substituted by one or more R¹ radicals, especially selected from the groups consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

At the same time, the alkyl groups in compounds that are processed by vacuum evaporation preferably have not more than five carbon atoms, more preferably not more than 4 carbon atoms, most preferably not more than 1 carbon atom. For compounds which are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is preferable when the compound does not contain any fused aryl or heteroaryl groups in which more than two six-membered rings are fused directly to one another. An exception to this is formed by phenanthrene and triphenylene which, because of their high triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of suitable compounds according to the above-detailed embodiments are the compounds listed below.

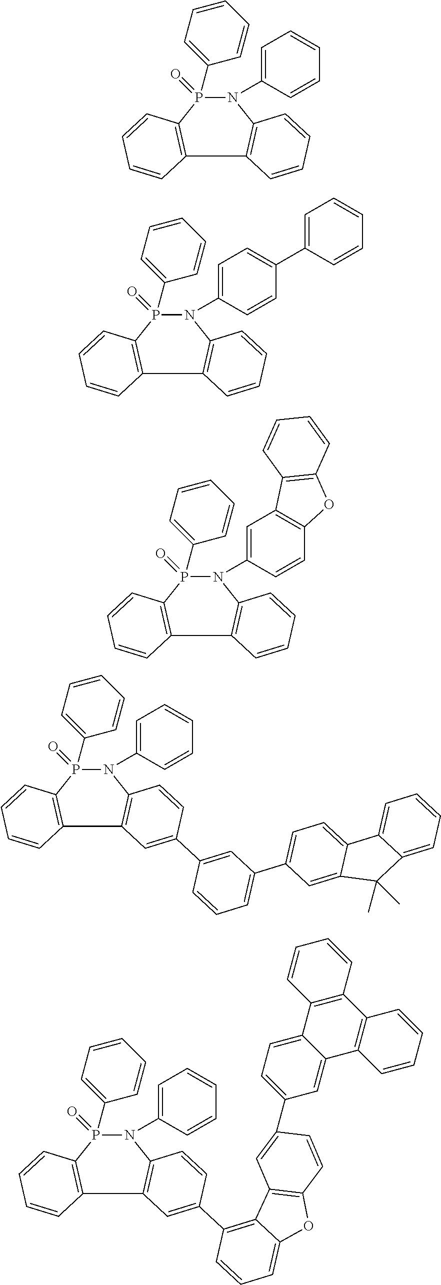

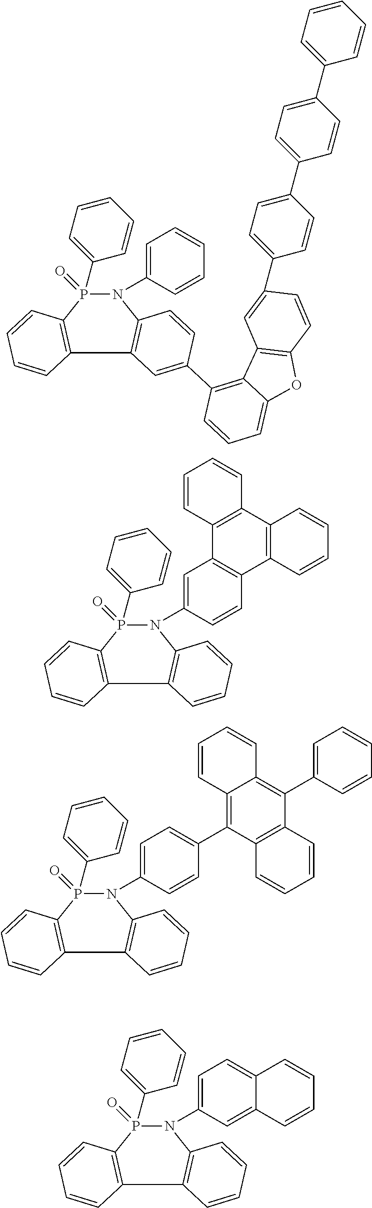

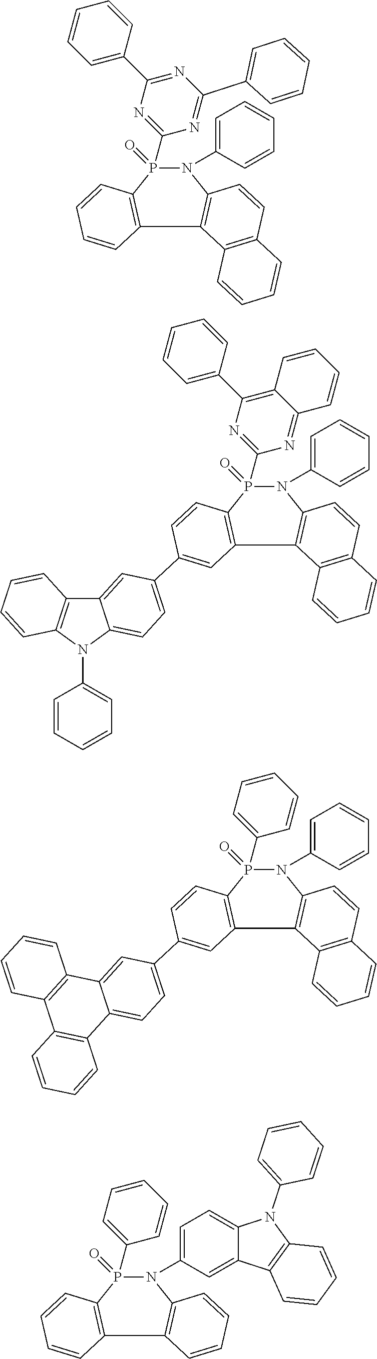

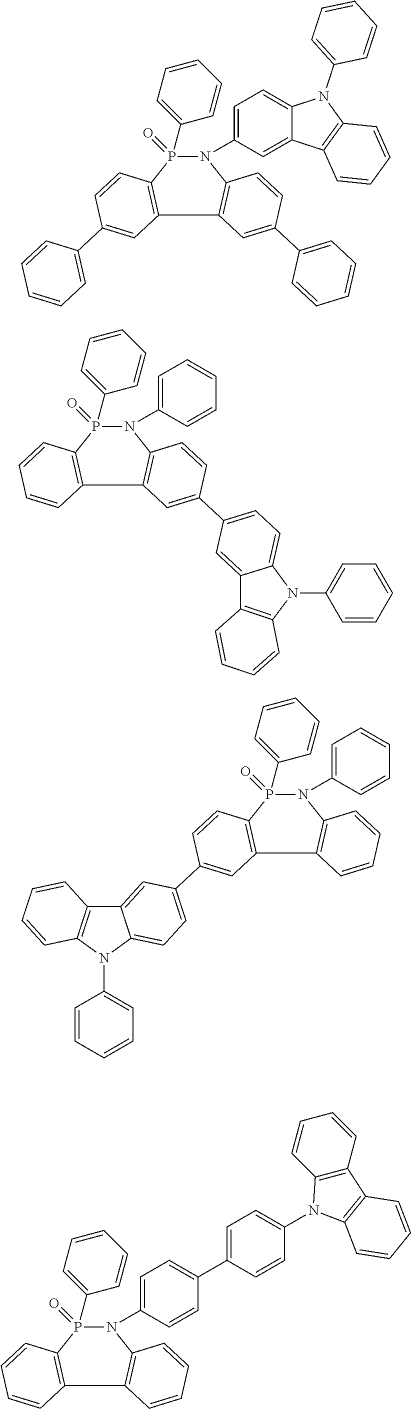

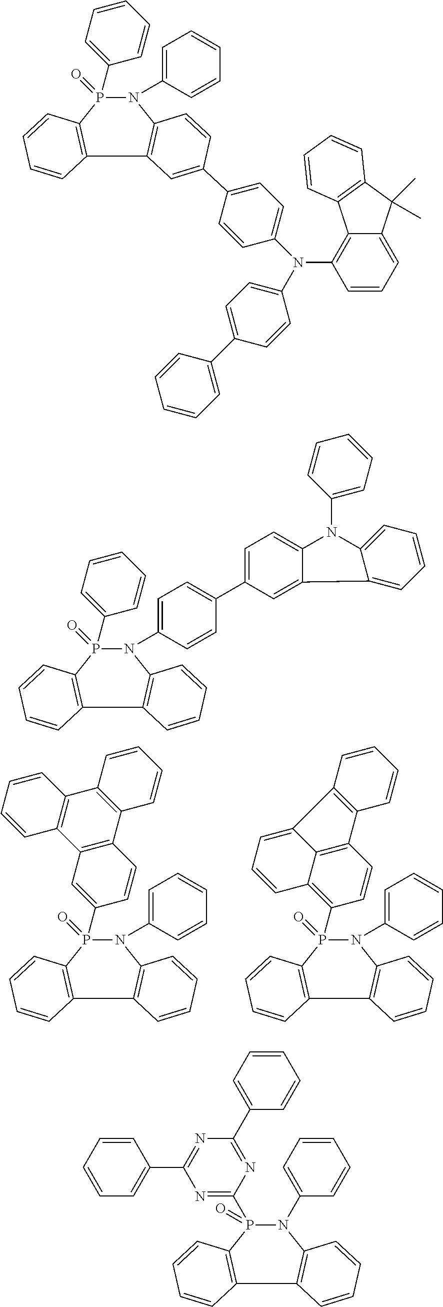

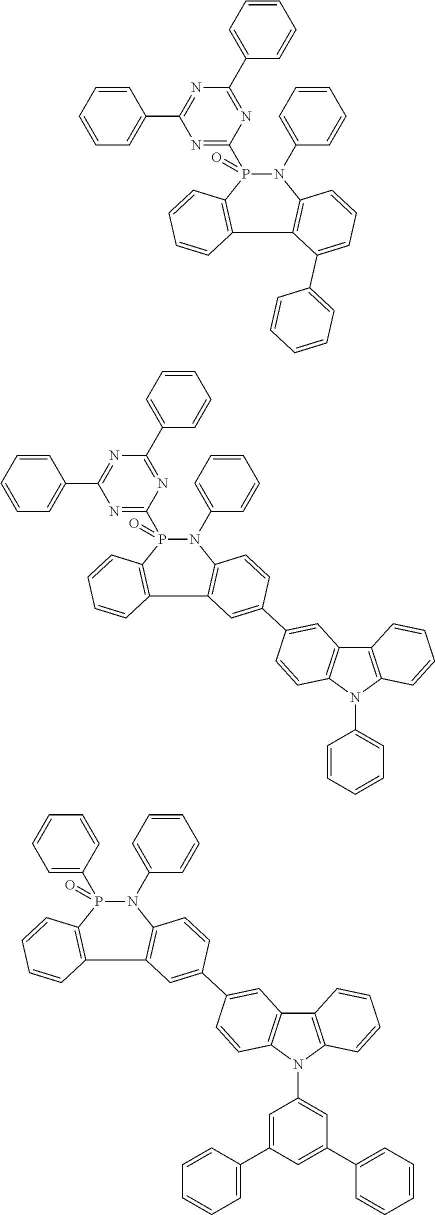 [SMILES: O=P1(c2nc(-c3ccccc3)nc(-c3ccccc3)n2)c2ccccc2-c2c(-c3ccccc3)cccc2N1c1ccccc1 ; O=P1(c2nc(-c3ccccc3)nc(-c3ccccc3)n2)c2ccccc2-c2cc(-c3ccc4c(c3)c3ccccc3n4-c3ccccc3)ccc2N1c1ccccc1 ; O=P1(c2ccccc2)c2ccccc2-c2cc(-c3ccc4c(c3)c3ccccc3n4-c3cc(-c4ccccc4)cc(-c4ccccc4)c3)ccc2N1c1ccccc1]

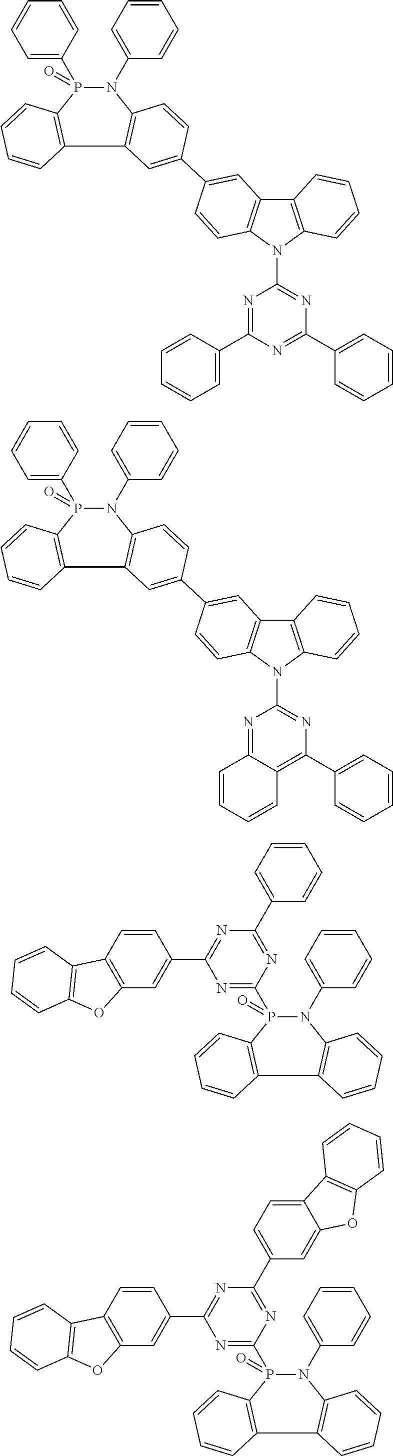

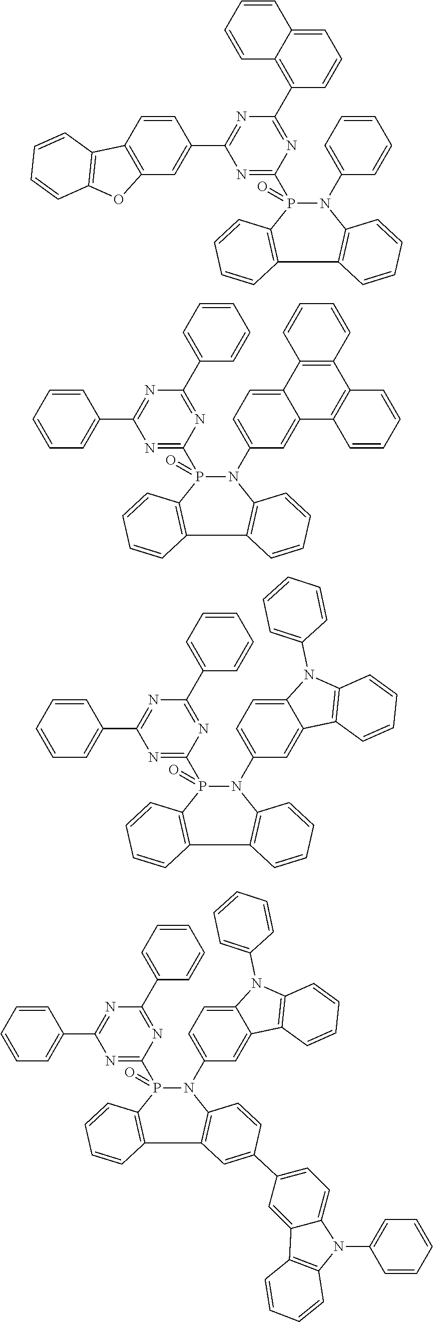

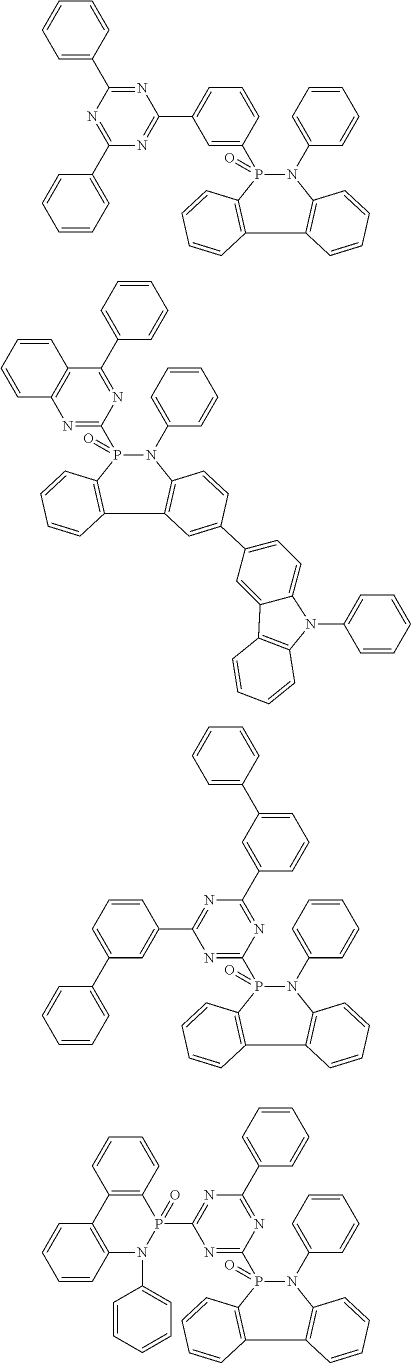

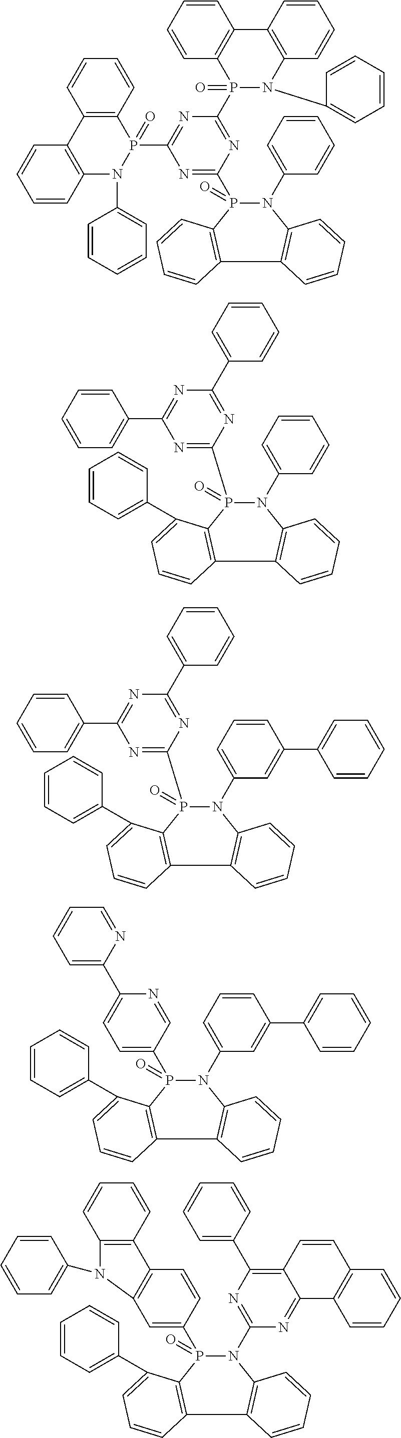

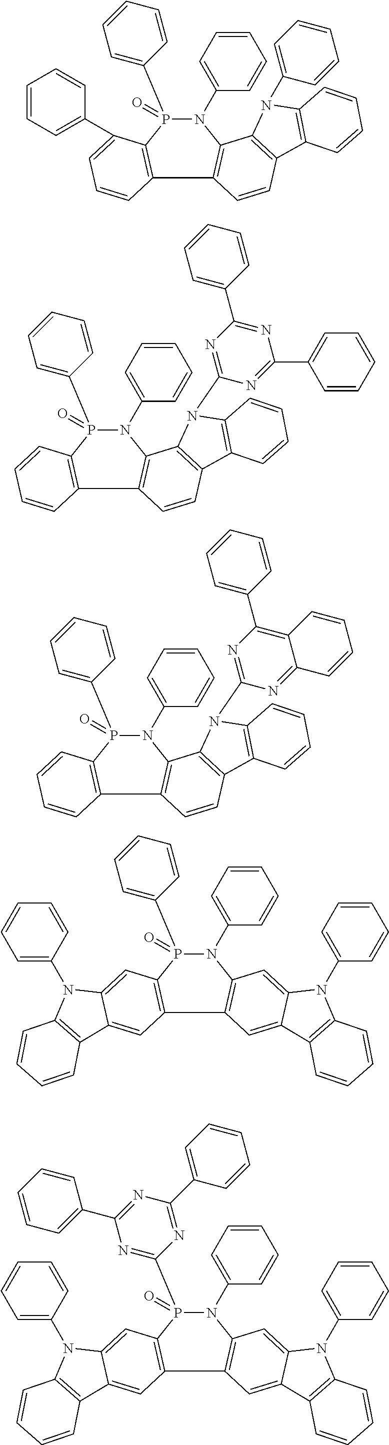

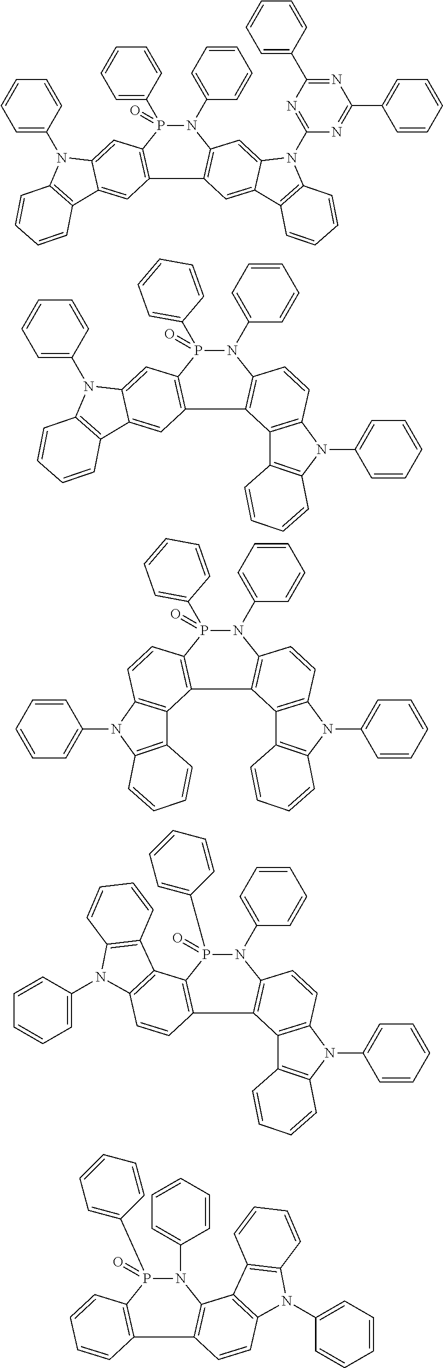

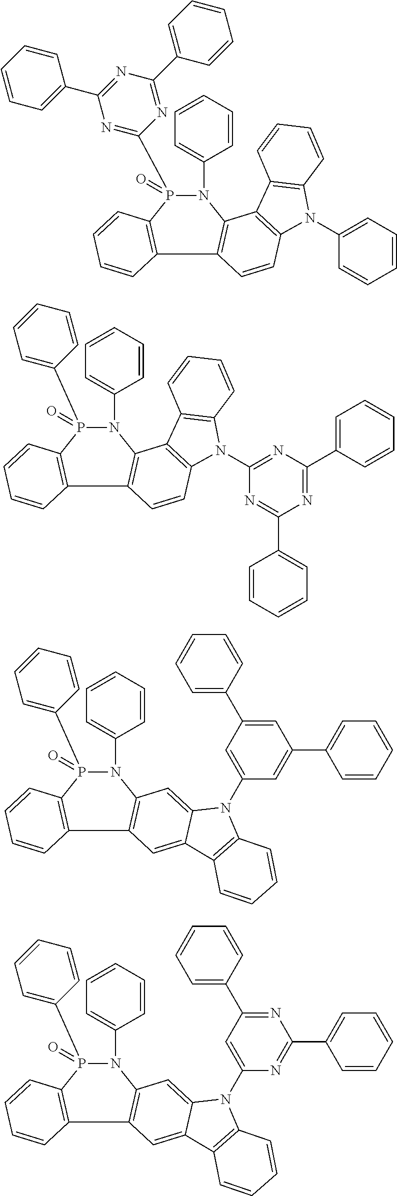

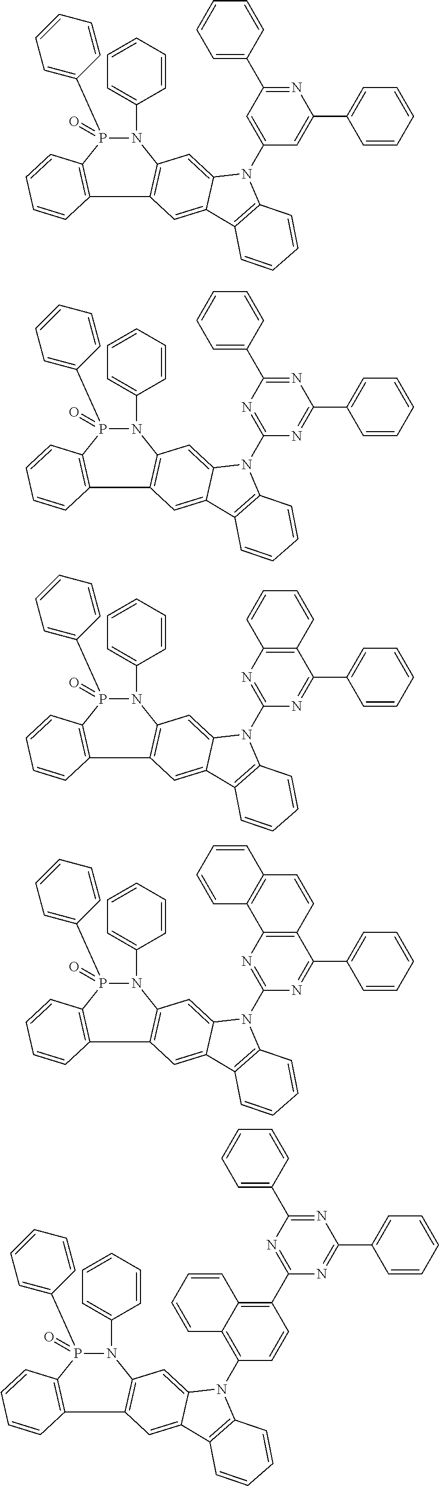

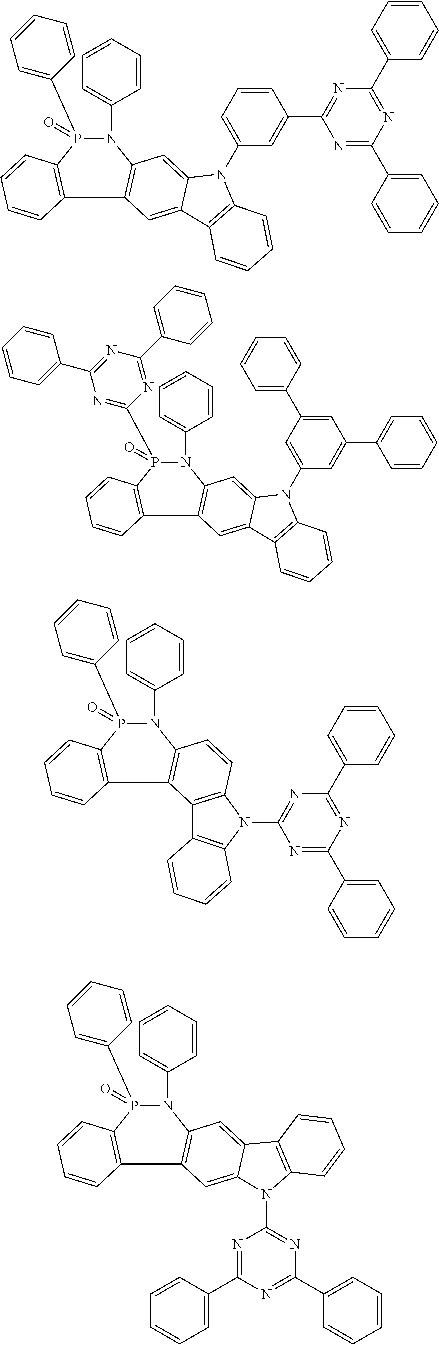

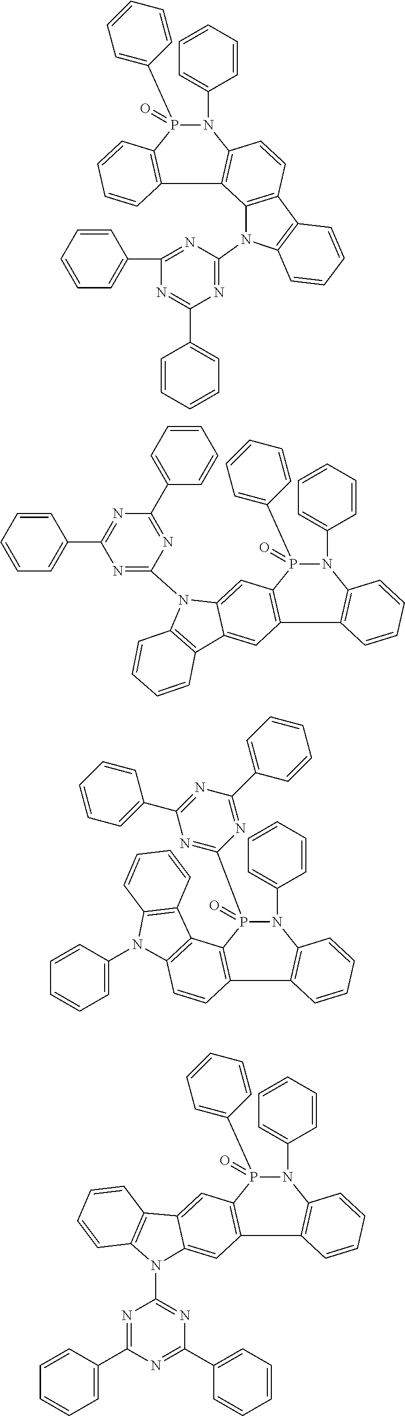

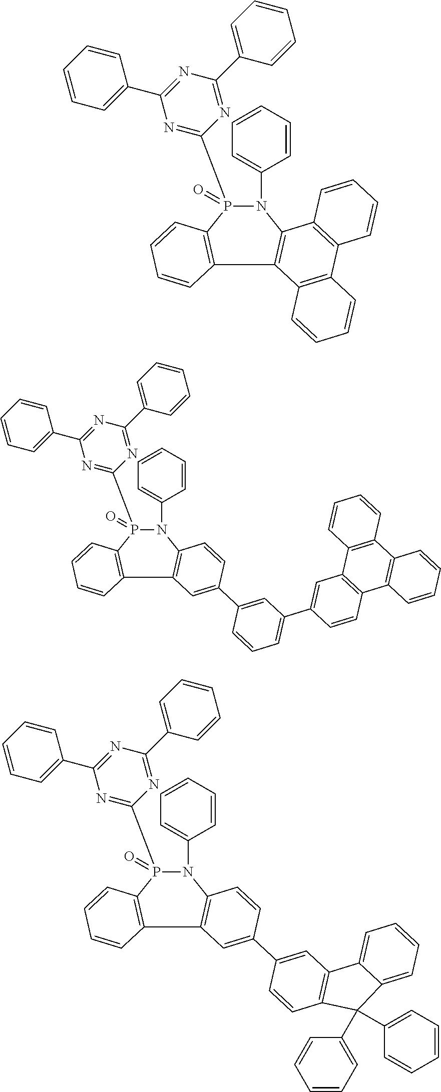 [SMILES: O=P1(c2nc(-c3ccccc3)nc(-c3ccccc3)n2)c2ccccc2-c2c(c3ccccc3c3ccccc23)N1c1ccccc1 ; O=P1(c2nc(-c3ccccc3)nc(-c3ccccc3)n2)c2ccccc2-c2cc(-c3cccc(-c4ccc5c6ccccc6c6ccccc6c5c4)c3)ccc2N1c1ccccc1 ; O=P1(c2nc(-c3ccccc3)nc(-c3ccccc3)n2)c2ccccc2-c2cc(-c3ccc4c(c3)-c3ccccc3C4(c3ccccc3)c3ccccc3)ccc2N1c1ccccc1]

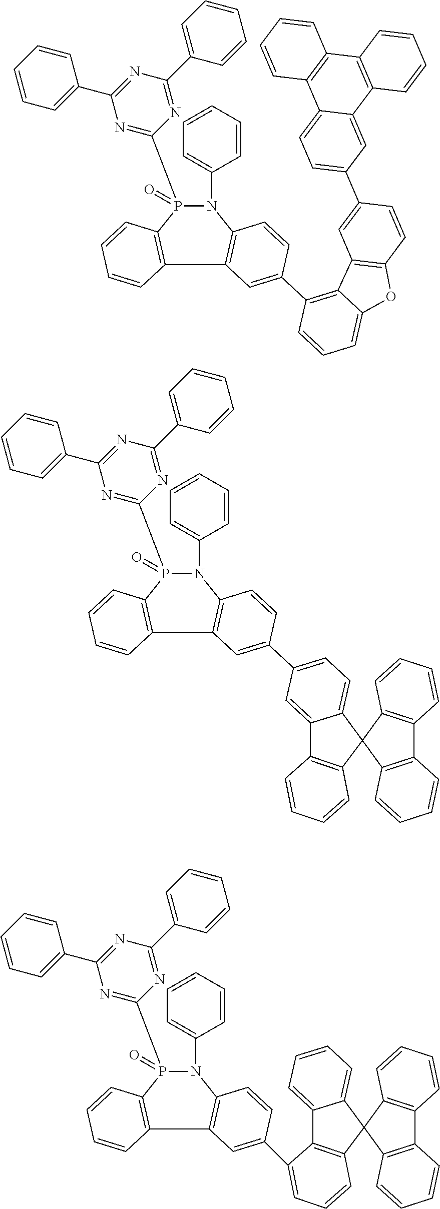 [SMILES: O=P1(c2nc(-c3ccccc3)nc(-c3ccccc3)n2)c2ccccc2-c2cc(-c3cccc4oc5ccc(-c6ccc7c8ccccc8c8ccccc8c7c6)cc5c34)ccc2N1c1ccccc1 ; O=P1(c2nc(-c3ccccc3)nc(-c3ccccc3)n2)c2ccccc2-c2cc(-c3ccc4c(c3)-c3ccccc3C43c4ccccc4-c4ccccc43)ccc2N1c1ccccc1 ; O=P1(c2nc(-c3ccccc3)nc(-c3ccccc3)n2)c2ccccc2-c2cc(-c3cccc4c3-c3ccccc3C43c4ccccc4-c4ccccc43)ccc2N1c1ccccc1]

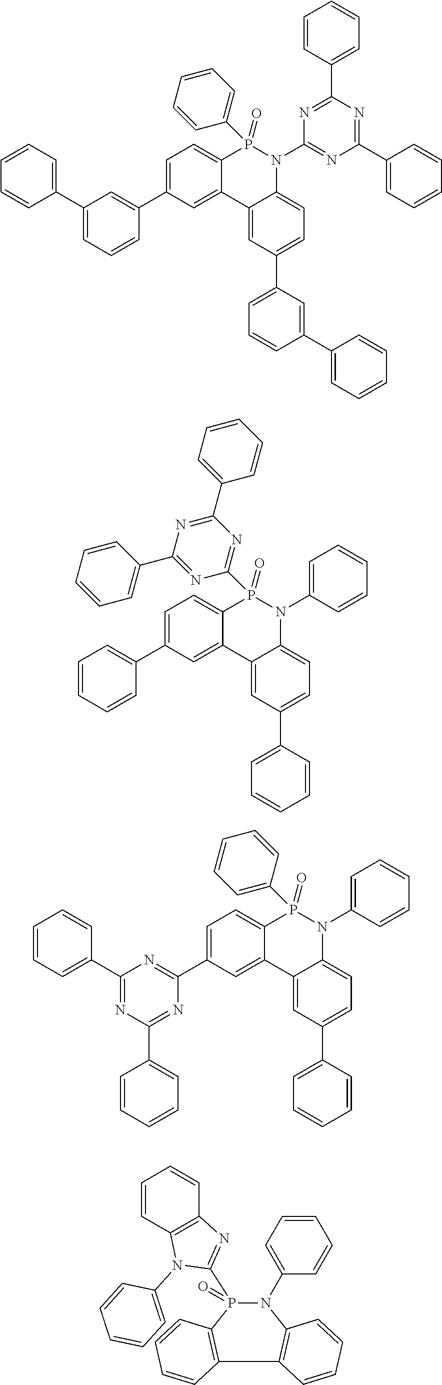

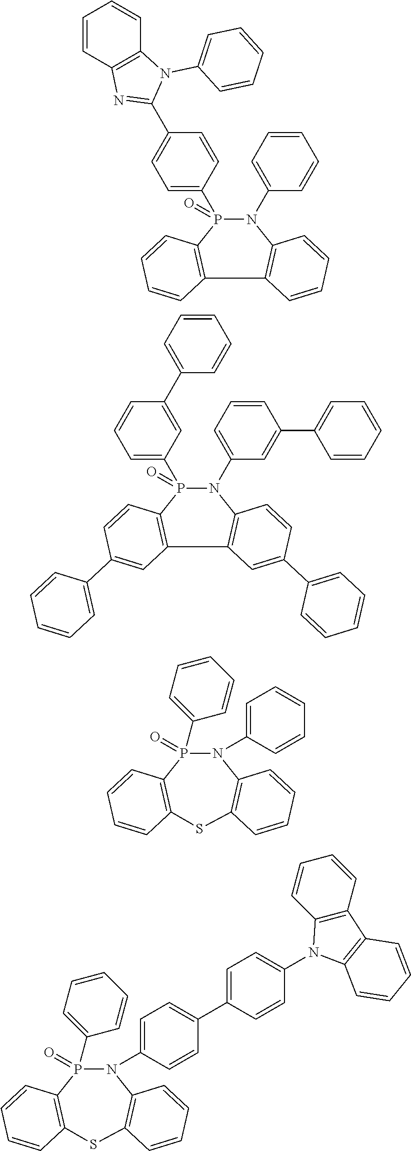

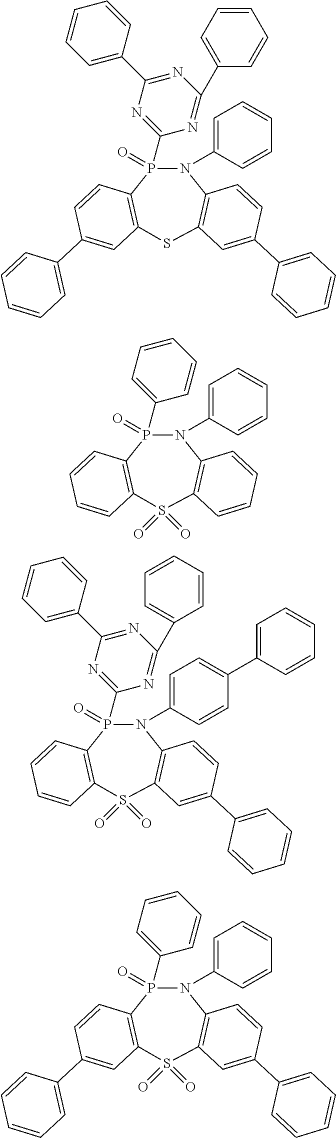

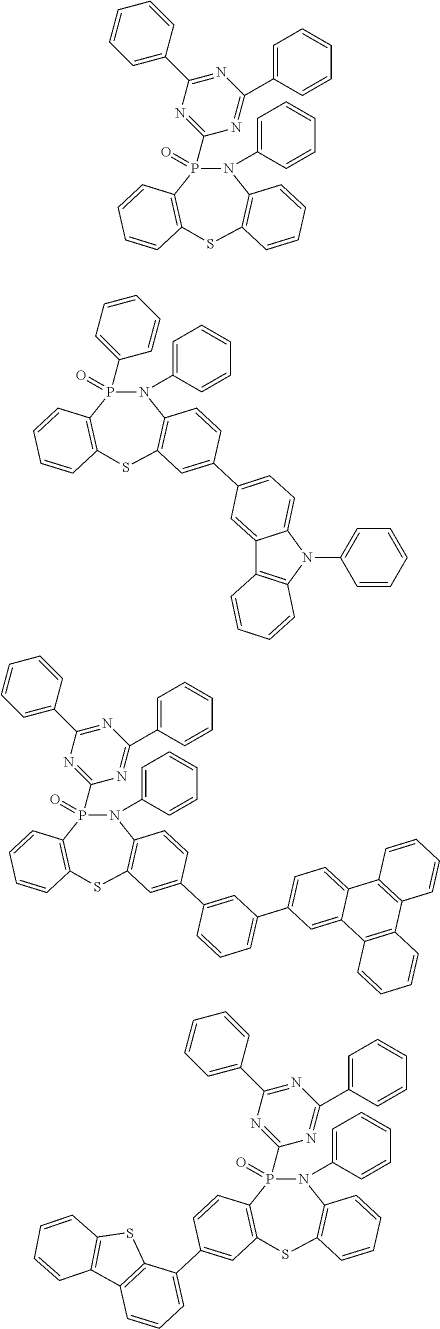

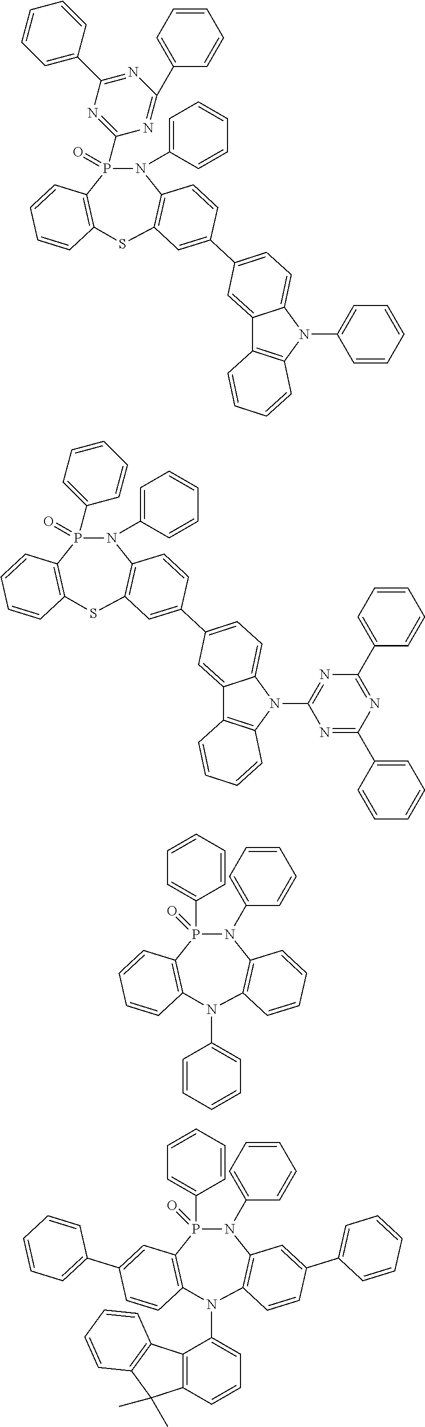

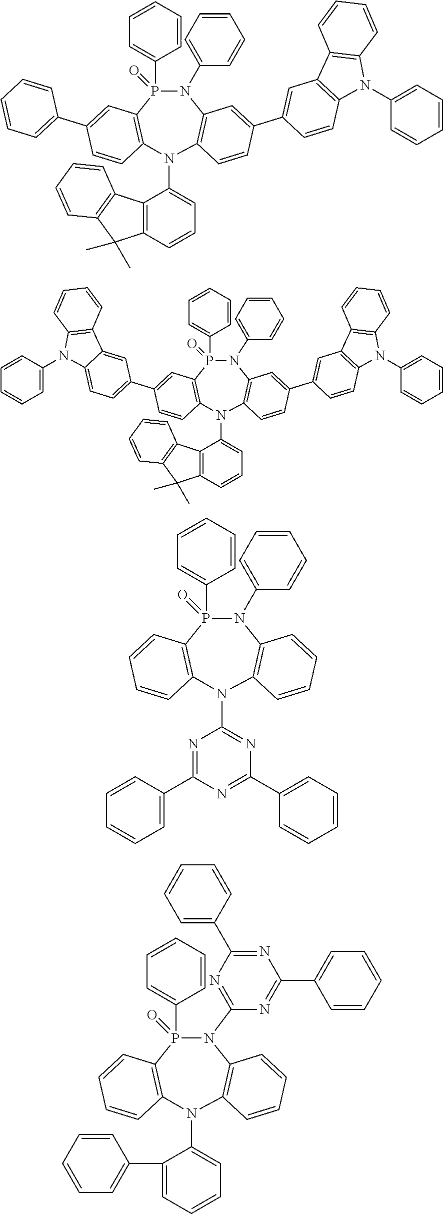

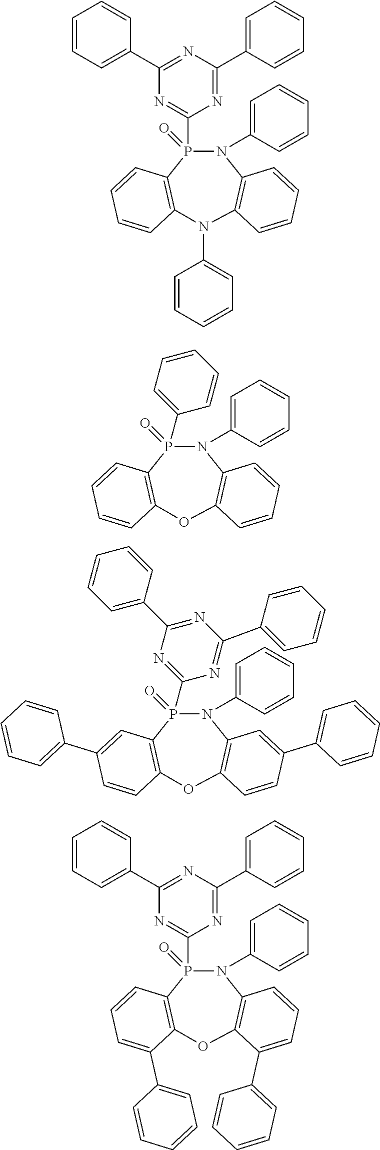

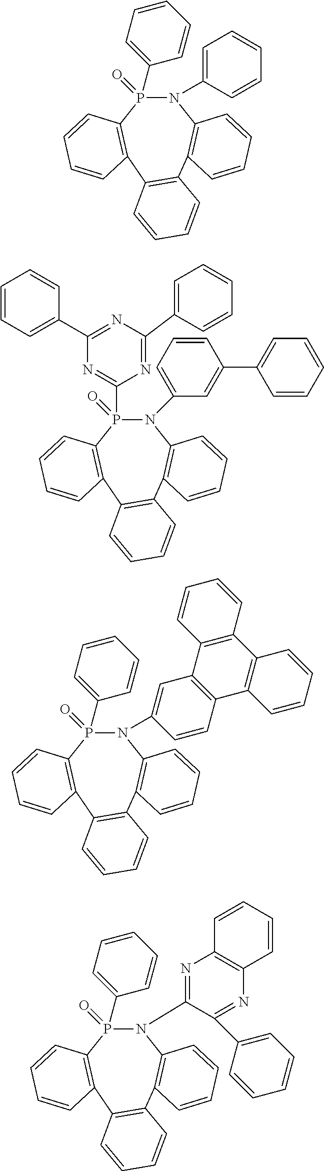

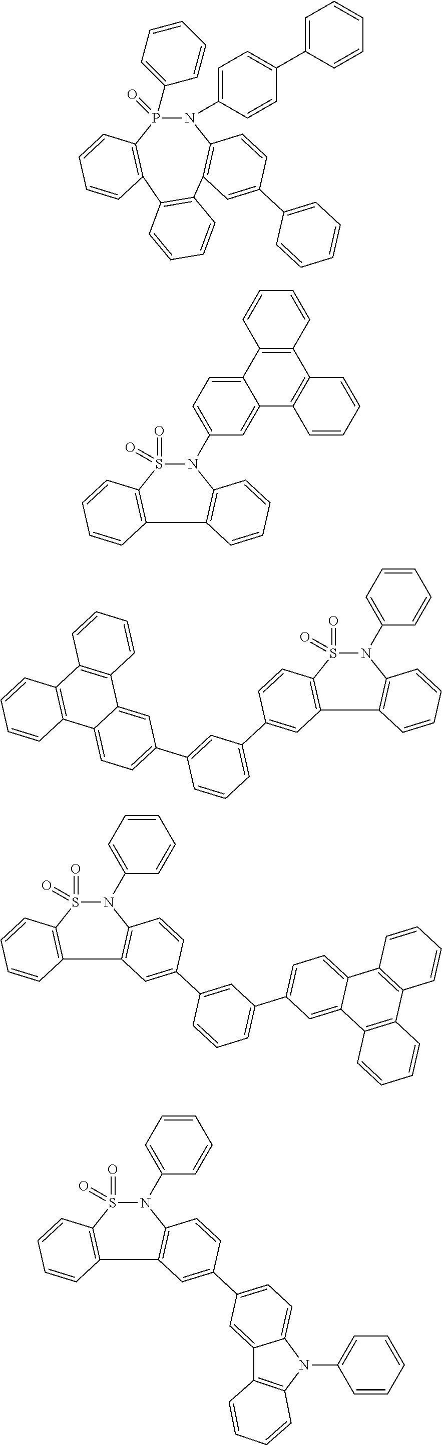

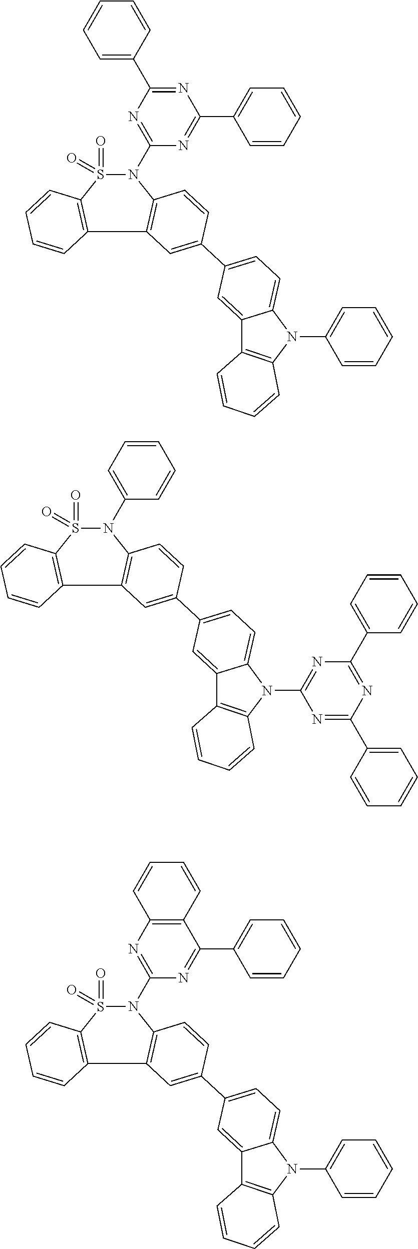 [SMILES: O=S1(=O)c2ccccc2-c2cc(-c3ccc4c(c3)c3ccccc3n4-c3ccccc3)ccc2N1c1nc(-c2ccccc2)nc(-c2ccccc2)n1 ; O=S1(=O)c2ccccc2-c2cc(-c3ccc4c(c3)c3ccccc3n4-c3nc(-c4ccccc4)nc(-c4ccccc4)n3)ccc2N1c1ccccc1 ; O=S1(=O)c2ccccc2-c2cc(-c3ccc4c(c3)c3ccccc3n4-c3ccccc3)ccc2N1c1nc(-c2ccccc2)c2ccccc2n1]

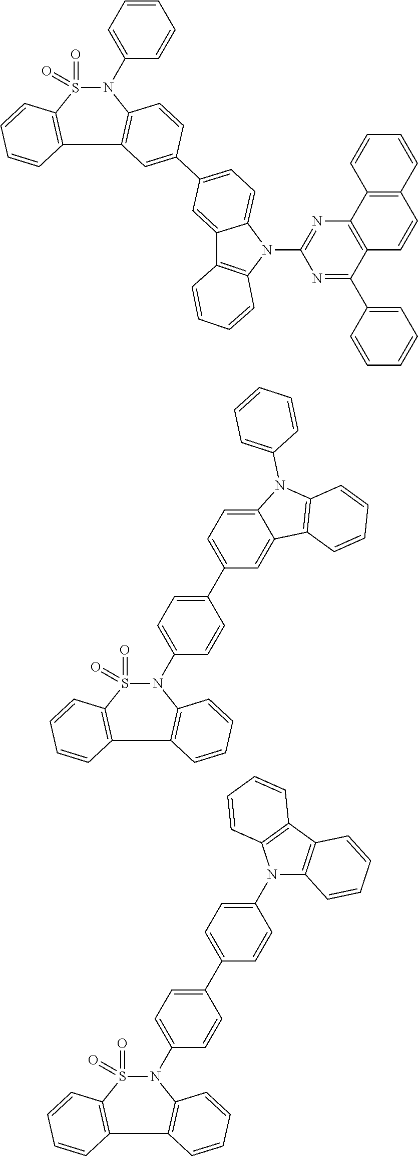

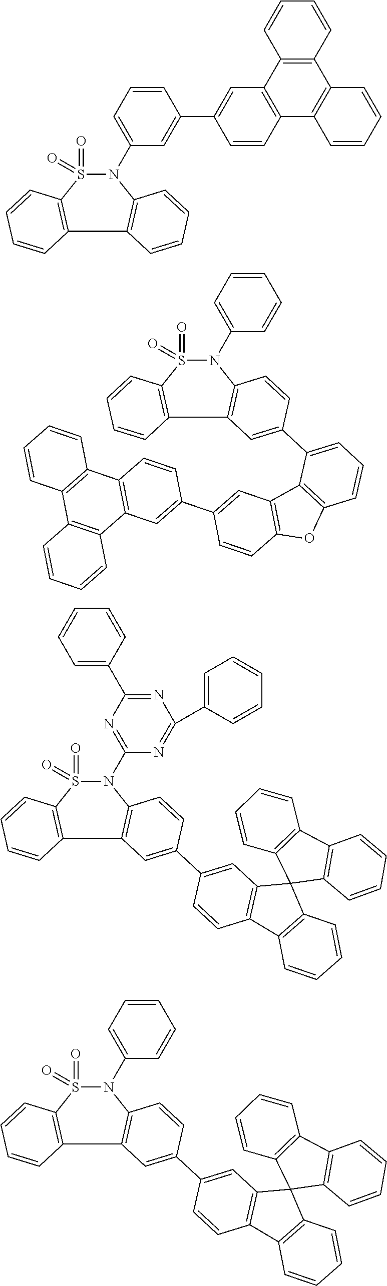

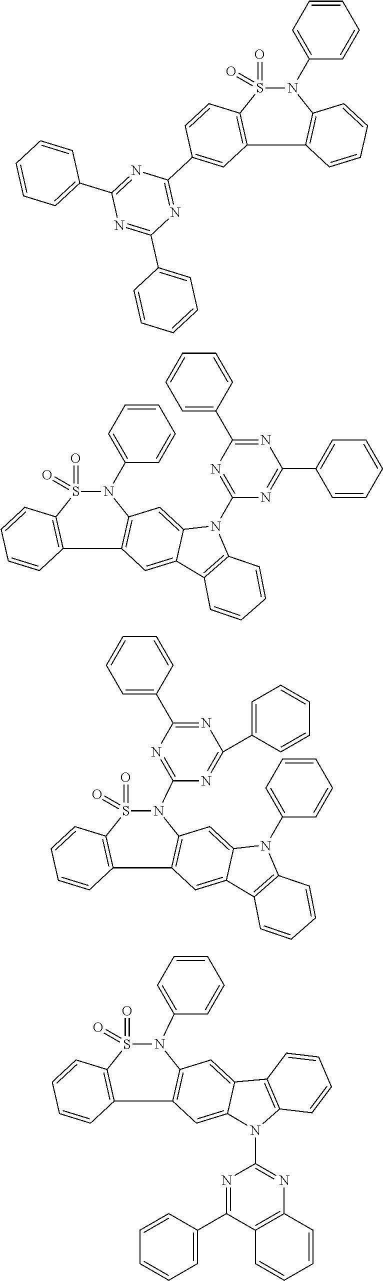

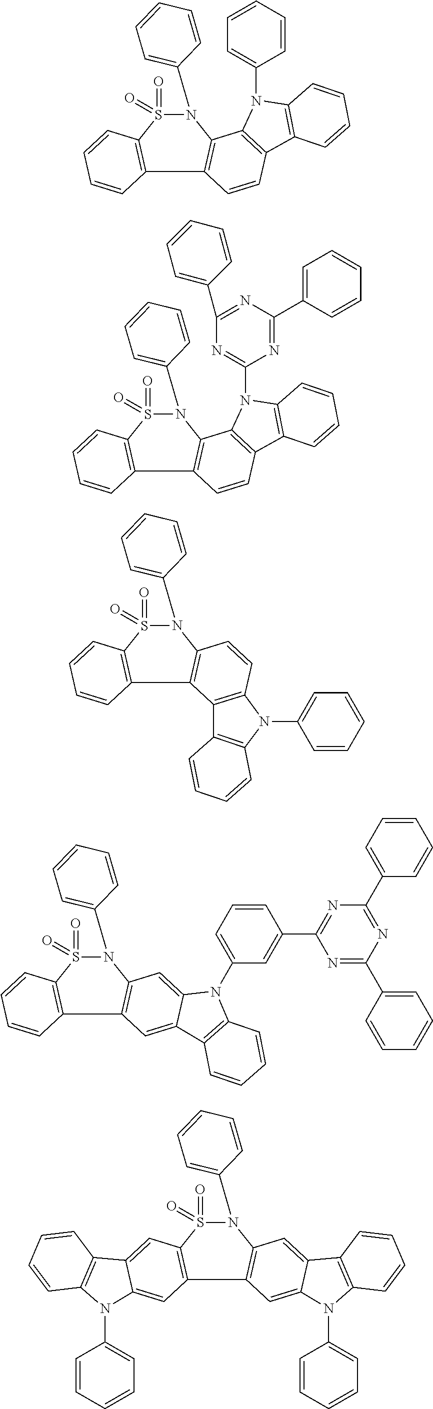

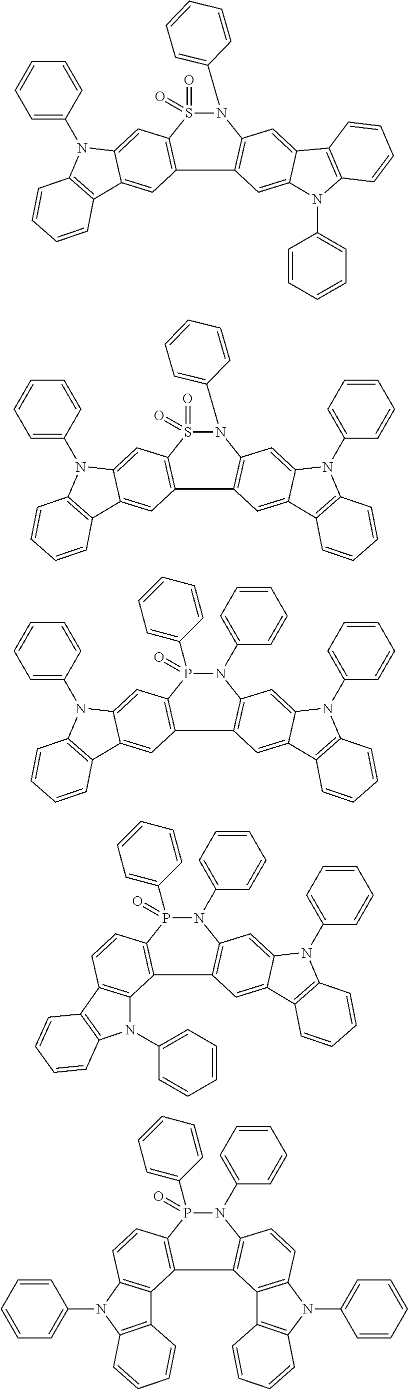

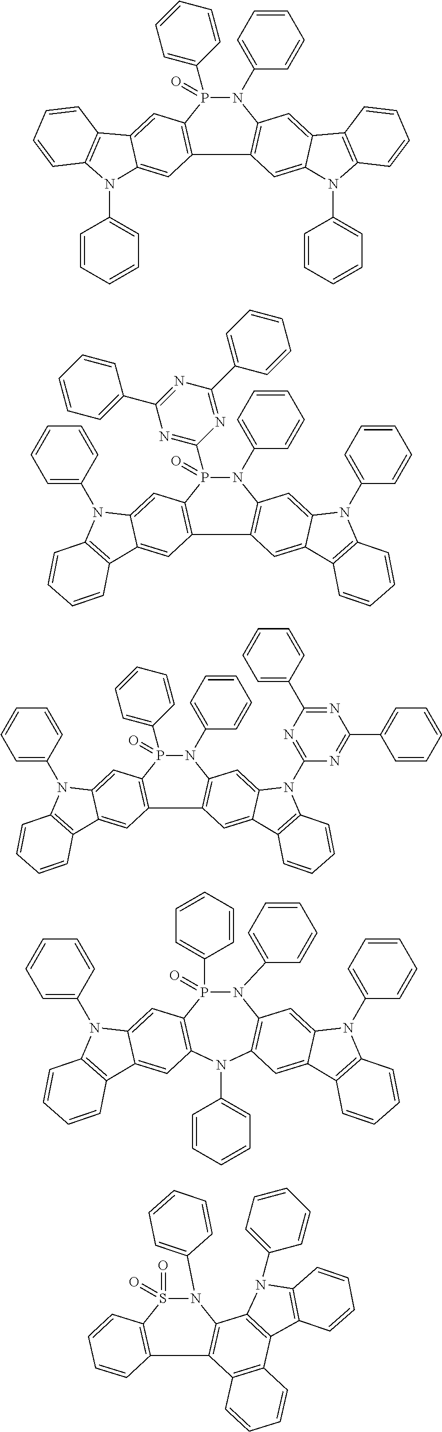

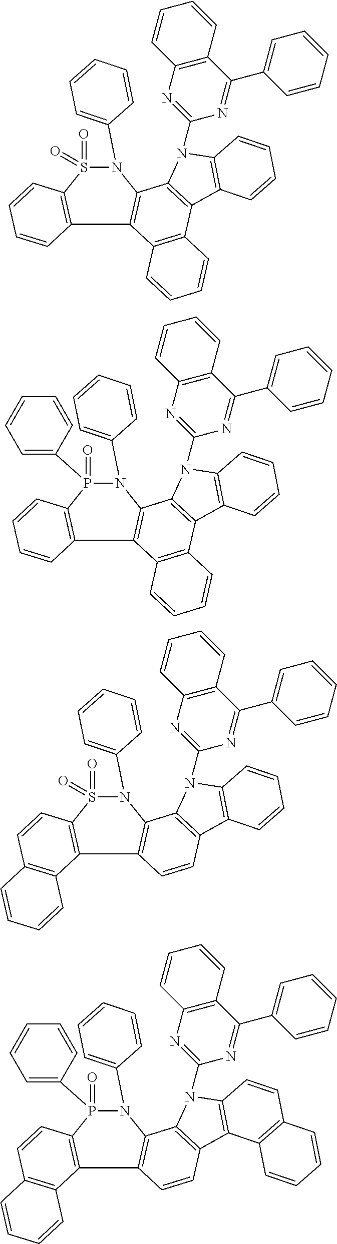

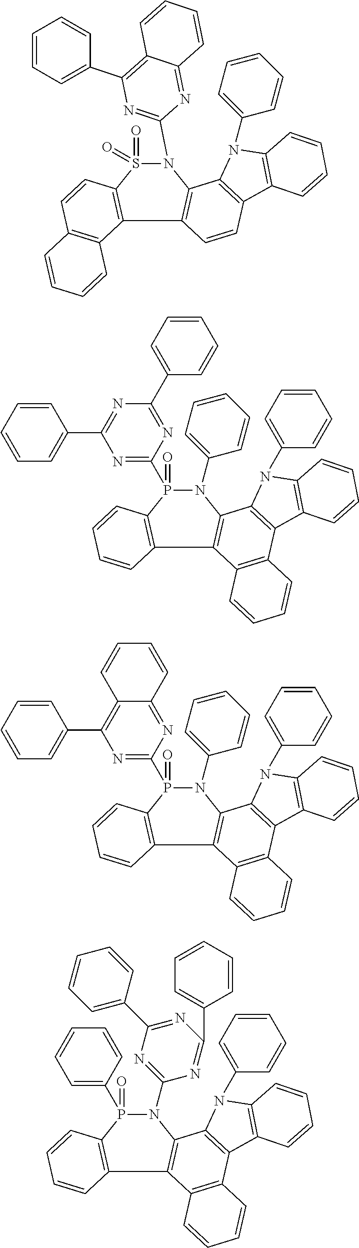

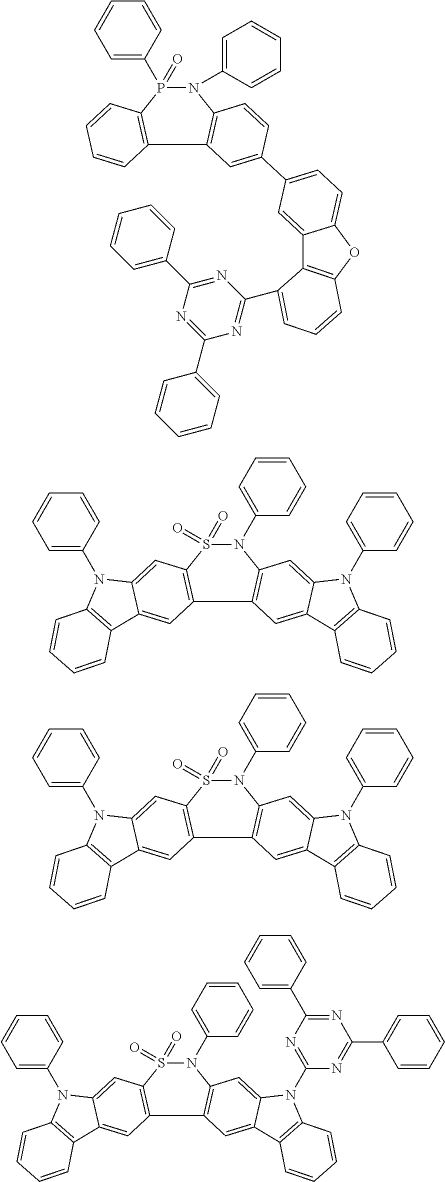 [SMILES: O=P1(c2ccccc2)c2ccccc2-c2cc(-c3ccc4oc5cccc(-c6nc(-c7ccccc7)nc(-c7ccccc7)n6)c5c4c3)ccc2N1c1ccccc1 ; O=S1(=O)c2cc3c(cc2-c2cc4c5ccccc5n(-c5ccccc5)c4cc2N1c1ccccc1)c1ccccc1n3-c1ccccc1 ; O=S1(=O)c2cc3c(cc2-c2cc4c5ccccc5n(-c5ccccc5)c4cc2N1c1ccccc1)c1ccccc1n3-c1ccccc1 ; O=S1(=O)c2cc3c(cc2-c2cc4c5ccccc5n(-c5nc(-c6ccccc6)nc(-c6ccccc6)n5)c4cc2N1c1ccccc1)c1ccccc1n3-c1ccccc1]

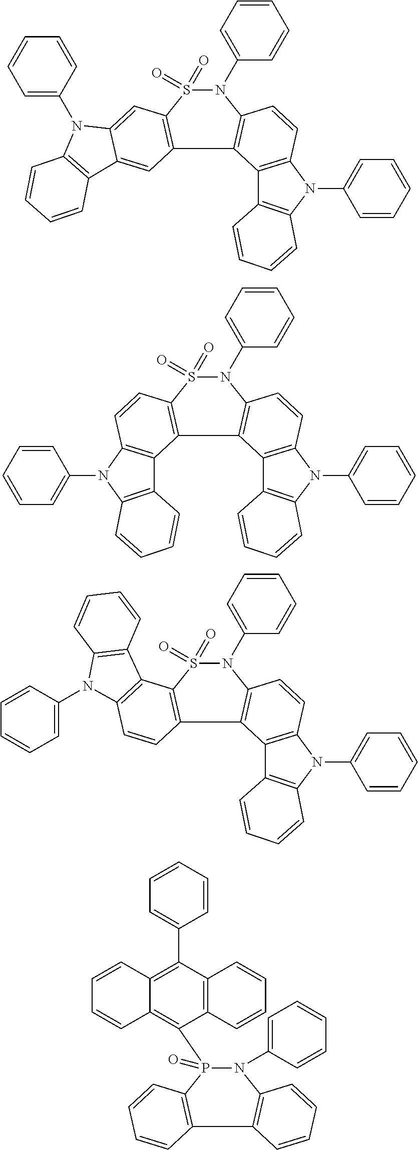

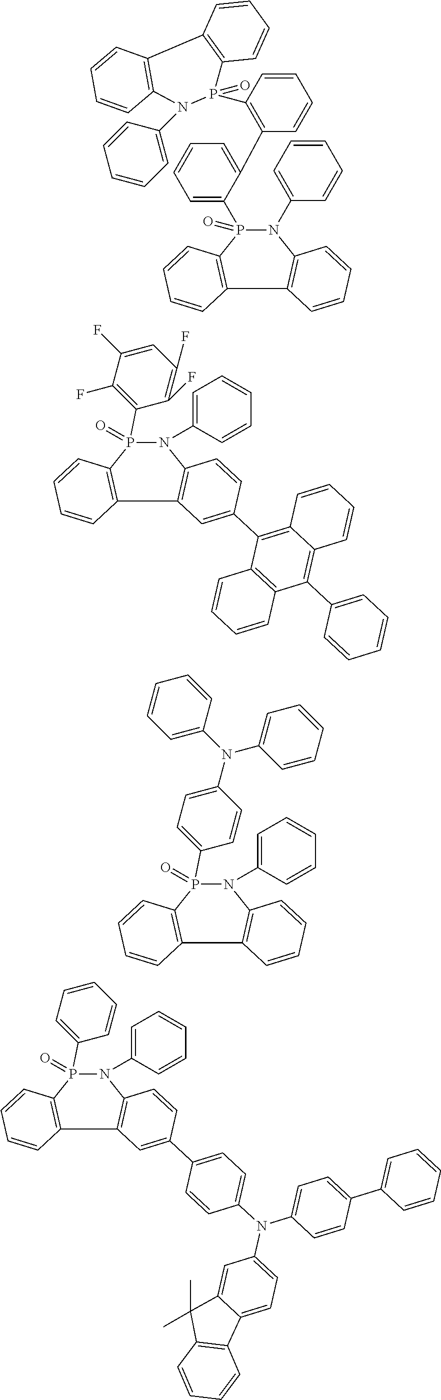

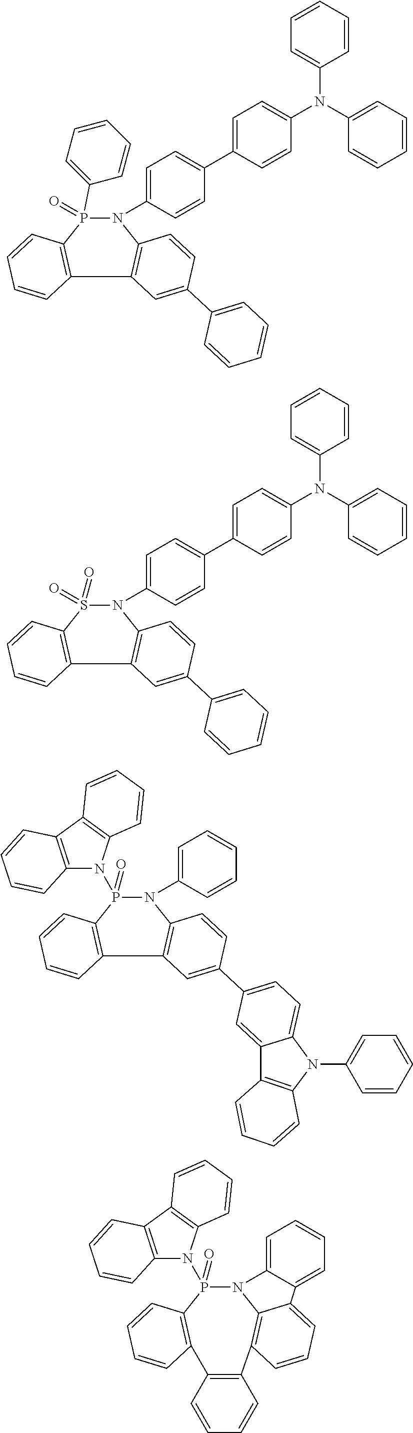

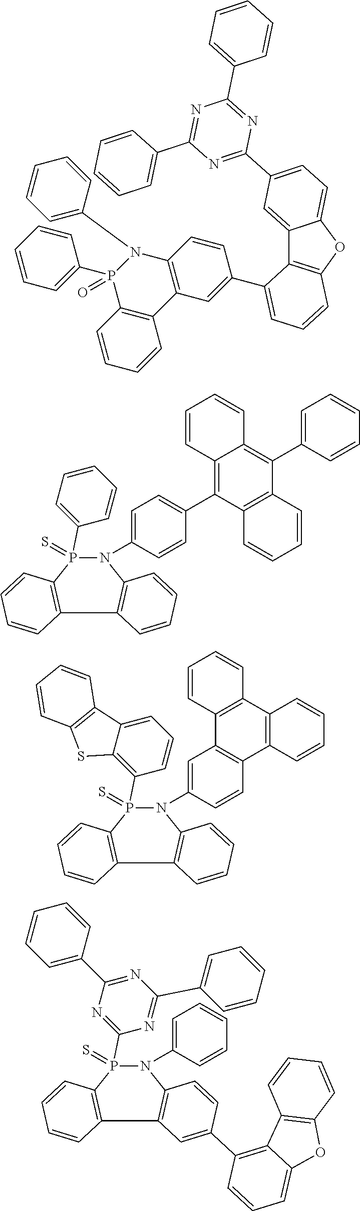

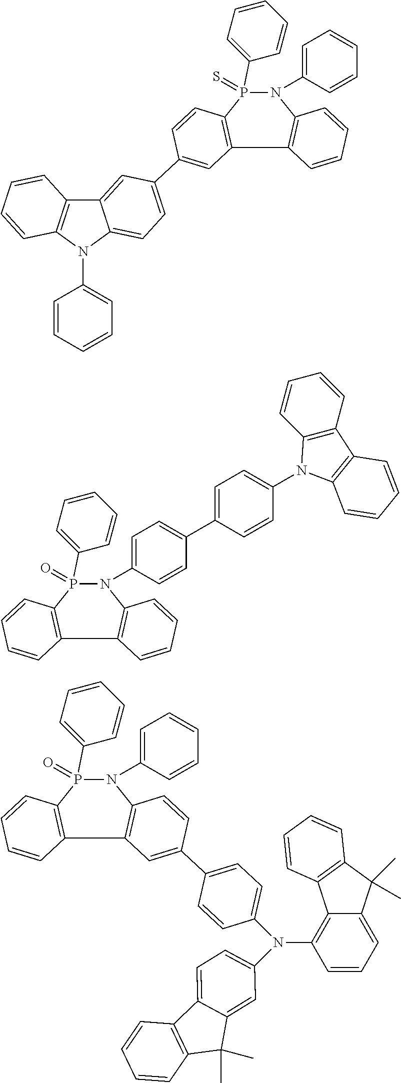

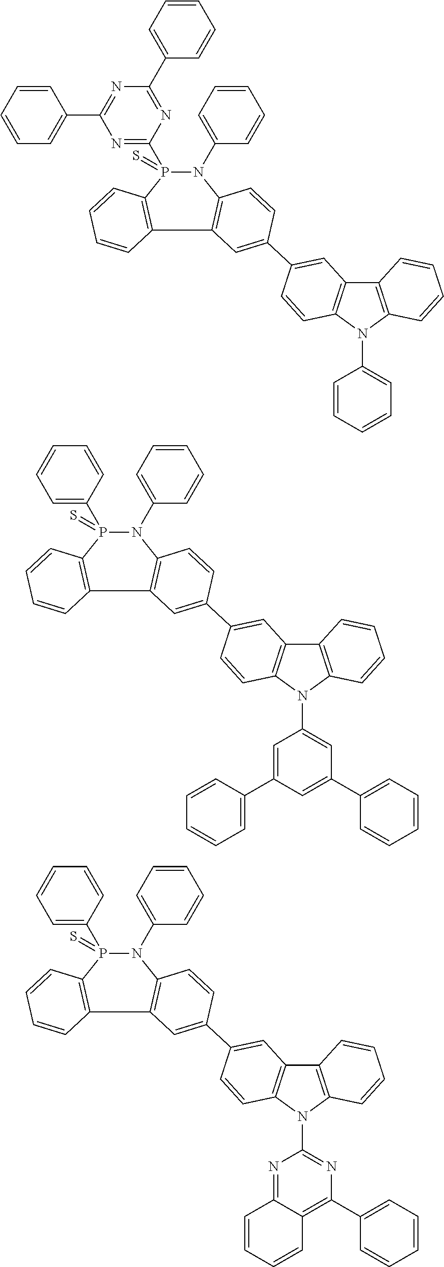

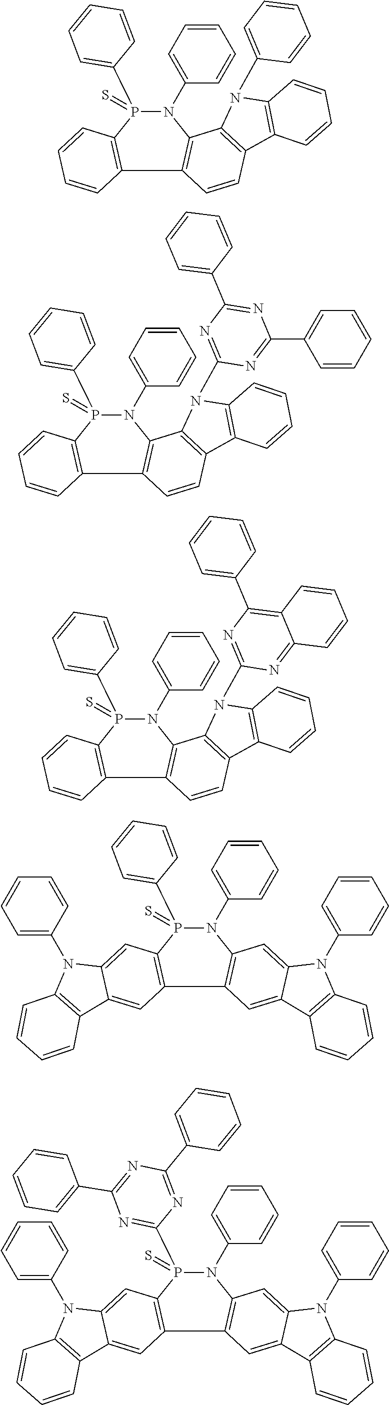

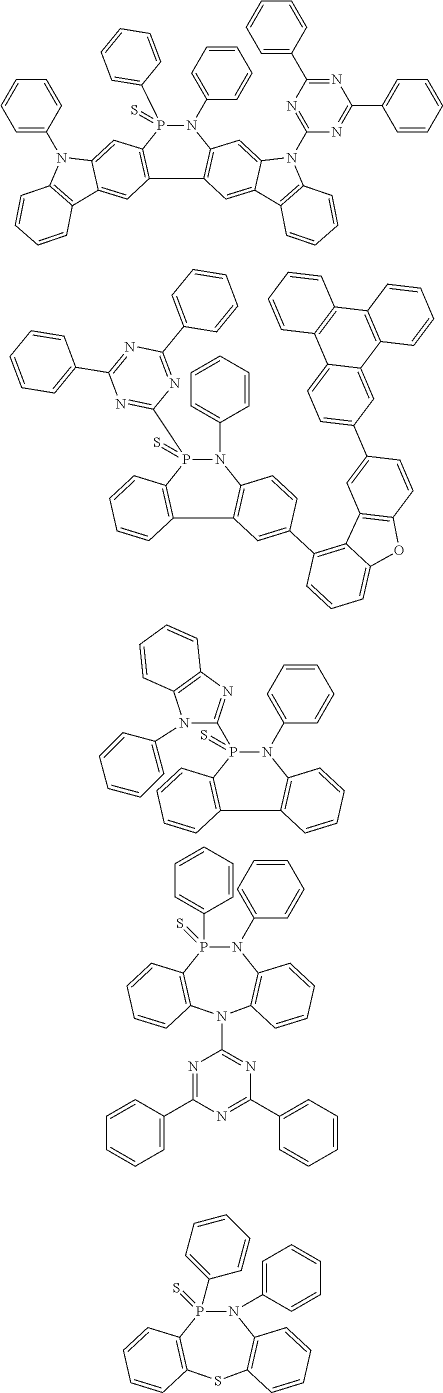

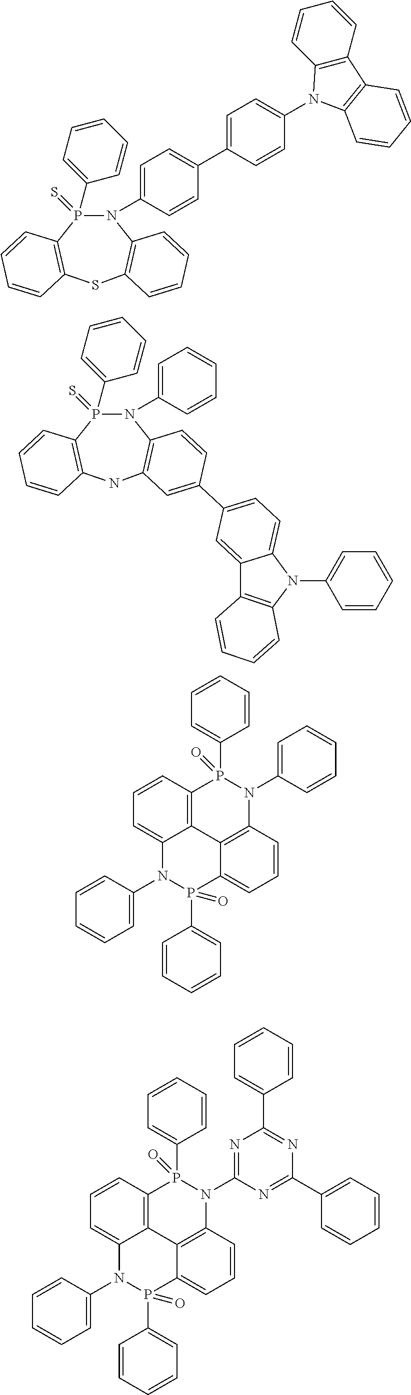

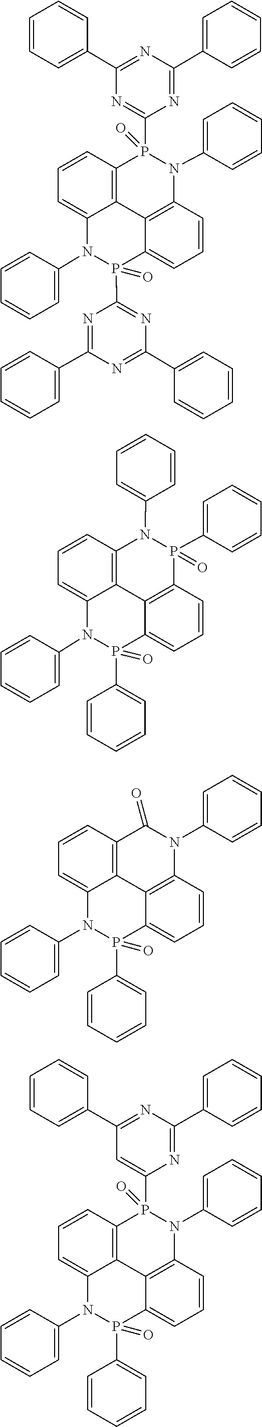

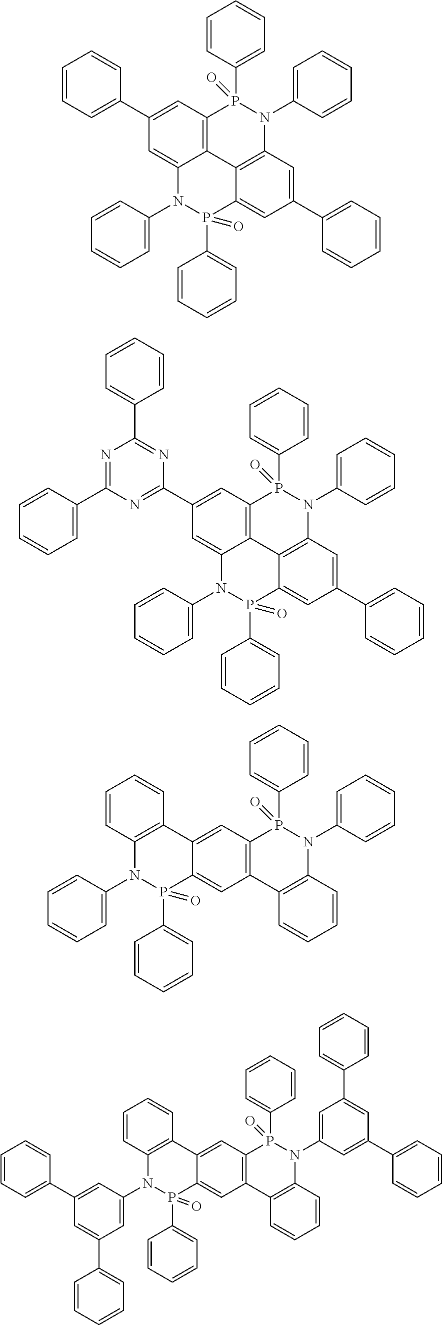

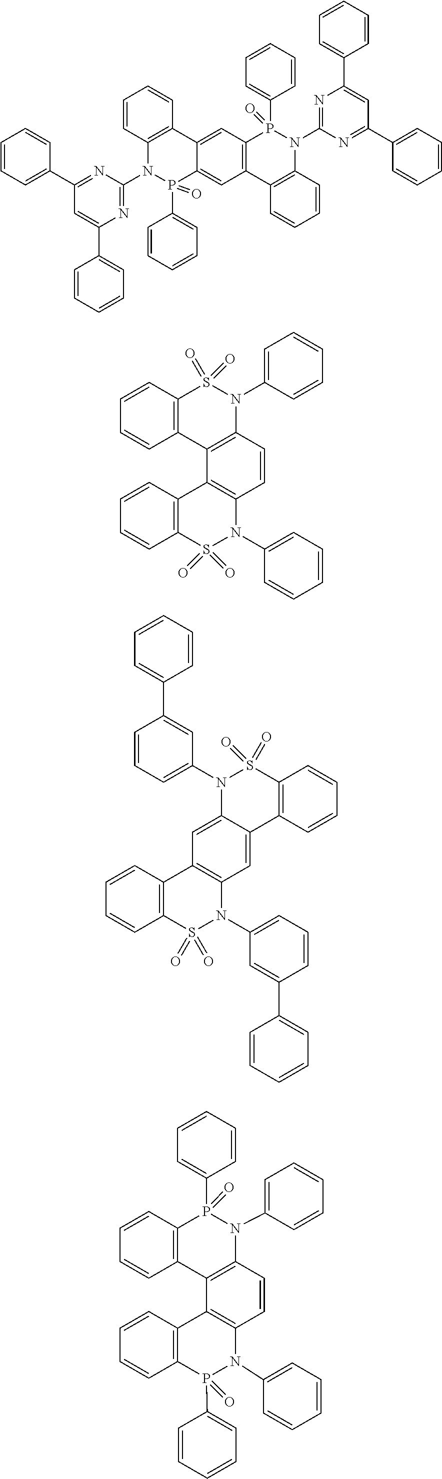 [SMILES: O=P1(c2ccccc2)c2cc3c(cc2-c2ccccc2N1c1nc(-c2ccccc2)cc(-c2ccccc2)n1)P(=O)(c1ccccc1)N(c1nc(-c2ccccc2)cc(-c2ccccc2)n1)c1ccccc1-3 ; O=S1(=O)c2ccccc2-c2c(ccc3c2-c2ccccc2S(=O)(=O)N3c2ccccc2)N1c1ccccc1 ; O=S1(=O)c2ccccc2-c2cc3c(cc2N1c1cccc(-c2ccccc2)c1)-c1ccccc1S(=O)(=O)N3c1cccc(-c2ccccc2)c1 ; O=P1(c2ccccc2)c2ccccc2-c2c(ccc3c2-c2ccccc2P(=O)(c2ccccc2)N3c2ccccc2)N1c1ccccc1]

The compounds of the invention can be synthesized according to the schemes below. Schemes 1 to 4 show the syntheses of the compounds with Z═PAr², and scheme 5 the synthesis of the compounds with Z═S(═O). The references cited describe analogous reactions, for example for compounds with an alkyl rather than an aryl or heteroaryl group on the nitrogen.

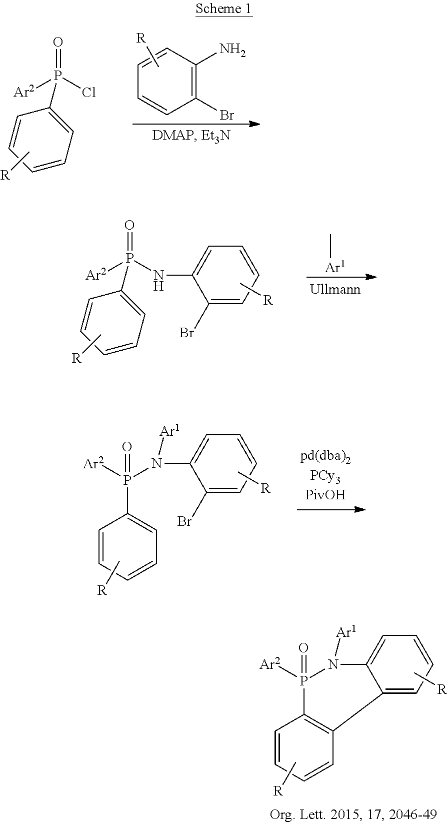

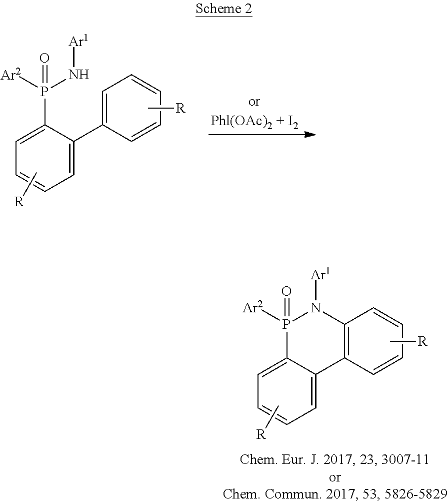

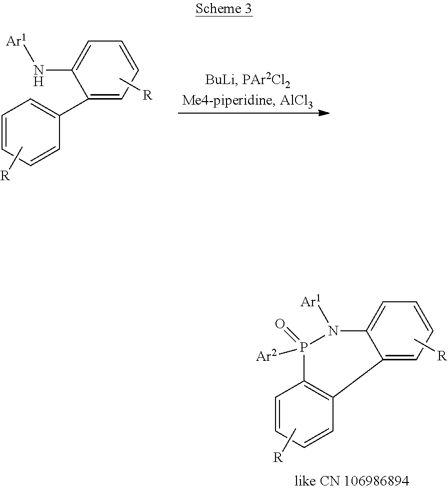

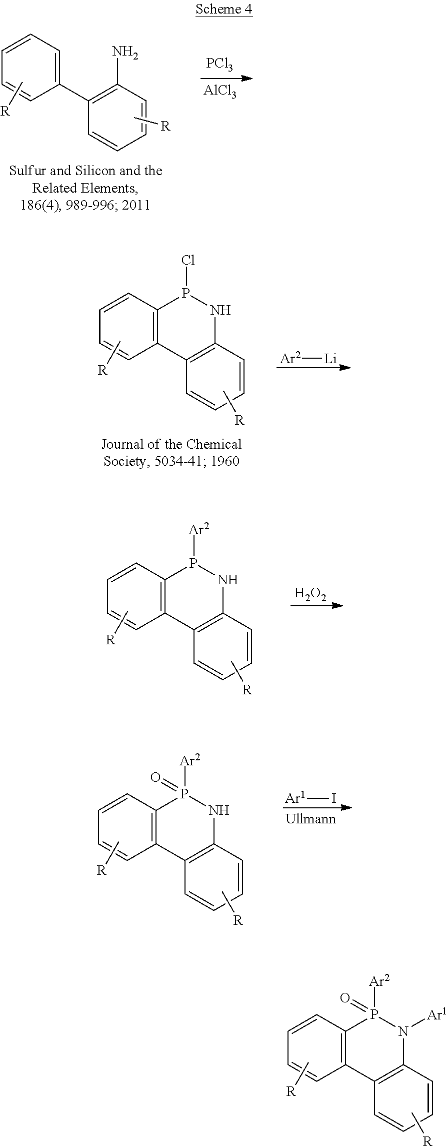

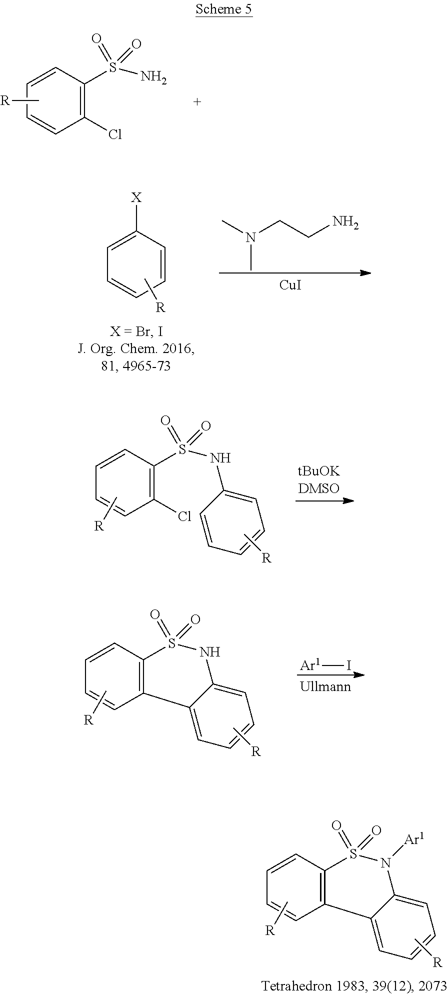

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. This further compound may also be polymeric.

The compounds of the invention are suitable for use in an electronic device, especially in an organic electroluminescent device.

The present invention therefore further provides for the use of a compound of the invention in an electronic device, especially in an organic electroluminescent device.

The present invention still further provides an electronic device comprising at least one compound of the invention.

An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound of the invention according to the above-detailed embodiments may be used in different layers, according to the exact structure. Preference is given to an organic electroluminescent device comprising a compound of formula (1) or the above-recited preferred embodiments in an emitting layer as matrix material for phosphorescent emitters or for emitters that exhibit TADF (thermally activated delayed fluorescence), especially for phosphorescent emitters. In this case, the organic electroluminescent device may contain an emitting layer, or it may contain a plurality of emitting layers, where at least one emitting layer contains at least one compound of the invention as matrix material. In addition, the compound of the invention can also be used in an electron transport layer and/or in a hole blocker layer and/or in a hole transport layer and/or in an exciton blocker layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2006/130598, WO 2009/021126, WO 2009/124627 and WO 2010/006680.

In a preferred embodiment of the invention, the materials are used in combination with a further matrix material. Preferred co-matrix materials are selected from the group of the biscarbazoles, the bridged carbazoles, the triarylamines, the dibenzofuran-carbazole derivatives or dibenzofuran-amine derivatives, the carbazoleamines and the triazine derivatives.

Preferred biscarbazoles are the structures of the following formulae (19) and (20):

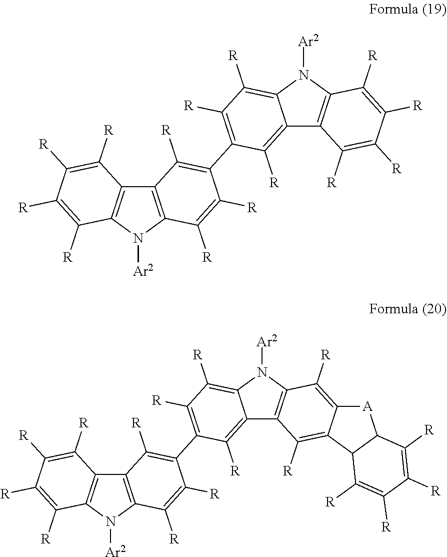

where Ar² and A have the definitions given above and R has the definitions given above, but R radicals here may also together form an aromatic or heteroaromatic ring system. In a preferred embodiment of the invention, A is CR₂.

Preferred embodiments of the compounds of the formulae (19) and (20) are the compounds of the following formulae (19a) and (20a):

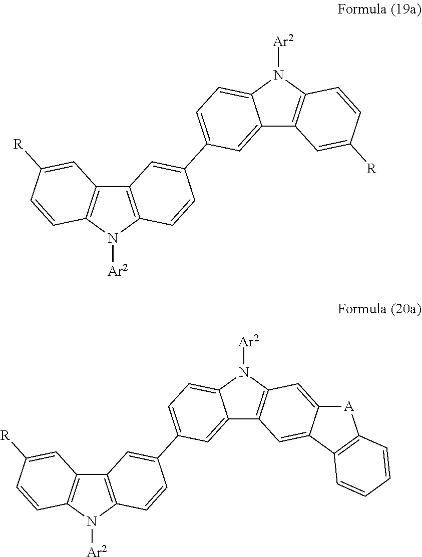

where the symbols used have the definitions given above.

Examples of suitable compounds of formulae (19) and (20) are the compounds depicted below:

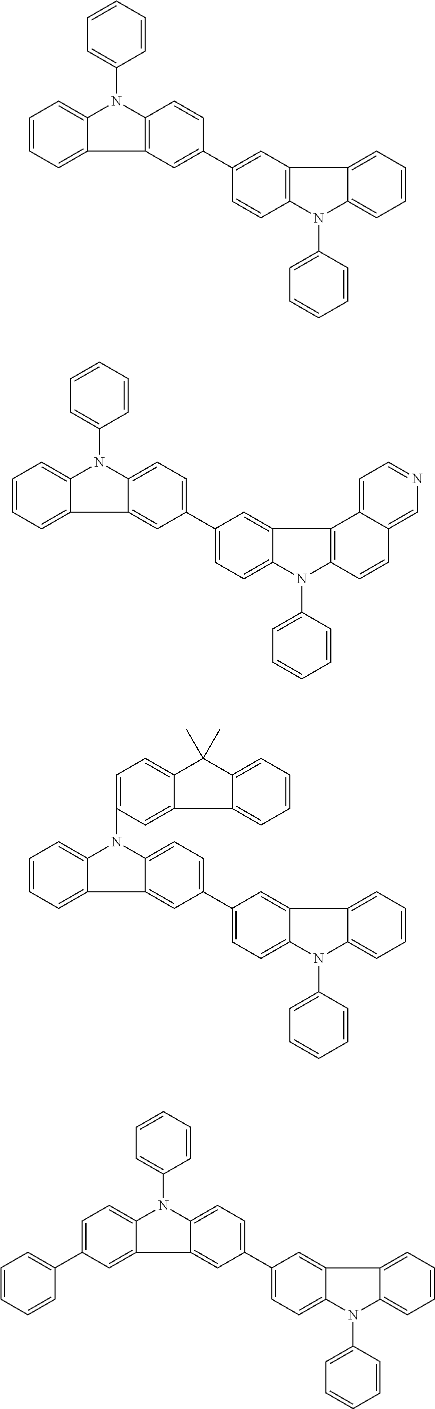 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4c6ccncc6ccc4n5-c4ccccc4)ccc32)cc1 ; CC1(C)c2ccccc2-c2cc(-n3c4ccccc4c4cc(-c5ccc6c(c5)c5ccccc5n6-c5ccccc5)ccc43)ccc21 ; c1ccc(-c2ccc3c(c2)c2cc(-c4ccc5c(c4)c4ccccc4n5-c4ccccc4)ccc2n3-c2ccccc2)cc1]

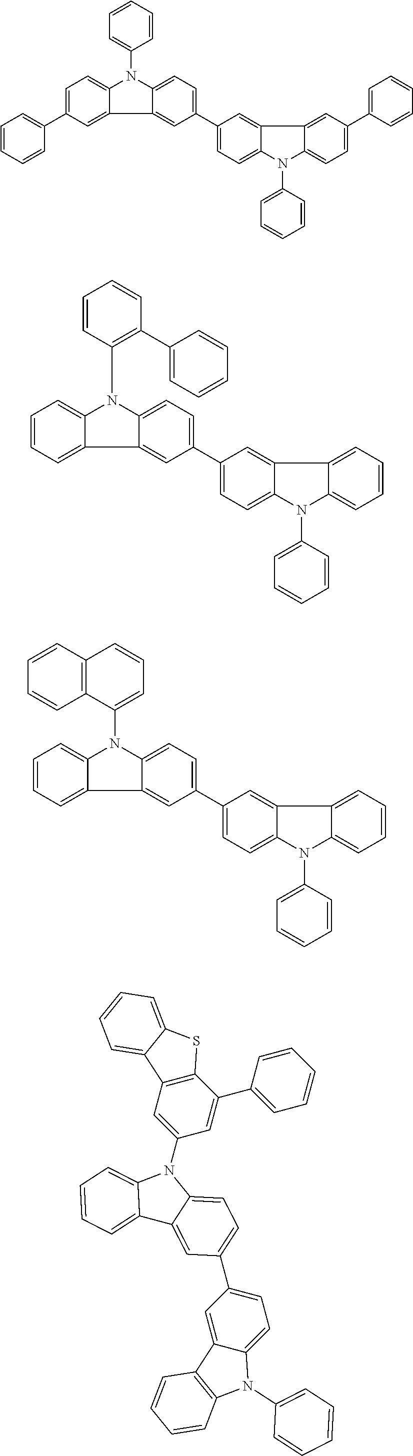 [SMILES: c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-c2cc(-n3c4ccccc4c4cc(-c5ccc6c(c5)c5ccccc5n6-c5ccccc5)ccc43)cc3c2sc2ccccc23)cc1]

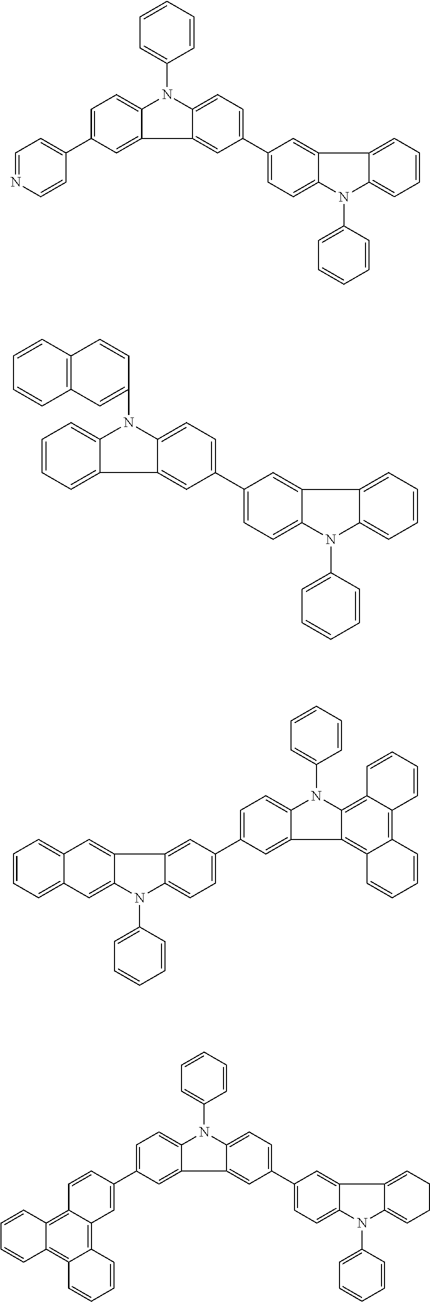 [SMILES: c1ccc(-n2c3ccccc3c3cc(-c4ccc5c(c4)c4cc(-c6ccncc6)ccc4n5-c4ccccc4)ccc32)cc1 ; c1ccc(-n2c3ccccc3c3cc(-c4ccc5c(c4)c4ccccc4n5-c4ccc5ccccc5c4)ccc32)cc1 ; c1ccc(-n2c3ccc(-c4ccc5c(c4)c4c6ccccc6c6ccccc6c4n5-c4ccccc4)cc3c3cc4ccccc4cc32)cc1 ; C1=c2c(n(-c3ccccc3)c3ccc(-c4ccc5c(c4)c4cc(-c6ccc7c8ccccc8c8ccccc8c7c6)ccc4n5-c4ccccc4)cc23)=CCC1]

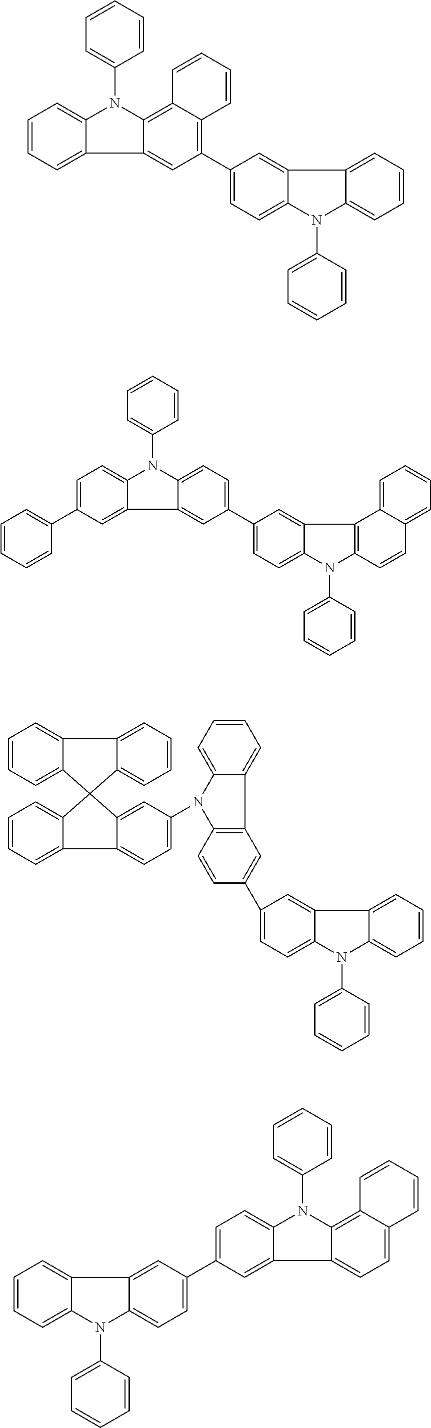 [SMILES: c1ccc(-n2c3ccccc3c3cc(-c4cc5c6ccccc6n(-c6ccccc6)c5c5ccccc45)ccc32)cc1 ; c1ccc(-c2ccc3c(c2)c2cc(-c4ccc5c(c4)c4c6ccccc6ccc4n5-c4ccccc4)ccc2n3-c2ccccc2)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccc6ccccc6c4n5-c4ccccc4)ccc32)cc1]

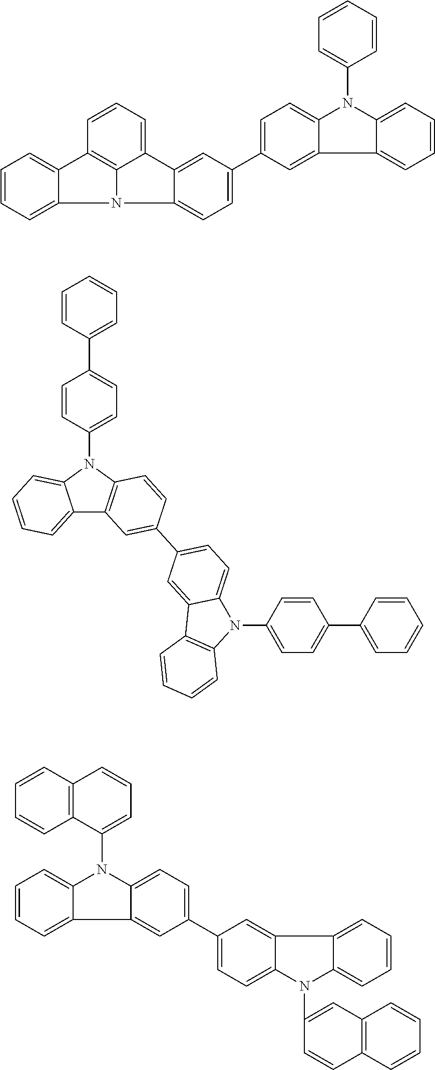 [SMILES: c1ccc(-n2c3ccccc3c3cc(-c4ccc5c(c4)c4cccc6c7ccccc7n5c64)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1]

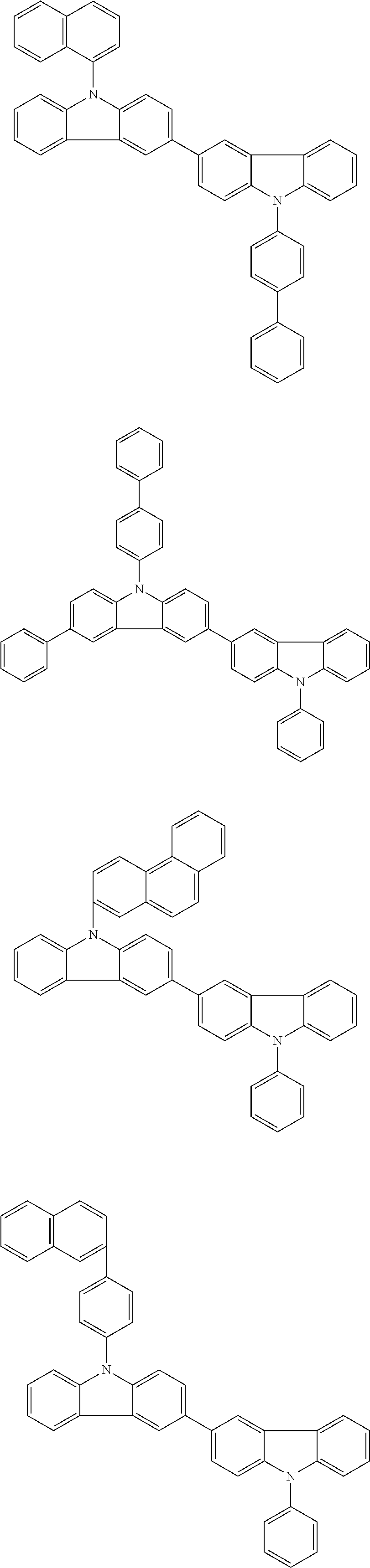 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

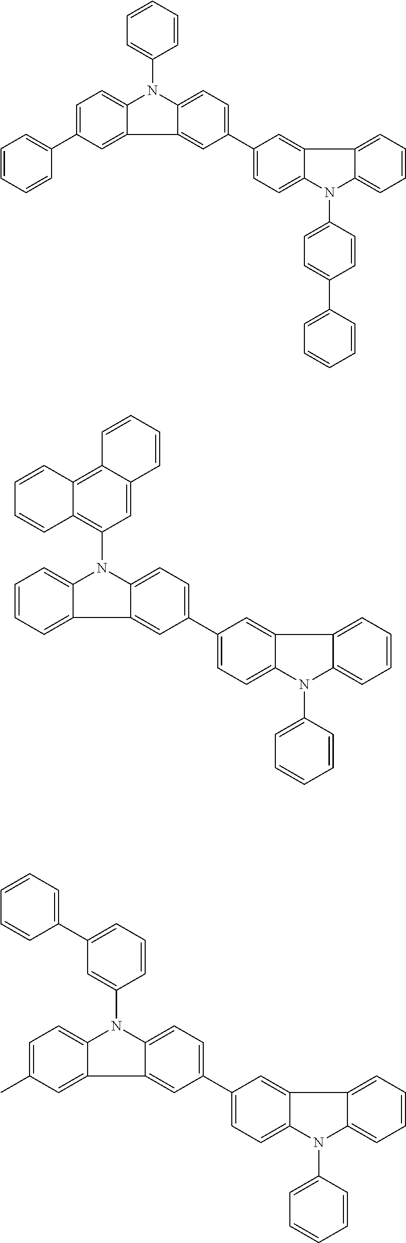 [SMILES: c1ccc(-c2ccc(-n3c4ccccc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4cc5ccccc5c5ccccc45)ccc32)cc1 ; Cc1ccc2c(c1)c1cc(-c3ccc4c(c3)c3ccccc3n4-c3ccccc3)ccc1n2-c1cccc(-c2ccccc2)c1]

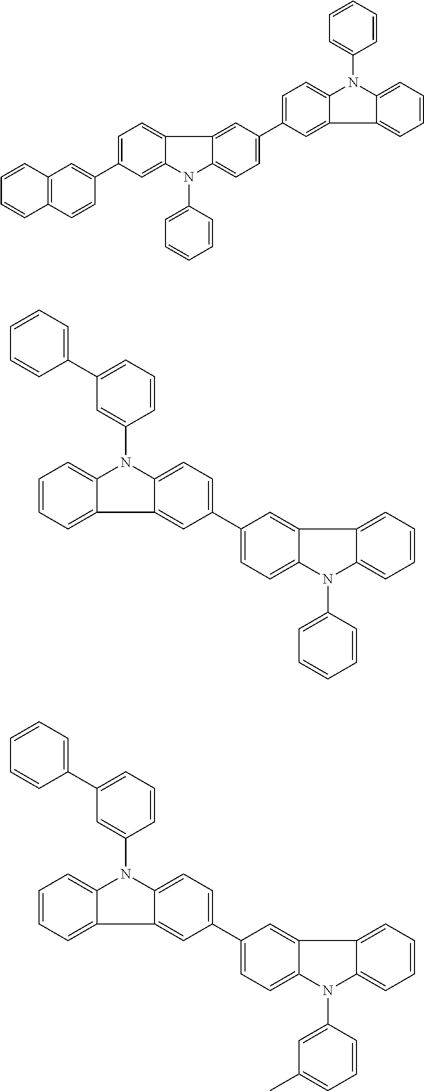 [SMILES: c1ccc(-n2c3ccccc3c3cc(-c4ccc5c(c4)c4ccc(-c6ccc7ccccc7c6)cc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; Cc1cccc(-n2c3ccccc3c3cc(-c4ccc5c(c4)c4ccccc4n5-c4cccc(-c5ccccc5)c4)ccc32)c1]

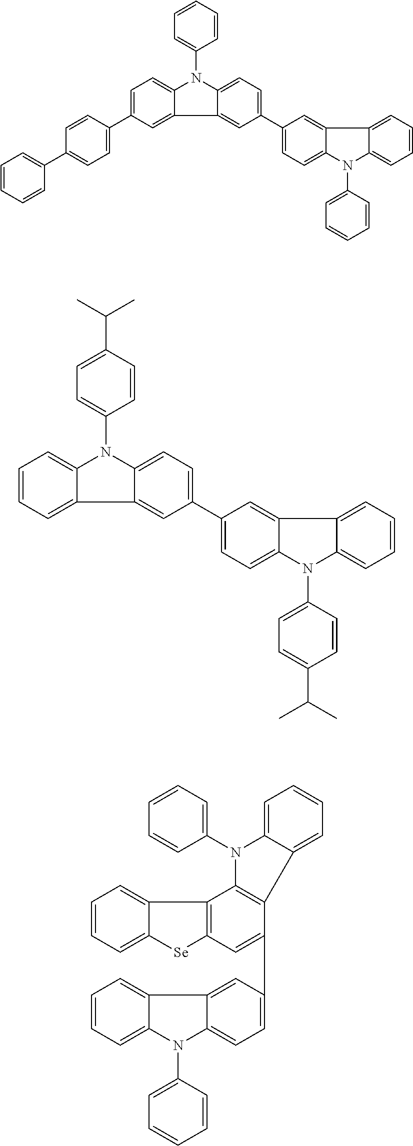 [SMILES: c1ccc(-c2ccc(-c3ccc4c(c3)c3cc(-c5ccc6c(c5)c5ccccc5n6-c5ccccc5)ccc3n4-c3ccccc3)cc2)cc1 ; CC(C)c1ccc(-n2c3ccccc3c3cc(-c4ccc5c(c4)c4ccccc4n5-c4ccc(C(C)C)cc4)ccc32)cc1 ; c1ccc(-n2c3ccccc3c3cc(-c4cc5[se]c6ccccc6c5c5c4c4ccccc4n5-c4ccccc4)ccc32)cc1]

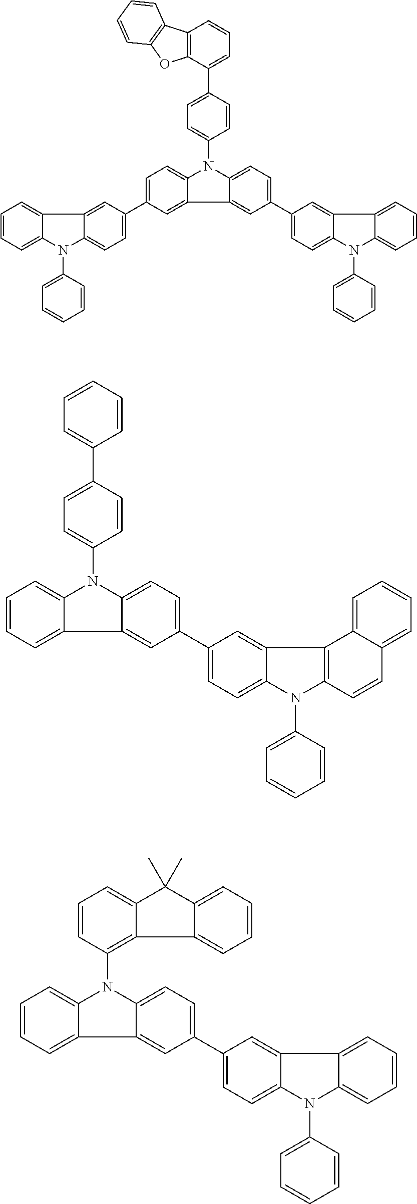 [SMILES: c1ccc(-n2c3ccccc3c3cc(-c4ccc5c(c4)c4cc(-c6ccc7c(c6)c6ccccc6n7-c6ccccc6)ccc4n5-c4ccc(-c5cccc6c5oc5ccccc56)cc4)ccc32)cc1 ; c1ccc(-c2ccc(-n3c4ccccc4c4cc(-c5ccc6c(c5)c5c7ccccc7ccc5n6-c5ccccc5)ccc43)cc2)cc1 ; CC1(C)c2ccccc2-c2c(-n3c4ccccc4c4cc(-c5ccc6c(c5)c5ccccc5n6-c5ccccc5)ccc43)cccc21]

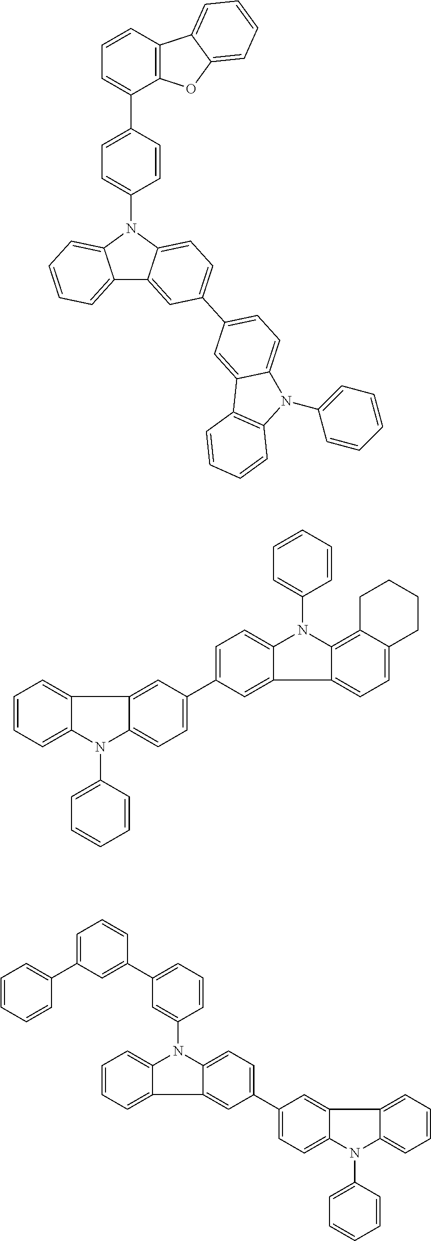 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccc6c(c4n5-c4ccccc4)CCCC6)ccc32)cc1 ; c1ccc(-c2cccc(-c3cccc(-n4c5ccccc5c5cc(-c6ccc7c(c6)c6ccccc6n7-c6ccccc6)ccc54)c3)c2)cc1]

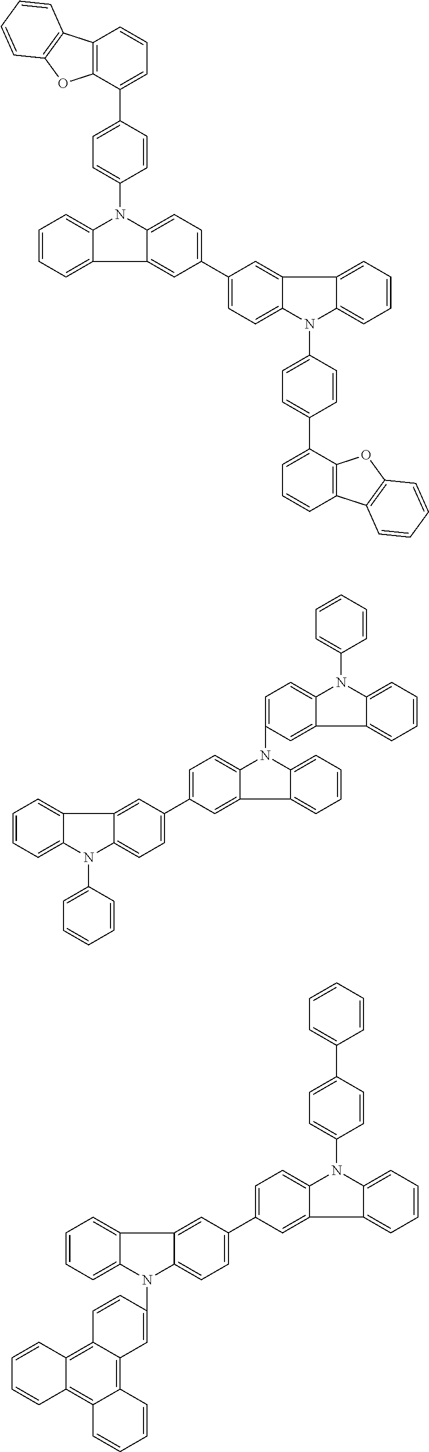 [SMILES: c1ccc2c(c1)oc1c(-c3ccc(-n4c5ccccc5c5cc(-c6ccc7c(c6)c6ccccc6n7-c6ccc(-c7cccc8c7oc7ccccc78)cc6)ccc54)cc3)cccc12 ; c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1]

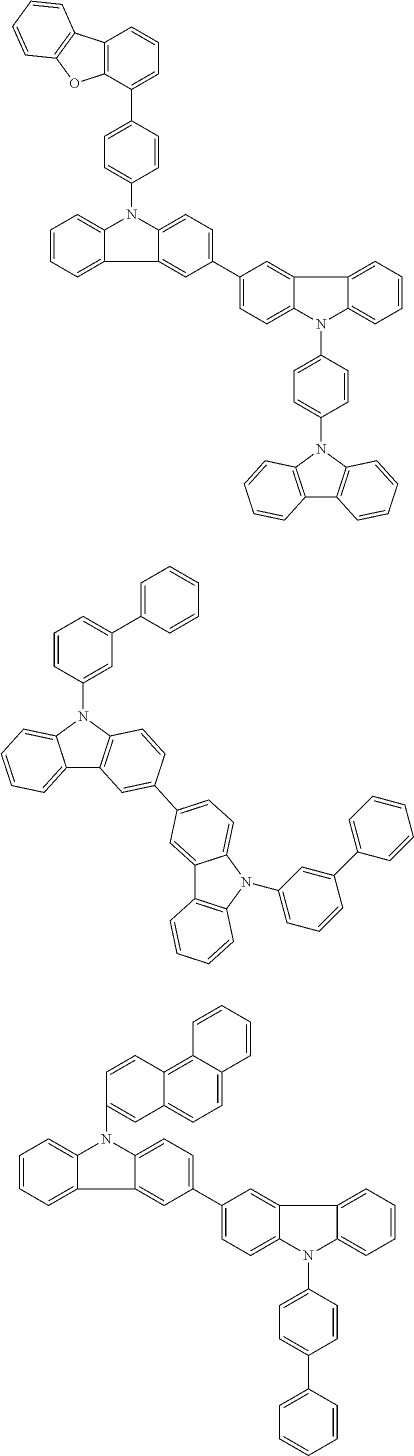

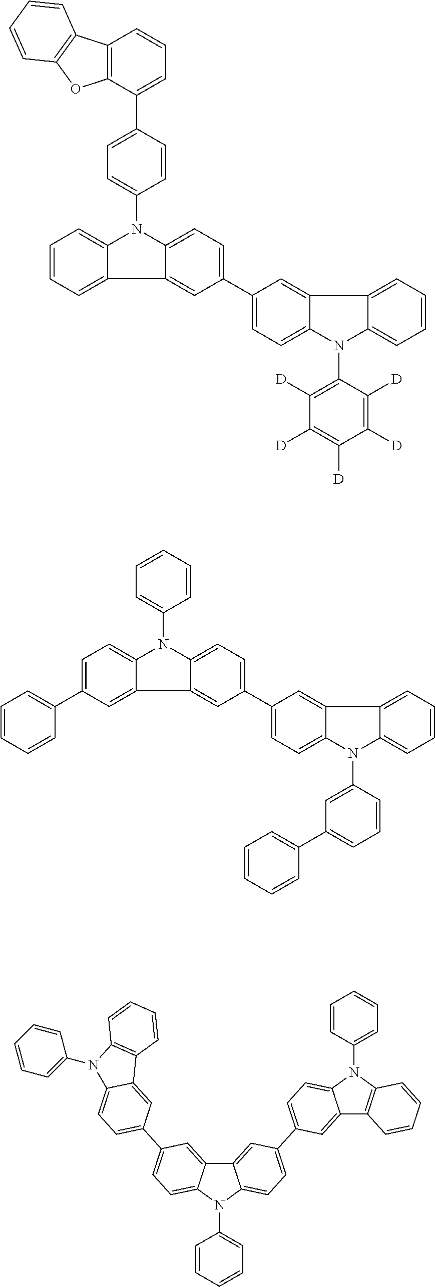 [SMILES: [2H]c1c([2H])c([2H])c(-n2c3ccccc3c3cc(-c4ccc5c(c4)c4ccccc4n5-c4ccc(-c5cccc6c5oc5ccccc56)cc4)ccc32)c([2H])c1[2H] ; c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

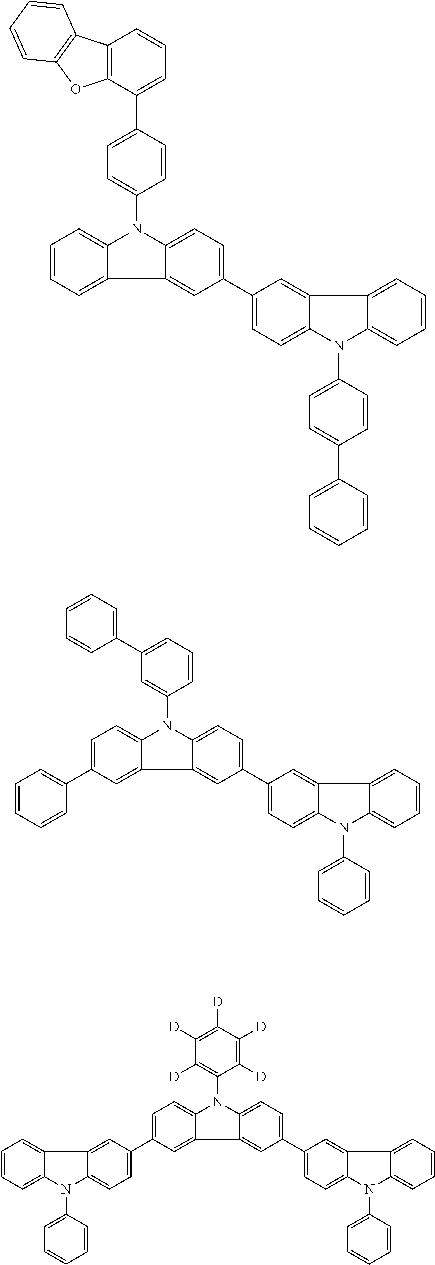 [SMILES: c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; [2H]c1c([2H])c([2H])c(-n2c3ccc(-c4ccc5c(c4)c4ccccc4n5-c4ccccc4)cc3c3cc(-c4ccc5c(c4)c4ccccc4n5-c4ccccc4)ccc32)c([2H])c1[2H]]

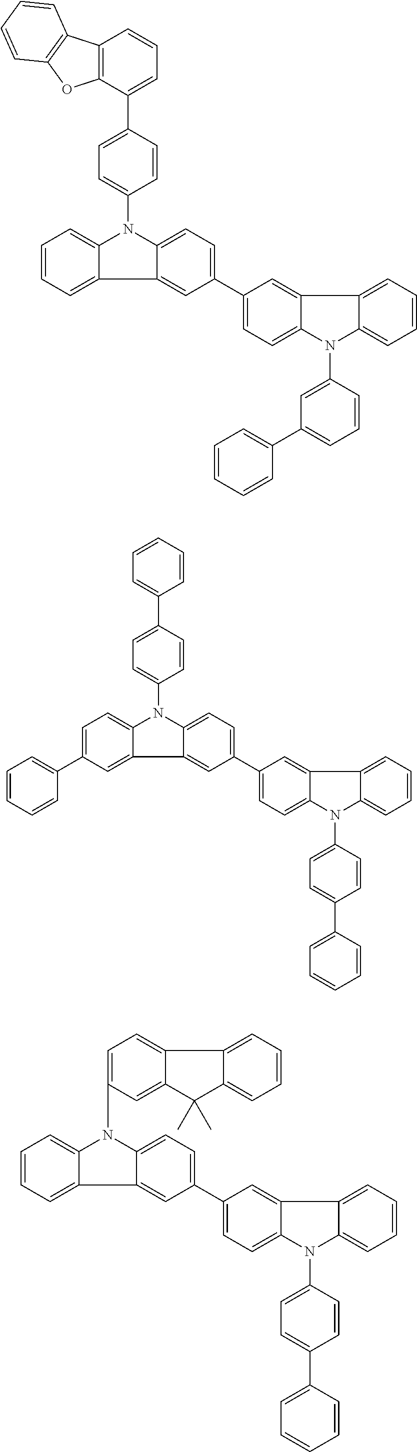 [SMILES: c1ccc(-c2cccc(-n3c4ccccc4c4cc(-c5ccc6c(c5)c5ccccc5n6-c5ccc(-c6cccc7c6oc6ccccc67)cc5)ccc43)c2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

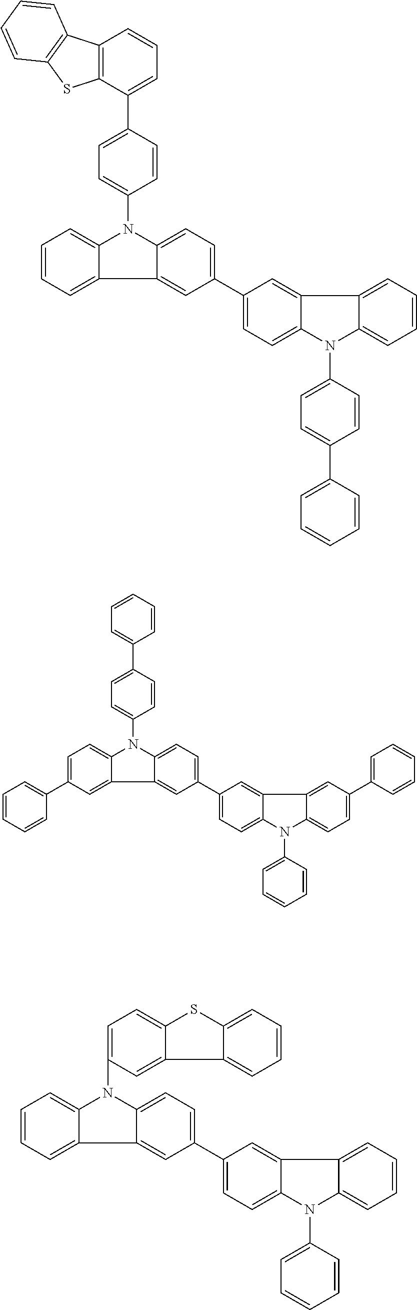 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4ccc5sc6ccccc6c5c4)ccc32)cc1]

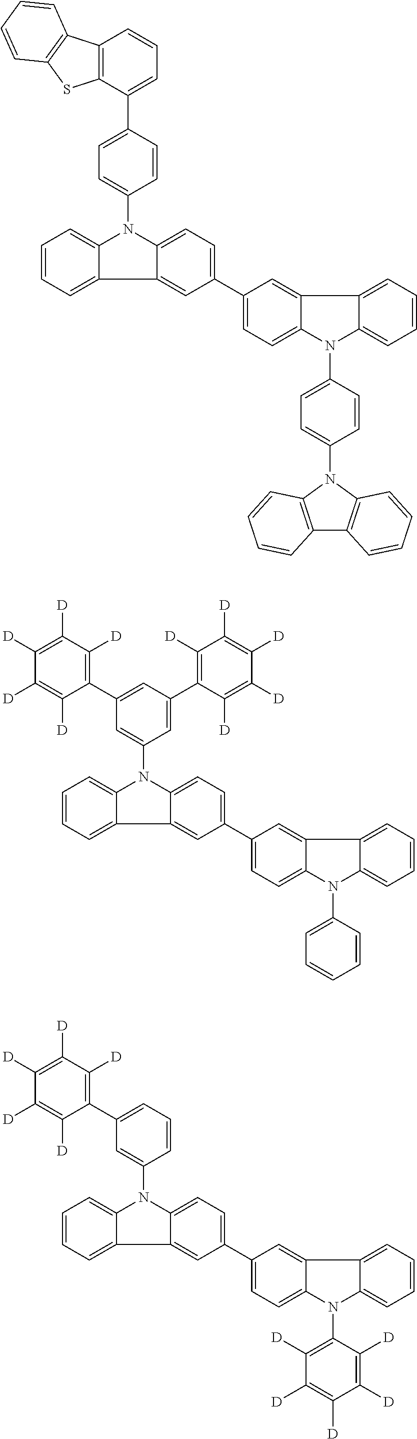 [SMILES: c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12 ; [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H] ; [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H]]

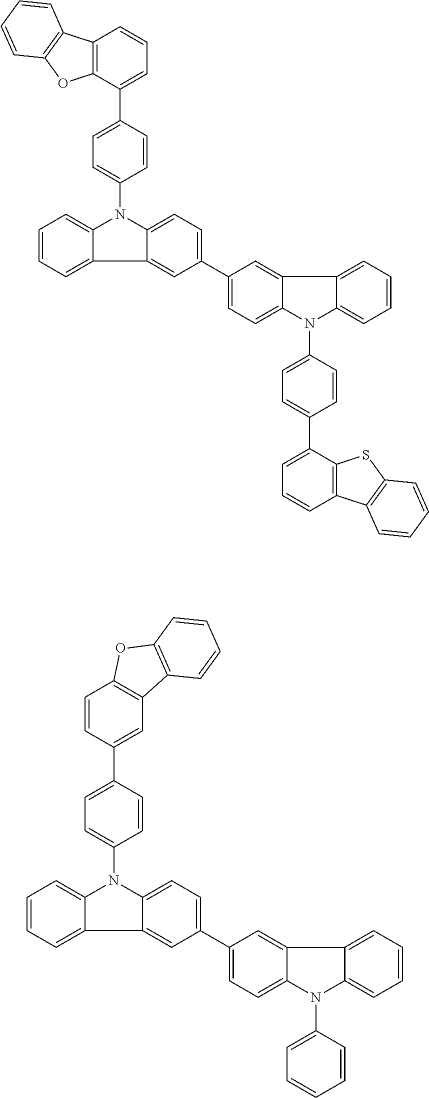 [SMILES: c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12 ; c1ccc(-n2c3ccccc3c3cc(-c4ccc5c(c4)c4ccccc4n5-c4ccc(-c5ccc6oc7ccccc7c6c5)cc4)ccc32)cc1]

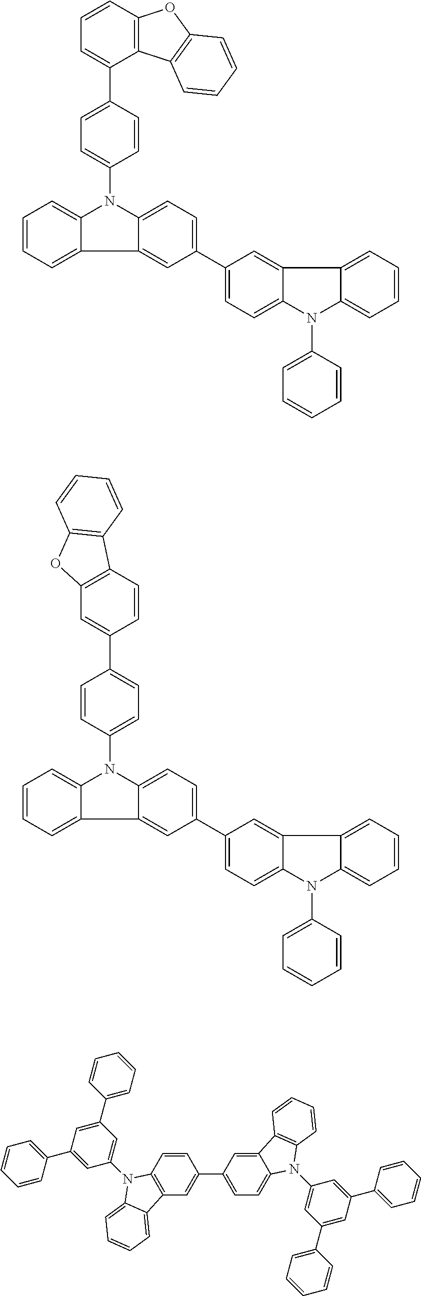 [SMILES: c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4ccc(-c5ccc6c(c5)oc5ccccc56)cc4)ccc32)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1]

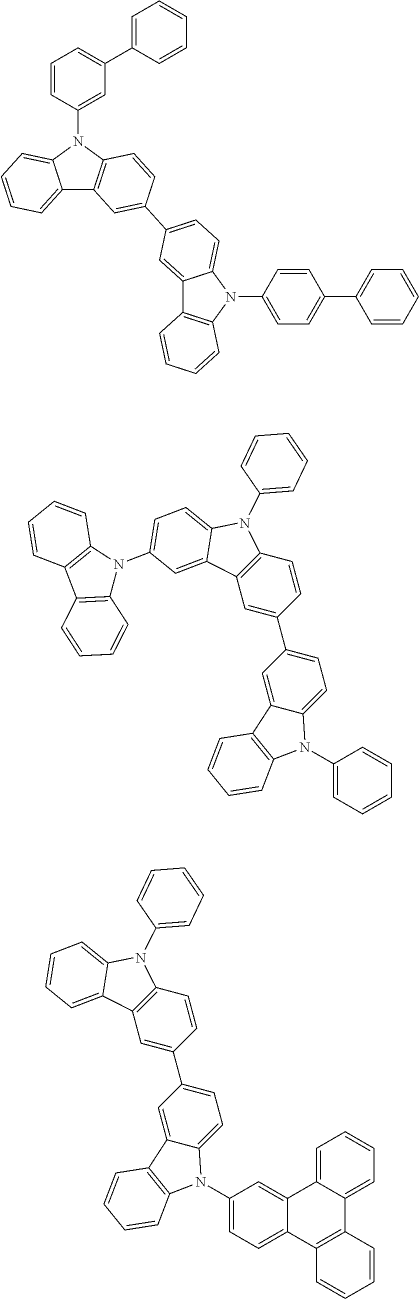 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3cc(-c4ccc5c(c4)c4cc(-n6c7ccccc7c7ccccc76)ccc4n5-c4ccccc4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

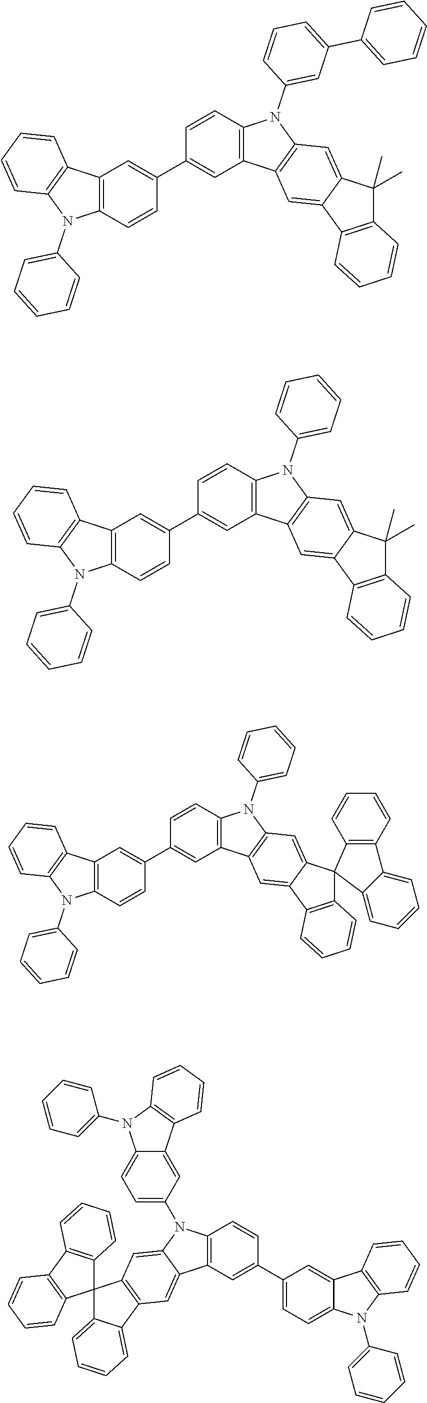 [SMILES: CC1(C)c2ccccc2-c2cc3c4cc(-c5ccc6c(c5)c5ccccc5n6-c5ccccc5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4ccccc4)c3cc21 ; c1ccc(-n2c3ccccc3c3cc(-c4ccc5c(c4)c4cc6c(cc4n5-c4ccccc4)C4(c5ccccc5-c5ccccc54)c4ccccc4-6)ccc32)cc1 ; c1ccc(-n2c3ccccc3c3cc(-c4ccc5c(c4)c4cc6c(cc4n5-c4ccc5c(c4)c4ccccc4n5-c4ccccc4)C4(c5ccccc5-c5ccccc54)c4ccccc4-6)ccc32)cc1]

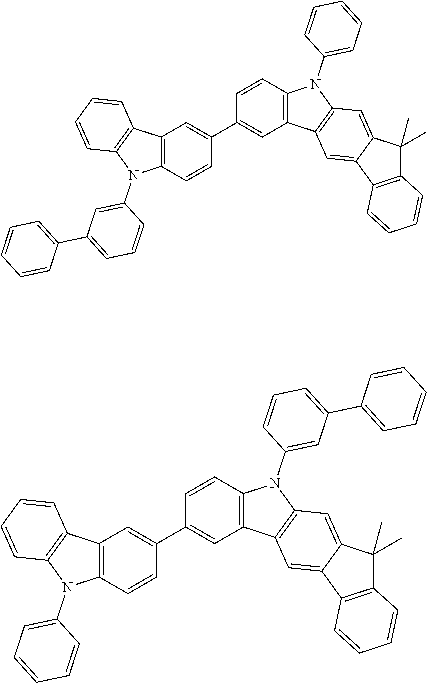 [SMILES: CC1(C)c2ccccc2-c2cc3c4cc(-c5ccc6c(c5)c5ccccc5n6-c5cccc(-c6ccccc6)c5)ccc4n(-c4ccccc4)c3cc21 ; CC1(C)c2ccccc2-c2cc3c4cc(-c5ccc6c(c5)c5ccccc5n6-c5ccccc5)ccc4n(-c4cccc(-c5ccccc5)c4)c3cc21]

Preferred bridged carbazoles are the structures of the following formula (21):

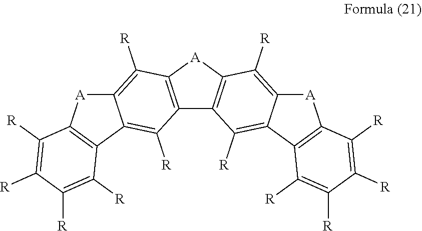

where A and R have the definitions given above and A is preferably the same or different at each instance and is selected from the group consisting of NAr² and CR₂.

A preferred embodiment of the formula (21) is the compounds of the formula (21a), particular preference being given to the compounds of the formula (21b), and very particular preference to the compounds of the formula (21c)

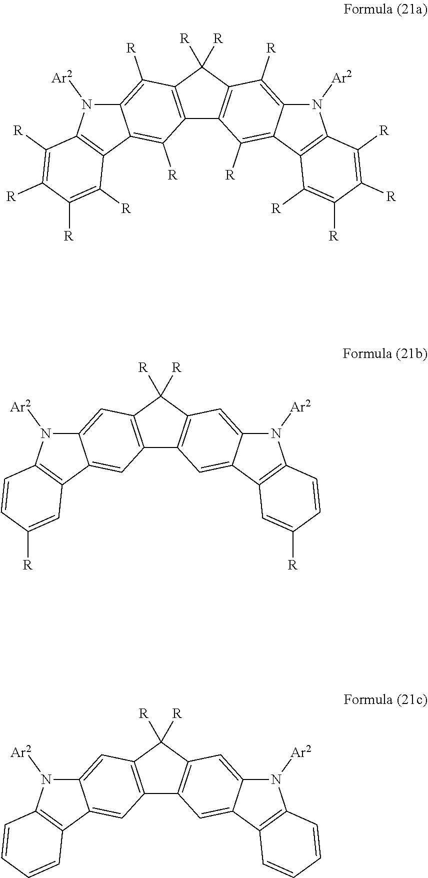

where Ar² and R have the definitions given above. Ar² here is preferably the same or different at each instance and is an aromatic ring system having 6 to 18 aromatic ring atoms, for example phenyl, ortho-, meta- or para-biphenyl or terphenyl. The R radical on the indene carbon atom is preferably the same or different at each instance and is an alkyl group having 1 to 5 carbon atoms, especially methyl, or an aromatic ring system having 6 to 18 aromatic ring atoms, especially phenyl.

Preferred triarylamines are the structures of the following formula (22):

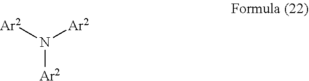

where Ar² has the definitions given above.

Preferred dibenzofuran derivatives are the compounds of the following formula (23):

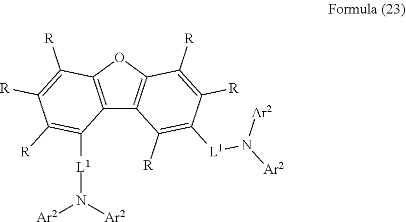

where the oxygen may also be replaced by sulfur so as to form a dibenzothiophene, L¹ is a single bond or an aromatic or heteroaromatic ring system which has 5 to 30 aromatic ring atoms and may also be substituted by one or more R radicals, and R and Ar² have the definitions given above. It is also possible here for the two Ar² groups that bind to the same nitrogen atom, or for one Ar² group and one L¹ group that bind to the same nitrogen atom, to be bonded to one another, for example to give a carbazole.

Preferred embodiments of the compounds of the formula (23) are the compounds of the following formulae (23a) and (23b):

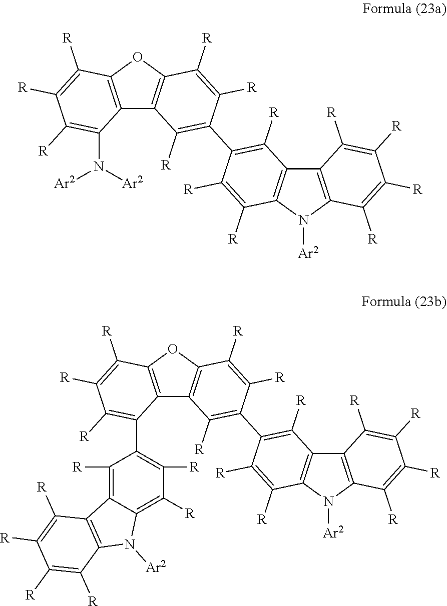

where the oxygen may also be replaced by sulfur so as to form a dibenzothiophene, and R and Ar² have the definitions given above, where two adjacent R radicals, especially on the carbazole, here may also together form an aromatic or heteroaromatic ring system.

Particularly preferred embodiments are the compounds of the following formulae (23c) and (23d):

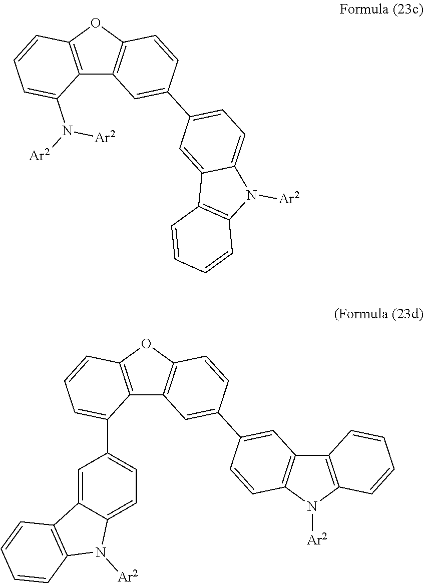

where the oxygen may also be replaced by sulfur so as to form a dibenzothiophene, and Ar² has the definitions given above, where two adjacent R radicals, especially on the carbazole, here may also together form an aromatic or heteroaromatic ring system.

Examples of suitable dibenzofuran derivatives are the compounds depicted below.

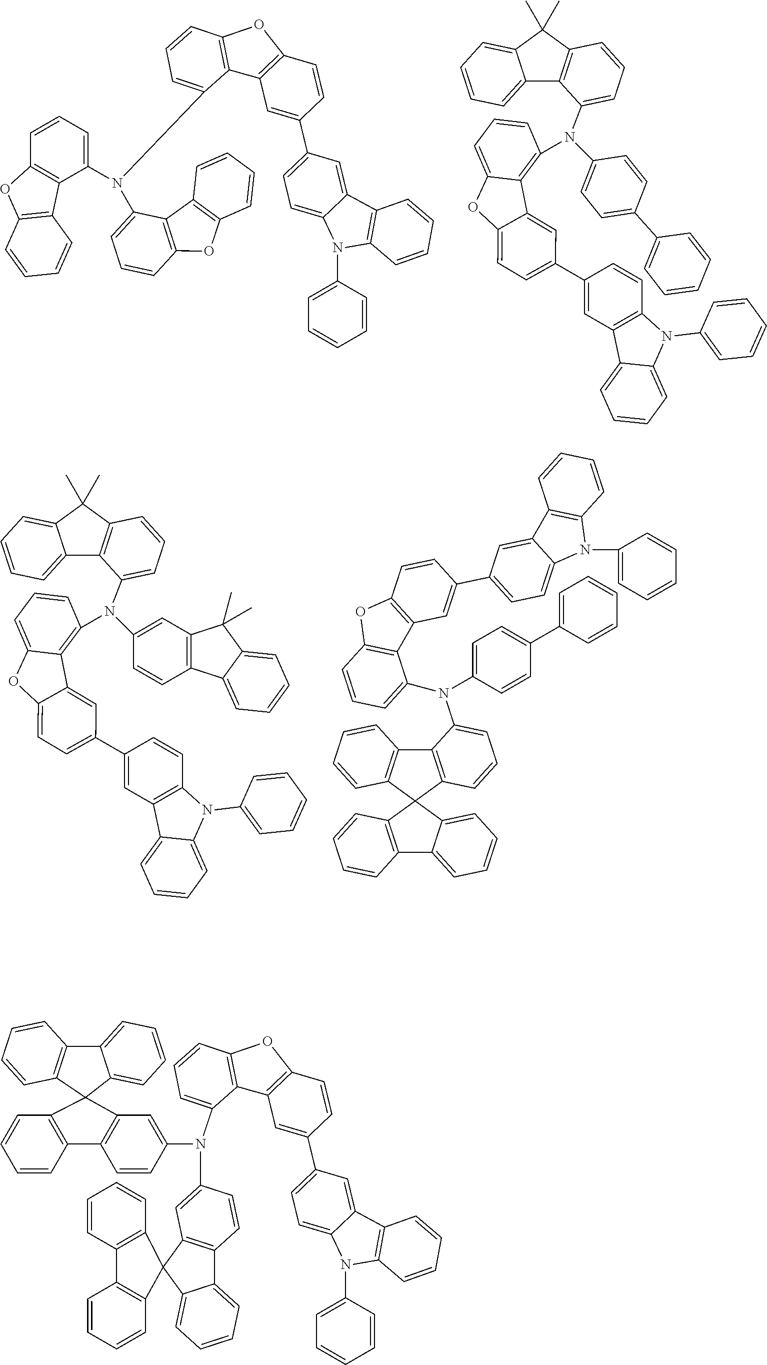

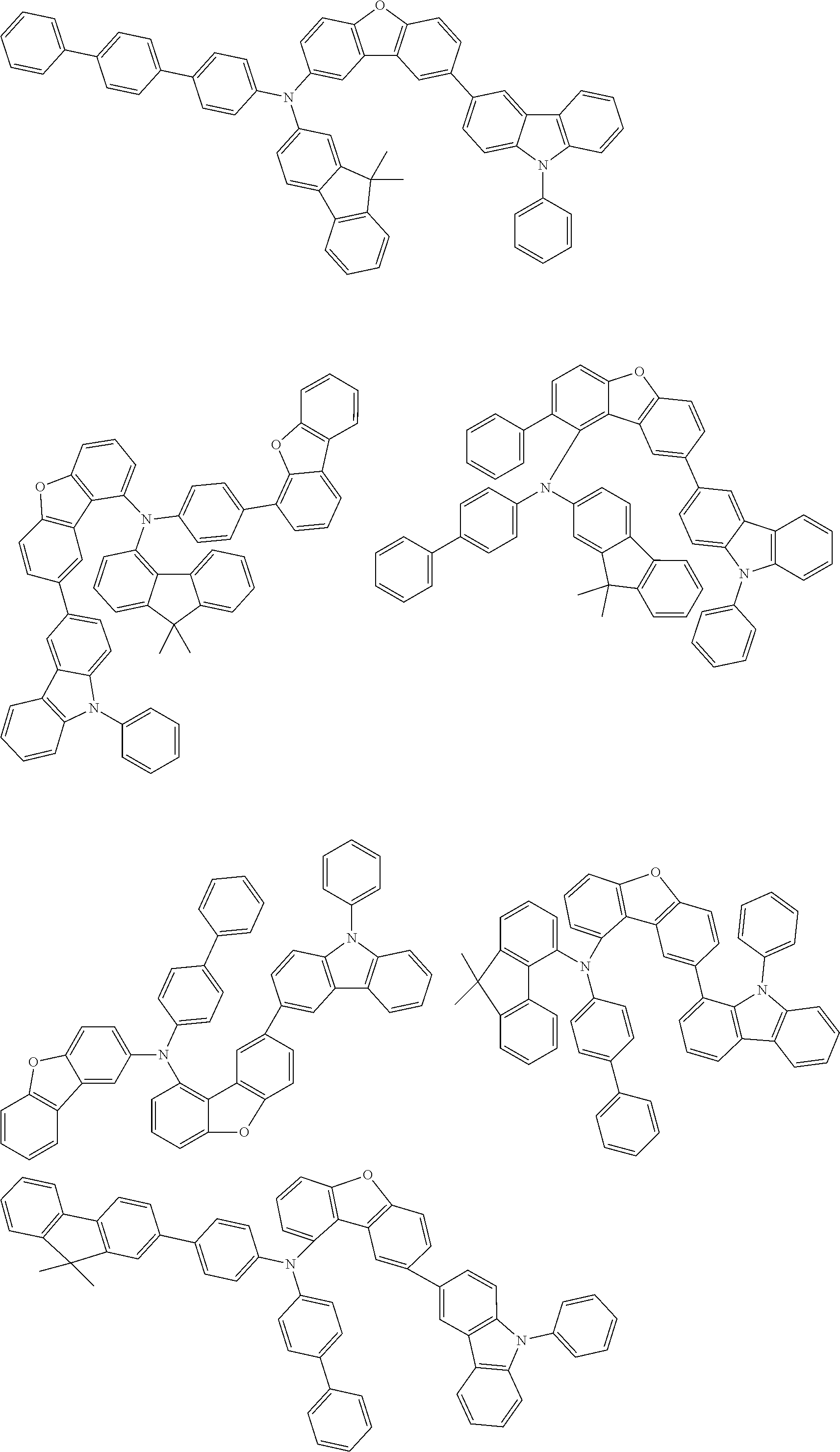

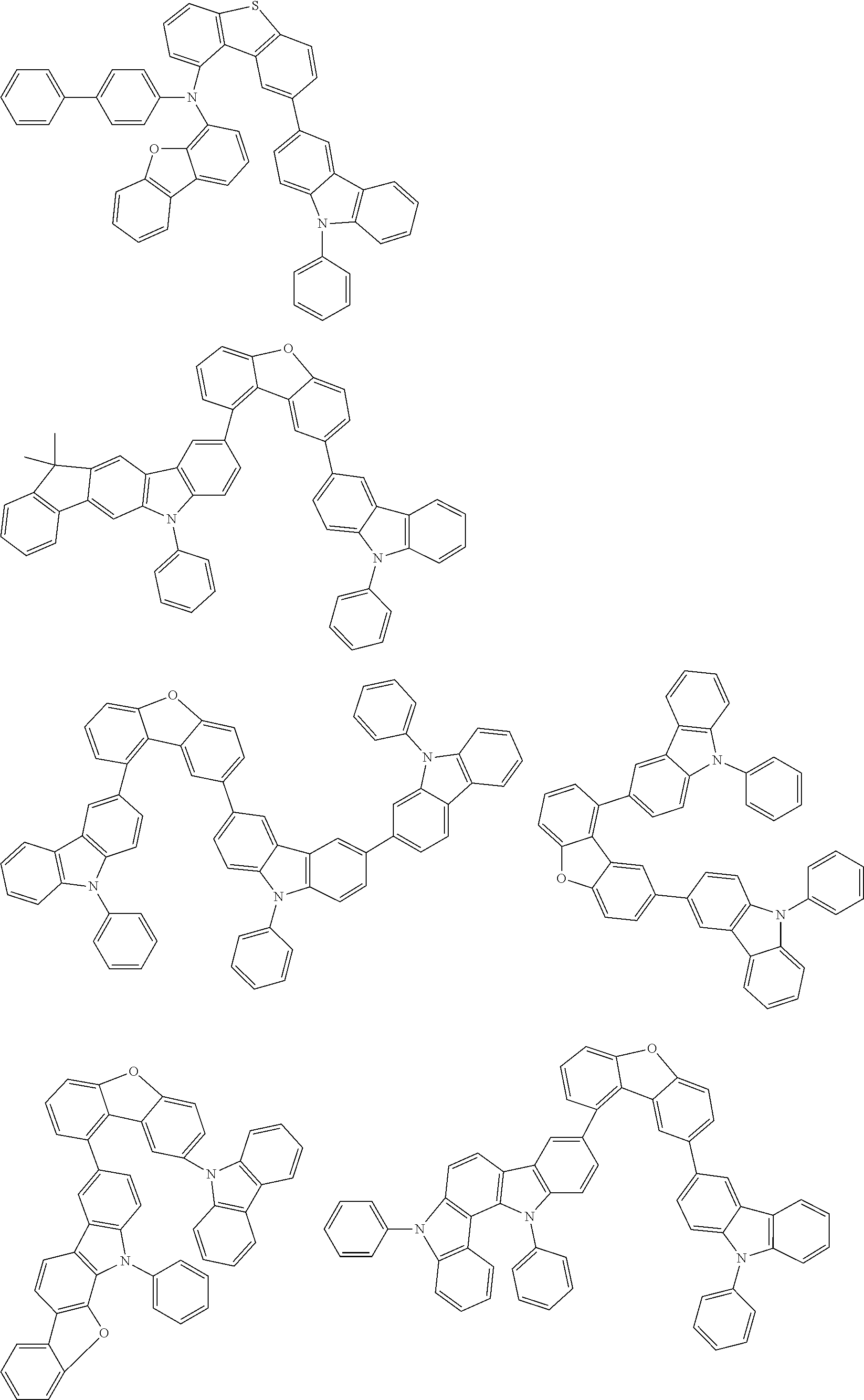

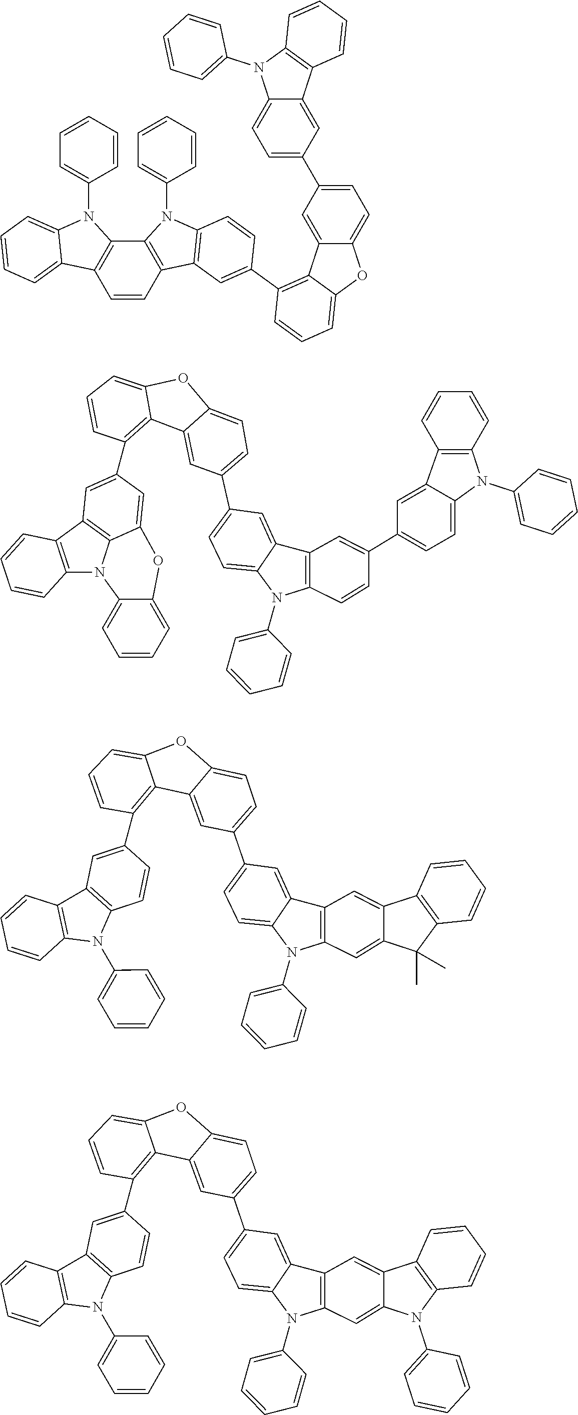 [SMILES: c1ccc(-n2c3ccccc3c3cc(-c4ccc5oc6cccc(-c7ccc8c(c7)c7ccc9c%10ccccc%10n(-c%10ccccc%10)c9c7n8-c7ccccc7)c6c5c4)ccc32)cc1 ; c1ccc(-n2c3ccccc3c3cc(-c4ccc5c(c4)c4cc(-c6ccc7oc8cccc(-c9cc%10c%11c(c9)c9ccccc9n%11-c9ccccc9O%10)c8c7c6)ccc4n5-c4ccccc4)ccc32)cc1 ; CC1(C)c2ccccc2-c2cc3c4cc(-c5ccc6oc7cccc(-c8ccc9c(c8)c8ccccc8n9-c8ccccc8)c7c6c5)ccc4n(-c4ccccc4)c3cc21 ; c1ccc(-n2c3ccccc3c3cc(-c4cccc5oc6ccc(-c7ccc8c(c7)c7cc9c%10ccccc%10n(-c%10ccccc%10)c9cc7n8-c7ccccc7)cc6c45)ccc32)cc1]

Preferred carbazoleamines are the structures of the following formulae (24), (25) and (26):

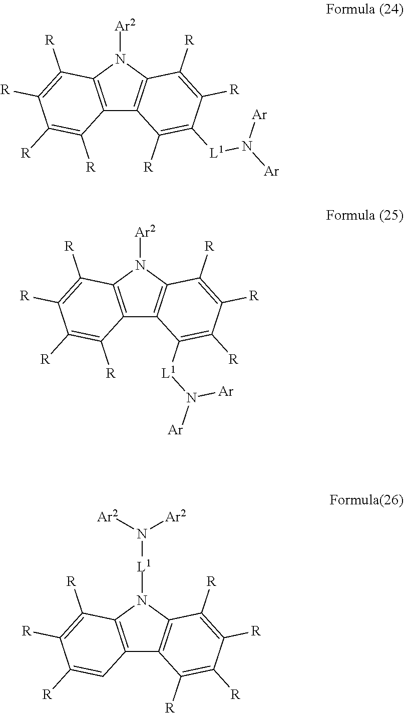

where L¹ is an aromatic or heteroaromatic ring system which has 5 to 30 aromatic ring atoms and may be substituted by one or more R radicals, and R and Ar² have the definitions given above, where two adjacent R radicals here too may form an aromatic ring system.

Examples of suitable carbazoleamine derivatives are the compounds depicted below.

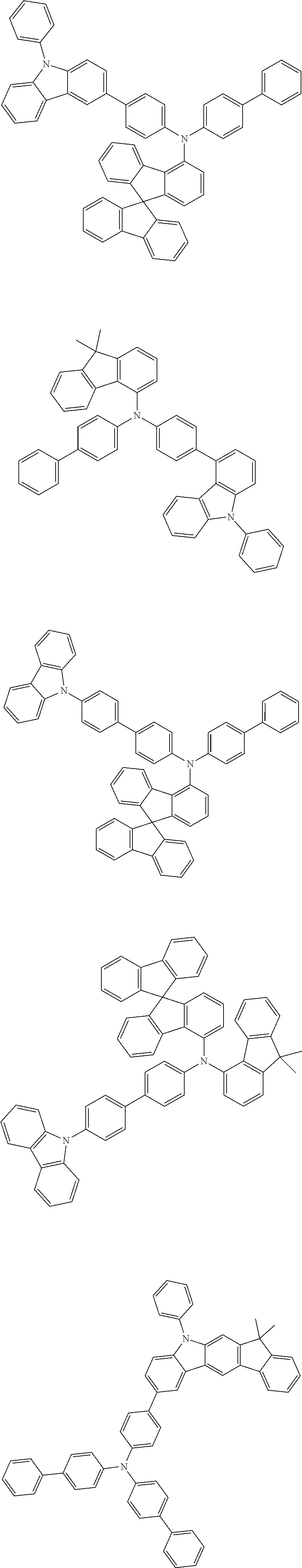

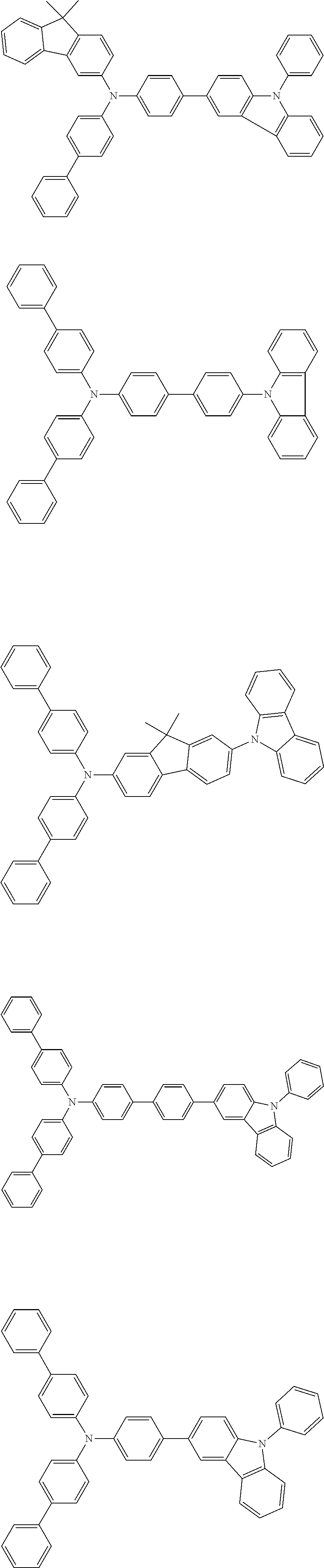 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

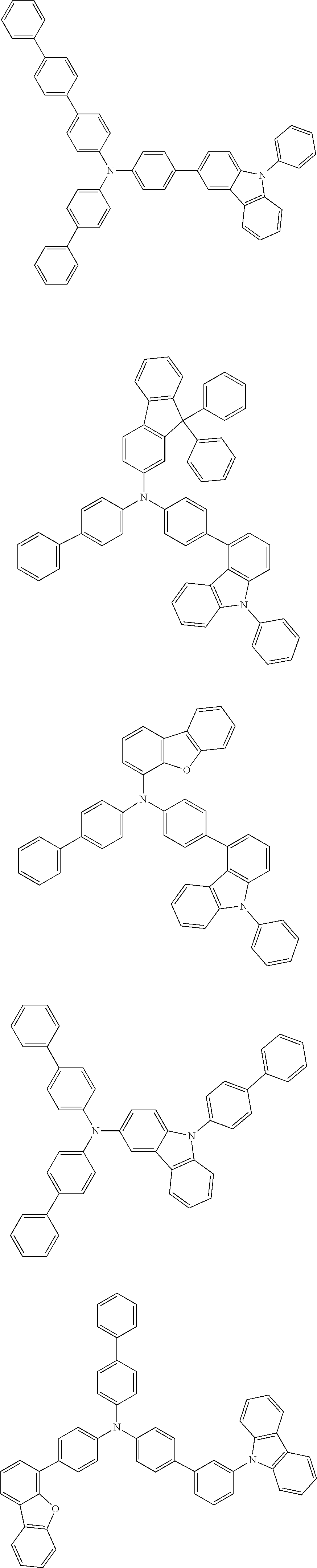 [SMILES: c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccc(N(c3ccc(-c4cccc5c4c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4cccc5c4c4ccccc4n5-c4ccccc4)cc3)c3cccc4c3oc3ccccc34)cc2)cc1 ; c1ccc(-c2ccc(N(c3ccc(-c4ccccc4)cc3)c3ccc4c(c3)c3ccccc3n4-c3ccc(-c4ccccc4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

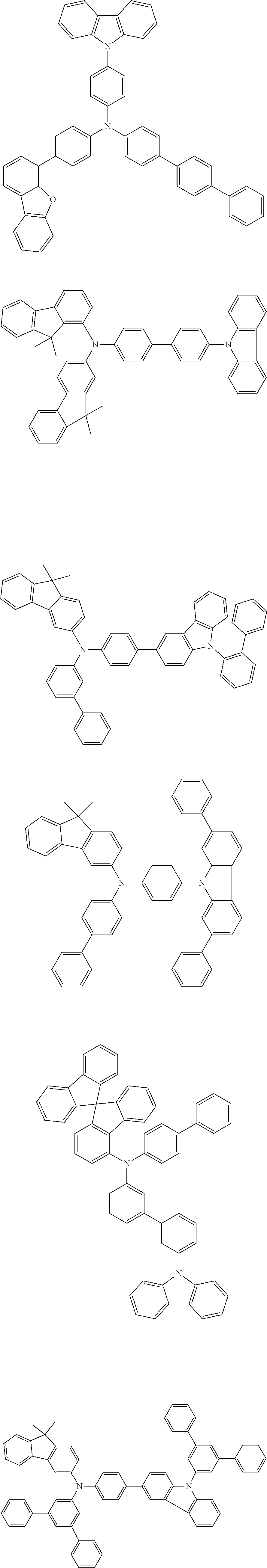

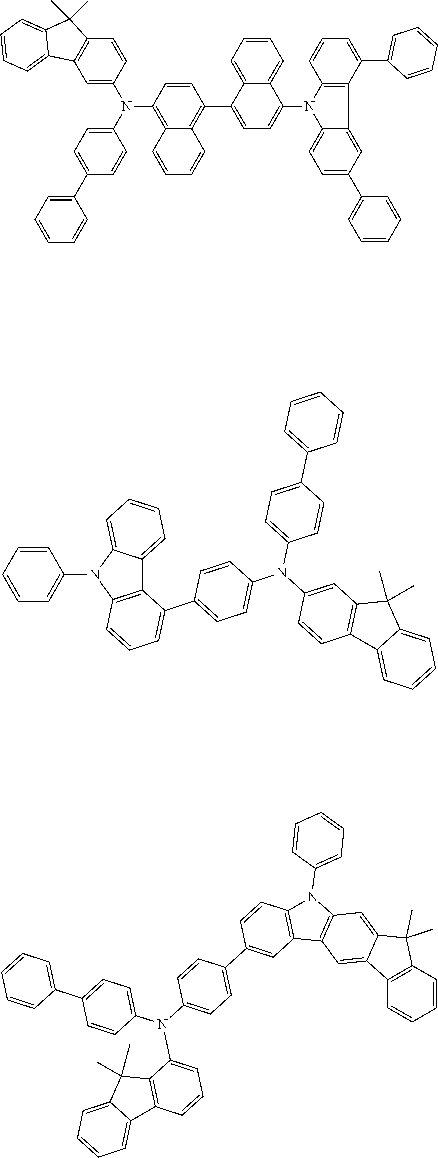 [SMILES: CC1(C)c2ccccc2-c2cc(N(c3ccc(-c4ccccc4)cc3)c3ccc(-c4ccc(-n5c6ccc(-c7ccccc7)cc6c6c(-c7ccccc7)cccc65)c5ccccc45)c4ccccc34)ccc21 ; CC1(C)c2ccccc2-c2ccc(N(c3ccc(-c4ccccc4)cc3)c3ccc(-c4cccc5c4c4ccccc4n5-c4ccccc4)cc3)cc21 ; CC1(C)c2ccccc2-c2cc3c4cc(-c5ccc(N(c6ccc(-c7ccccc7)cc6)c6cccc7c6C(C)(C)c6ccccc6-7)cc5)ccc4n(-c4ccccc4)c3cc21]

Preferred triazine or pyrimidine derivatives that can be used as a mixture together with the compounds of the invention are the compounds of the following formulae (27) and (28):

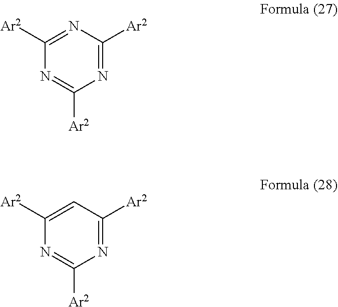

where Ar² has the definitions given above. The preferred embodiments detailed above for Ar² are also applicable here to the compounds of the formulae (27) and (28). Particular preference is given to the triazine derivatives of the formula (27).

Examples of triazine derivatives that may be used as matrix materials together with the compounds of the invention are the compounds listed below:

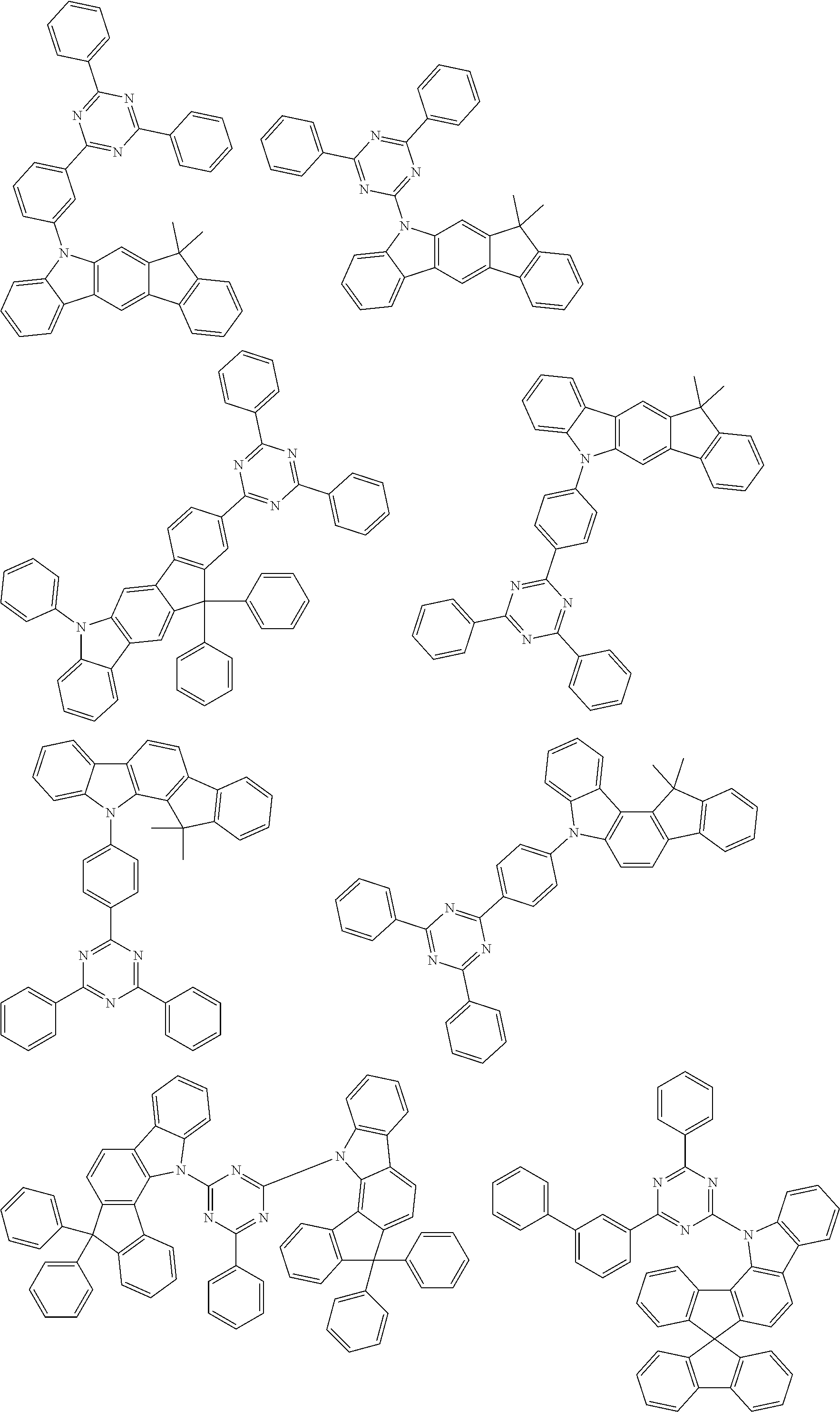

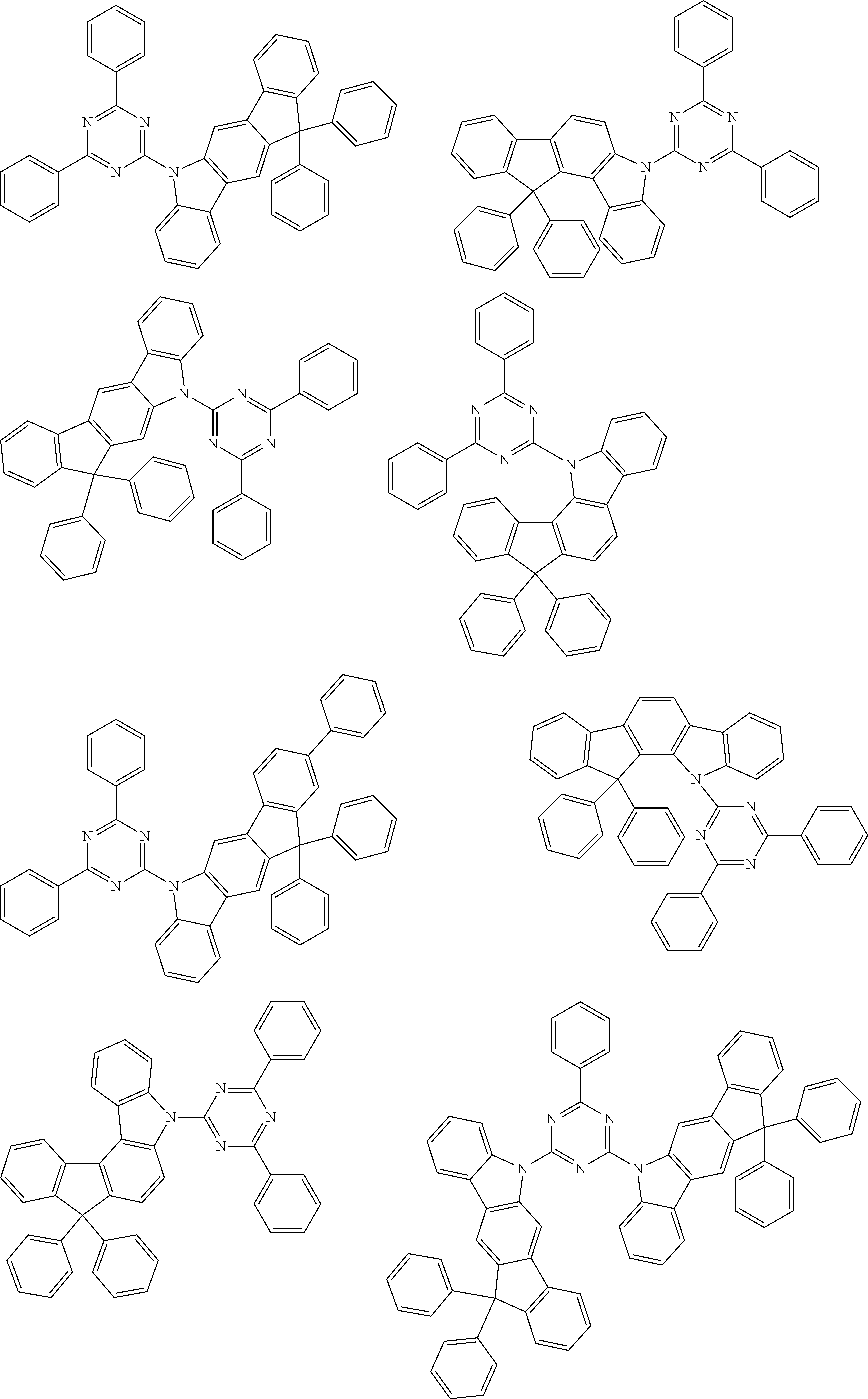

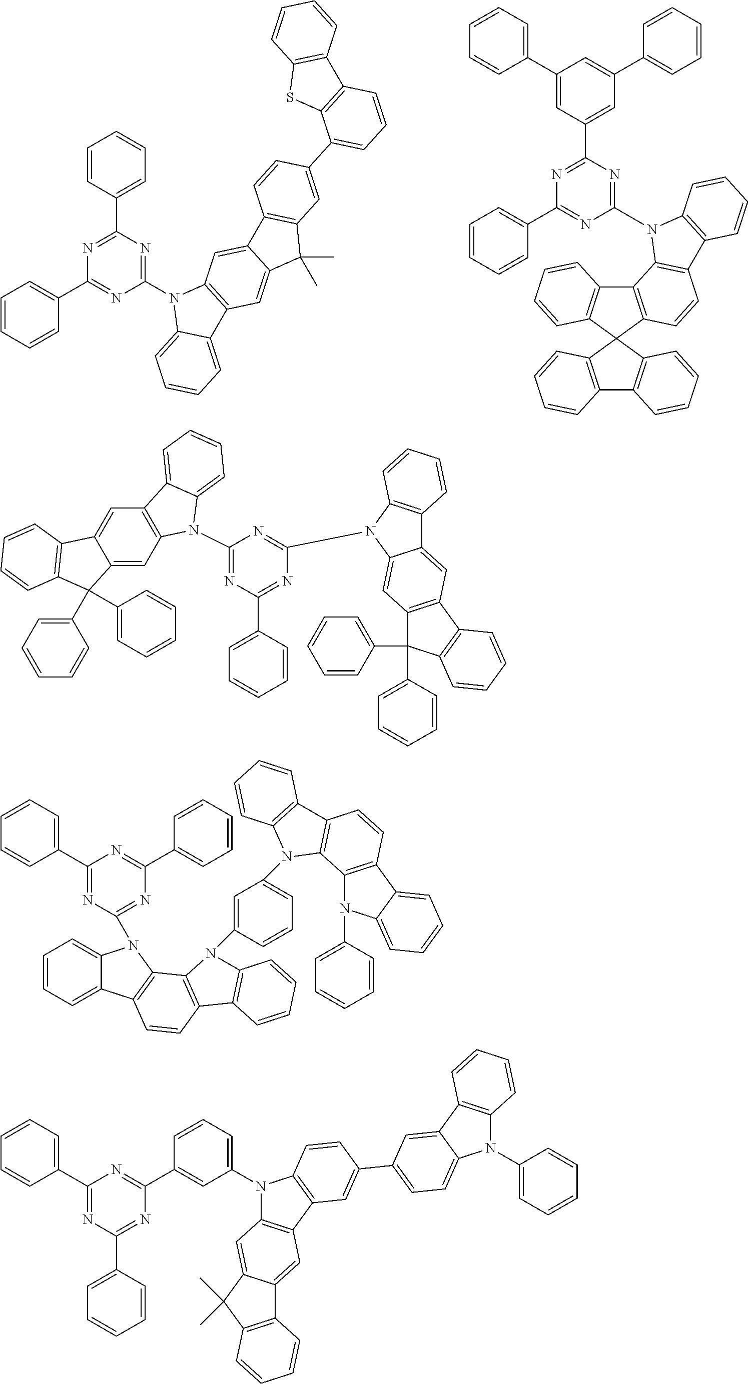

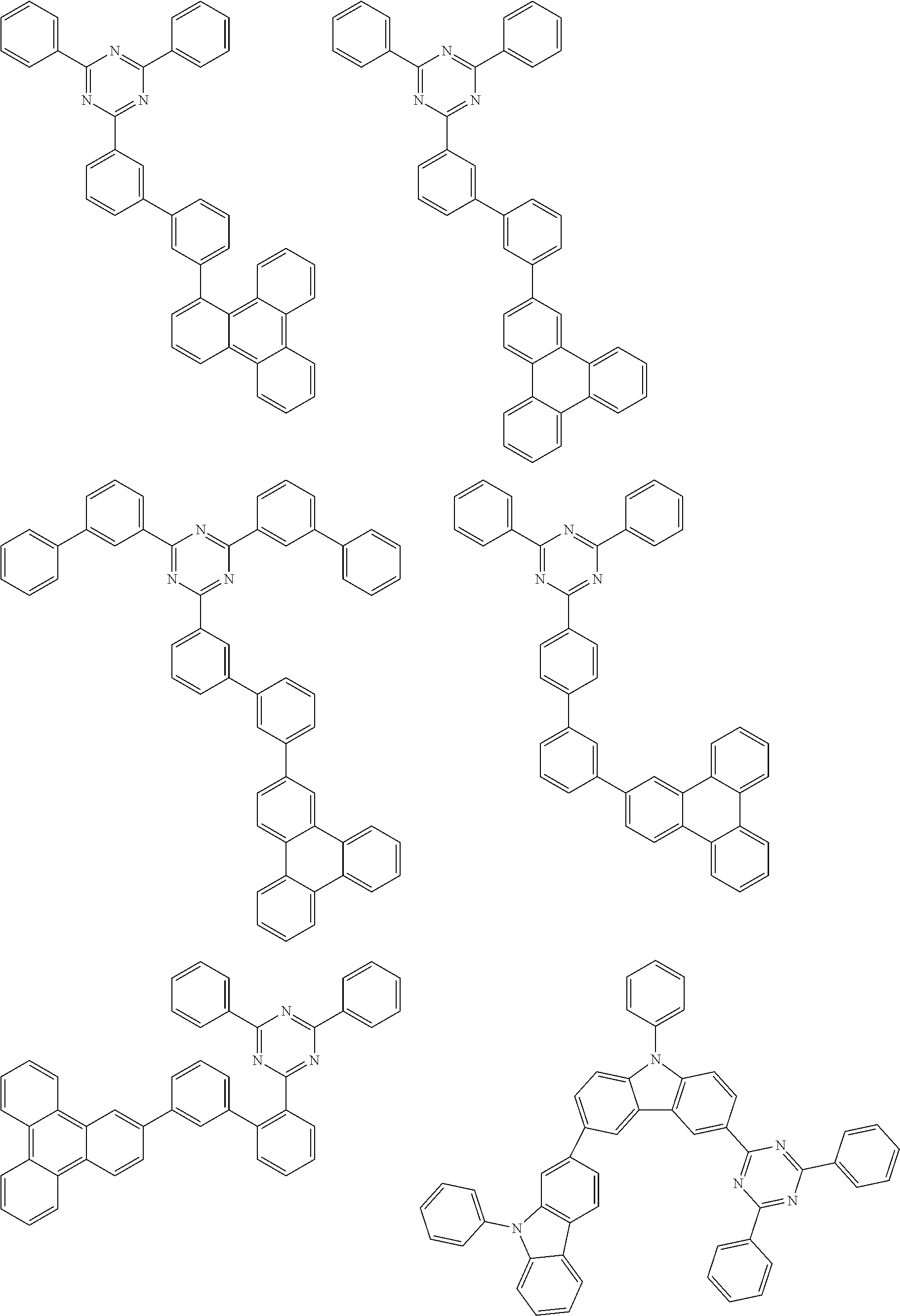

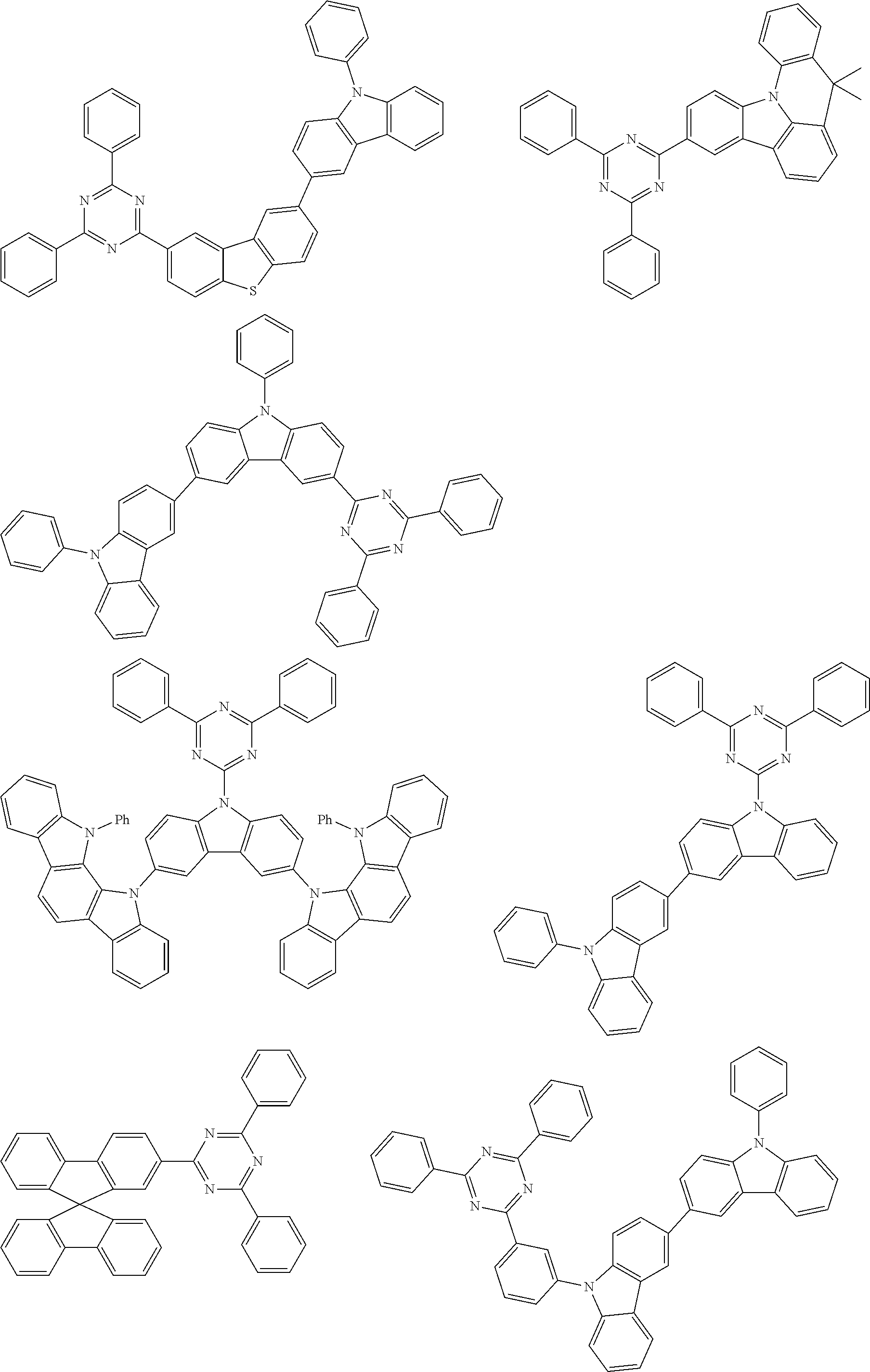

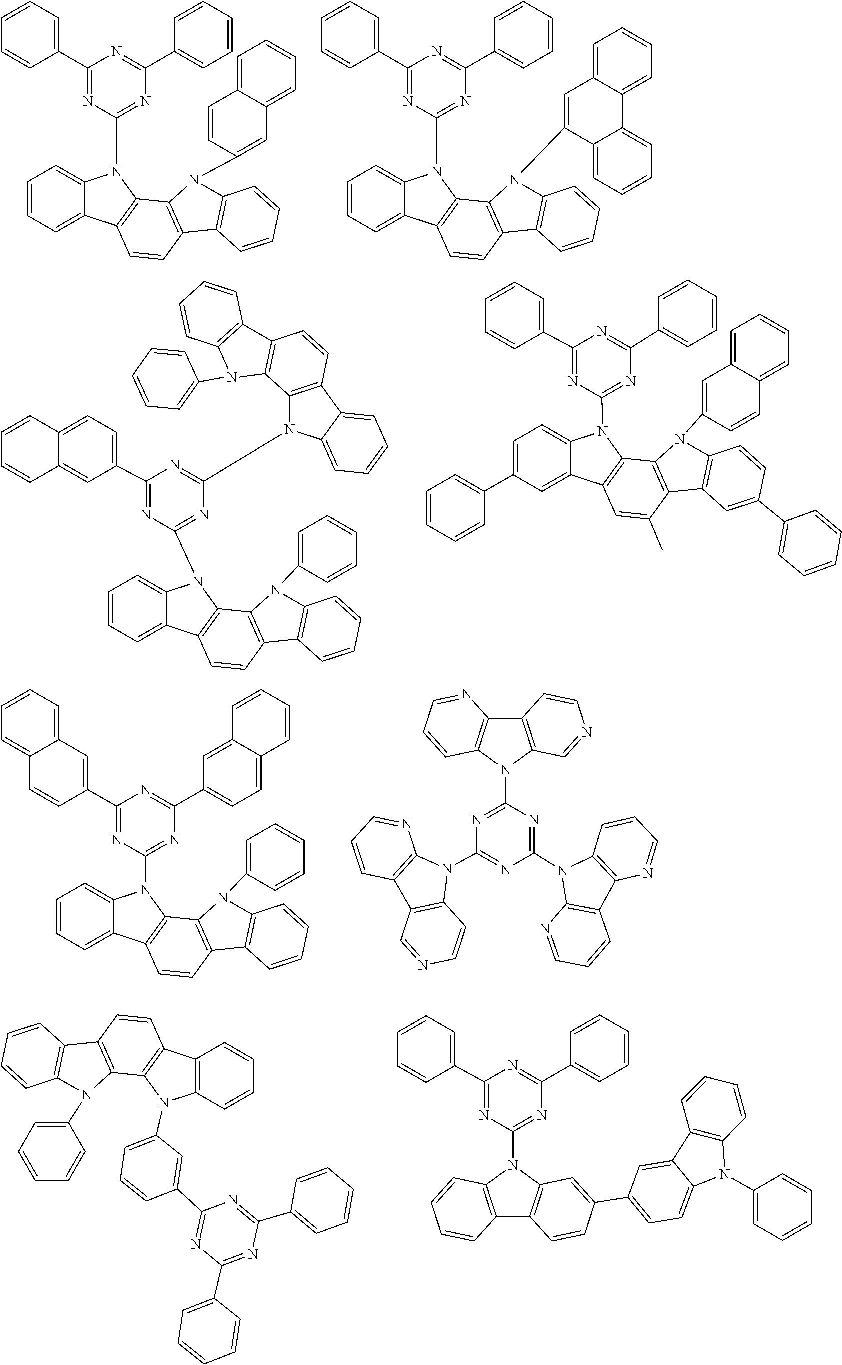

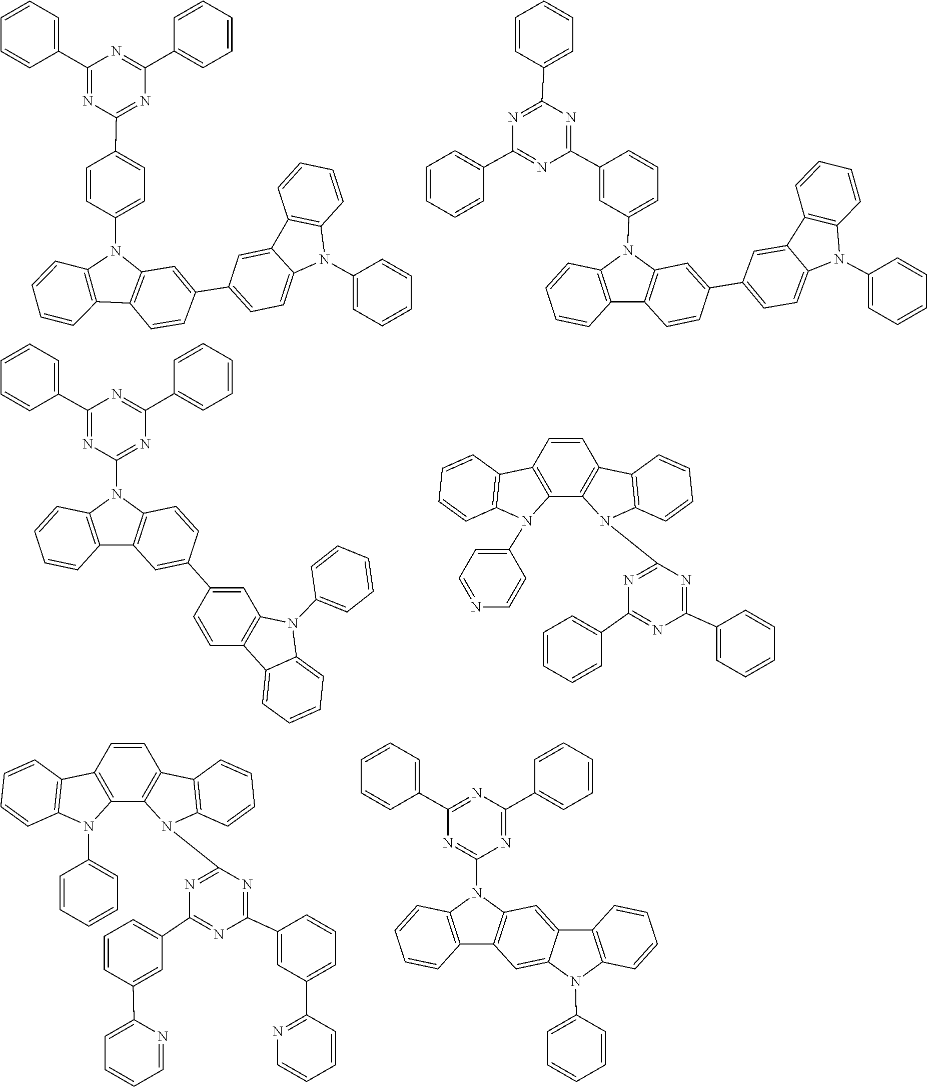

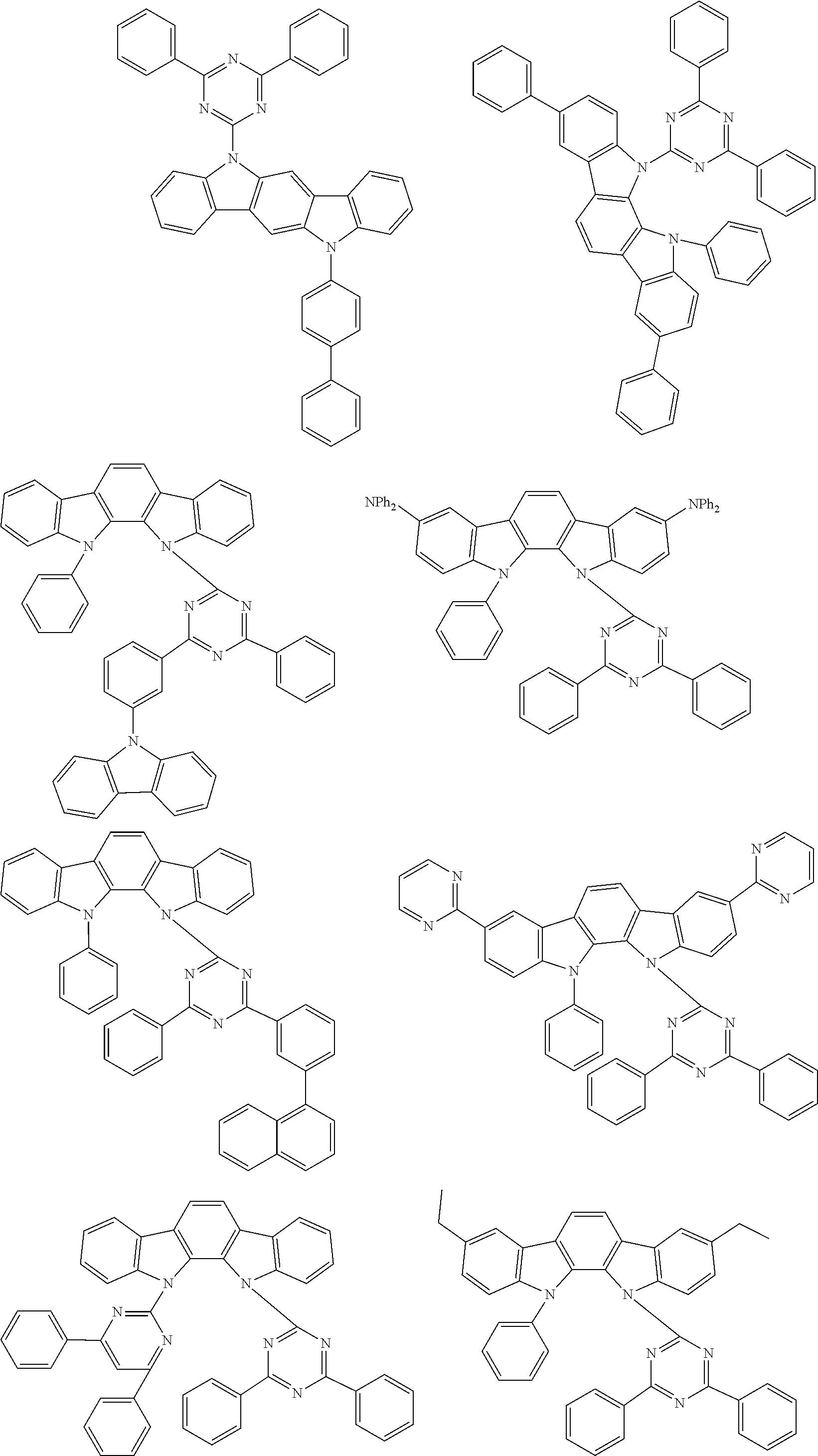

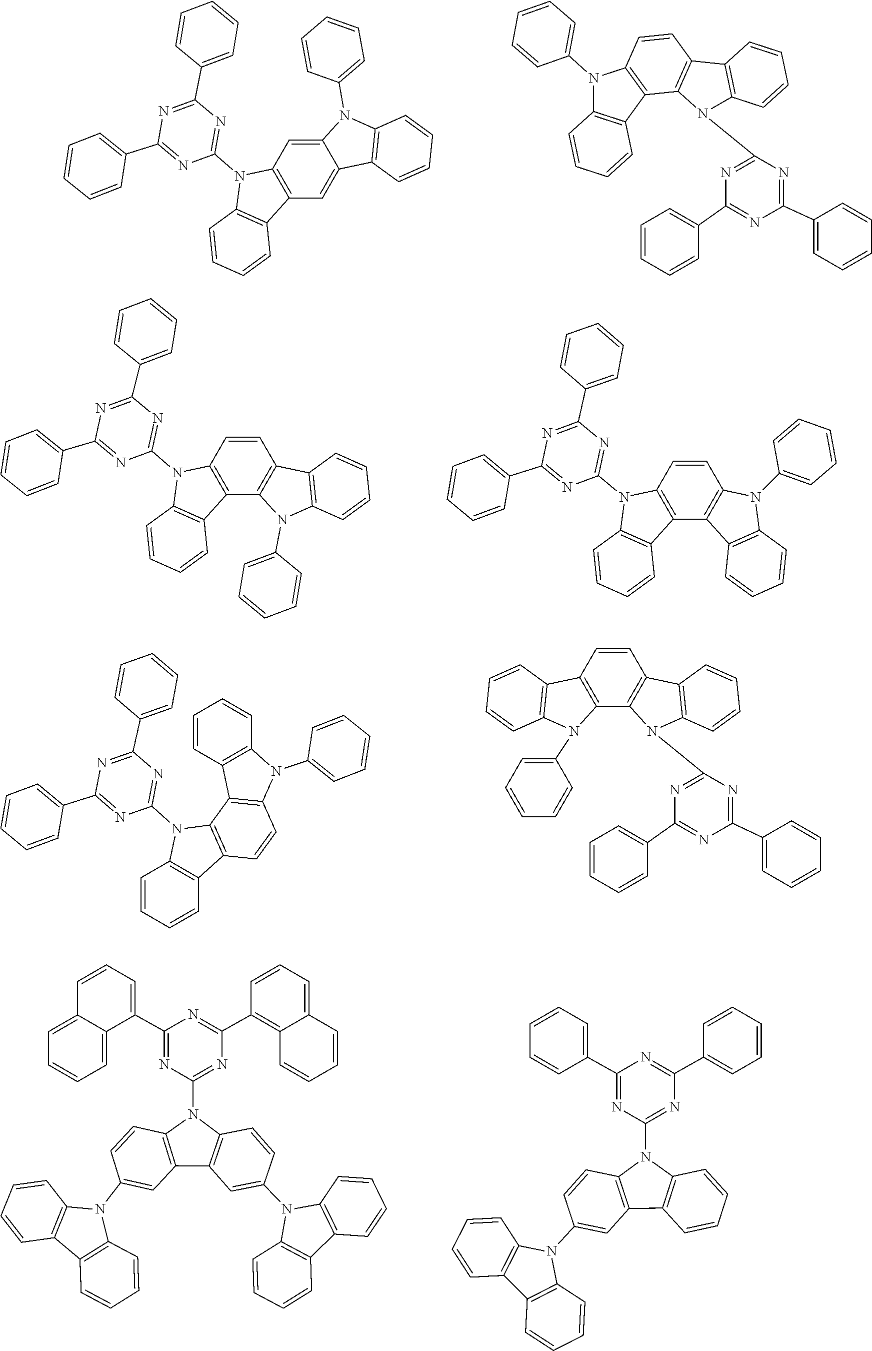

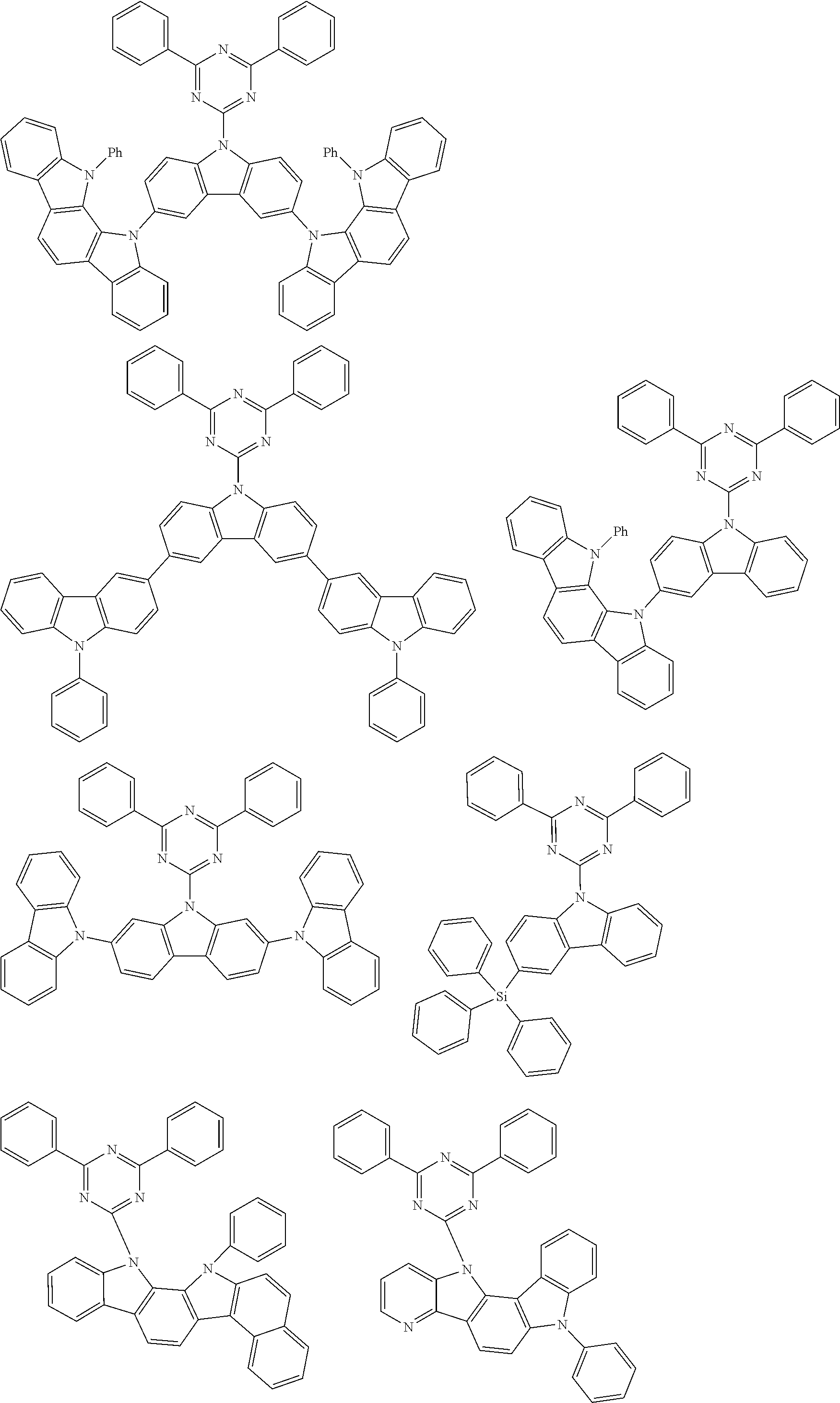

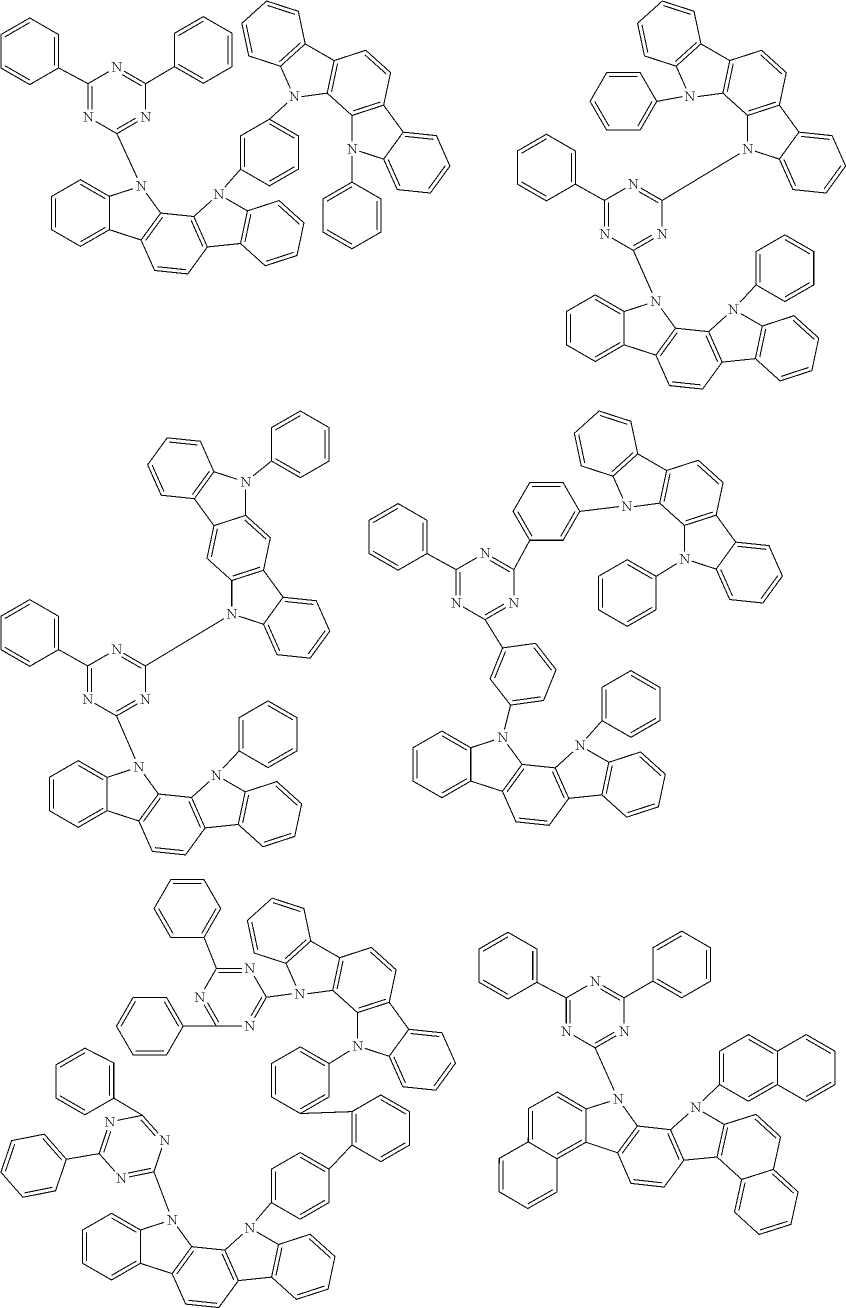

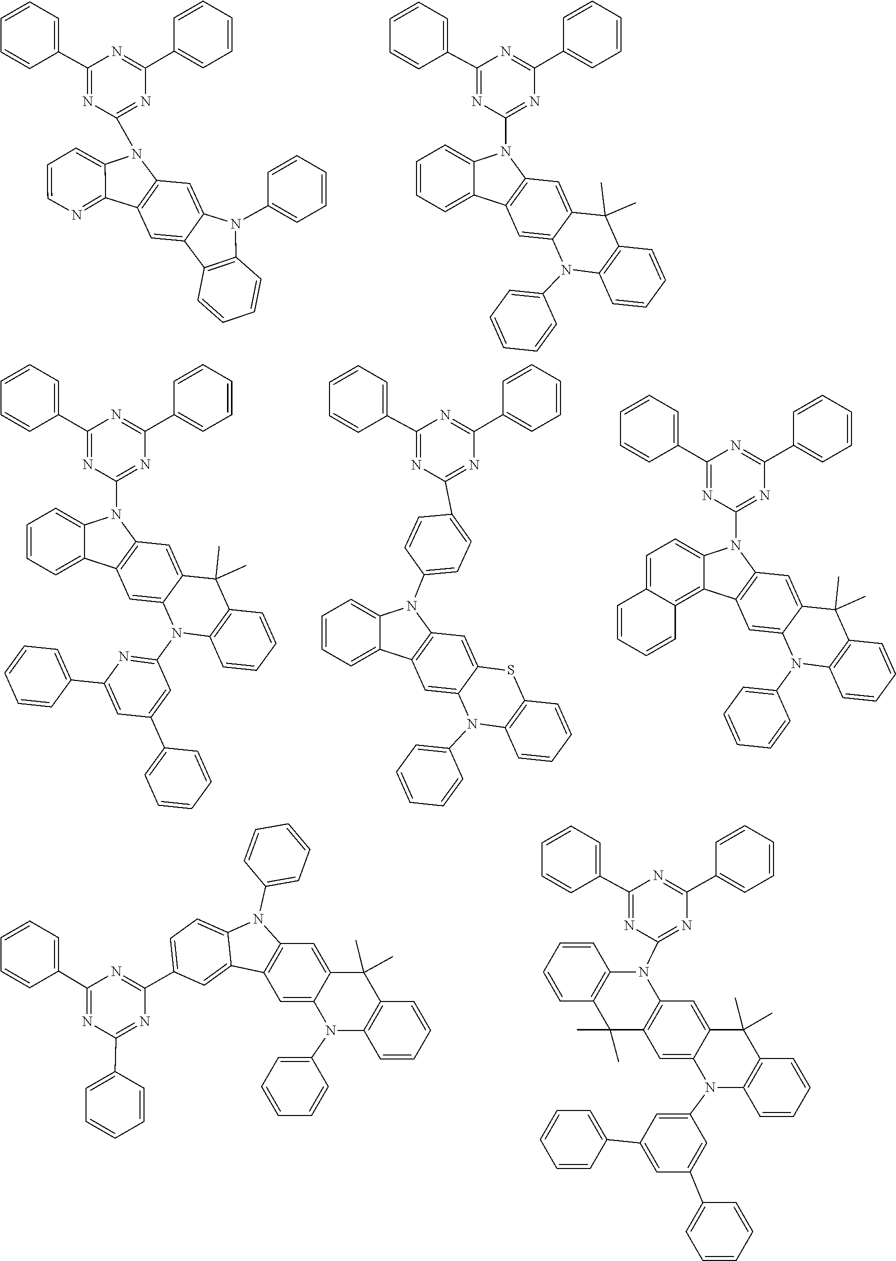

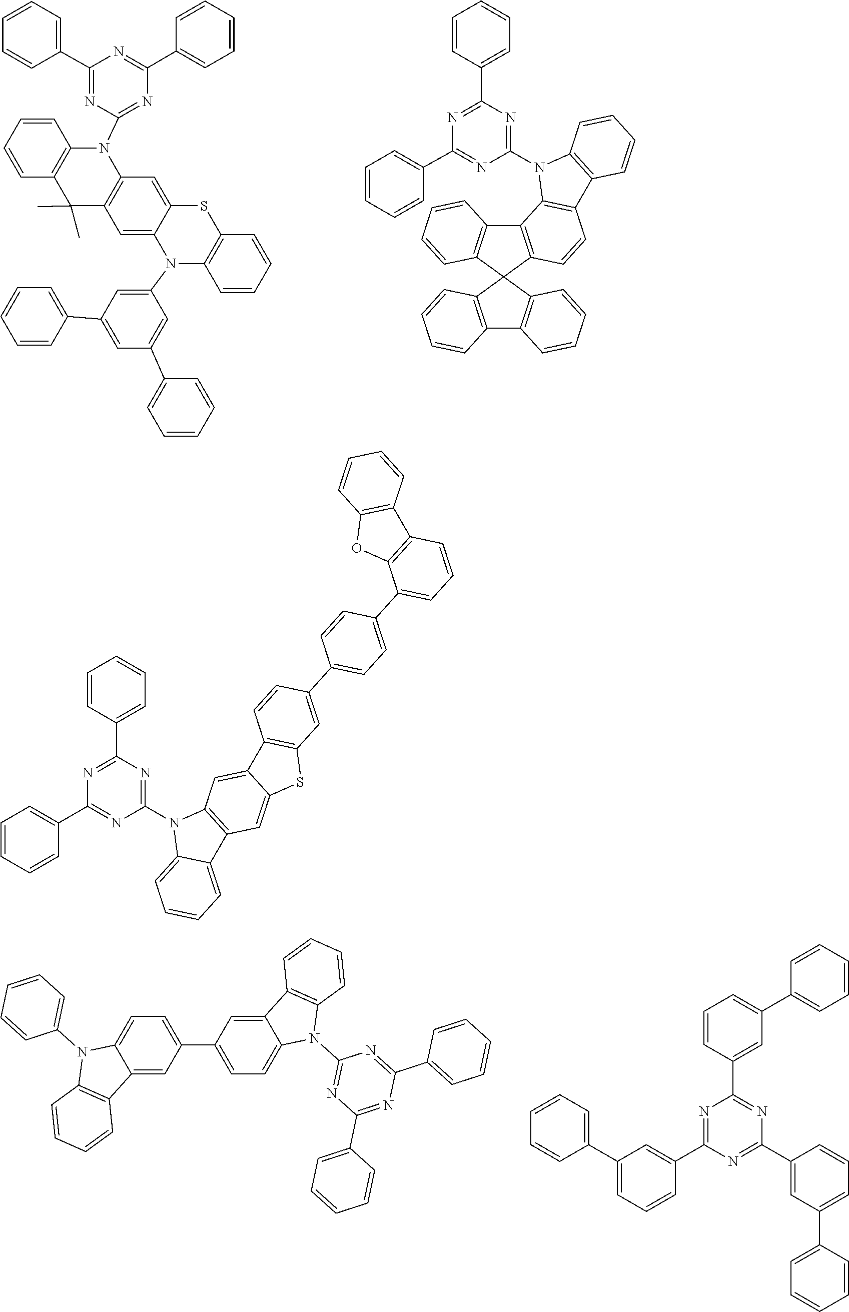 [SMILES: CC1(C)c2ccccc2N(c2nc(-c3ccccc3)nc(-c3ccccc3)n2)c2cc3c(cc21)N(c1cc(-c2ccccc2)cc(-c2ccccc2)c1)c1ccccc1S3 ; c1ccc(-c2nc(-c3ccccc3)nc(-n3c4ccccc4c4ccc5c(c43)-c3ccccc3C53c4ccccc4-c4ccccc43)n2)cc1 ; c1ccc(-c2nc(-c3ccccc3)nc(-n3c4ccccc4c4cc5sc6cc(-c7ccc(-c8cccc9c8oc8ccccc89)cc7)ccc6c5cc43)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-c3nc(-c4cccc(-c5ccccc5)c4)nc(-c4cccc(-c5ccccc5)c4)n3)c2)cc1]

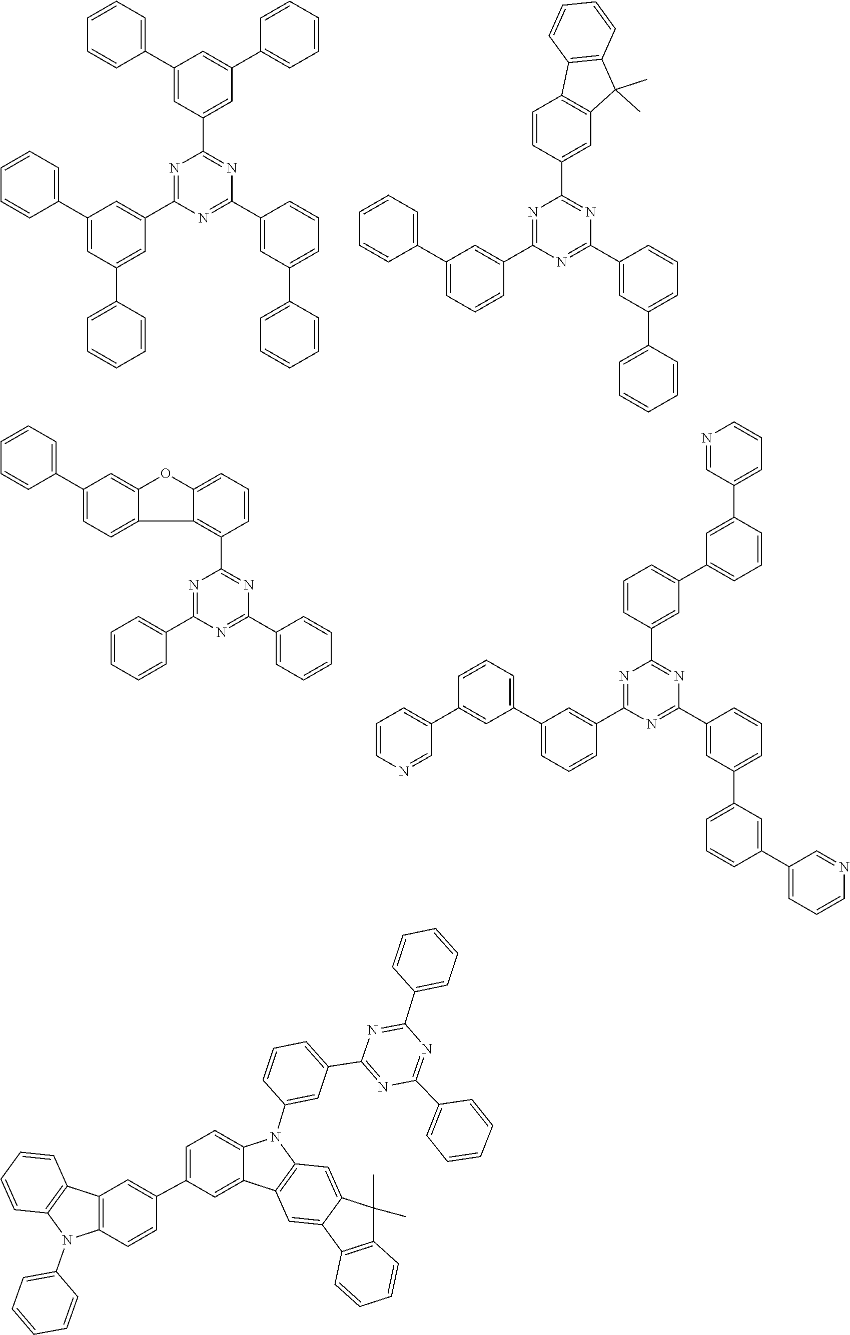

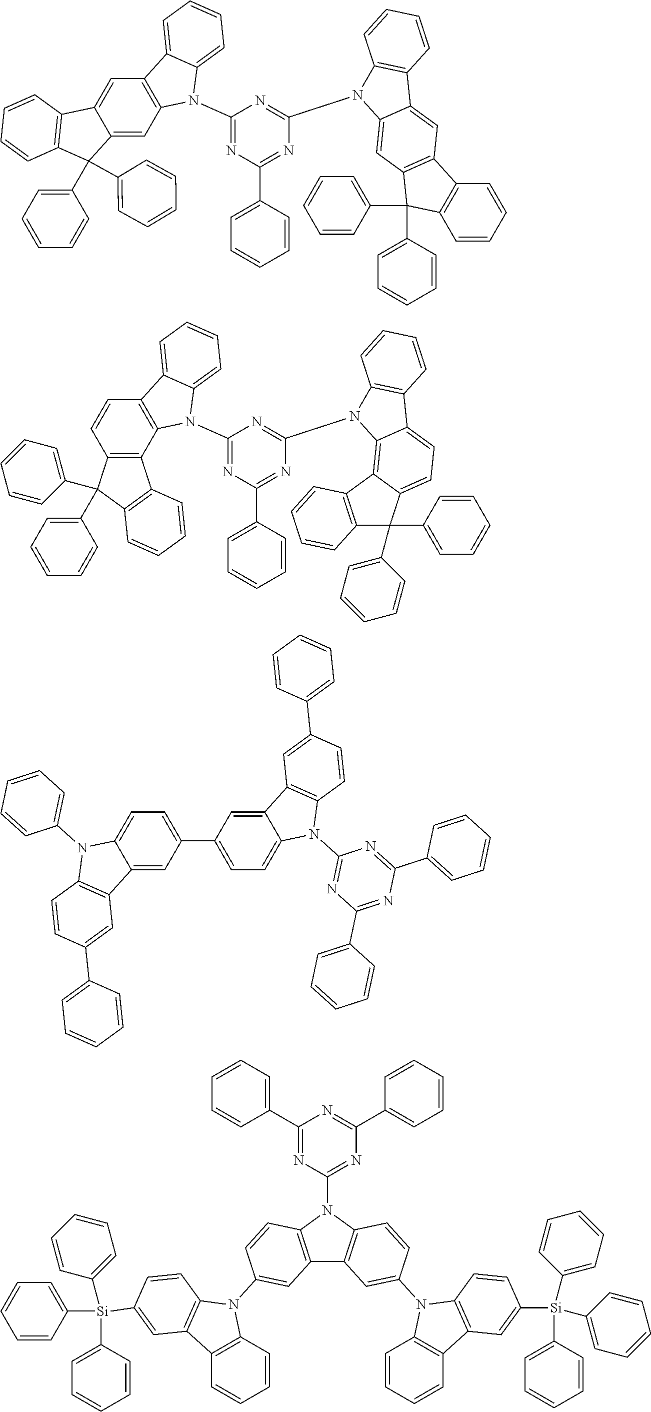

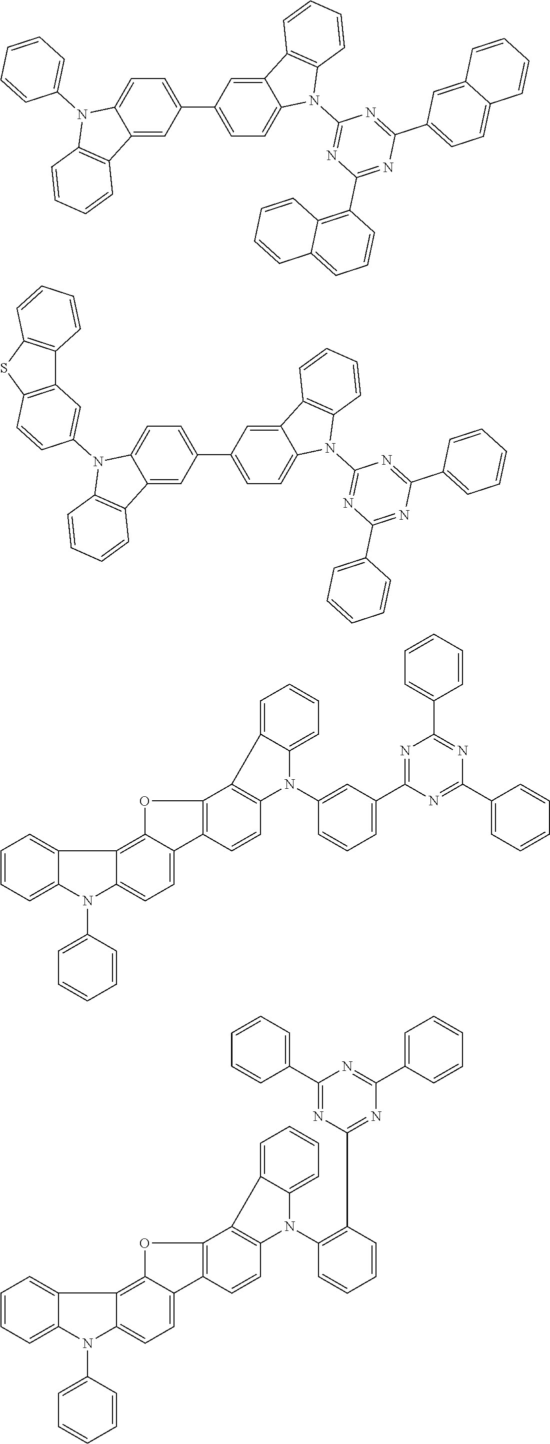

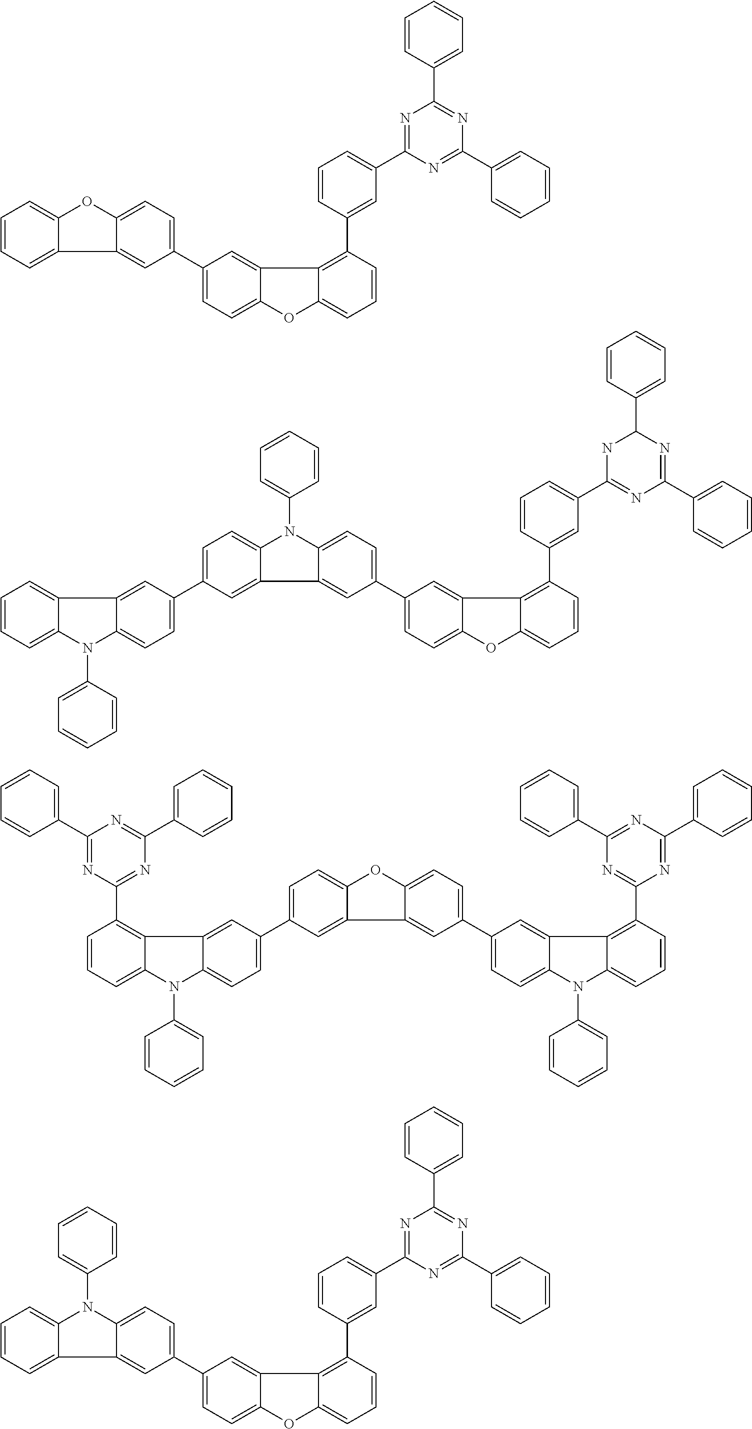

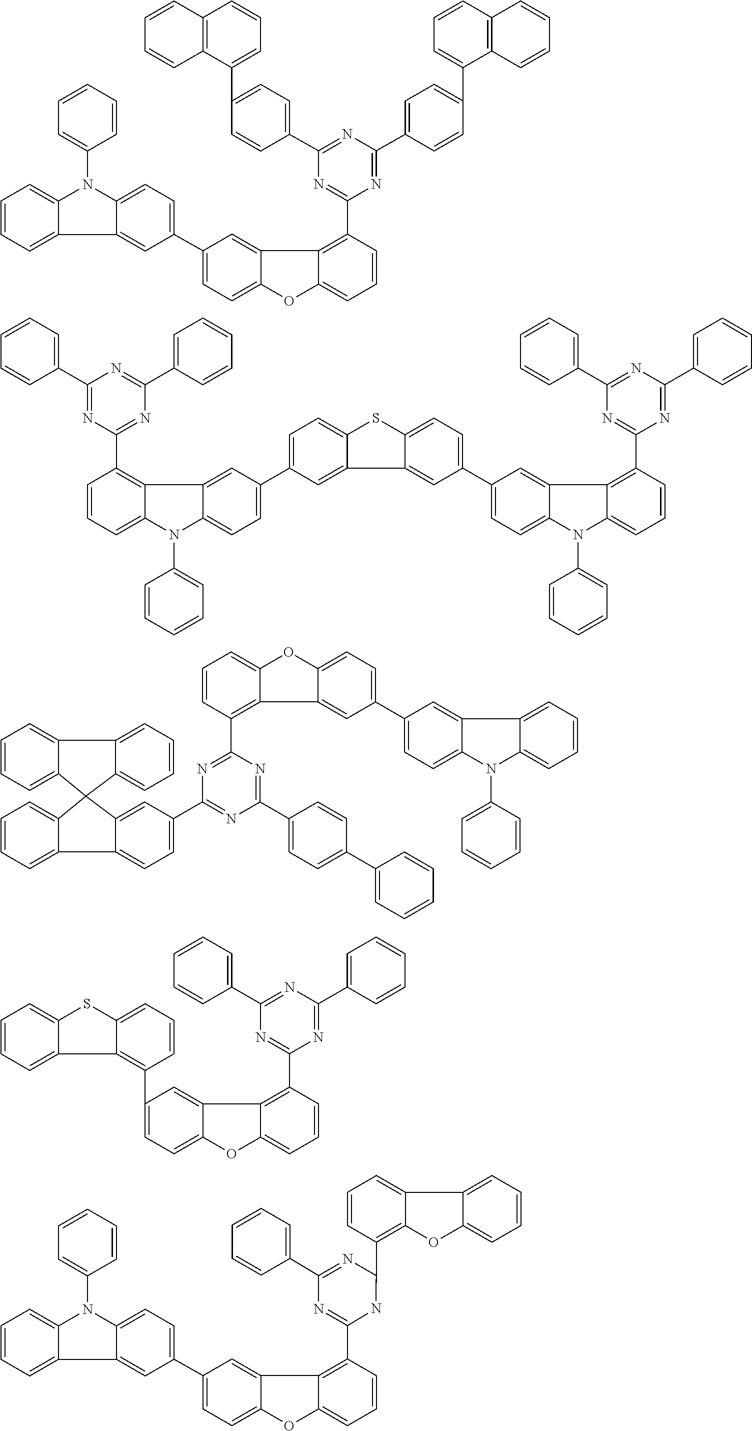

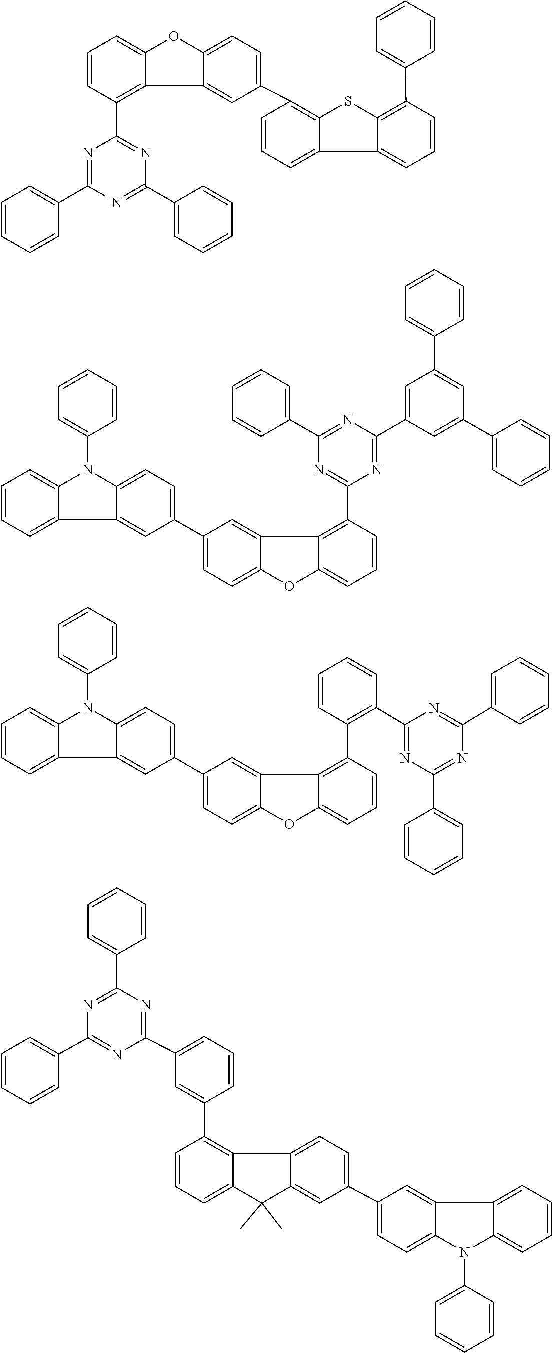

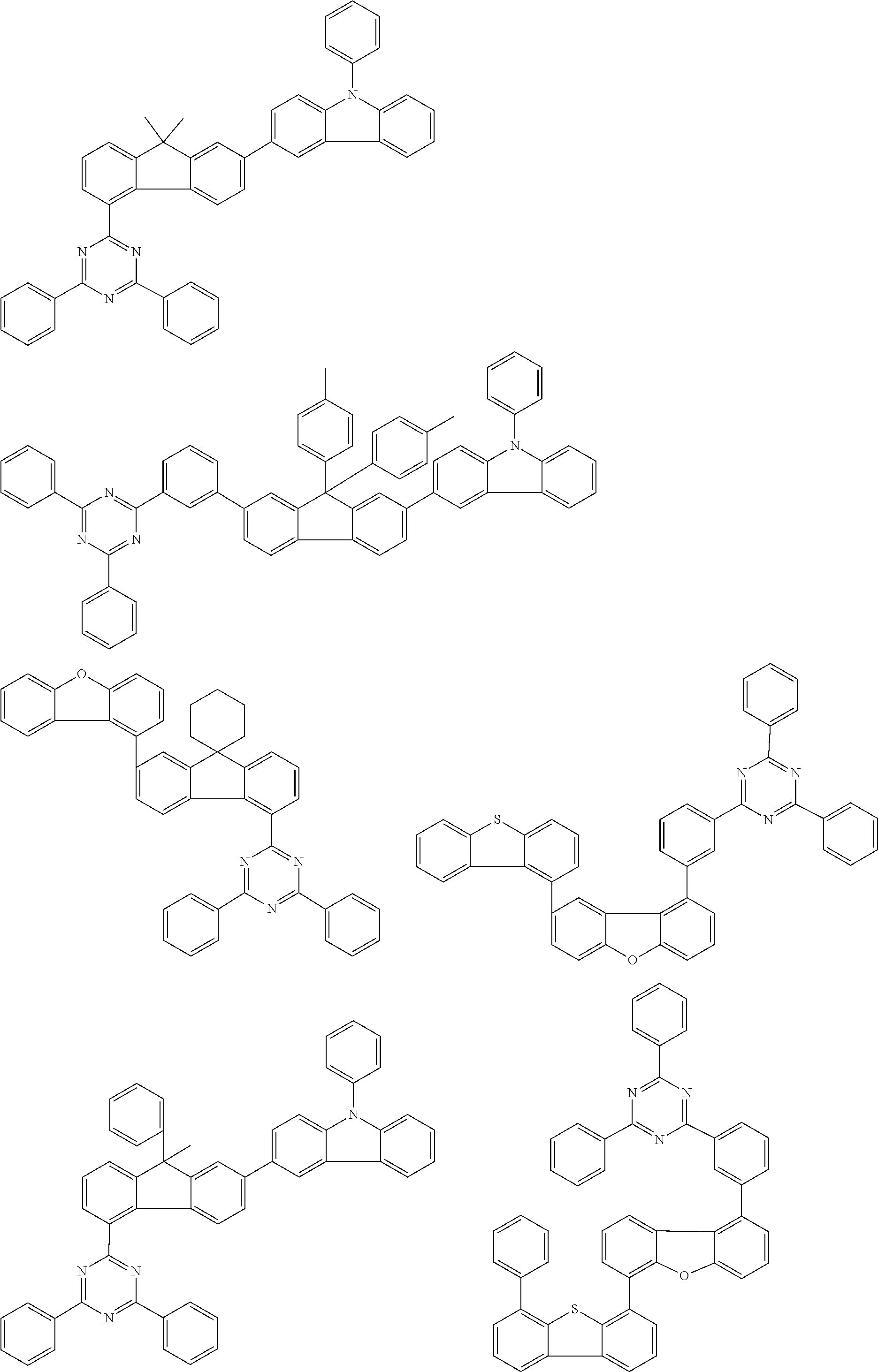

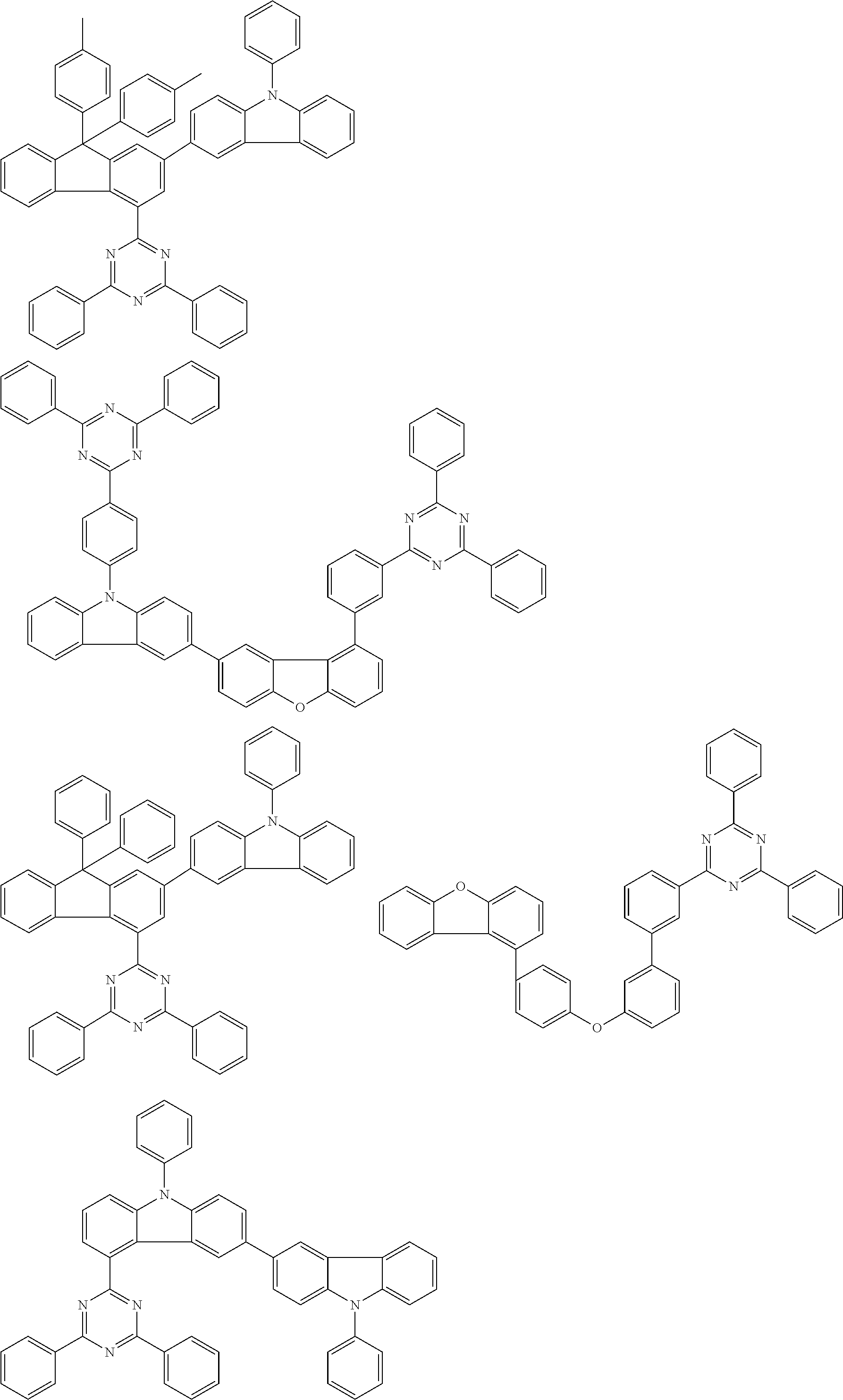

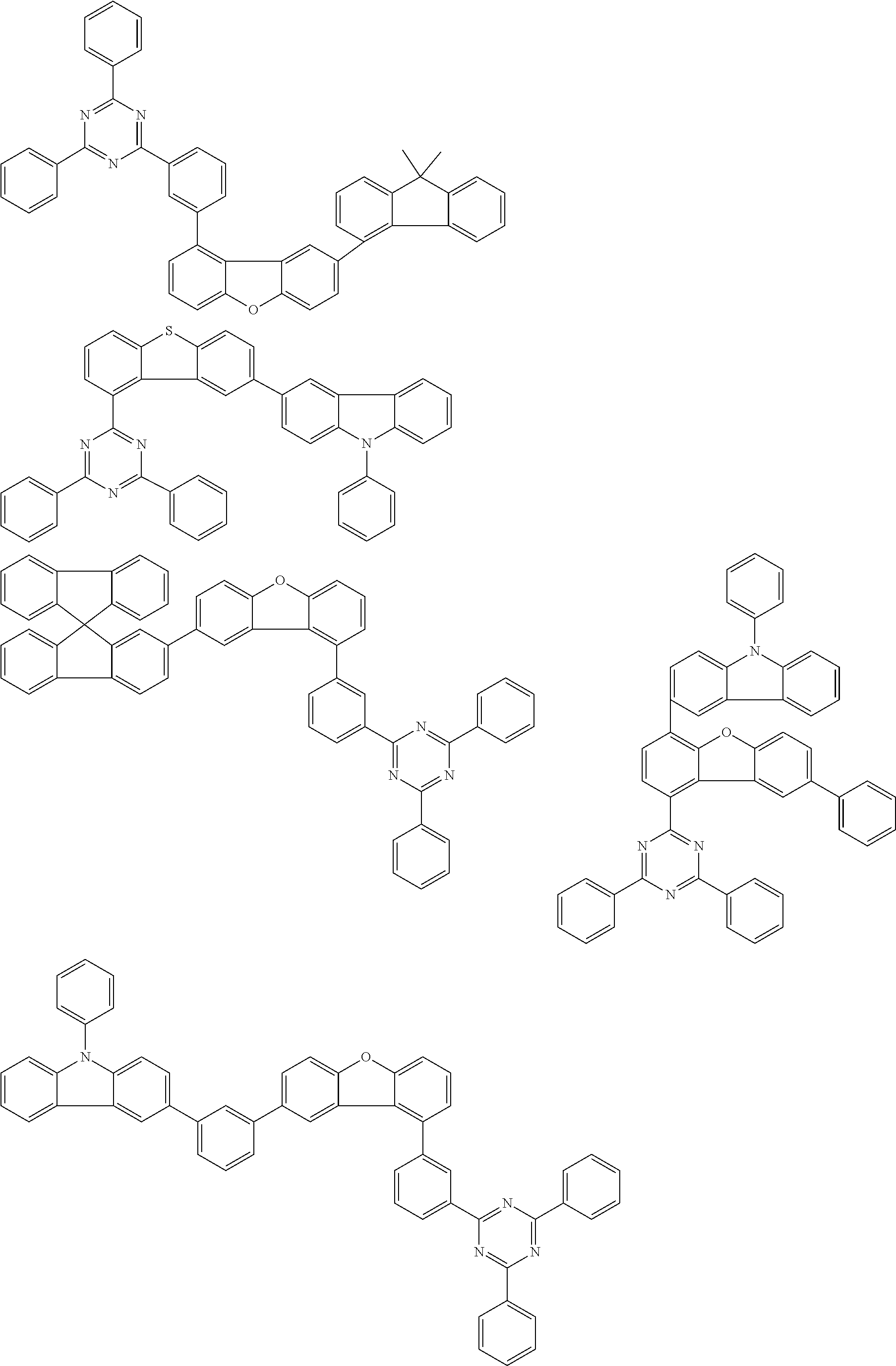

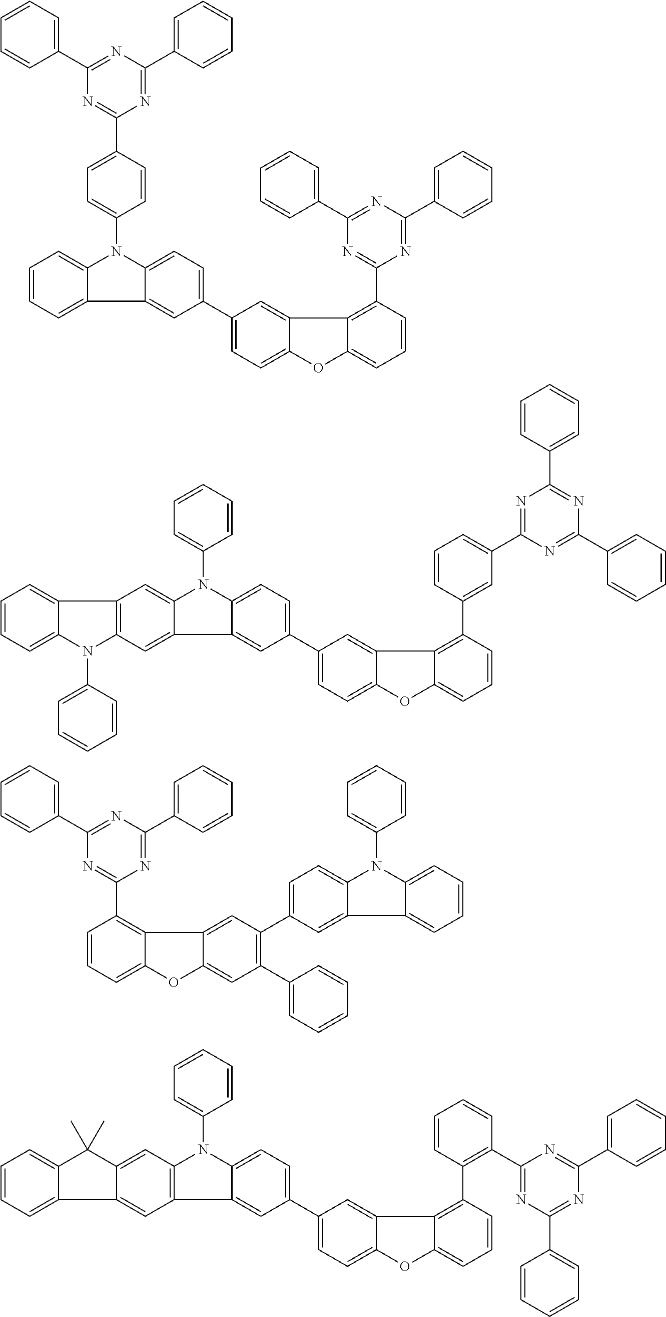

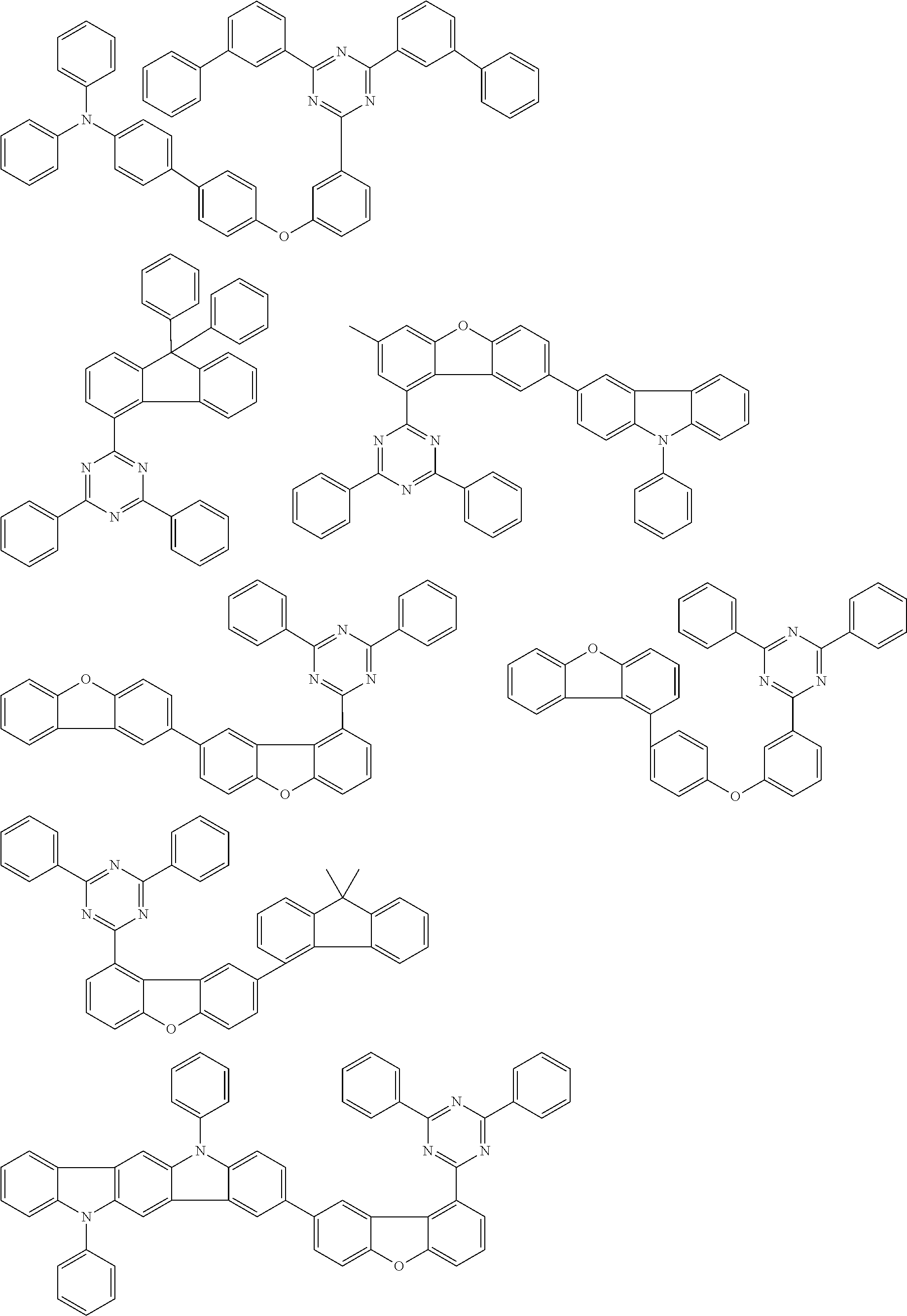

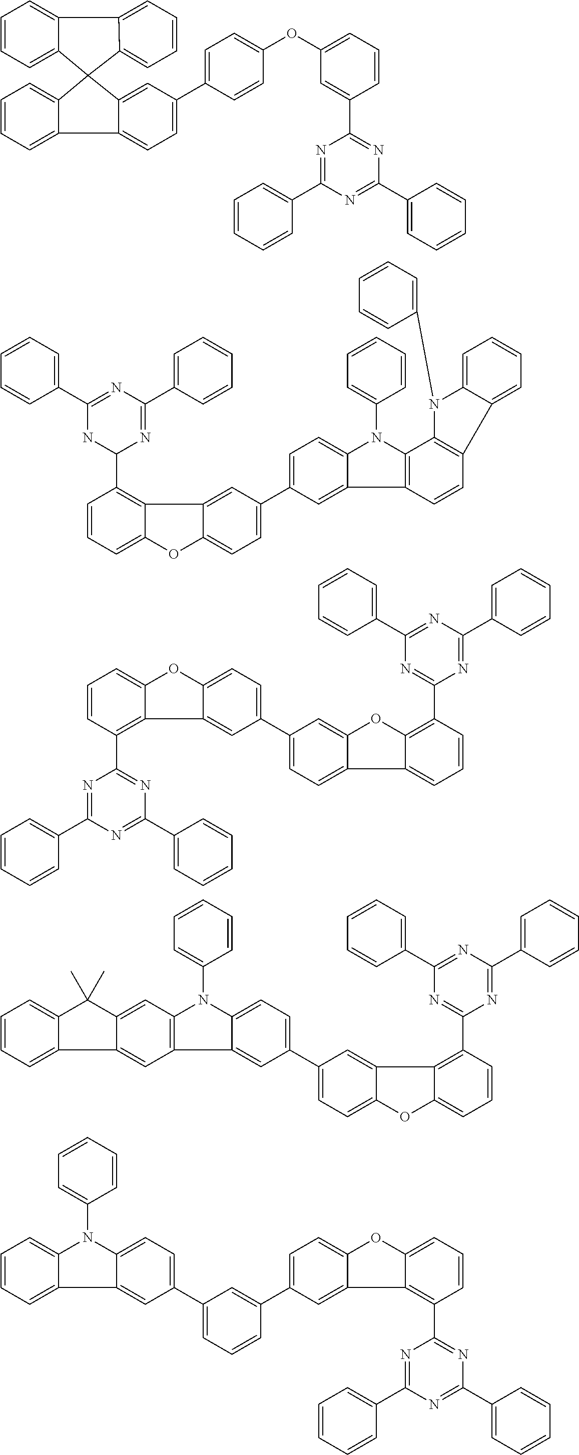

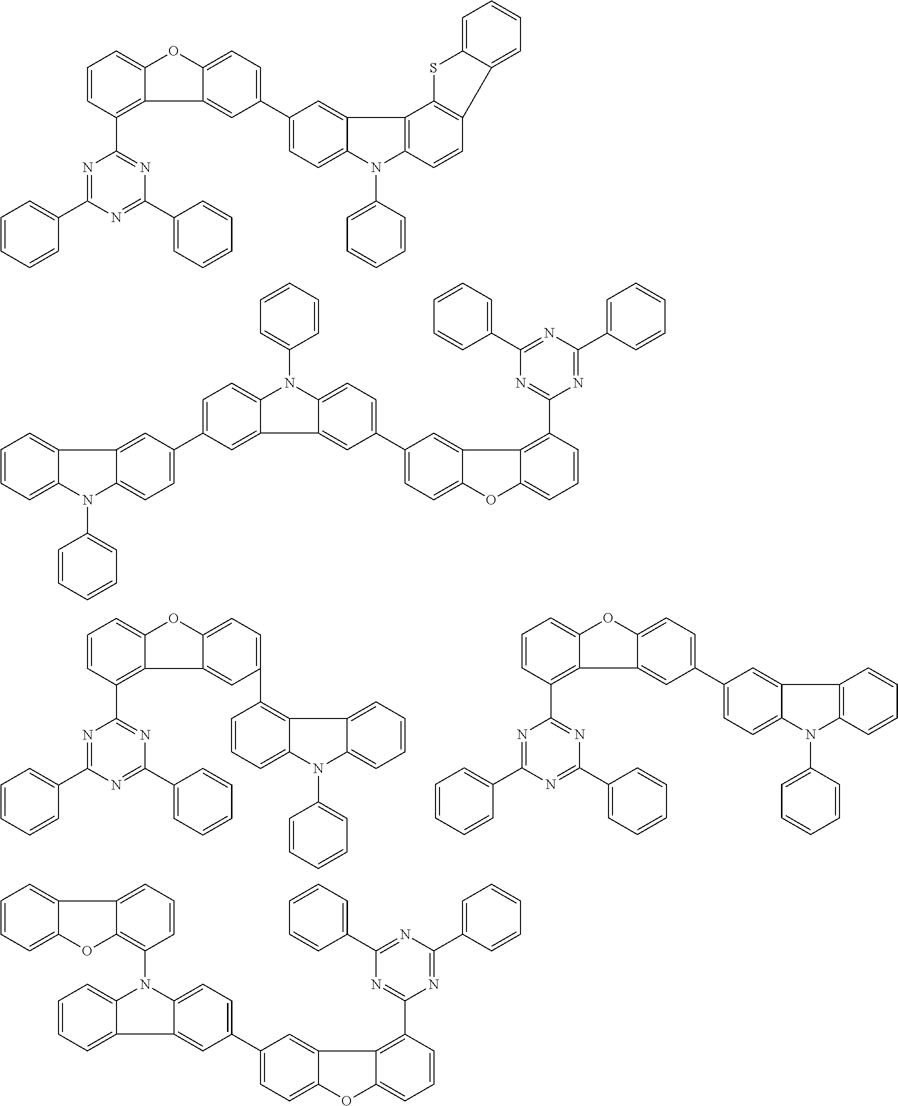

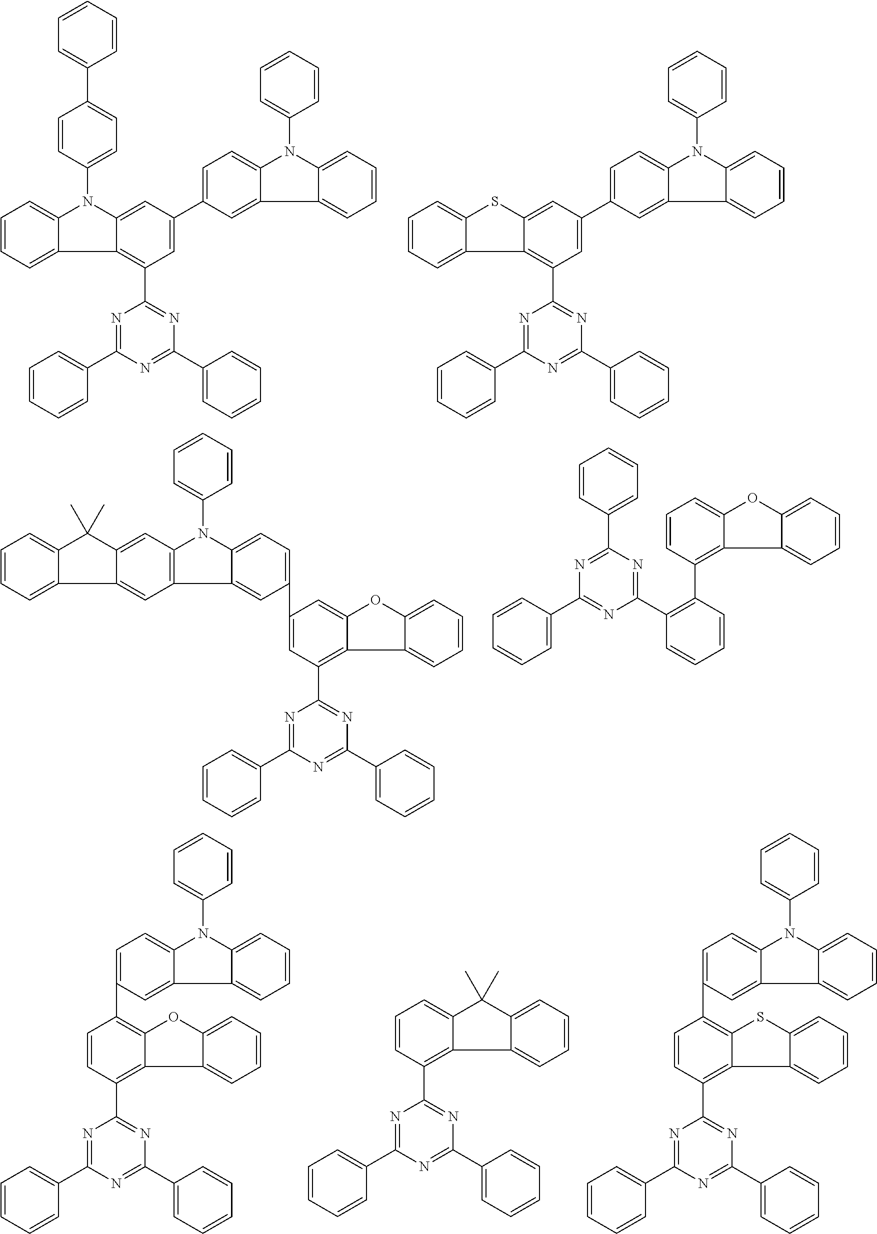

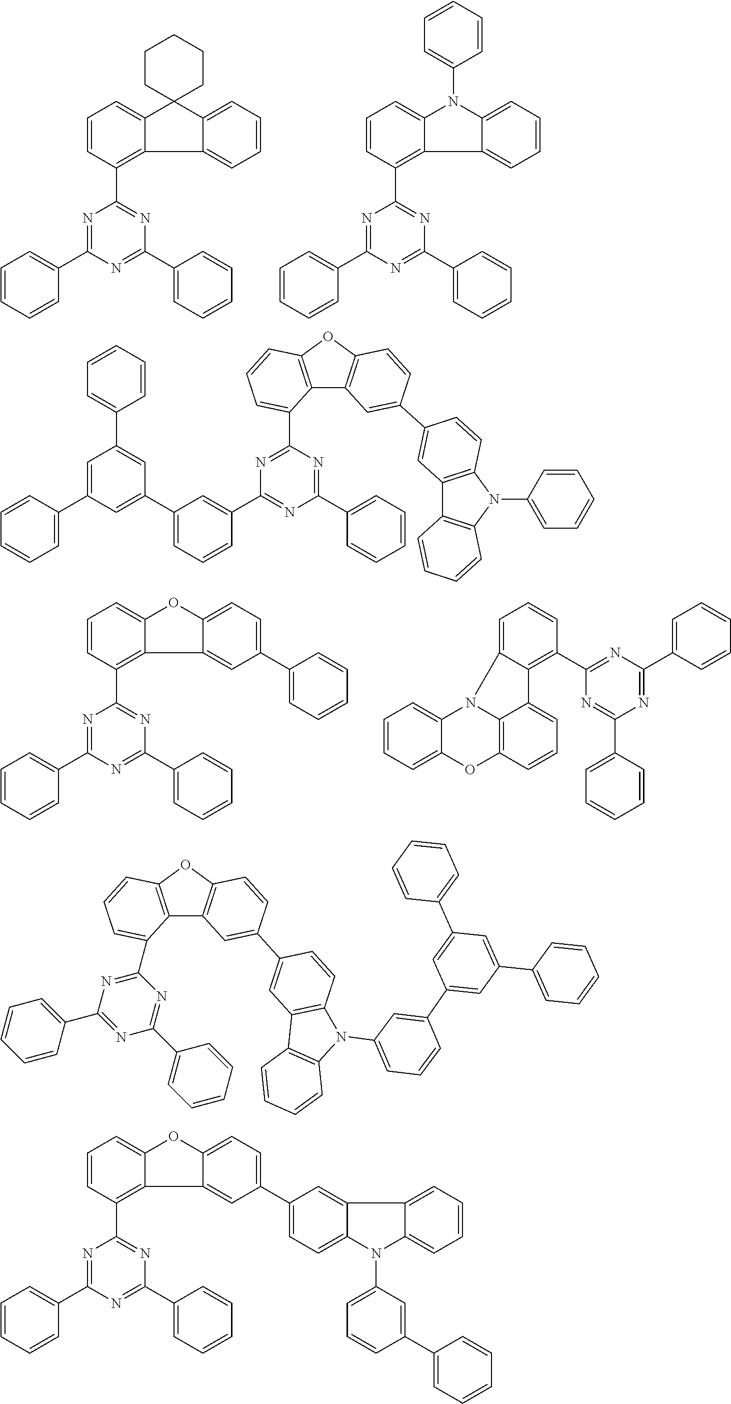

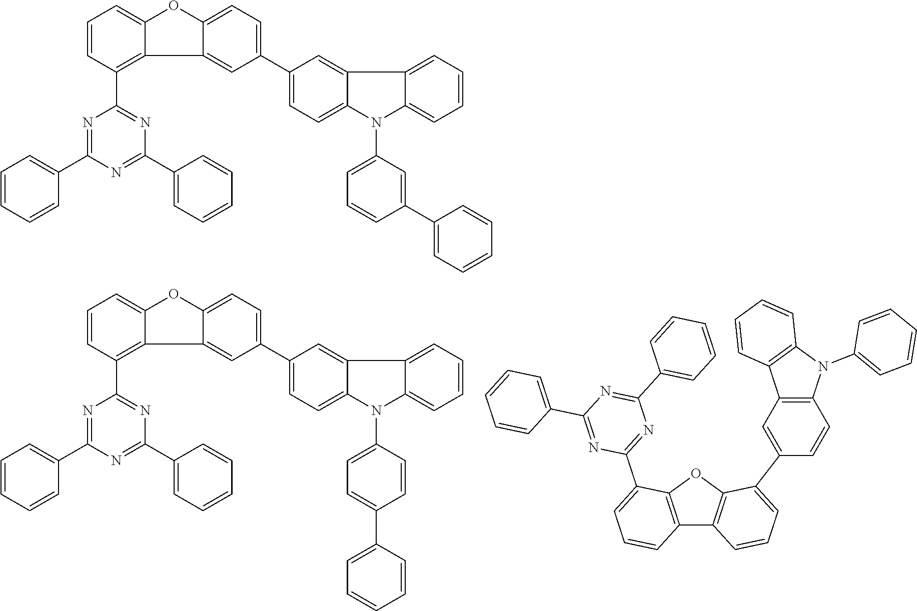

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186 and WO 2018/041769, WO 2019/020538, WO 2018/178001, WO 2019/115423, and the as yet unpublished patent application EP 18156388.3. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are adduced below.

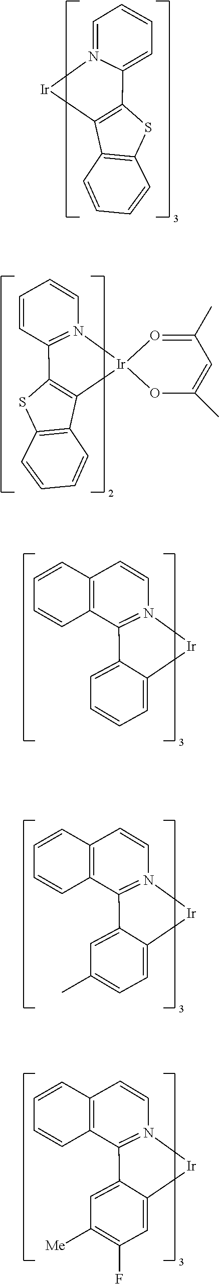

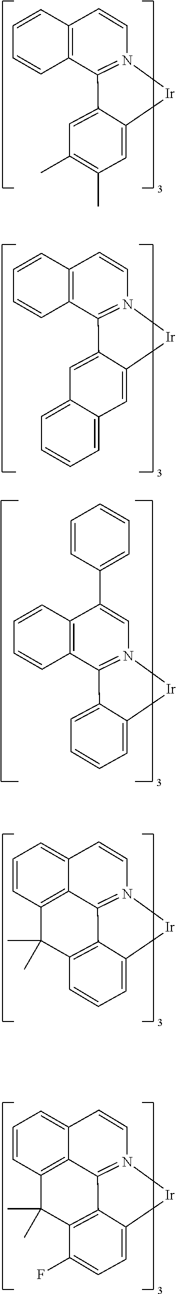

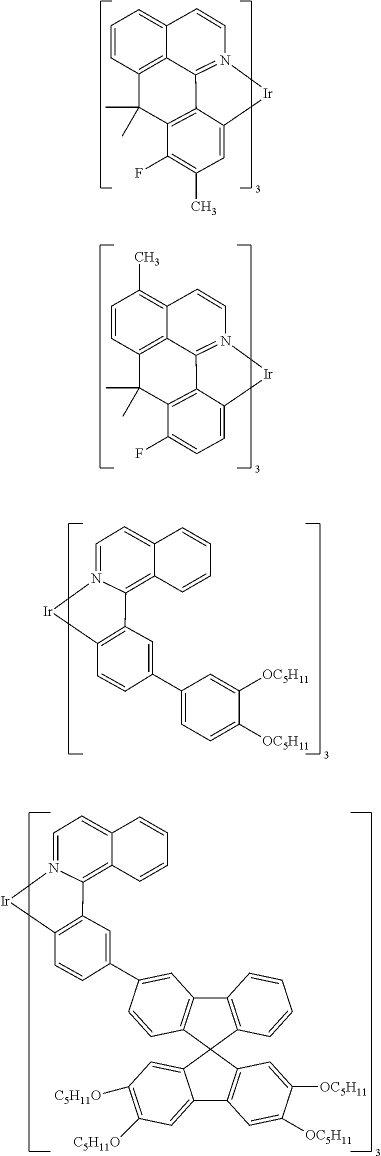

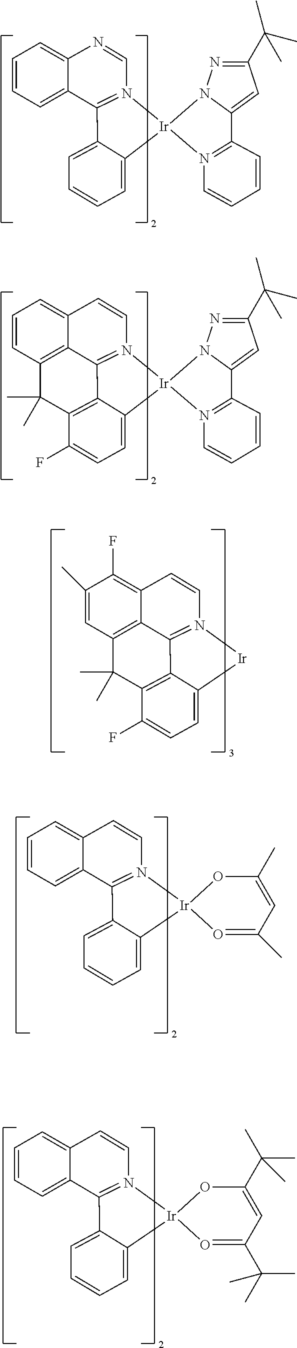

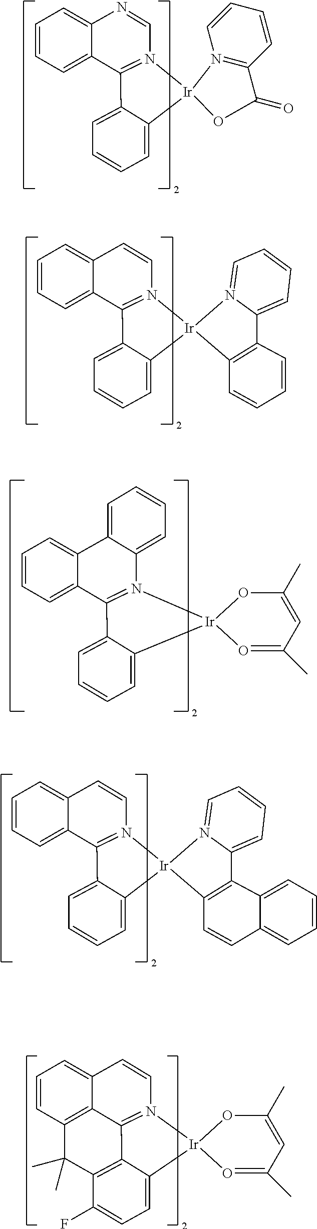

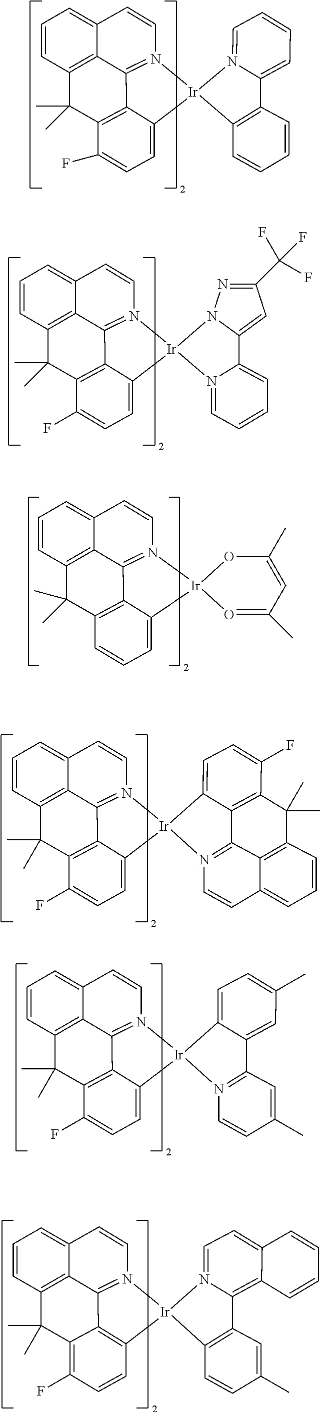

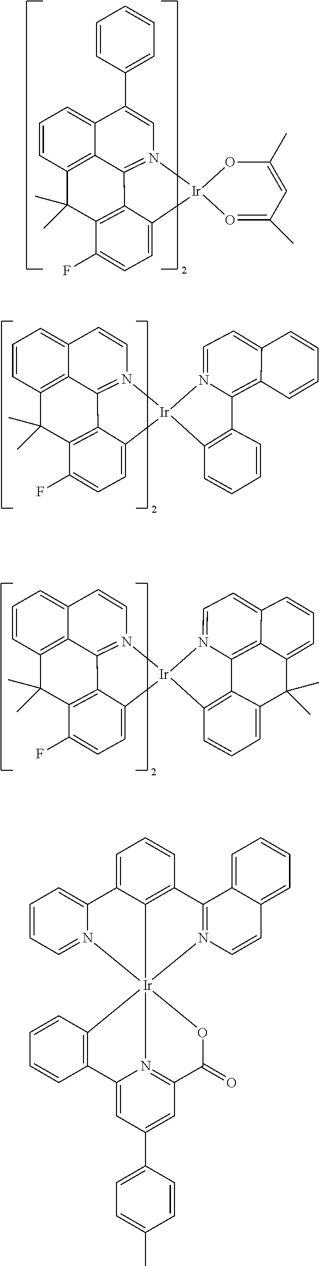

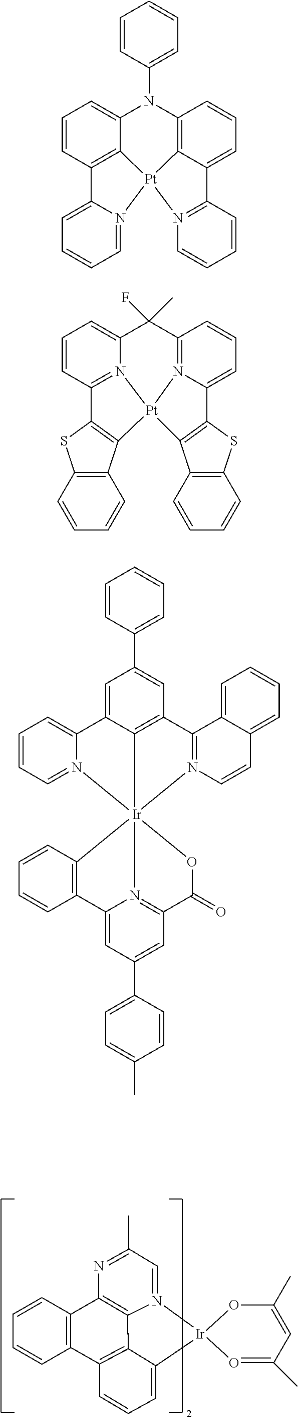

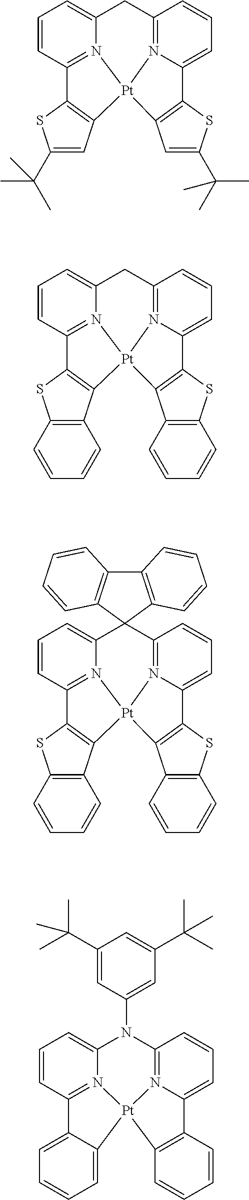

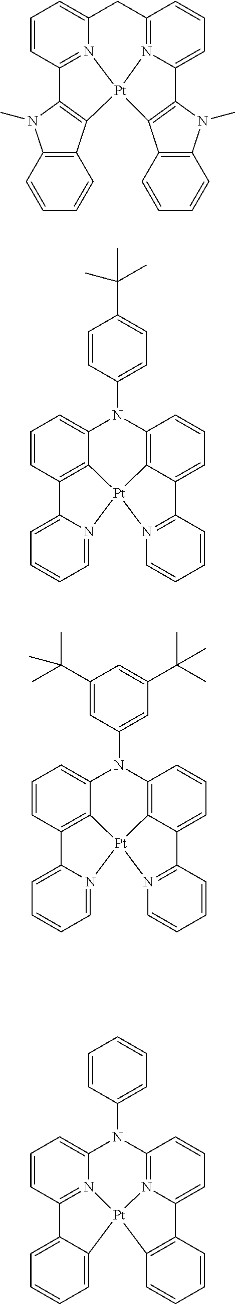

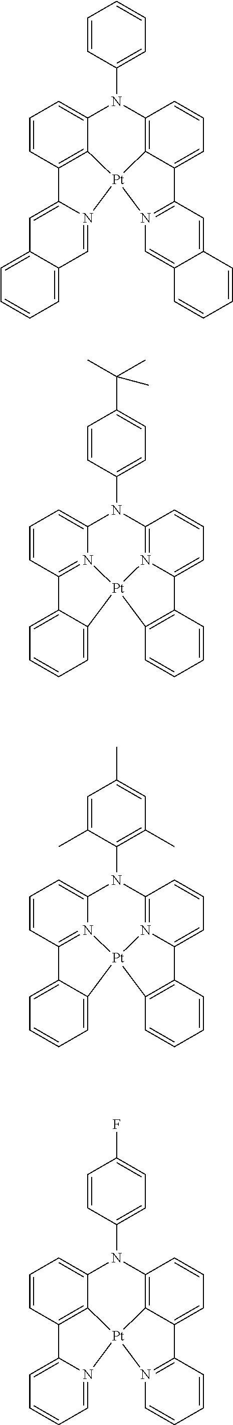

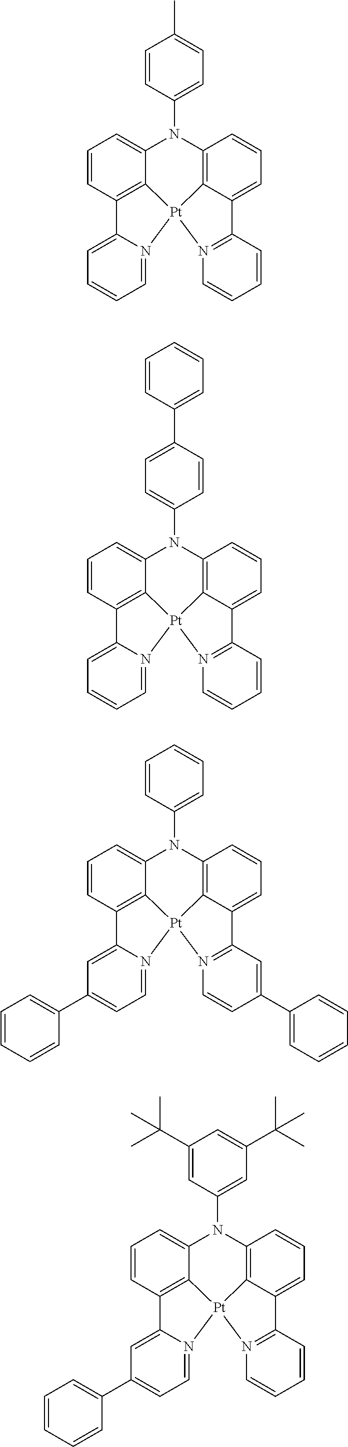

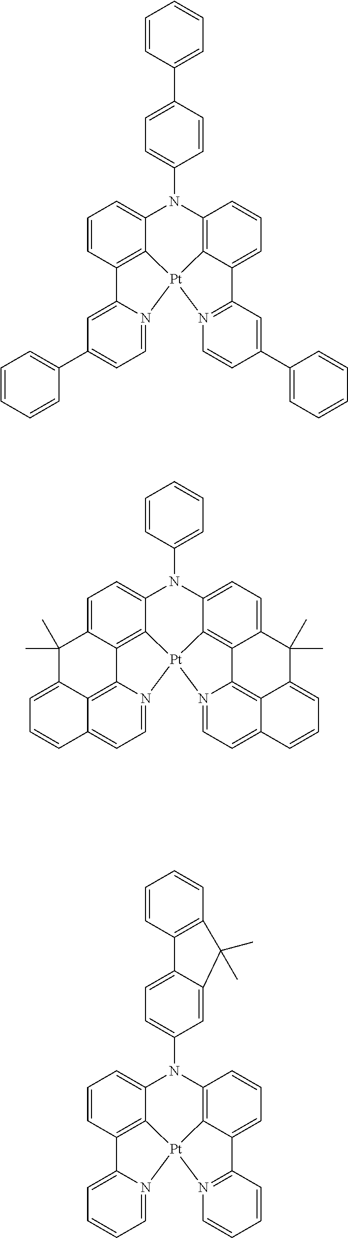

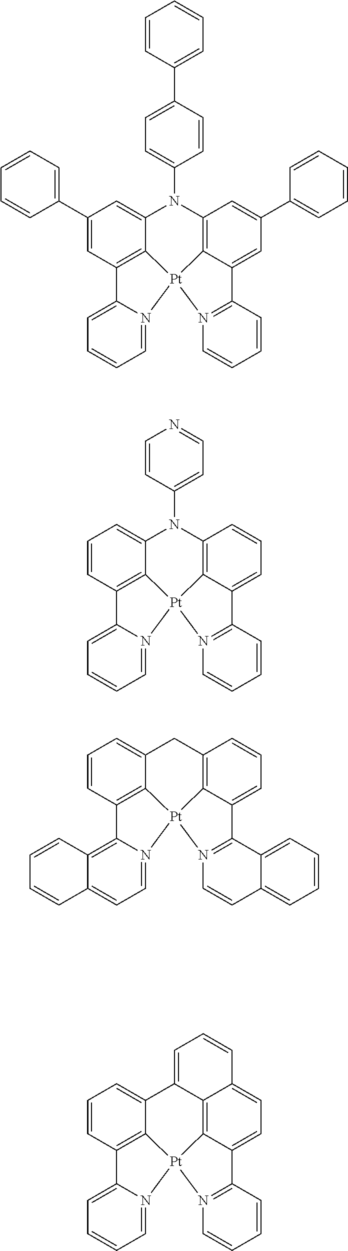

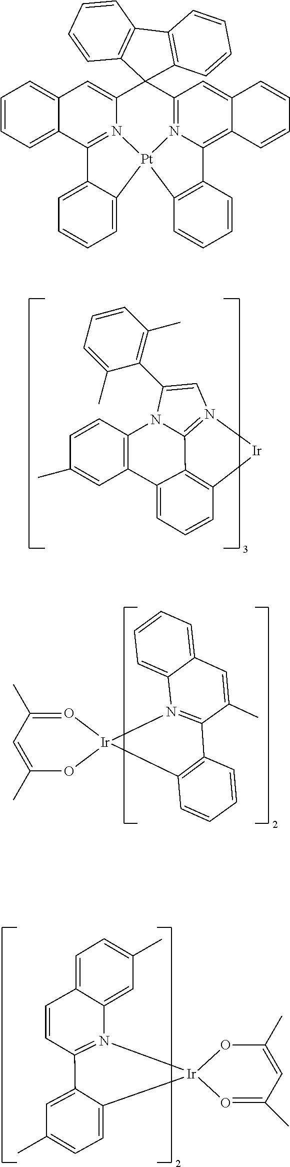

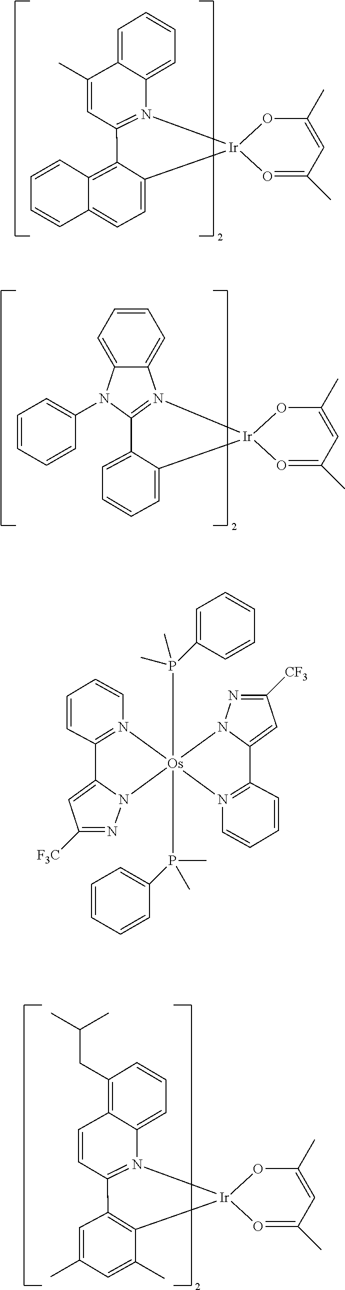

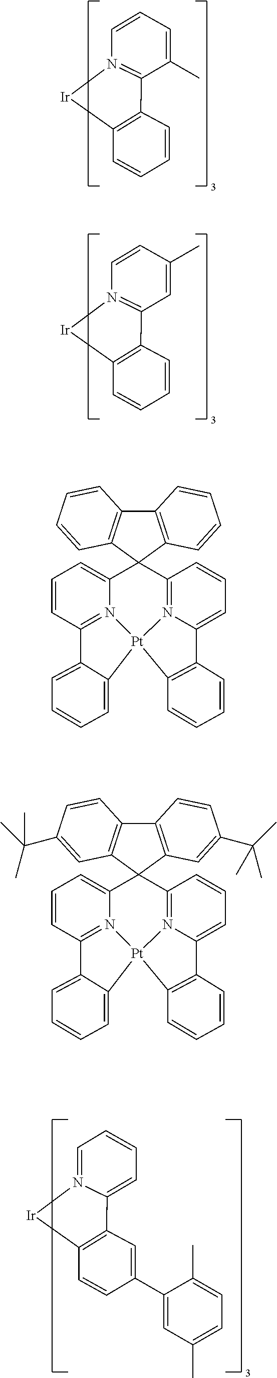

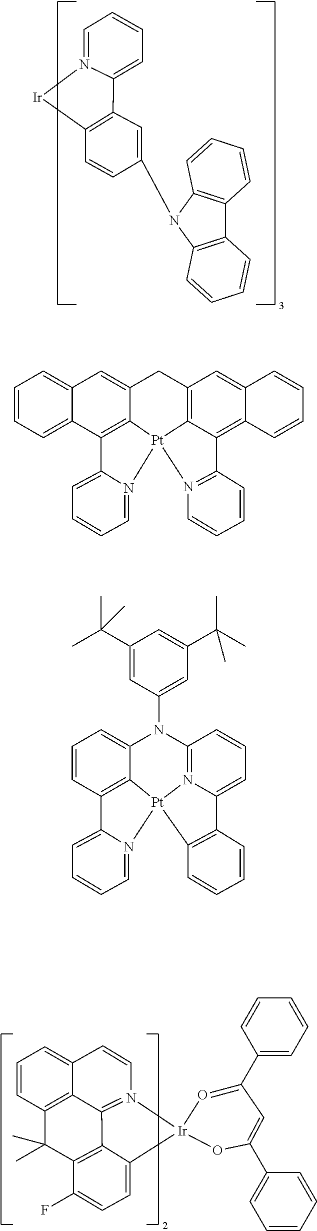

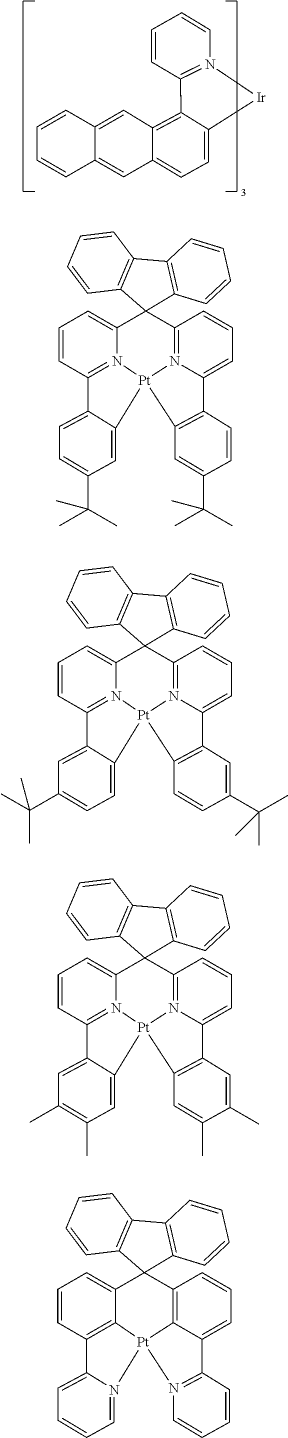

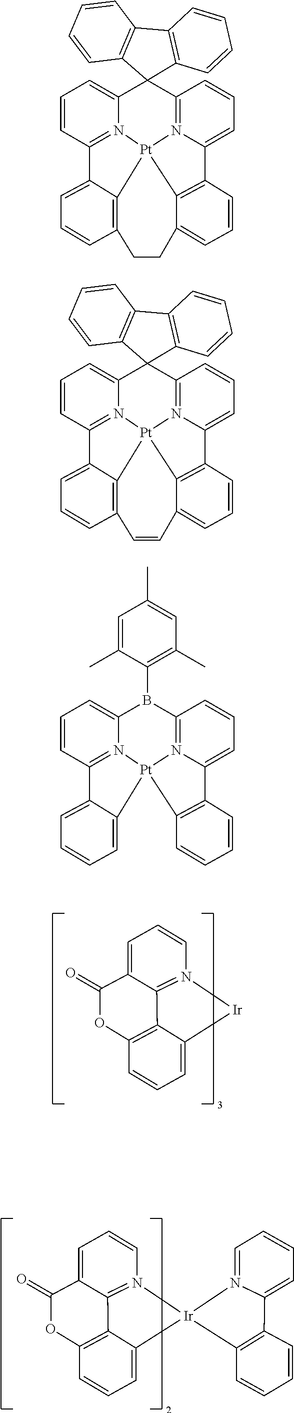

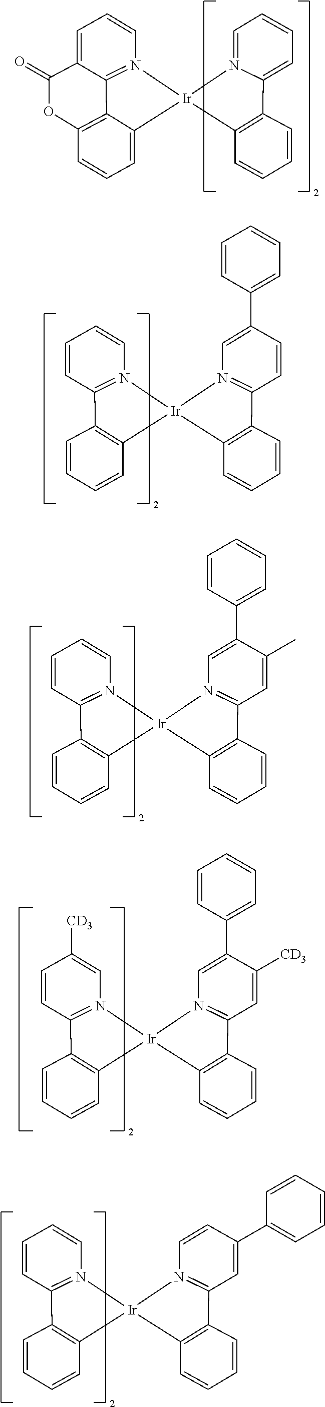

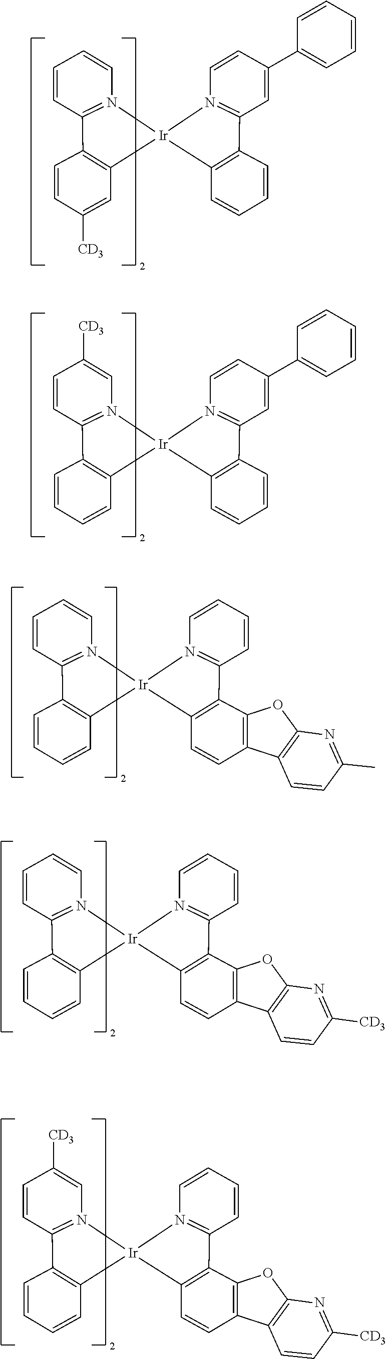

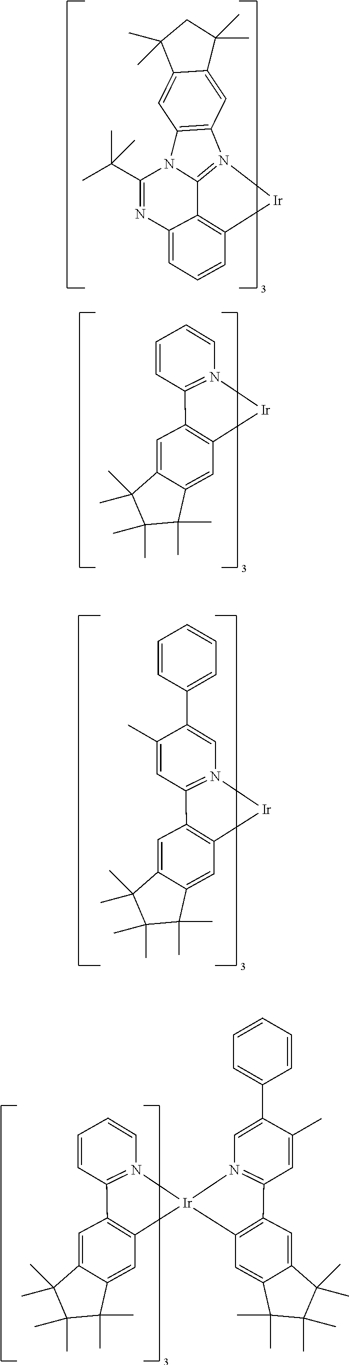

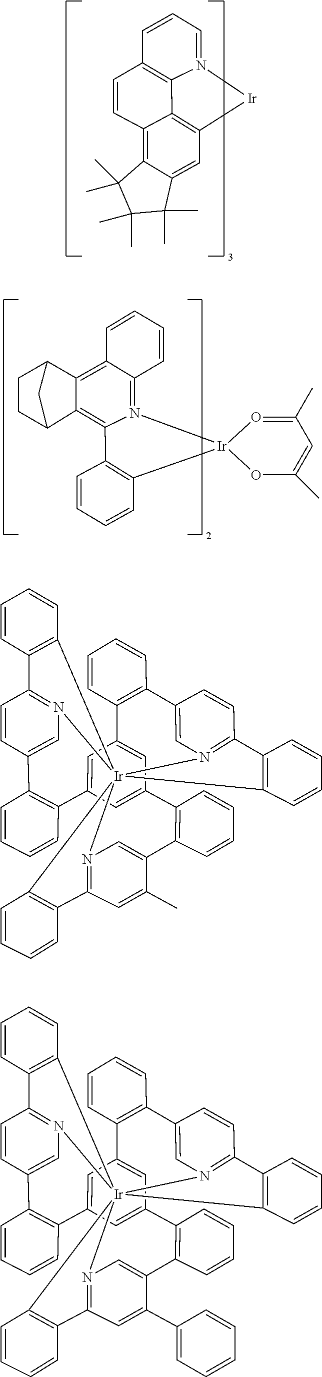

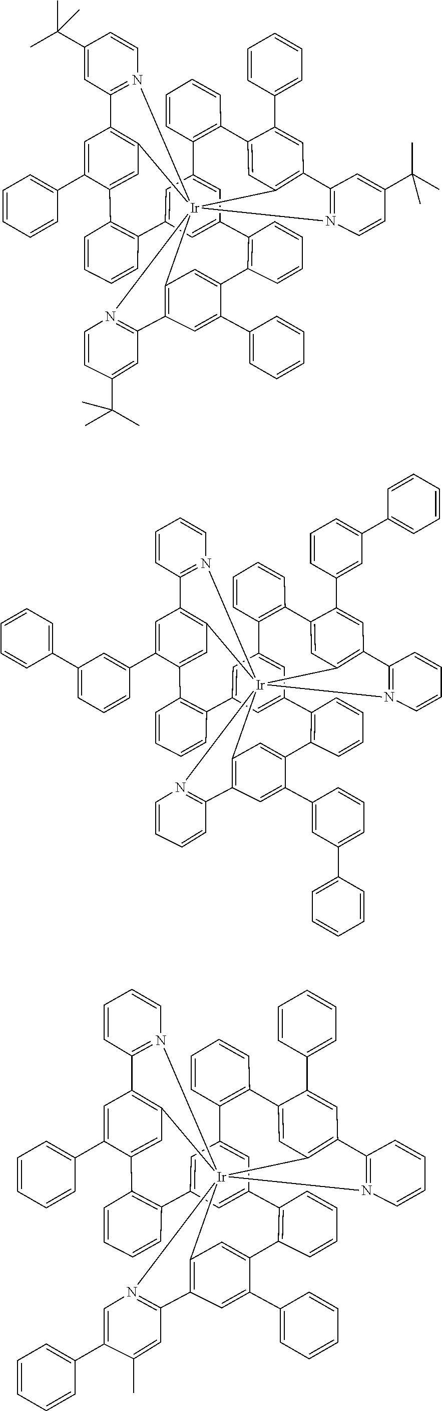

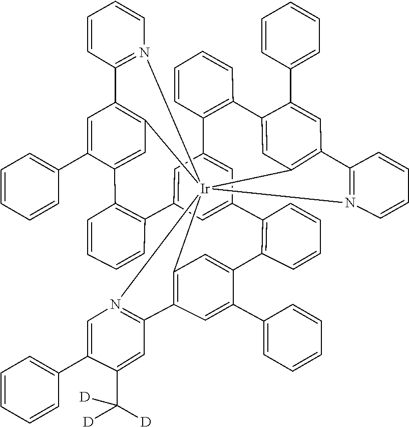

The compounds of the invention are also suitable as matrix materials for phosphorescent emitters in organic electroluminescent devices, as described, for example, in WO 98/24271, US 2011/0248247 and US 2012/0223633. In these multicolor display components, an additional blue emission layer is applied by vapor deposition over the full area to all pixels, including those having a color other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or (2) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

These methods are known in general terms to those skilled in the art and can be applied by those skilled in the art without exercising inventive skill to organic electroluminescent devices comprising the compounds of the invention.

The compounds of the invention and the organic electroluminescent devices of the invention are notable for one or more of the following surprising advantages over the prior art:
1. The compounds of the invention, used as matrix material for phosphorescent emitters, lead to long lifetimes.
2. The compounds of the invention lead to high efficiencies. This is especially true when the compounds are used as matrix material for a phosphorescent emitter.
3. The compounds of the invention lead to low operating voltages. This is especially true when the compounds are used as matrix material for a phosphorescent emitter.

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

### Synthesis Examples

The syntheses which follow, unless stated otherwise, are conducted under a protective gas atmosphere in dried solvents. The solvents and reagents can be purchased from ALDRICH or ABCR. The numbers given for the reactants that are not commercially available are the corresponding CAS numbers.

### a) 6-Chloro-5H-benzo[c][2,1]benzazaphosphinin

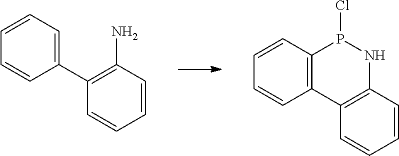 [SMILES: Nc1ccccc1-c1ccccc1 ; ClP1Nc2ccccc2-c2ccccc21]

8 g (50 mmol) of 2-aminobiphenyl in 70 ml of phosphorus trichloride is heated under reflux for 8 h, then the residual PCl₃ is distilled off under reduced pressure, and the residue (Ar—NH—PCl₂) is heated with 0.5 g of AlCl₃ to 180-220° C. for 6 h. The mixture is dissolved in toluene and filtered through glass wool and hence separated from salts. Purification is effected by sublimation at 180-190° C. (0.05 mm) in the form of white needles that are very sensitive to hydrolysis. Yield: 4.2 g (24 mmol); 42% of theory; purity: 97% by NMR.

The following compounds can be prepared in an analogous manner:

| Ex. | Reactant | Product | Yield |
|---|---|---|---|
| 1a | 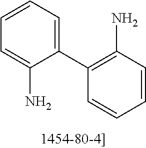 [SMILES: Nc1ccccc1-c1ccccc1N] | 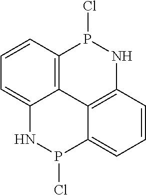 [SMILES: Clp1[nH]c2cccc3c2-c2c(cccc21)[nH]p3Cl] | 30% |
| 2a | 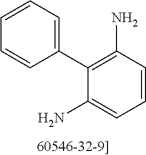 [SMILES: Nc1cccc(N)c1-c1ccccc1] | 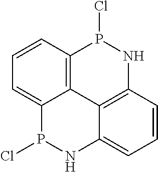 [SMILES: Clp1[nH]c2cccc3[nH]p(Cl)c4cccc1c4-c23] | 34% |
| 3a | 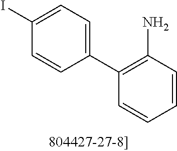 [SMILES: Nc1ccccc1-c1ccc(I)cc1] | 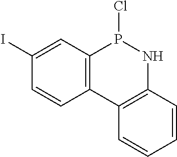 [SMILES: ClP1Nc2ccccc2-c2ccc(I)cc21] | 47% |
| 4a | 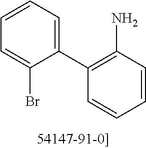 [SMILES: Nc1ccccc1-c1ccccc1Br] | 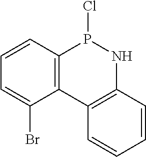 [SMILES: ClP1Nc2ccccc2-c2c(Br)cccc21] | 41% |
| 5a | 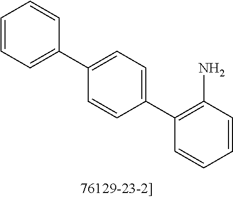 [SMILES: Nc1ccccc1-c1ccc(-c2ccccc2)cc1] | 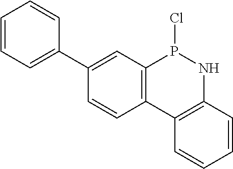 [SMILES: ClP1Nc2ccccc2-c2ccc(-c3ccccc3)cc21] | 45% |
| 6a | 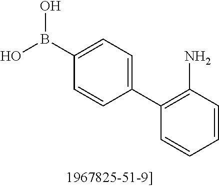 [SMILES: Nc1ccccc1-c1ccc(B(O)O)cc1] | 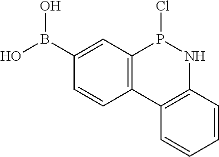 [SMILES: OB(O)c1ccc2c(c1)P(Cl)Nc1ccccc1-2] | 38% |
| 7a | 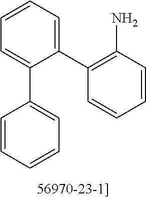 [SMILES: Nc1ccccc1-c1ccccc1-c1ccccc1] | 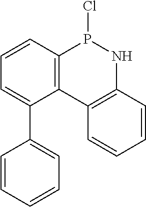 [SMILES: ClP1Nc2ccccc2-c2c(-c3ccccc3)cccc21] | 42% |
| 8a | 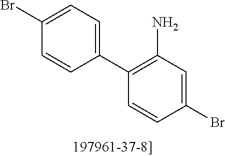 [SMILES: Nc1cc(Br)ccc1-c1ccc(Br)cc1] | 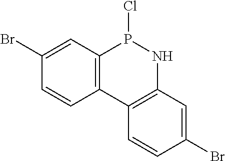 [SMILES: ClP1Nc2cc(Br)ccc2-c2ccc(Br)cc21] | 36% |
| 9a | 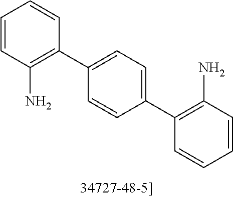 [SMILES: Nc1ccccc1-c1ccc(-c2ccccc2N)cc1] | 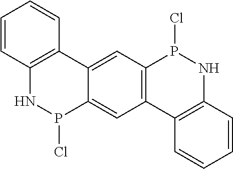 [SMILES: Clp1[nH]c2ccccc2c2cc3c(cc21)c1ccccc1[nH]p3Cl] | 32% |
| 10a | 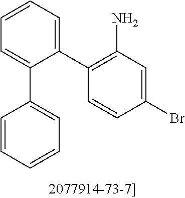 [SMILES: Nc1cc(Br)ccc1-c1ccccc1-c1ccccc1] | 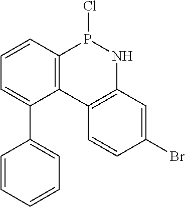 [SMILES: ClP1Nc2cc(Br)ccc2-c2c(-c3ccccc3)cccc21] | 41% |
| 11a | 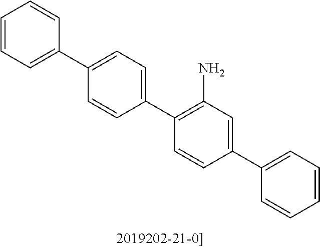 [SMILES: Nc1cc(-c2ccccc2)ccc1-c1ccc(-c2ccccc2)cc1] | 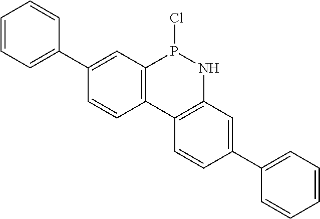 [SMILES: ClP1Nc2cc(-c3ccccc3)ccc2-c2ccc(-c3ccccc3)cc21] | 48% |

### b) 6-Phenyl-5H-benzo[c][2,1]benzazaphosphinin

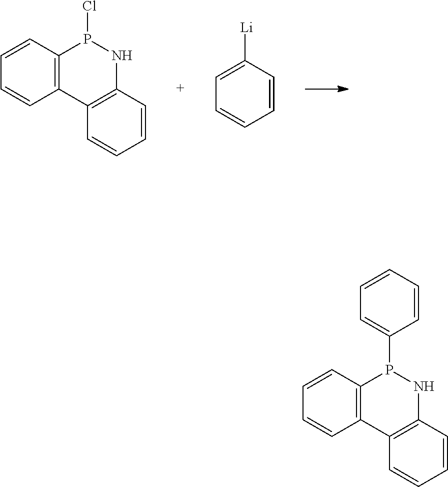

Under protective gas, 4.2 g (24 mmol) of 6-chloro-5H-benzo[c][2,1]benzazaphosphinin is dissolved in 120 ml of dry toluene. This solution is added within 15 min to a phenyllithium solution, synthesized from 16.2 g (103 mmol) of bromobenzene and 1.5 g of lithium in 100 ml of ether, and boiled under reflux for 1 h. After cooling, the mixture is added to ice-water, and the organic phase is separated off. The organic phase is concentrated under reduced pressure, and the product is recrystallized from toluene. Yield: 3 g (11 mmol); 61% of theory; purity: 98% by NMR.

The following compounds can be prepared in an analogous manner:

| | | Reactant | | |
|---|---|---|---|---|
| Ex. | Reactant 1 | 2 | Product | Yield |
| 1b | 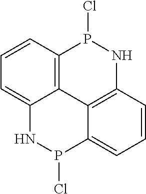 [SMILES: Clp1[nH]c2cccc3c2-c2c(cccc21)[nH]p3Cl] | 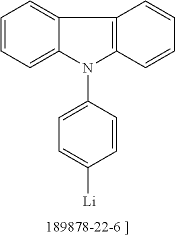 | 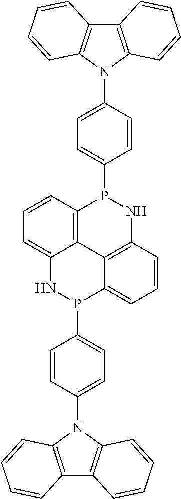 [SMILES: c1ccc2c(c1)c1ccccc1n2-c1ccc(-p2[nH]c3cccc4c3-c3c(cccc32)[nH]p4-c2ccc(-n3c4ccccc4c4ccccc43)cc2)cc1] | 64% |
| 2b | 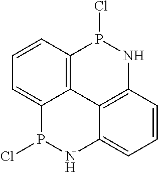 [SMILES: Clp1[nH]c2cccc3[nH]p(Cl)c4cccc1c4-c23] | 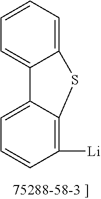 | 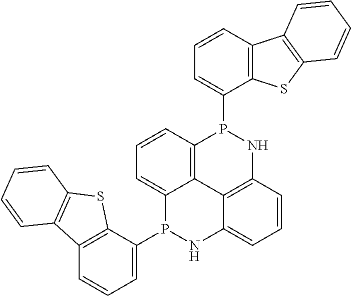 [SMILES: c1cc2[nH]p(-c3cccc4c3sc3ccccc34)c3cccc4c3-c2c(c1)[nH]p4-c1cccc2c1sc1ccccc12] | 61% |
| 3b | 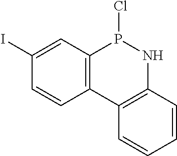 [SMILES: ClP1Nc2ccccc2-c2ccc(I)cc21] | 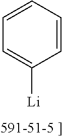 | 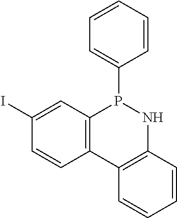 [SMILES: Ic1ccc2c(c1)P(c1ccccc1)Nc1ccccc1-2] | 56% |
| 4b | 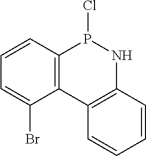 [SMILES: ClP1Nc2ccccc2-c2c(Br)cccc21] | 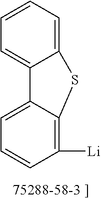 | 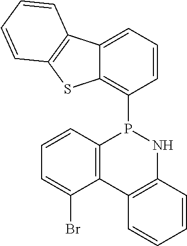 [SMILES: Brc1cccc2c1-c1ccccc1NP2c1cccc2c1sc1ccccc12] | 50% |
| 5b | 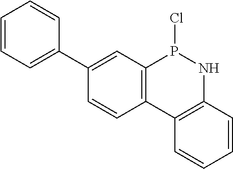 [SMILES: ClP1Nc2ccccc2-c2ccc(-c3ccccc3)cc21] | 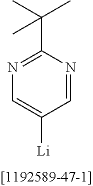 | 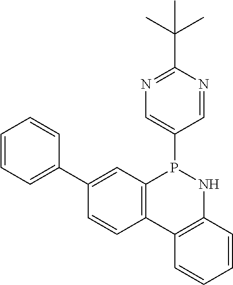 [SMILES: CC(C)(C)c1ncc(P2Nc3ccccc3-c3ccc(-c4ccccc4)cc32)cn1] | 62% |
| 6b | 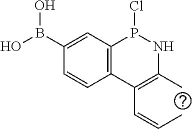 [SMILES: C/C=C\C1=C(C)NP(Cl)c2cc(B(O)O)ccc21] | 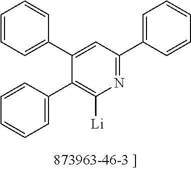 | 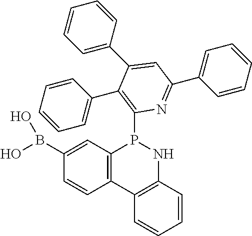 [SMILES: OB(O)c1ccc2c(c1)P(c1nc(-c3ccccc3)cc(-c3ccccc3)c1-c1ccccc1)Nc1ccccc1-2] | 47% |
| 7b | 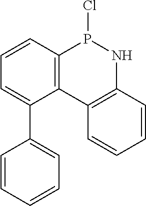 [SMILES: ClP1Nc2ccccc2-c2c(-c3ccccc3)cccc21] | 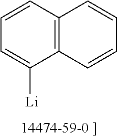 | 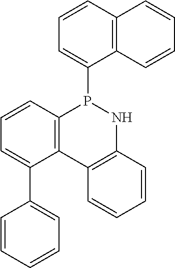 [SMILES: c1ccc(-c2cccc3c2-c2ccccc2NP3c2cccc3ccccc23)cc1] | 63% |
| 8b | 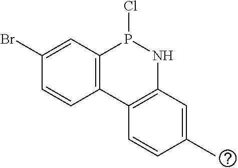 [SMILES: Cc1ccc2c(c1)NP(Cl)c1cc(Br)ccc1-2] | 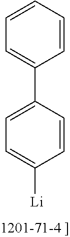 | 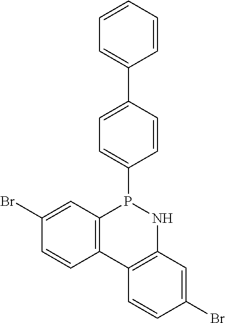 [SMILES: Brc1ccc2c(c1)NP(c1ccc(-c3ccccc3)cc1)c1cc(Br)ccc1-2] | 59% |
| 9b | 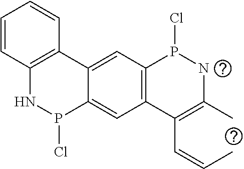 [SMILES: C/C=C\c1c(C)[nH]p(Cl)c2cc3c4ccccc4[nH]p(Cl)c3cc12] | 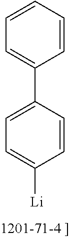 | 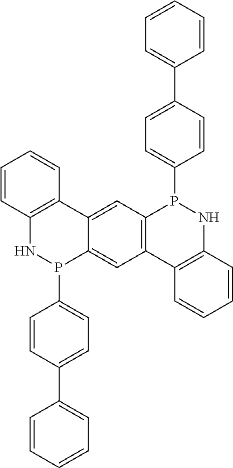 [SMILES: c1ccc(-c2ccc(-p3[nH]c4ccccc4c4cc5c(cc43)c3ccccc3[nH]p5-c3ccc(-c4ccccc4)cc3)cc2)cc1] | 66% |
| 10b | 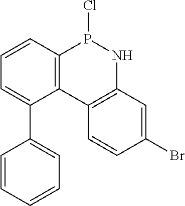 [SMILES: ClP1Nc2cc(Br)ccc2-c2c(-c3ccccc3)cccc21] | 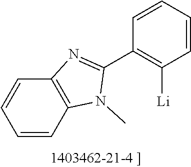 | 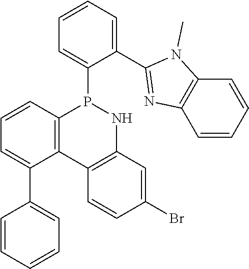 [SMILES: Cn1c(-c2ccccc2P2Nc3cc(Br)ccc3-c3c(-c4ccccc4)cccc32)nc2ccccc21] | 48% |
| 11b | 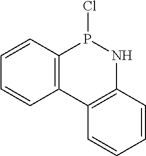 [SMILES: ClP1Nc2ccccc2-c2ccccc21] | 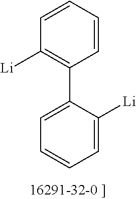 | 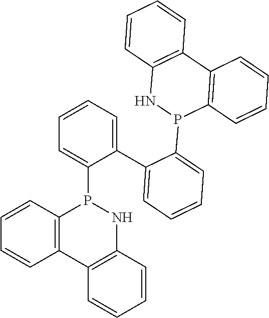 [SMILES: c1ccc2c(c1)NP(c1ccccc1-c1ccccc1P1Nc3ccccc3-c3ccccc31)c1ccccc1-2] | 62% |

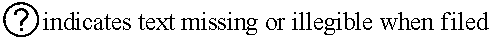

### c) 5,6-Diphenylbenzo[c][2,1]benzazaphosphinin

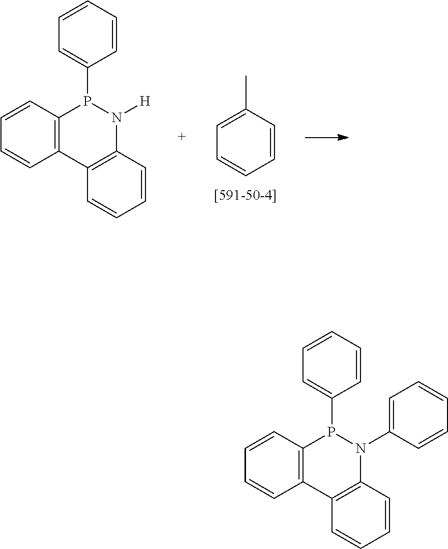

An initial charge of 6.8 g (25 mmol, 1.00 eq.) of 6-phenyl-5H-benzo[c][2,1]benzazaphosphinin, 21.3 ml (128 mmol, 5.2 eq.) of iodobenzene and 7.20 g of potassium carbonate (52.1 mmol, 2.10 eq.) in 220 ml of dry DMF is inertized under argon. Subsequently, 0.62 g (2.7 mmol, 0.11 eq) of 1,3-di(2-pyridyl)propane-1,3-dione and 0.52 g (2.7 mmol, 0.11 eq) of copper(I) iodide are added and the mixture is heated at 140° C. for three days. After the reaction has ended, the mixture is concentrated cautiously on a rotary evaporator, and the precipitated solids are filtered off with suction and washed with water and ethanol. The crude product is purified twice by means of hot extraction (toluene/heptane 1:1), and the solids obtained are recrystallized from toluene. Yield: 4.5 g (12.8 mmol); 52% of theory.

The following compounds can be prepared in an analogous manner:

| | | Reactant | | |
|---|---|---|---|---|
| Ex. | Reactant 1 | 2 | Product | Yield |
| 1c | 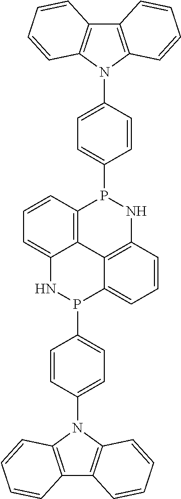 [SMILES: c1ccc2c(c1)c1ccccc1n2-c1ccc(-p2[nH]c3cccc4c3-c3c(cccc32)[nH]p4-c2ccc(-n3c4ccccc4c4ccccc43)cc2)cc1] | 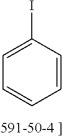 | 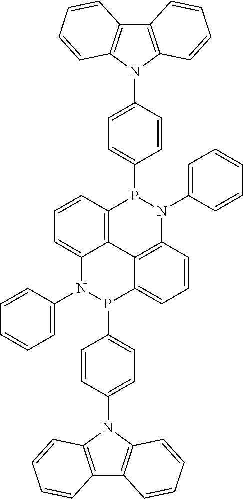 [SMILES: c1ccc(-n2c3cccc4c3-c3c(cccc3p2-c2ccc(-n3c5ccccc5c5ccccc53)cc2)n(-c2ccccc2)p4-c2ccc(-n3c4ccccc4c4ccccc43)cc2)cc1] | 55% |
| 2c | 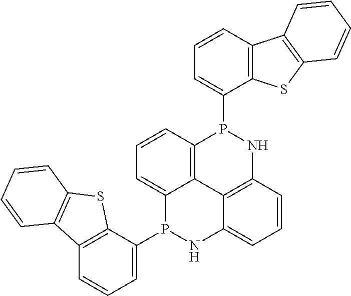 [SMILES: c1cc2[nH]p(-c3cccc4c3sc3ccccc34)c3cccc4c3-c2c(c1)[nH]p4-c1cccc2c1sc1ccccc12] | 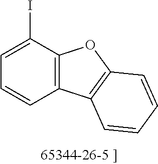 [SMILES: Ic1cccc2c1oc1ccccc12] | 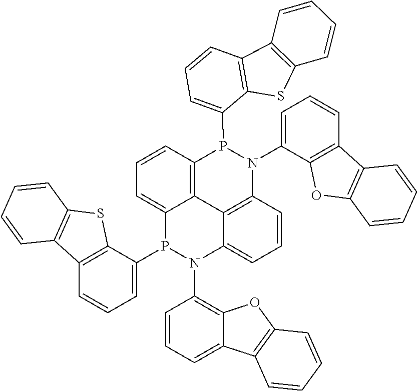 [SMILES: c1ccc2c(c1)oc1c(-n3c4cccc5c4-c4c(cccc4p(-c4cccc6c4sc4ccccc46)n5-c4cccc5c4oc4ccccc45)p3-c3cccc4c3sc3ccccc34)cccc12] | 51% |
| 3c | 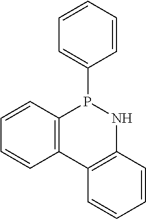 [SMILES: c1ccc(P2Nc3ccccc3-c3ccccc32)cc1] | 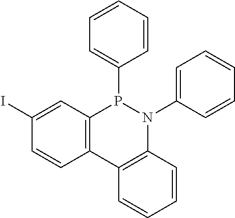 [SMILES: Ic1ccc2c(c1)P(c1ccccc1)N(c1ccccc1)c1ccccc1-2] | 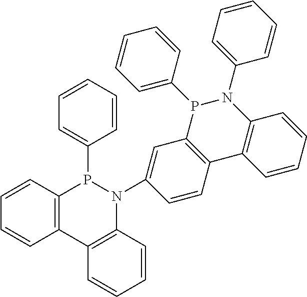 [SMILES: c1ccc(N2c3ccccc3-c3ccc(N4c5ccccc5-c5ccccc5P4c4ccccc4)cc3P2c2ccccc2)cc1] | 60% |
| 4c | 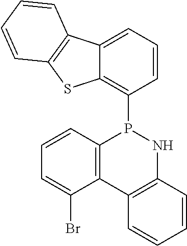 [SMILES: Brc1cccc2c1-c1ccccc1NP2c1cccc2c1sc1ccccc12] | 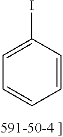 | 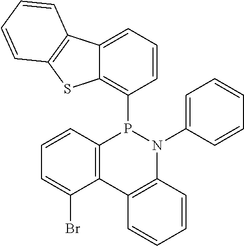 [SMILES: Brc1cccc2c1-c1ccccc1N(c1ccccc1)P2c1cccc2c1sc1ccccc12] | 46% |
| 5c | 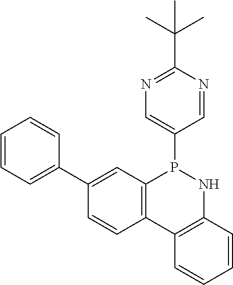 [SMILES: CC(C)(C)c1ncc(P2Nc3ccccc3-c3ccc(-c4ccccc4)cc32)cn1] | 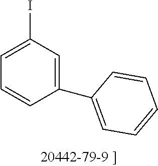 [SMILES: Ic1cccc(-c2ccccc2)c1] | 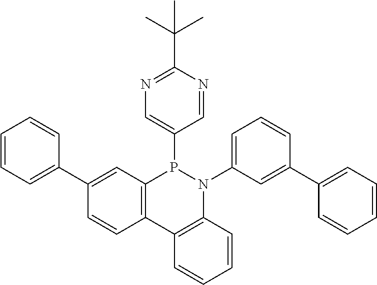 [SMILES: CC(C)(C)c1ncc(P2c3cc(-c4ccccc4)ccc3-c3ccccc3N2c2cccc(-c3ccccc3)c2)cn1] | 50% |
| 6c | 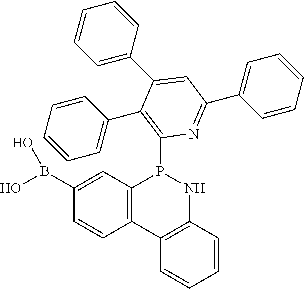 [SMILES: OB(O)c1ccc2c(c1)P(c1nc(-c3ccccc3)cc(-c3ccccc3)c1-c1ccccc1)Nc1ccccc1-2] | 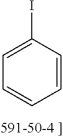 | 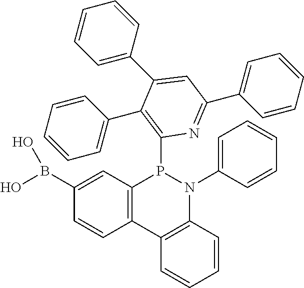 [SMILES: OB(O)c1ccc2c(c1)P(c1nc(-c3ccccc3)cc(-c3ccccc3)c1-c1ccccc1)N(c1ccccc1)c1ccccc1-2] | 34% |
| 7c | 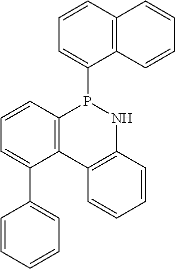 [SMILES: c1ccc(-c2cccc3c2-c2ccccc2NP3c2cccc3ccccc23)cc1] | 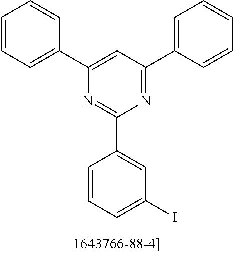 [SMILES: Ic1cccc(-c2nc(-c3ccccc3)cc(-c3ccccc3)n2)c1] | 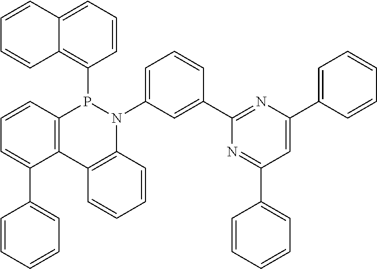 [SMILES: c1ccc(-c2cc(-c3ccccc3)nc(-c3cccc(N4c5ccccc5-c5c(-c6ccccc6)cccc5P4c4cccc5ccccc45)c3)n2)cc1] | 49% |
| 8c | 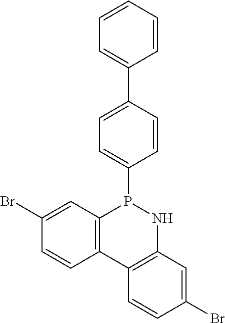 [SMILES: Brc1ccc2c(c1)NP(c1ccc(-c3ccccc3)cc1)c1cc(Br)ccc1-2] | 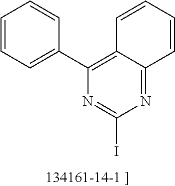 [SMILES: Ic1nc(-c2ccccc2)c2ccccc2n1] | 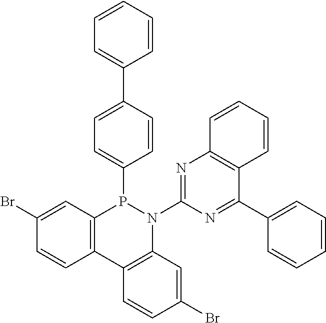 [SMILES: Brc1ccc2c(c1)N(c1nc(-c3ccccc3)c3ccccc3n1)P(c1ccc(-c3ccccc3)cc1)c1cc(Br)ccc1-2] | 51% |
| 9c | 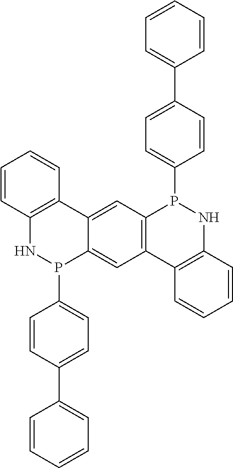 [SMILES: c1ccc(-c2ccc(-p3[nH]c4ccccc4c4cc5c(cc43)c3ccccc3[nH]p5-c3ccc(-c4ccccc4)cc3)cc2)cc1] | 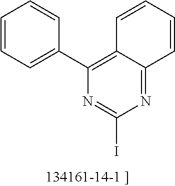 [SMILES: Ic1nc(-c2ccccc2)c2ccccc2n1] | 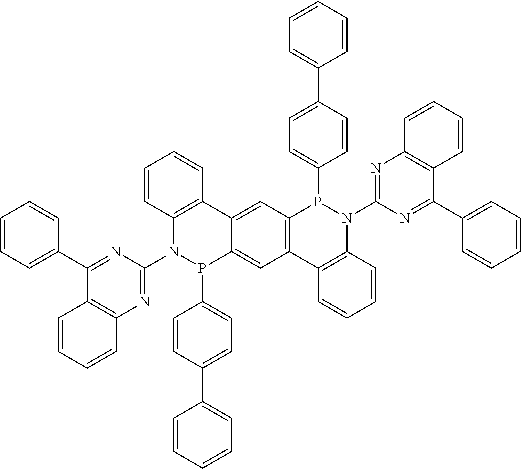 [SMILES: c1ccc(-c2ccc(-p3c4cc5c6ccccc6n(-c6nc(-c7ccccc7)c7ccccc7n6)p(-c6ccc(-c7ccccc7)cc6)c5cc4c4ccccc4n3-c3nc(-c4ccccc4)c4ccccc4n3)cc2)cc1] | 50% |
| 10c | 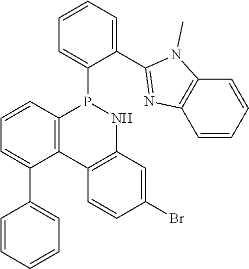 [SMILES: Cn1c(-c2ccccc2P2Nc3cc(Br)ccc3-c3c(-c4ccccc4)cccc32)nc2ccccc21] | 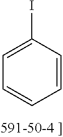 | 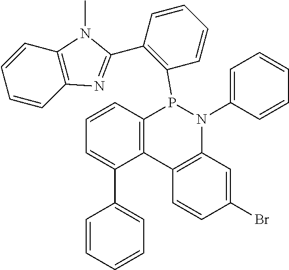 [SMILES: Cn1c(-c2ccccc2P2c3cccc(-c4ccccc4)c3-c3ccc(Br)cc3N2c2ccccc2)nc2ccccc21] | 47% |
| 11c | 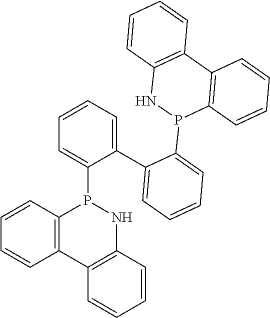 [SMILES: c1ccc2c(c1)NP(c1ccccc1-c1ccccc1P1Nc3ccccc3-c3ccccc31)c1ccccc1-2] | 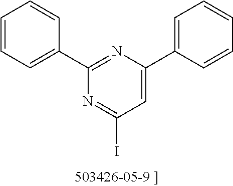 [SMILES: Ic1cc(-c2ccccc2)nc(-c2ccccc2)n1] | 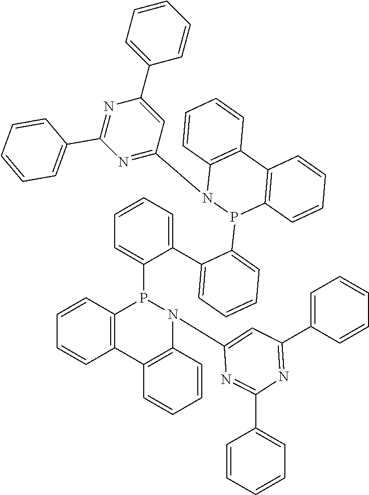 [SMILES: c1ccc(-c2cc(N3c4ccccc4-c4ccccc4P3c3ccccc3-c3ccccc3P3c4ccccc4-c4ccccc4N3c3cc(-c4ccccc4)nc(-c4ccccc4)n3)nc(-c3ccccc3)n2)cc1] | 44% |
| 12c | 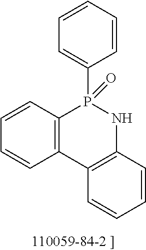 [SMILES: O=P1(c2ccccc2)Nc2ccccc2-c2ccccc21] | 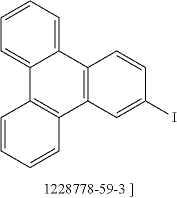 [SMILES: Ic1ccc2c3ccccc3c3ccccc3c2c1] | 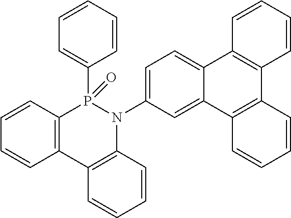 [SMILES: O=P1(c2ccccc2)c2ccccc2-c2ccccc2N1c1ccc2c3ccccc3c3ccccc3c2c1] | 56% |
| 13c | 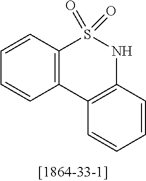 [SMILES: O=S1(=O)Nc2ccccc2-c2ccccc21] | 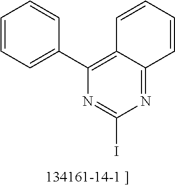 [SMILES: Ic1nc(-c2ccccc2)c2ccccc2n1] | 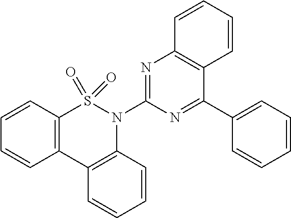 [SMILES: O=S1(=O)c2ccccc2-c2ccccc2N1c1nc(-c2ccccc2)c2ccccc2n1] | 55% |
| 14c | 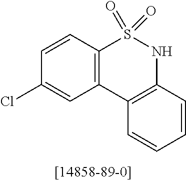 [SMILES: O=S1(=O)Nc2ccccc2-c2cc(Cl)ccc21] | 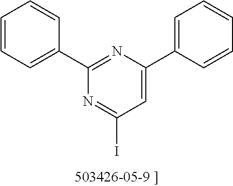 [SMILES: Ic1cc(-c2ccccc2)nc(-c2ccccc2)n1] | 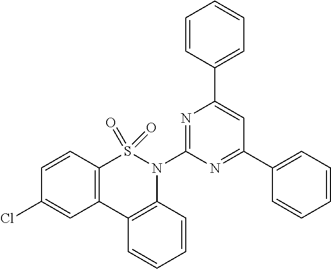 [SMILES: O=S1(=O)c2ccc(Cl)cc2-c2ccccc2N1c1nc(-c2ccccc2)cc(-c2ccccc2)n1] | 61% |
| 15c | 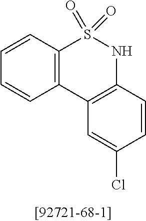 [SMILES: O=S1(=O)Nc2ccc(Cl)cc2-c2ccccc21] | 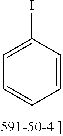 | 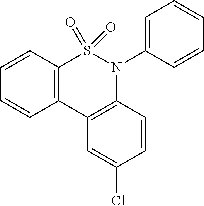 [SMILES: O=S1(=O)c2ccccc2-c2cc(Cl)ccc2N1c1ccccc1] | 60% |
| 16c | 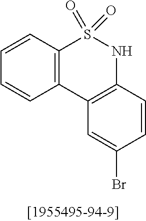 [SMILES: O=S1(=O)Nc2ccc(Br)cc2-c2ccccc21] | 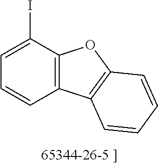 [SMILES: Ic1cccc2c1oc1ccccc12] | 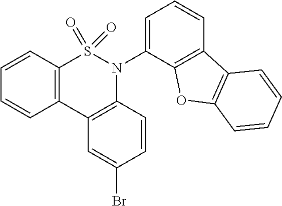 [SMILES: O=S1(=O)c2ccccc2-c2cc(Br)ccc2N1c1cccc2c1oc1ccccc12] | 57% |
| 17c | 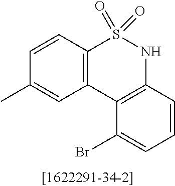 [SMILES: Cc1ccc2c(c1)-c1c(Br)cccc1NS2(=O)=O] | 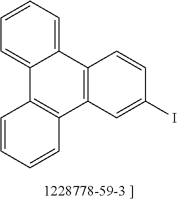 [SMILES: Ic1ccc2c3ccccc3c3ccccc3c2c1] | 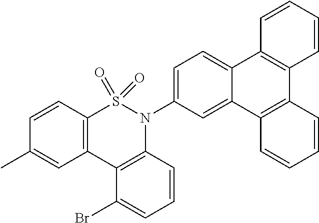 [SMILES: Cc1ccc2c(c1)-c1c(Br)cccc1N(c1ccc3c4ccccc4c4ccccc4c3c1)S2(=O)=O] | 53% |
| 18c | 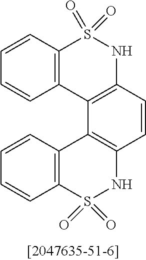 [SMILES: O=S1(=O)Nc2ccc3c(c2-c2ccccc21)-c1ccccc1S(=O)(=O)N3] | 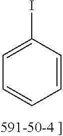 | 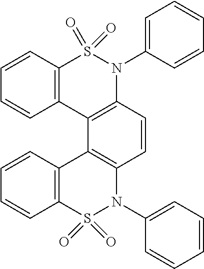 [SMILES: O=S1(=O)c2ccccc2-c2c(ccc3c2-c2ccccc2S(=O)(=O)N3c2ccccc2)N1c1ccccc1] | 54% |
| 19c | 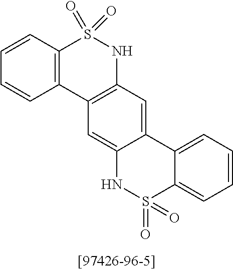 [SMILES: O=S1(=O)Nc2cc3c(cc2-c2ccccc21)NS(=O)(=O)c1ccccc1-3] | 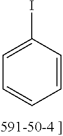 | 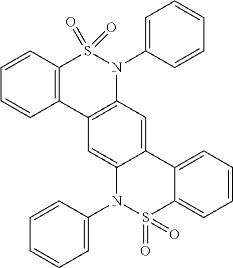 [SMILES: O=S1(=O)c2ccccc2-c2cc3c(cc2N1c1ccccc1)-c1ccccc1S(=O)(=O)N3c1ccccc1] | 50% |
| 20c | 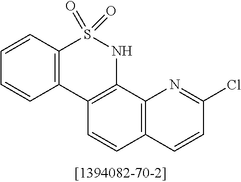 [SMILES: O=S1(=O)Nc2c(ccc3ccc(Cl)nc23)-c2ccccc21] | 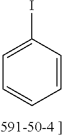 | 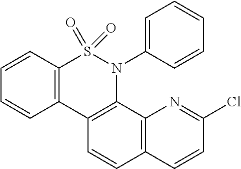 [SMILES: O=S1(=O)c2ccccc2-c2ccc3ccc(Cl)nc3c2N1c1ccccc1] | 47% |
| 21c | 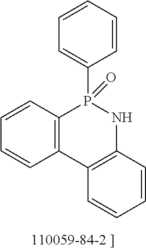 [SMILES: O=P1(c2ccccc2)Nc2ccccc2-c2ccccc21] | 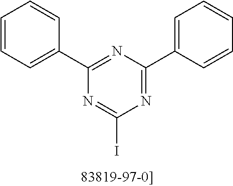 [SMILES: Ic1nc(-c2ccccc2)nc(-c2ccccc2)n1] | 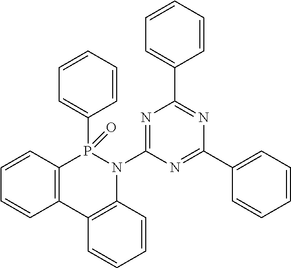 [SMILES: O=P1(c2ccccc2)c2ccccc2-c2ccccc2N1c1nc(-c2ccccc2)nc(-c2ccccc2)n1] | 50% |
| 22c | 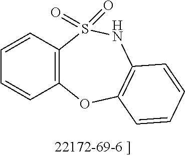 [SMILES: O=S1(=O)Nc2ccccc2Oc2ccccc21] | 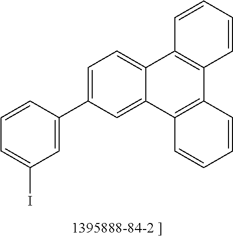 [SMILES: Ic1cccc(-c2ccc3c4ccccc4c4ccccc4c3c2)c1] | 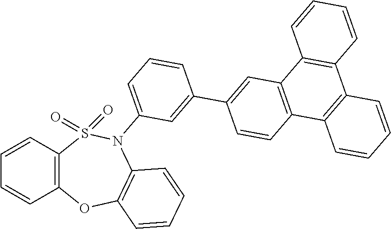 [SMILES: O=S1(=O)c2ccccc2Oc2ccccc2N1c1cccc(-c2ccc3c4ccccc4c4ccccc4c3c2)c1] | 45% |
| 23c | 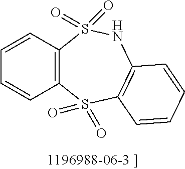 [SMILES: O=S1(=O)Nc2ccccc2S(=O)(=O)c2ccccc21] | 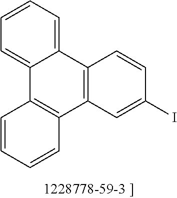 [SMILES: Ic1ccc2c3ccccc3c3ccccc3c2c1] | 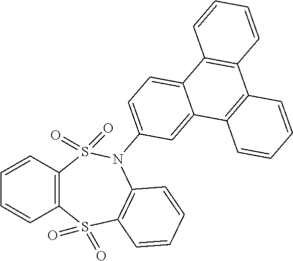 [SMILES: O=S1(=O)c2ccccc2N(c2ccc3c4ccccc4c4ccccc4c3c2)S(=O)(=O)c2ccccc21] | 48% |
| 24c | 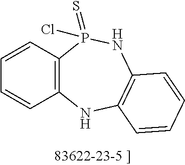 [SMILES: S=P1(Cl)Nc2ccccc2Nc2ccccc21] | 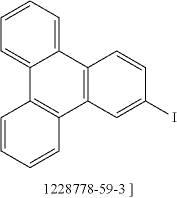 [SMILES: Ic1ccc2c3ccccc3c3ccccc3c2c1] | 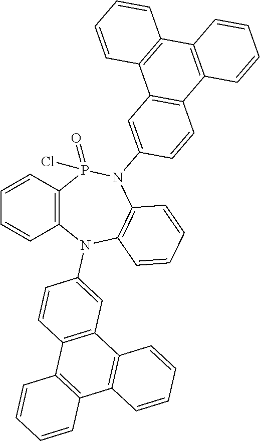 [SMILES: O=P1(Cl)c2ccccc2N(c2ccc3c4ccccc4c4ccccc4c3c2)c2ccccc2N1c1ccc2c3ccccc3c3ccccc3c2c1] | 42% |
| 25c | 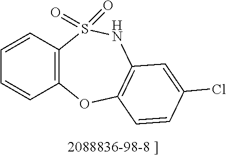 [SMILES: O=S1(=O)Nc2cc(Cl)ccc2Oc2ccccc21] | 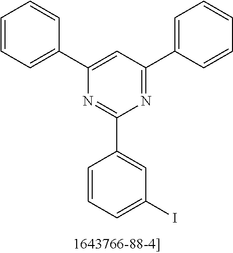 [SMILES: Ic1cccc(-c2nc(-c3ccccc3)cc(-c3ccccc3)n2)c1] | 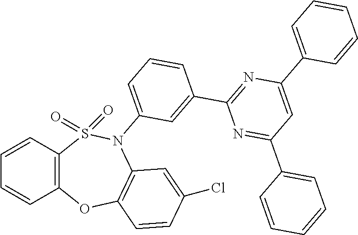 [SMILES: O=S1(=O)c2ccccc2Oc2ccc(Cl)cc2N1c1cccc(-c2nc(-c3ccccc3)cc(-c3ccccc3)n2)c1] | 50% |

Compounds 19c, 20c, and 22c-24c are purified by sublimation to a purity of 99.9%.

### d) 5,6-Diphenylbenzo[c][2,1]benzazaphosphinin 6-oxide

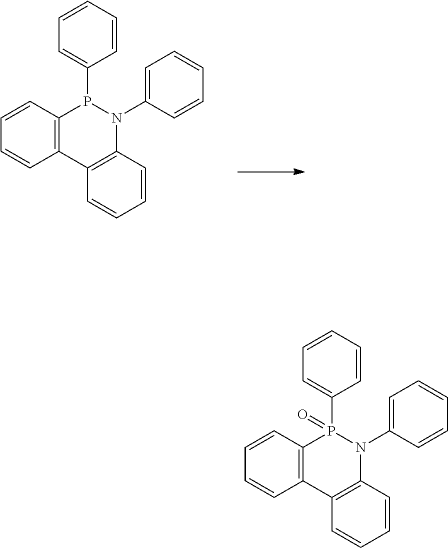 [SMILES: c1ccc(N2c3ccccc3-c3ccccc3P2c2ccccc2)cc1 ; O=P1(c2ccccc2)c2ccccc2-c2ccccc2N1c1ccccc1]

4.5 g (12.8 mmol) is dissolved in 40 ml of ethanol at room temperature, and 80 ml of H₂O₂ (30%) is added dropwise within 30 min. After stirring at 7000 for 1 h, 100 ml of dichloromethane is added to the solution, the phases are separated, the organic phase is concentrated and the product is crystallized from heptane/dichloromethane 2:1. Yield: 3.9 g (10.5 mmol); 93% of theory.

| Ex. | Reactant | Product | Yield |
|---|---|---|---|
| 1d | 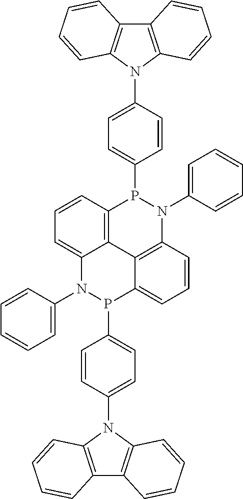 [SMILES: c1ccc(-n2c3cccc4c3-c3c(cccc3p2-c2ccc(-n3c5ccccc5c5ccccc53)cc2)n(-c2ccccc2)p4-c2ccc(-n3c4ccccc4c4ccccc43)cc2)cc1] | 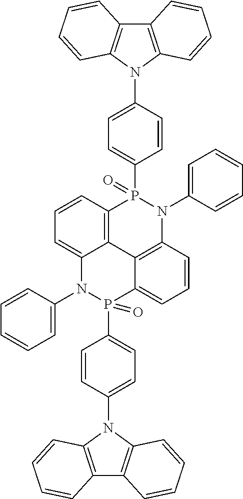 [SMILES: O=P1(c2ccc(-n3c4ccccc4c4ccccc43)cc2)c2cccc3c2-c2c(cccc2P(=O)(c2ccc(-n4c5ccccc5c5ccccc54)cc2)N3c2ccccc2)N1c1ccccc1] | 94% |
| 2d | 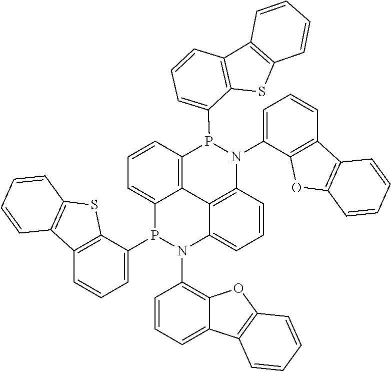 [SMILES: c1ccc2c(c1)oc1c(-n3c4cccc5c4-c4c(cccc4p(-c4cccc6c4sc4ccccc46)n5-c4cccc5c4oc4ccccc45)p3-c3cccc4c3sc3ccccc34)cccc12] | 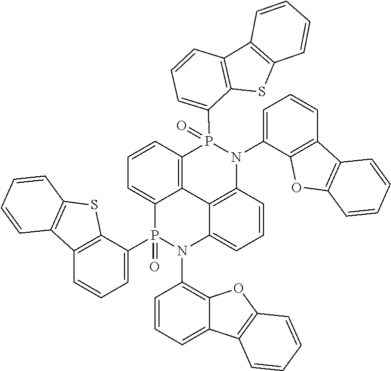 [SMILES: O=P1(c2cccc3c2sc2ccccc23)c2cccc3c2-c2c(cccc2N(c2cccc4c2oc2ccccc24)P3(=O)c2cccc3c2sc2ccccc23)N1c1cccc2c1oc1ccccc12] | 91% |
| 3d | 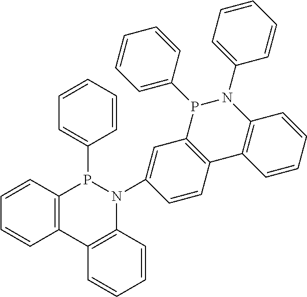 [SMILES: c1ccc(N2c3ccccc3-c3ccc(N4c5ccccc5-c5ccccc5P4c4ccccc4)cc3P2c2ccccc2)cc1] | 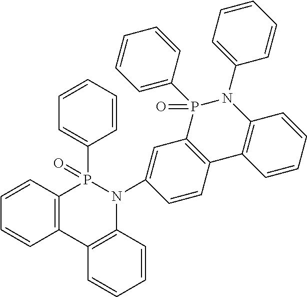 [SMILES: O=P1(c2ccccc2)c2ccccc2-c2ccccc2N1c1ccc2c(c1)P(=O)(c1ccccc1)N(c1ccccc1)c1ccccc1-2] | 94% |
| 4d | 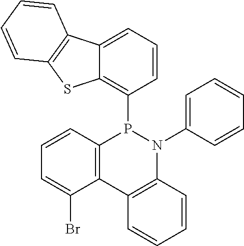 [SMILES: Brc1cccc2c1-c1ccccc1N(c1ccccc1)P2c1cccc2c1sc1ccccc12] | 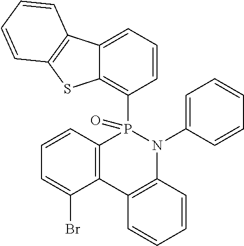 [SMILES: O=P1(c2cccc3c2sc2ccccc23)c2cccc(Br)c2-c2ccccc2N1c1ccccc1] | 90% |
| 5d | 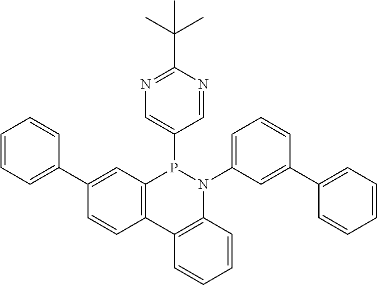 [SMILES: CC(C)(C)c1ncc(P2c3cc(-c4ccccc4)ccc3-c3ccccc3N2c2cccc(-c3ccccc3)c2)cn1] | 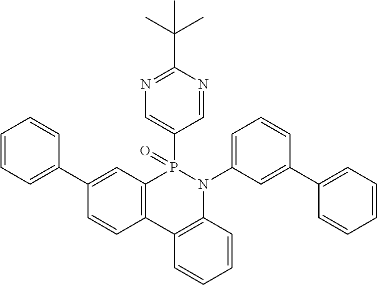 [SMILES: CC(C)(C)c1ncc(P2(=O)c3cc(-c4ccccc4)ccc3-c3ccccc3N2c2cccc(-c3ccccc3)c2)cn1] | 89% |
| 6d | 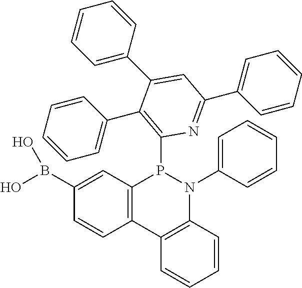 [SMILES: OB(O)c1ccc2c(c1)P(c1nc(-c3ccccc3)cc(-c3ccccc3)c1-c1ccccc1)N(c1ccccc1)c1ccccc1-2] | 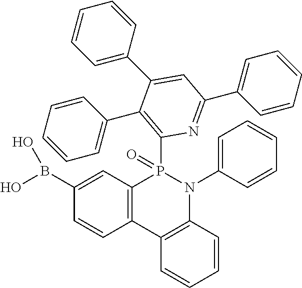 [SMILES: O=P1(c2nc(-c3ccccc3)cc(-c3ccccc3)c2-c2ccccc2)c2cc(B(O)O)ccc2-c2ccccc2N1c1ccccc1] | 74% |
| 7d | 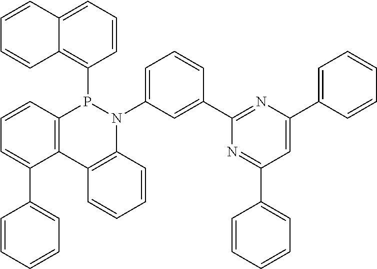 [SMILES: c1ccc(-c2cc(-c3ccccc3)nc(-c3cccc(N4c5ccccc5-c5c(-c6ccccc6)cccc5P4c4cccc5ccccc45)c3)n2)cc1] | 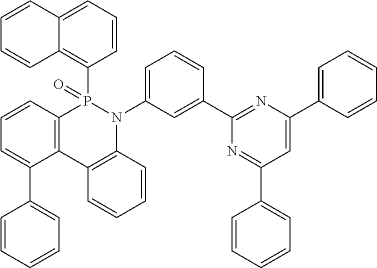 [SMILES: O=P1(c2cccc3ccccc23)c2cccc(-c3ccccc3)c2-c2ccccc2N1c1cccc(-c2nc(-c3ccccc3)cc(-c3ccccc3)n2)c1] | 90% |
| 8d | 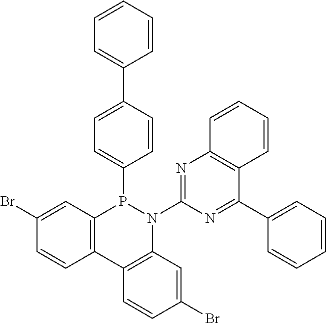 [SMILES: Brc1ccc2c(c1)N(c1nc(-c3ccccc3)c3ccccc3n1)P(c1ccc(-c3ccccc3)cc1)c1cc(Br)ccc1-2] | 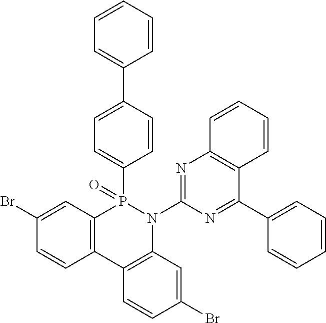 [SMILES: O=P1(c2ccc(-c3ccccc3)cc2)c2cc(Br)ccc2-c2ccc(Br)cc2N1c1nc(-c2ccccc2)c2ccccc2n1] | 89% |
| 9d | 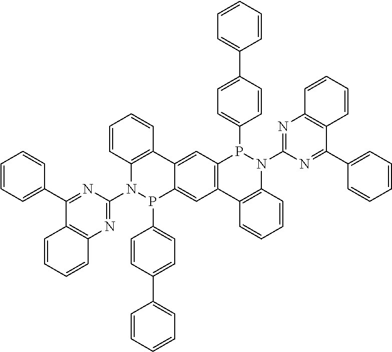 [SMILES: c1ccc(-c2ccc(-p3c4cc5c6ccccc6n(-c6nc(-c7ccccc7)c7ccccc7n6)p(-c6ccc(-c7ccccc7)cc6)c5cc4c4ccccc4n3-c3nc(-c4ccccc4)c4ccccc4n3)cc2)cc1] | 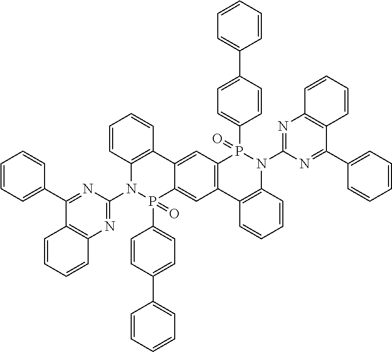 [SMILES: O=P1(c2ccc(-c3ccccc3)cc2)c2cc3c(cc2-c2ccccc2N1c1nc(-c2ccccc2)c2ccccc2n1)P(=O)(c1ccc(-c2ccccc2)cc1)N(c1nc(-c2ccccc2)c2ccccc2n1)c1ccccc1-3] | 92% |
| 10c | 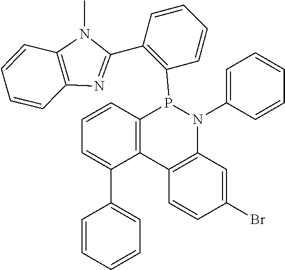 [SMILES: Cn1c(-c2ccccc2P2c3cccc(-c4ccccc4)c3-c3ccc(Br)cc3N2c2ccccc2)nc2ccccc21] | 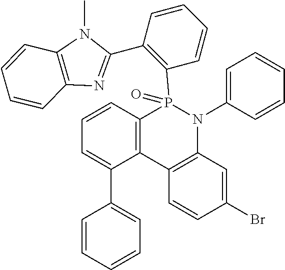 [SMILES: Cn1c(-c2ccccc2P2(=O)c3cccc(-c4ccccc4)c3-c3ccc(Br)cc3N2c2ccccc2)nc2ccccc21] | 93% |
| 11d | 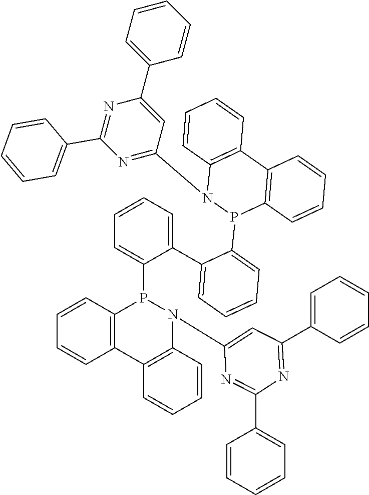 [SMILES: c1ccc(-c2cc(N3c4ccccc4-c4ccccc4P3c3ccccc3-c3ccccc3P3c4ccccc4-c4ccccc4N3c3cc(-c4ccccc4)nc(-c4ccccc4)n3)nc(-c3ccccc3)n2)cc1] | 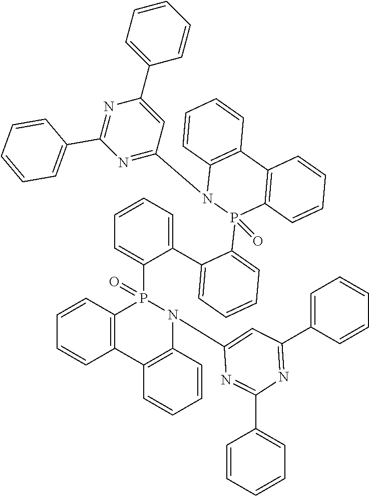 [SMILES: O=P1(c2ccccc2-c2ccccc2P2(=O)c3ccccc3-c3ccccc3N2c2cc(-c3ccccc3)nc(-c3ccccc3)n2)c2ccccc2-c2ccccc2N1c1cc(-c2ccccc2)nc(-c2ccccc2)n1] | 91% |

Compounds 1d-3d, 5d, 7d, 9d and 10d are recrystallized from toluene and finally fractionally sublimed twice (p about 10⁻⁶ mbar, T=330-450° C.).

### e) 2-Bromo-5,6-diphenylbenzo[c][1,2]benzazaphosphinin 6-oxide

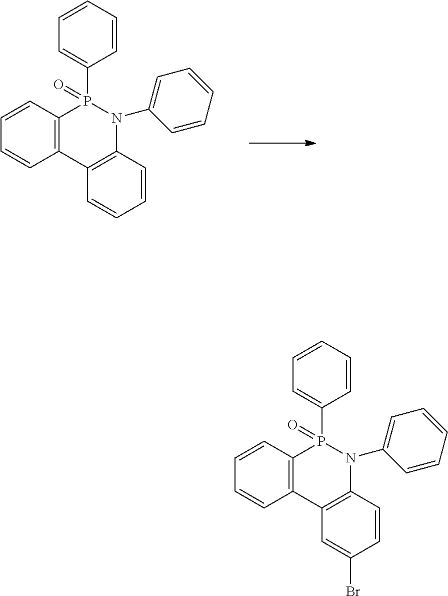 [SMILES: O=P1(c2ccccc2)c2ccccc2-c2ccccc2N1c1ccccc1 ; O=P1(c2ccccc2)c2ccccc2-c2cc(Br)ccc2N1c1ccccc1]

To a solution of 56 g (154 mmol) of 5,6-diphenylbenzo[c][2,1]benzazaphosphinin 6-oxide in chloroform (1000 ml) is added N-bromosuccinimide (24.7 g, 139 mmol) in portions at 0° C. with exclusion of light, and the mixture is stirred at this temperature for 2 h. The reaction is ended by addition of sodium sulfite solution and the mixture is stirred at room temperature for a further 30 min. After phase separation, the organic phase is washed with water and the aqueous phase is extracted with dichloromethane. The combined organic phases are dried over sodium sulfate and concentrated under reduced pressure. The residue is dissolved in toluene and filtered through silica gel. Subsequently, the crude product is recrystallized from toluene/heptane. Yield: 44 g (99 mmol); 65% of theory of a colorless solid.

The following compounds can be prepared in an analogous manner:

| | Reactant | Product | Yield |
|---|---|---|---|
| 1e | 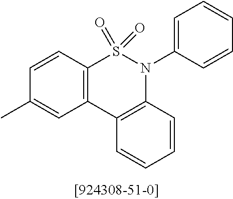 [SMILES: Cc1ccc2c(c1)-c1ccccc1N(c1ccccc1)S2(=O)=O] | 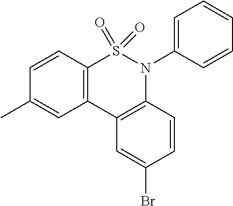 [SMILES: Cc1ccc2c(c1)-c1cc(Br)ccc1N(c1ccccc1)S2(=O)=O] | 67% |
| 2e | 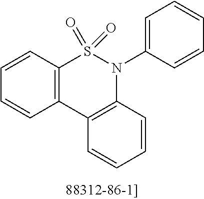 [SMILES: O=S1(=O)c2ccccc2-c2ccccc2N1c1ccccc1] | 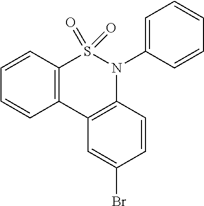 [SMILES: O=S1(=O)c2ccccc2-c2cc(Br)ccc2N1c1ccccc1] | 68% |
| 3e | 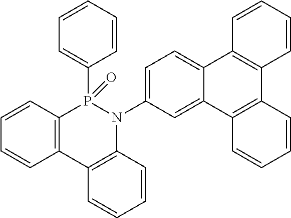 [SMILES: O=P1(c2ccccc2)c2ccccc2-c2ccccc2N1c1ccc2c3ccccc3c3ccccc3c2c1] | 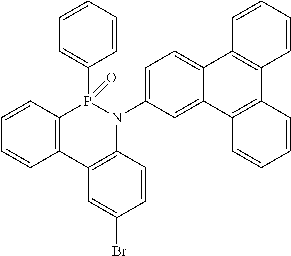 [SMILES: O=P1(c2ccccc2)c2ccccc2-c2cc(Br)ccc2N1c1ccc2c3ccccc3c3ccccc3c2c1] | 80% |
| 4e | 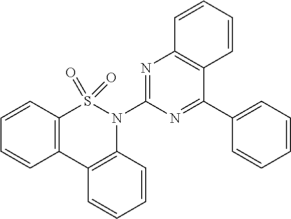 [SMILES: O=S1(=O)c2ccccc2-c2ccccc2N1c1nc(-c2ccccc2)c2ccccc2n1] | 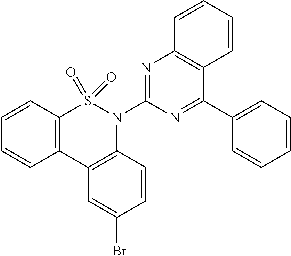 [SMILES: O=S1(=O)c2ccccc2-c2cc(Br)ccc2N1c1nc(-c2ccccc2)c2ccccc2n1] | 79% |
| 5e | 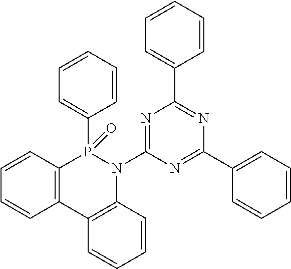 [SMILES: O=P1(c2ccccc2)c2ccccc2-c2ccccc2N1c1nc(-c2ccccc2)nc(-c2ccccc2)n1] | 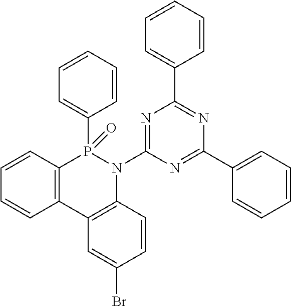 [SMILES: O=P1(c2ccccc2)c2ccccc2-c2cc(Br)ccc2N1c1nc(-c2ccccc2)nc(-c2ccccc2)n1] | 77% |

### f) 5,6-Diphenyl-2-(9-phenylcarbazol-3-yl)benzo[c][1,2]benzazaphosphinin 6-oxide

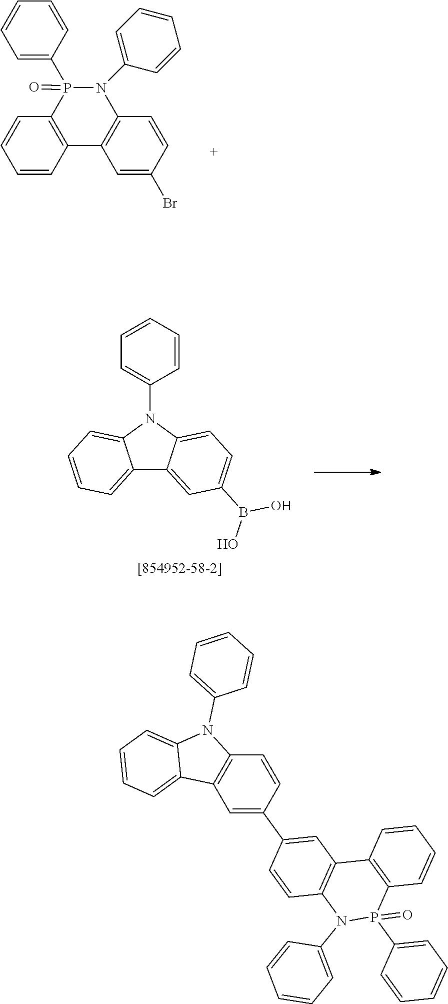 [SMILES: O=P1(c2ccccc2)c2ccccc2-c2cc(Br)ccc2N1c1ccccc1 ; OB(O)c1ccc2c(c1)c1ccccc1n2-c1ccccc1 ; O=P1(c2ccccc2)c2ccccc2-c2cc(-c3ccc4c(c3)c3ccccc3n4-c3ccccc3)ccc2N1c1ccccc1]

31 (70 mmol) of 2-bromo-5,6-diphenylbenzo[c][1,2]benzazaphosphinin 6-oxide, 20.8 g (75 mmol) of phenylcarbazole-3-boronic acid and 14.7 g (139 mmol) of sodium carbonate are suspended in 200 ml of toluene, 52 ml of ethanol and 100 ml of water. 80 mg (0.69 mmol) of tetrakistriphenylphosphinepalladium(0) are added to this suspension, and the reaction mixture is heated under reflux for 16 h. After cooling, the organic phase is removed, filtered through silica gel, washed three times with 200 ml of water and then concentrated to dryness. The residue is recrystallized from heptane/dichloromethane. The yield is 33 g (55 mmol); 79% of theory.

The following compounds can be obtained in an analogous manner:

| | Reactant 1 | Reactant 2 | Product | Yield |
|---|---|---|---|---|
| 1f | 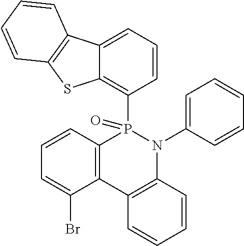 [SMILES: O=P1(c2cccc3c2sc2ccccc23)c2cccc(Br)c2-c2ccccc2N1c1ccccc1] | 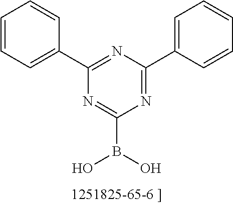 [SMILES: OB(O)c1nc(-c2ccccc2)nc(-c2ccccc2)n1] | 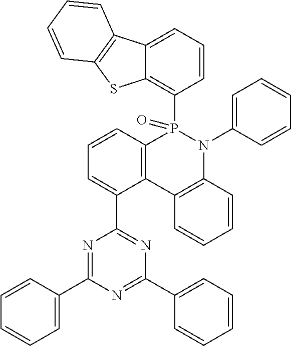 [SMILES: O=P1(c2cccc3c2sc2ccccc23)c2cccc(-c3nc(-c4ccccc4)nc(-c4ccccc4)n3)c2-c2ccccc2N1c1ccccc1] | 75% |
| 2f | 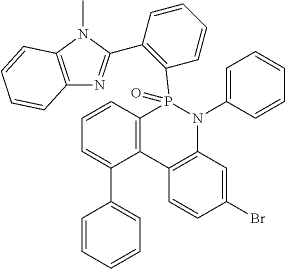 [SMILES: Cn1c(-c2ccccc2P2(=O)c3cccc(-c4ccccc4)c3-c3ccc(Br)cc3N2c2ccccc2)nc2ccccc21] | 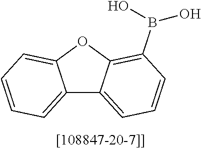 [SMILES: OB(O)c1cccc2c1oc1ccccc12] | 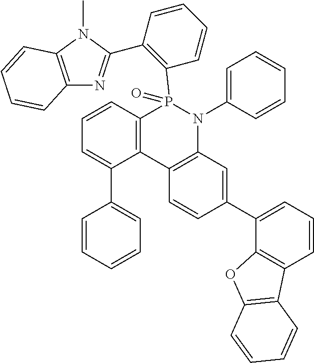 [SMILES: Cn1c(-c2ccccc2P2(=O)c3cccc(-c4ccccc4)c3-c3ccc(-c4cccc5c4oc4ccccc45)cc3N2c2ccccc2)nc2ccccc21] | 64% |
| 3f | 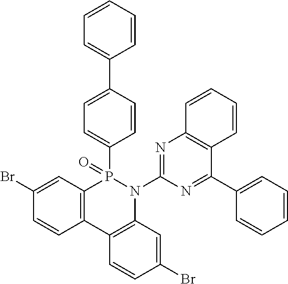 [SMILES: O=P1(c2ccc(-c3ccccc3)cc2)c2cc(Br)ccc2-c2ccc(Br)cc2N1c1nc(-c2ccccc2)c2ccccc2n1] | 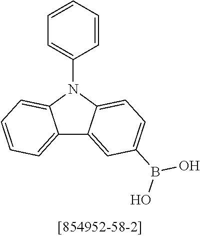 [SMILES: OB(O)c1ccc2c(c1)c1ccccc1n2-c1ccccc1] | 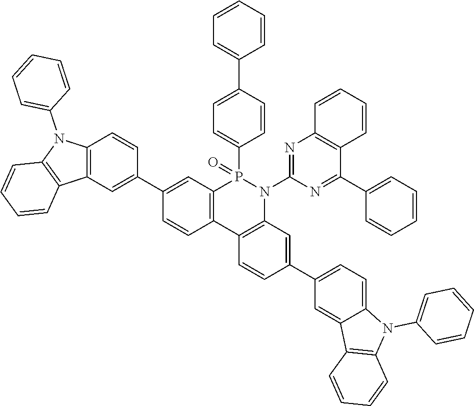 [SMILES: O=P1(c2ccc(-c3ccccc3)cc2)c2cc(-c3ccc4c(c3)c3ccccc3n4-c3ccccc3)ccc2-c2ccc(-c3ccc4c(c3)c3ccccc3n4-c3ccccc3)cc2N1c1nc(-c2ccccc2)c2ccccc2n1] | 76% |
| 4f | 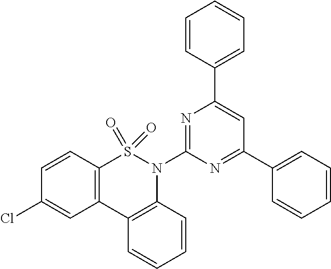 [SMILES: O=S1(=O)c2ccc(Cl)cc2-c2ccccc2N1c1nc(-c2ccccc2)cc(-c2ccccc2)n1] | 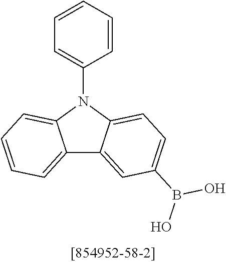 [SMILES: OB(O)c1ccc2c(c1)c1ccccc1n2-c1ccccc1] | 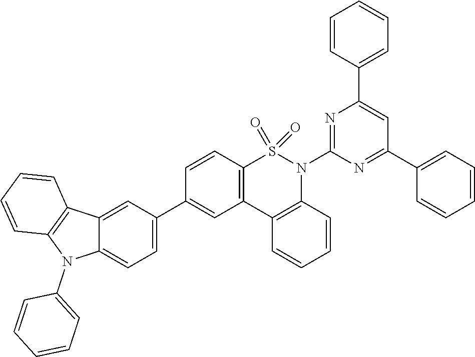 [SMILES: O=S1(=O)c2ccc(-c3ccc4c(c3)c3ccccc3n4-c3ccccc3)cc2-c2ccccc2N1c1nc(-c2ccccc2)cc(-c2ccccc2)n1] | 81% |
| 5f | 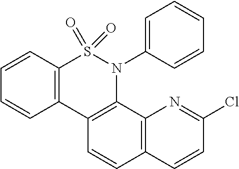 [SMILES: O=S1(=O)c2ccccc2-c2ccc3ccc(Cl)nc3c2N1c1ccccc1] | 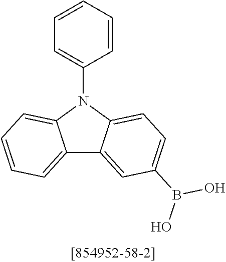 [SMILES: OB(O)c1ccc2c(c1)c1ccccc1n2-c1ccccc1] | 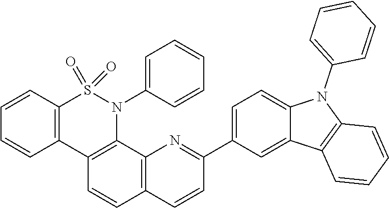 [SMILES: O=S1(=O)c2ccccc2-c2ccc3ccc(-c4ccc5c(c4)c4ccccc4n5-c4ccccc4)nc3c2N1c1ccccc1] | 63% |
| 6f | 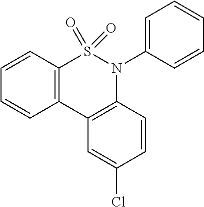 [SMILES: O=S1(=O)c2ccccc2-c2cc(Cl)ccc2N1c1ccccc1] | 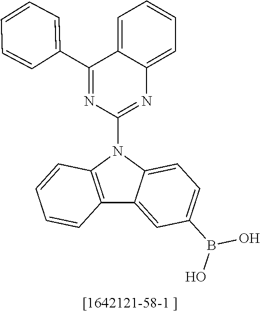 [SMILES: OB(O)c1ccc2c(c1)c1ccccc1n2-c1nc(-c2ccccc2)c2ccccc2n1] | 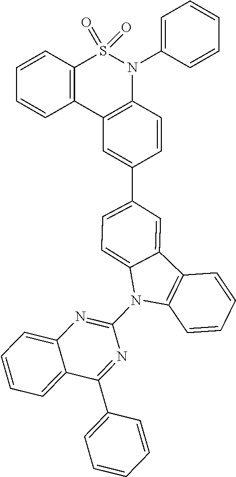 [SMILES: O=S1(=O)c2ccccc2-c2cc(-c3ccc4c(c3)c3ccccc3n4-c3nc(-c4ccccc4)c4ccccc4n3)ccc2N1c1ccccc1] | 76% |
| 7f | 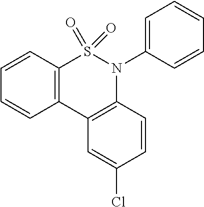 [SMILES: O=S1(=O)c2ccccc2-c2cc(Cl)ccc2N1c1ccccc1] | 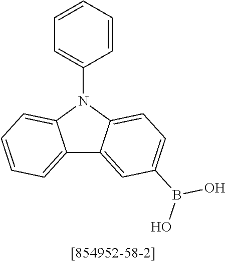 [SMILES: OB(O)c1ccc2c(c1)c1ccccc1n2-c1ccccc1] | 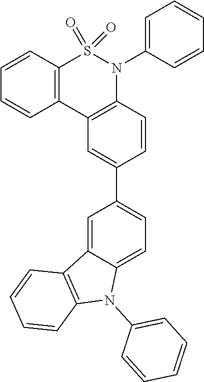 [SMILES: O=S1(=O)c2ccccc2-c2cc(-c3ccc4c(c3)c3ccccc3n4-c3ccccc3)ccc2N1c1ccccc1] | 80% |
| 8f | 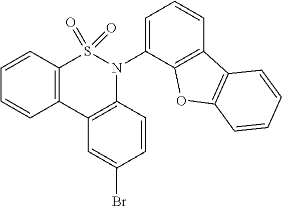 [SMILES: O=S1(=O)c2ccccc2-c2cc(Br)ccc2N1c1cccc2c1oc1ccccc12] | 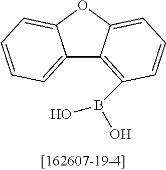 [SMILES: OB(O)c1cccc2oc3ccccc3c12] | 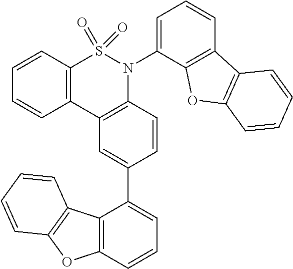 [SMILES: O=S1(=O)c2ccccc2-c2cc(-c3cccc4oc5ccccc5c34)ccc2N1c1cccc2c1oc1ccccc12] | 74% |
| 9f | 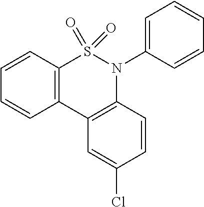 [SMILES: O=S1(=O)c2ccccc2-c2cc(Cl)ccc2N1c1ccccc1] | 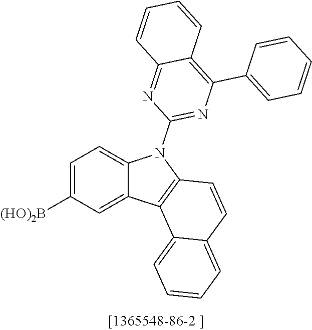 [SMILES: OB(O)c1ccc2c(c1)c1c3ccccc3ccc1n2-c1nc(-c2ccccc2)c2ccccc2n1] | 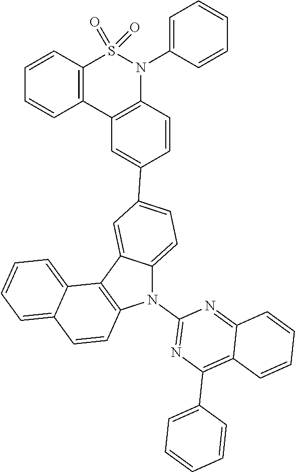 [SMILES: O=S1(=O)c2ccccc2-c2cc(-c3ccc4c(c3)c3c5ccccc5ccc3n4-c3nc(-c4ccccc4)c4ccccc4n3)ccc2N1c1ccccc1] | 77% |
| 10f | 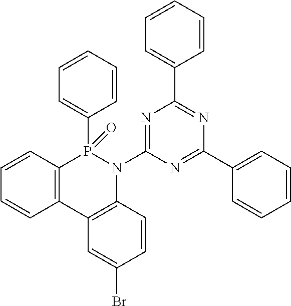 [SMILES: O=P1(c2ccccc2)c2ccccc2-c2cc(Br)ccc2N1c1nc(-c2ccccc2)nc(-c2ccccc2)n1] | 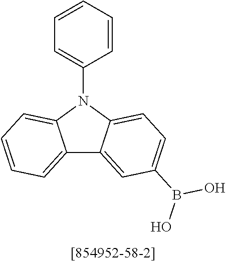 [SMILES: OB(O)c1ccc2c(c1)c1ccccc1n2-c1ccccc1] | 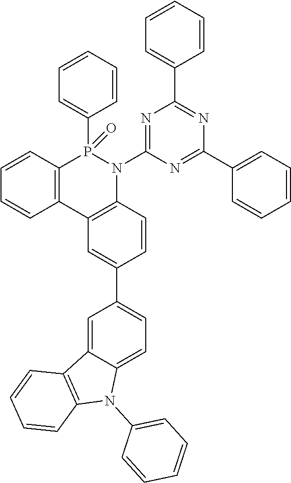 [SMILES: O=P1(c2ccccc2)c2ccccc2-c2cc(-c3ccc4c(c3)c3ccccc3n4-c3ccccc3)ccc2N1c1nc(-c2ccccc2)nc(-c2ccccc2)n1] | 54% |
| 11f | 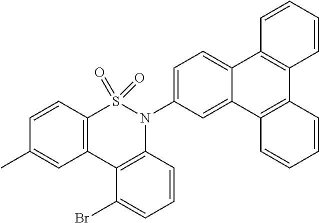 [SMILES: Cc1ccc2c(c1)-c1c(Br)cccc1N(c1ccc3c4ccccc4c4ccccc4c3c1)S2(=O)=O] | 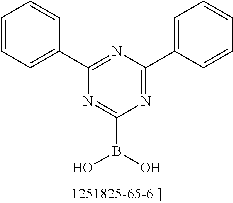 [SMILES: OB(O)c1nc(-c2ccccc2)nc(-c2ccccc2)n1] | 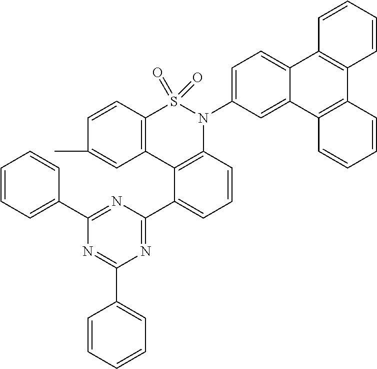 [SMILES: Cc1ccc2c(c1)-c1c(-c3nc(-c4ccccc4)nc(-c4ccccc4)n3)cccc1N(c1ccc3c4ccccc4c4ccccc4c3c1)S2(=O)=O] | 76% |
| 12f | 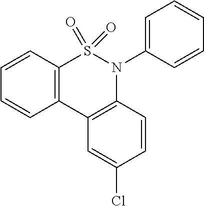 [SMILES: O=S1(=O)c2ccccc2-c2cc(Cl)ccc2N1c1ccccc1] | 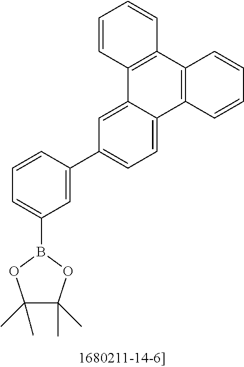 [SMILES: CC1(C)OB(c2cccc(-c3ccc4c5ccccc5c5ccccc5c4c3)c2)OC1(C)C] | 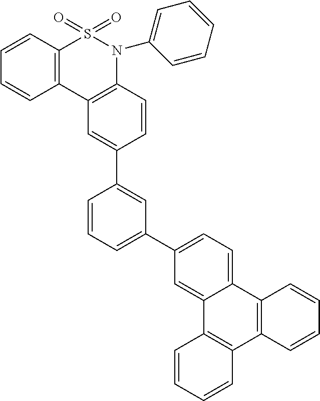 [SMILES: O=S1(=O)c2ccccc2-c2cc(-c3cccc(-c4ccc5c6ccccc6c6ccccc6c5c4)c3)ccc2N1c1ccccc1] | 70% |
| 13f | 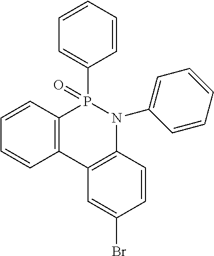 [SMILES: O=P1(c2ccccc2)c2ccccc2-c2cc(Br)ccc2N1c1ccccc1] | 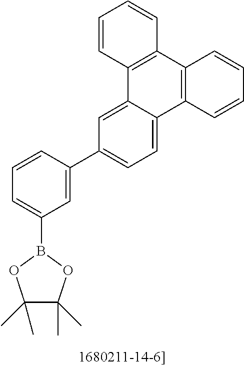 [SMILES: CC1(C)OB(c2cccc(-c3ccc4c5ccccc5c5ccccc5c4c3)c2)OC1(C)C] | 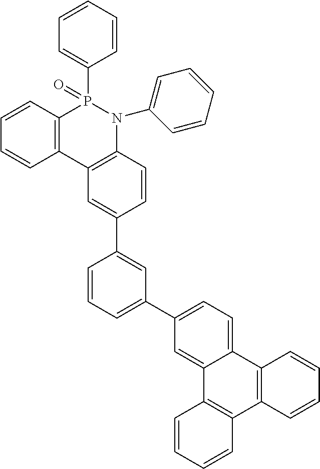 [SMILES: O=P1(c2ccccc2)c2ccccc2-c2cc(-c3cccc(-c4ccc5c6ccccc6c6ccccc6c5c4)c3)ccc2N1c1ccccc1] | 68% |
| 14f | 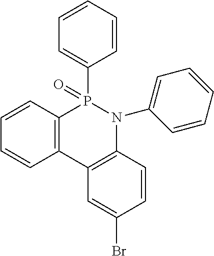 [SMILES: O=P1(c2ccccc2)c2ccccc2-c2cc(Br)ccc2N1c1ccccc1] | 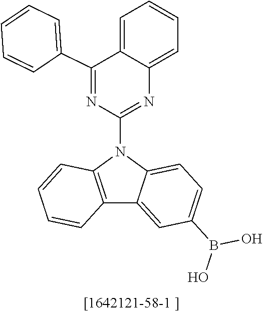 [SMILES: OB(O)c1ccc2c(c1)c1ccccc1n2-c1nc(-c2ccccc2)c2ccccc2n1] | 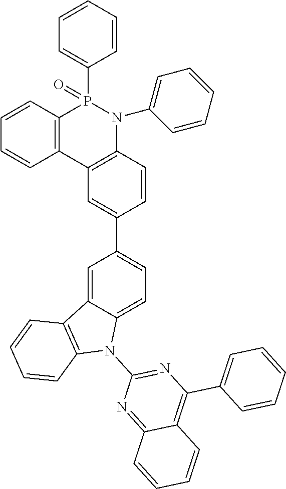 [SMILES: O=P1(c2ccccc2)c2ccccc2-c2cc(-c3ccc4c(c3)c3ccccc3n4-c3nc(-c4ccccc4)c4ccccc4n3)ccc2N1c1ccccc1] | 67% |
| 15f | 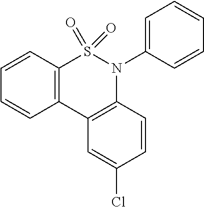 [SMILES: O=S1(=O)c2ccccc2-c2cc(Cl)ccc2N1c1ccccc1] | 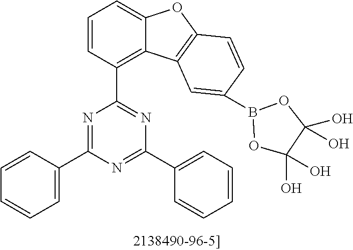 [SMILES: OC1(O)OB(c2ccc3oc4cccc(-c5nc(-c6ccccc6)nc(-c6ccccc6)n5)c4c3c2)OC1(O)O] | 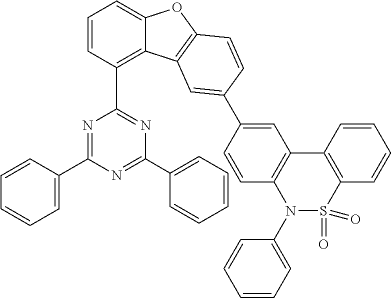 [SMILES: O=S1(=O)c2ccccc2-c2cc(-c3ccc4oc5cccc(-c6nc(-c7ccccc7)nc(-c7ccccc7)n6)c5c4c3)ccc2N1c1ccccc1] | 66% |
| 16f | 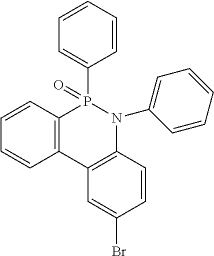 [SMILES: O=P1(c2ccccc2)c2ccccc2-c2cc(Br)ccc2N1c1ccccc1] | 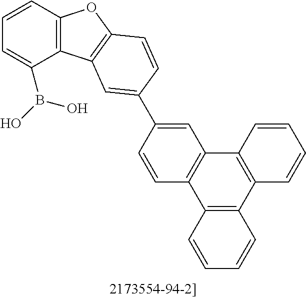 [SMILES: OB(O)c1cccc2oc3ccc(-c4ccc5c6ccccc6c6ccccc6c5c4)cc3c12] | 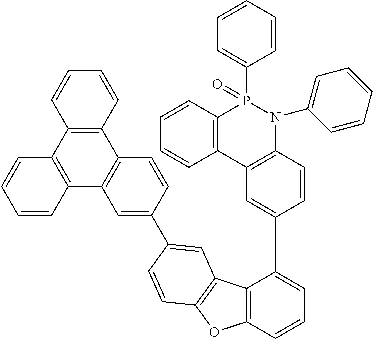 [SMILES: O=P1(c2ccccc2)c2ccccc2-c2cc(-c3cccc4oc5ccc(-c6ccc7c8ccccc8c8ccccc8c7c6)cc5c34)ccc2N1c1ccccc1] | 73% |
| 17f | 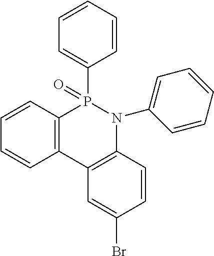 [SMILES: O=P1(c2ccccc2)c2ccccc2-c2cc(Br)ccc2N1c1ccccc1] | 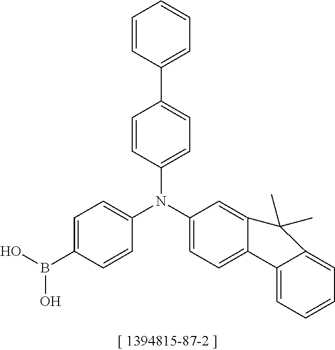 [SMILES: CC1(C)c2ccccc2-c2ccc(N(c3ccc(B(O)O)cc3)c3ccc(-c4ccccc4)cc3)cc21] | 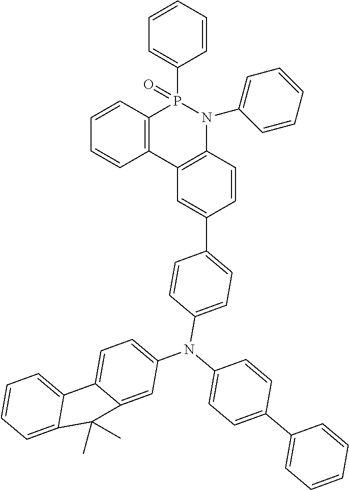 [SMILES: CC1(C)c2ccccc2-c2ccc(N(c3ccc(-c4ccccc4)cc3)c3ccc(-c4ccc5c(c4)-c4ccccc4P(=O)(c4ccccc4)N5c4ccccc4)cc3)cc21] | 71% |
| 18f | 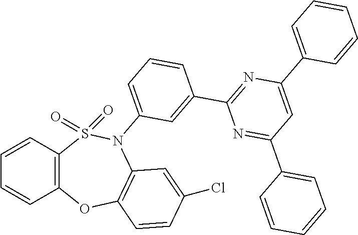 [SMILES: O=S1(=O)c2ccccc2Oc2ccc(Cl)cc2N1c1cccc(-c2nc(-c3ccccc3)cc(-c3ccccc3)n2)c1] | 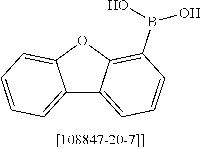 [SMILES: OB(O)c1cccc2c1oc1ccccc12] | 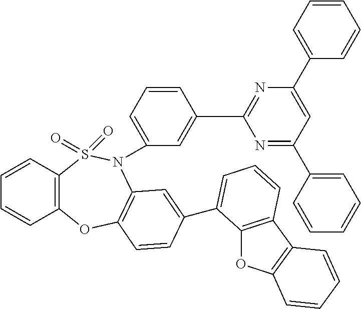 [SMILES: O=S1(=O)c2ccccc2Oc2ccc(-c3cccc4c3oc3ccccc34)cc2N1c1cccc(-c2nc(-c3ccccc3)cc(-c3ccccc3)n2)c1] | 65% |
| 19f | 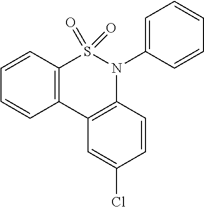 [SMILES: O=S1(=O)c2ccccc2-c2cc(Cl)ccc2N1c1ccccc1] | 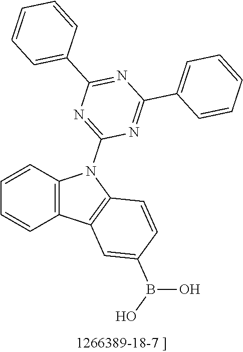 [SMILES: OB(O)c1ccc2c(c1)c1ccccc1n2-c1nc(-c2ccccc2)nc(-c2ccccc2)n1] | 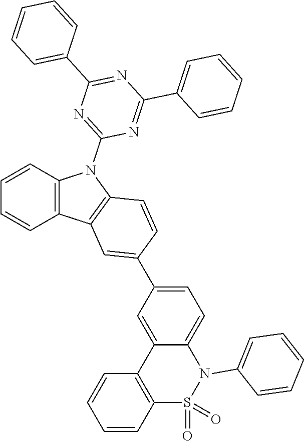 [SMILES: O=S1(=O)c2ccccc2-c2cc(-c3ccc4c(c3)c3ccccc3n4-c3nc(-c4ccccc4)nc(-c4ccccc4)n3)ccc2N1c1ccccc1] | 68% |
| 20f | 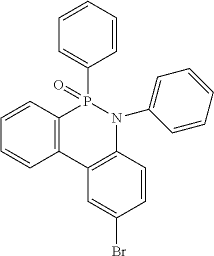 [SMILES: O=P1(c2ccccc2)c2ccccc2-c2cc(Br)ccc2N1c1ccccc1] | 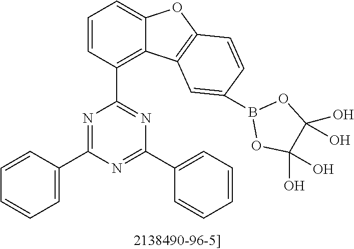 [SMILES: OC1(O)OB(c2ccc3oc4cccc(-c5nc(-c6ccccc6)nc(-c6ccccc6)n5)c4c3c2)OC1(O)O] | 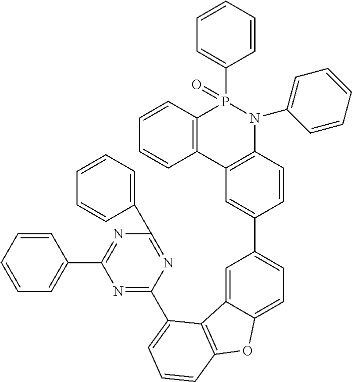 [SMILES: O=P1(c2ccccc2)c2ccccc2-c2cc(-c3ccc4oc5cccc(-c6nc(-c7ccccc7)nc(-c7ccccc7)n6)c5c4c3)ccc2N1c1ccccc1] | 71% |

### Production of the OLEDs

Examples E1 to E9 which follow (see table 1) present the use of the materials of the invention in OLEDs.

### Pretreatment for Examples E1-E9

Glass plaques coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as IC1:IC2:TEG1 (55%:35%:10%) mean here that the material IC1 is present in the layer in a proportion by volume of 55%, IC2 in a proportion by volume of 35% and TEG1 in a proportion by volume of 10%. Analogously, the electron transport layer may also consist of a mixture of two materials.

The OLEDs are characterized in a standard manner. The electroluminescence spectra are determined at a luminance of 1000 cd/m², and the CIE 1931 x and y color coordinates are calculated therefrom.

### Use of Mixtures of the Invention in OLEDs

The materials of the invention can be used as matrix material in the emission layer of phosphorescent green OLEDs. The results are collated in table 3.

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| E1 | HATCN | SpMA1 | SpMA2 | 18c:IC2:TEG1 | ST2 | ST2:LiQ | LiQ |
| | 5 nm | 230 nm | 20 nm | (64%:29%:7%) | 5 nm | (50%:50%) | 1 nm |
| | | | | 40 nm | | 30 nm | |
| E2 | HATCN | SpMA1 | SpMA2 | IC1:f:TEG1 | ST2 | ST2:LiQ | LiQ |
| | 5 nm | 230 nm | 20 nm | (47%:46%:7%) | 5 nm | (50%:50%) | 1 nm |
| | | | | 40 nm | | 30 nm | |
| E3 | HATCN | SpMA1 | SpMA2 | CbzT4:8f:TEG1 | ST2 | ST2:LiQ | LiQ |
| | 5 nm | 230 nm | 20 nm | (66%:22%:12%) | 5 nm | (50%:50%) | 1 nm |
| | | | | 40 nm | | 30 nm | |
| E4 | HATCN | SpMA1 | SpMA2 | IC1:10f:TEG1 | ST2 | ST2:LiQ | LiQ |
| | 5 nm | 230 nm | 20 nm | (66%:22%:12%) | 5 nm | (50%:50%) | 1 nm |
| | | | | 40 nm | | 30 nm | |
| E5 | HATCN | SpMA1 | SpMA2 | CbzT4:13f:TEG1 | ST2 | ST2:LiQ | LiQ |
| | 5 nm | 230 nm | 20 nm | (47%:46%:7%) | 5 nm | (50%:50%) | 1 nm |
| | | | | 40 nm | | 30 nm | |
| E6 | HATCN | SpMA1 | SpMA2 | 15f:IC2:TEG1 | ST2 | ST2:LiQ | LiQ |
| | 5 nm | 230 nm | 20 nm | (47%:46%:7%) | 5 nm | (50%:50%) | 1 nm |
| | | | | 40 nm | | 30 nm | |
| E7 | HATCN | SpMA1 | SpMA2 | IC1:17f:TEG1 | ST2 | ST2:LiQ | LiQ |
| | 5 nm | 230 nm | 20 nm | (44%:44%:12%) | 5 nm | (50%:50%) | 1 nm |
| | | | | 40 nm | | 30 nm | |
| E8 | HATCN | SpMA1 | SpMA2 | CbzT4:18f:TEG1 | ST2 | ST2:LiQ | LiQ |
| | 5 nm | 230 nm | 20 nm | (47%:46%:7%) | 5 nm | (50%:50%) | 1 nm |
| | | | | 40 nm | | 30 nm | |
| E9 | HATCN | SpMA1 | SpMA2 | 19f:IC2:TEG1 | ST2 | ST2:LiQ | LiQ |
| | 5 nm | 230 nm | 20 nm | (34%:59%:7%) | 5 nm | (50%:50%) | 1 nm |
| | | | | 40 nm | | 30 nm | |

TABLE 2
Structural formulae of the materials for the OLEDs

| | |
|---|---|
| 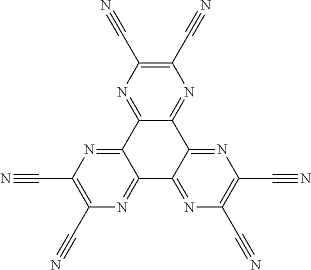 [SMILES: N#Cc1nc2c3nc(C#N)c(C#N)nc3c3nc(C#N)c(C#N)nc3c2nc1C#N] | HATCN |
| 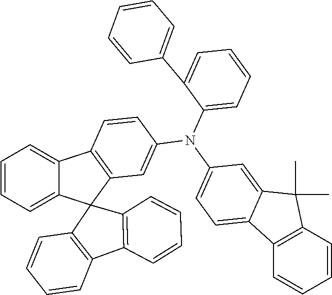 [SMILES: CC1(C)c2ccccc2-c2ccc(N(c3ccc4c(c3)C3(c5ccccc5-c5ccccc53)c3ccccc3-4)c3ccccc3-c3ccccc3)cc21] | SpMA1 |
| 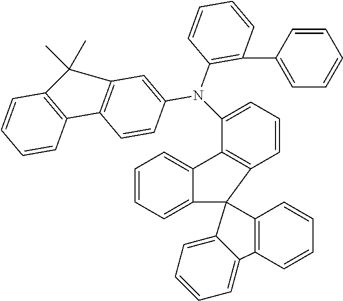 [SMILES: CC1(C)c2ccccc2-c2ccc(N(c3ccccc3-c3ccccc3)c3cccc4c3-c3ccccc3C43c4ccccc4-c4ccccc43)cc21] | SpMA3 |
| 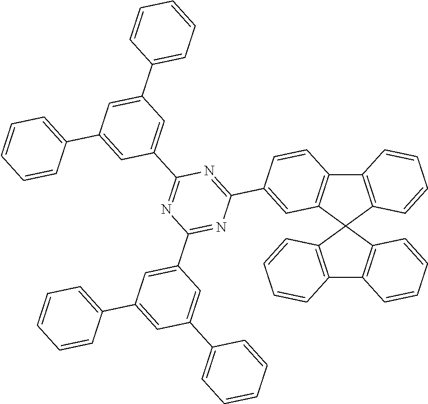 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ST2 |
| 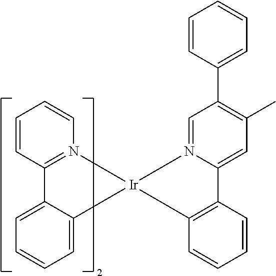 | TEG1 |
| 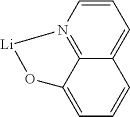 | LiQ |
| 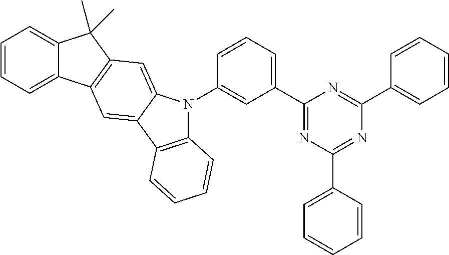 [SMILES: CC1(C)c2ccccc2-c2cc3c4ccccc4n(-c4cccc(-c5nc(-c6ccccc6)nc(-c6ccccc6)n5)c4)c3cc21] | IC1 |
| 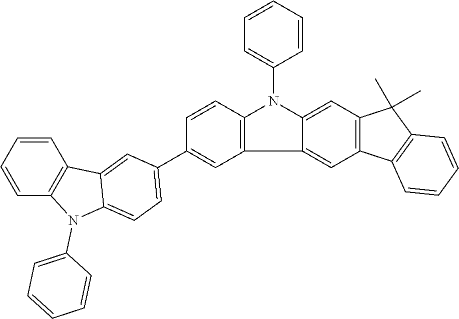 [SMILES: CC1(C)c2ccccc2-c2cc3c4cc(-c5ccc6c(c5)c5ccccc5n6-c5ccccc5)ccc4n(-c4ccccc4)c3cc21] | IC2 |
| 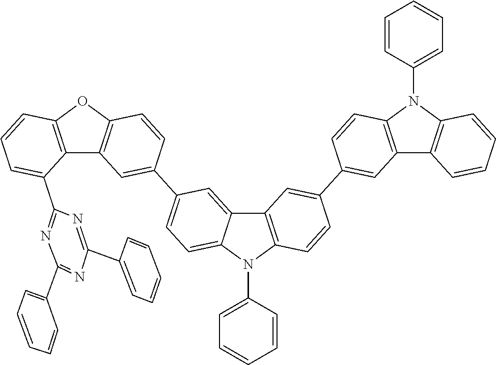 [SMILES: c1ccc(-c2nc(-c3ccccc3)nc(-c3cccc4oc5ccc(-c6ccc7c(c6)c6cc(-c8ccc9c(c8)c8ccccc8n9-c8ccccc8)ccc6n7-c6ccccc6)cc5c34)n2)cc1] | CbzT4 |
| 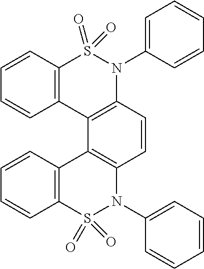 [SMILES: O=S1(=O)c2ccccc2-c2c(ccc3c2-c2ccccc2S(=O)(=O)N3c2ccccc2)N1c1ccccc1] | 18c |
| 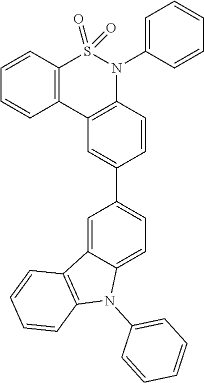 [SMILES: O=S1(=O)c2ccccc2-c2cc(-c3ccc4c(c3)c3ccccc3n4-c3ccccc3)ccc2N1c1ccccc1] | f |
| 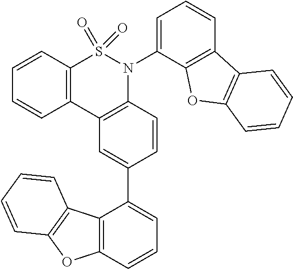 [SMILES: O=S1(=O)c2ccccc2-c2cc(-c3cccc4oc5ccccc5c34)ccc2N1c1cccc2c1oc1ccccc12] | 8f |
| 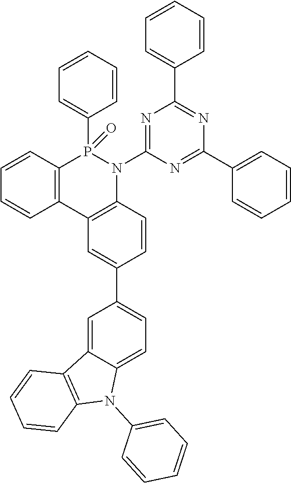 [SMILES: O=P1(c2ccccc2)c2ccccc2-c2cc(-c3ccc4c(c3)c3ccccc3n4-c3ccccc3)ccc2N1c1nc(-c2ccccc2)nc(-c2ccccc2)n1] | 10f |
| 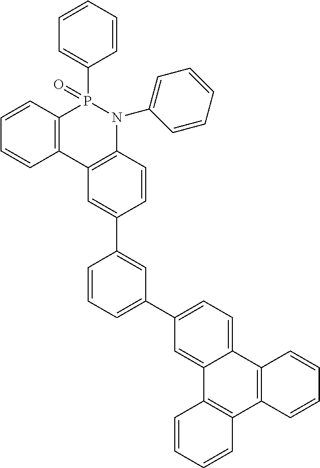 [SMILES: O=P1(c2ccccc2)c2ccccc2-c2cc(-c3cccc(-c4ccc5c6ccccc6c6ccccc6c5c4)c3)ccc2N1c1ccccc1] | 13f |
| 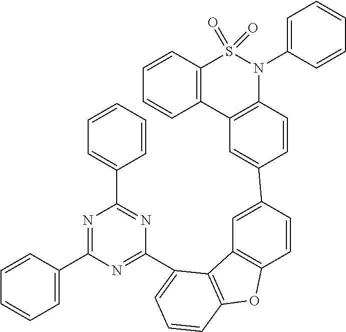 [SMILES: O=S1(=O)c2ccccc2-c2cc(-c3ccc4oc5cccc(-c6nc(-c7ccccc7)nc(-c7ccccc7)n6)c5c4c3)ccc2N1c1ccccc1] | 15f |
| 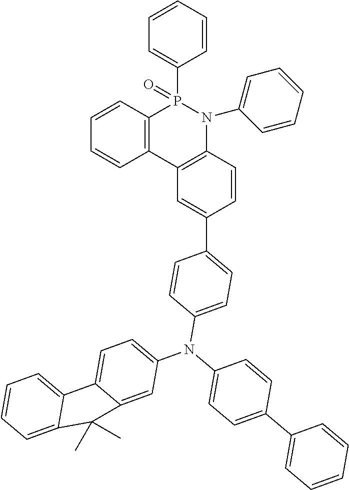 [SMILES: CC1(C)c2ccccc2-c2ccc(N(c3ccc(-c4ccccc4)cc3)c3ccc(-c4ccc5c(c4)-c4ccccc4P(=O)(c4ccccc4)N5c4ccccc4)cc3)cc21] | 17f |
| 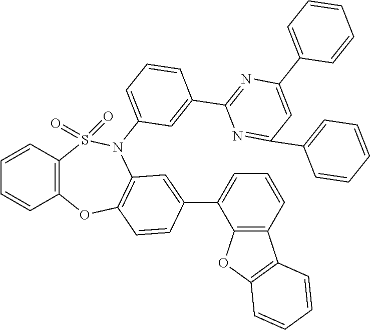 [SMILES: O=S1(=O)c2ccccc2Oc2ccc(-c3cccc4c3oc3ccccc34)cc2N1c1cccc(-c2nc(-c3ccccc3)cc(-c3ccccc3)n2)c1] | 18f |
| 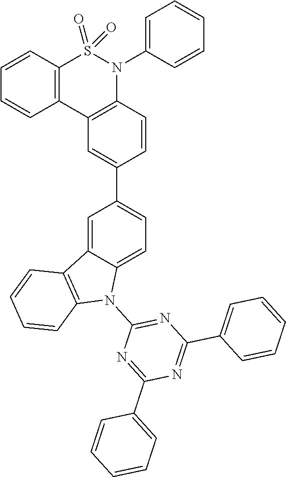 [SMILES: O=S1(=O)c2ccccc2-c2cc(-c3ccc4c(c3)c3ccccc3n4-c3nc(-c4ccccc4)nc(-c4ccccc4)n3)ccc2N1c1ccccc1] | 19f |

TABLE 3
Results for the electroluminescent devices

| | U1000 | SE1000 | EQE 1000 | CIE x/y at |
|---|---|---|---|---|
| Ex. | (V) | (cd/A) | (%) | 1000 cd/m² |
| E1 | 3.3 | 68 | 19 | 0.36/0.61 |
| E2 | 3.5 | 73 | 18 | 0.35/0.62 |
| E3 | 3.4 | 72 | 17 | 0.34/0.62 |
| E4 | 3.5 | 69 | 18 | 0.35/0.61 |
| E5 | 3.2 | 73 | 17 | 0.35/0.62 |
| E6 | 3.4 | 73 | 17 | 0.35/0.62 |
| E7 | 3.7 | 65 | 16 | 0.32/0.63 |
| E8 | 3.5 | 63 | 16 | 0.33/0.63 |
| E9 | 3.1 | 60 | 19 | 0.33/0.63 |

1. A compound of formula (1) 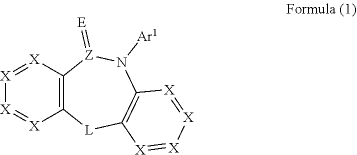
where the symbols used are as follows:
Z is the same or different at each instance and is PAr² or S(═O);
E is the same or different at each instance and is O or S when the symbol Z to which this E binds is PAr², and O when the symbol Z to which this E binds is S(═O);
L is selected from the group consisting of a single bond, NAr², O, S, S(═O)₂, P(═O)Ar², —X═X— and —C(═O)—NAr²—;
X is the same or different at each instance and is CR or N, where not more than two X groups per cycle are N; or two adjacent X are a group of the following formula (2), (3) or (4): 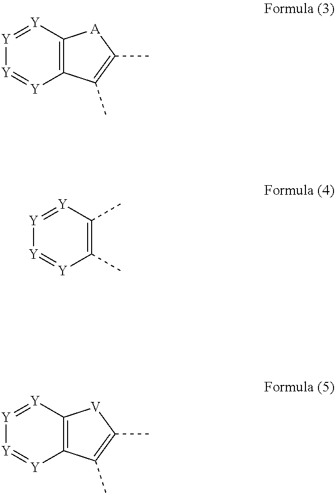
where the dotted bonds indicate the linkage of this group in the formula (1);
Y is the same or different at each instance and is CR or N, where not more than two Y groups per cycle are N;
Ar¹, Ar² is the same or different at each instance and is an aromatic or heteroaromatic ring system which has 5 to 40 aromatic ring atoms and may be substituted by one or more R radicals, where Ar¹ and Ar² are not joined to one another, with the proviso that the substituents R that bind to Ar¹ are not fluorine;
A is the same or different at each instance and is NAr², O, S or CR₂;
V is the same or different at each instance and is —NAr²—Z(=E)-;
R is the same or different at each instance and is H, D, F, Cl, Br, I, N(Ar³)₂, OAr³, SAr³, CN, NO₂, NAr³R¹, N(R¹)₂, OR¹, SR¹, COOR¹, C(═O)N(R¹)₂, Si(R¹)₃, B(OR¹)₂, C(═O)R¹, P(═O)(R¹)₂, S(═O)R¹, S(═O)₂R¹, OSO₂R¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹ radicals, where one or more nonadjacent CH₂ groups may be replaced by Si(R¹)₂, C═O, NR¹, O, S or CONR¹, or an aromatic or heteroaromatic ring system which has 5 to 40 aromatic ring atoms and may be substituted in each case by one or more R¹ radicals; at the same time, two R radicals together may also form an aliphatic or heteroaliphatic ring system;
Ar³ is the same or different at each instance and is an aromatic or heteroaromatic ring system which has 5 to 30 aromatic ring atoms and may be substituted by one or more R¹ radicals;
R¹ is the same or different at each instance and is H, D, F, Cl, Br, I, N(R²)₂, CN, NO₂, OR², SR², Si(R²)₃, B(OR²)₂, C(═O)R², P(═O)(R²)₂, S(═O)R², OSO₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals, where one or more nonadjacent CH₂ groups may be replaced by Si(R²)₂, C═O, NR², O, S or CONR², or an aromatic or heteroaromatic ring system which has 5 to 40 aromatic ring atoms and may be substituted in each case by one or more R² radicals; at the same time, two or more R¹ radicals together may form an aliphatic ring system;
R² is the same or different at each instance and is H, D, F, CN or an aliphatic, aromatic or heteroaromatic organic radical, especially a hydrocarbyl radical, having 1 to 20 carbon atoms, in which one or more hydrogen atoms may also be replaced by F;
where the following compounds are excluded from the invention:

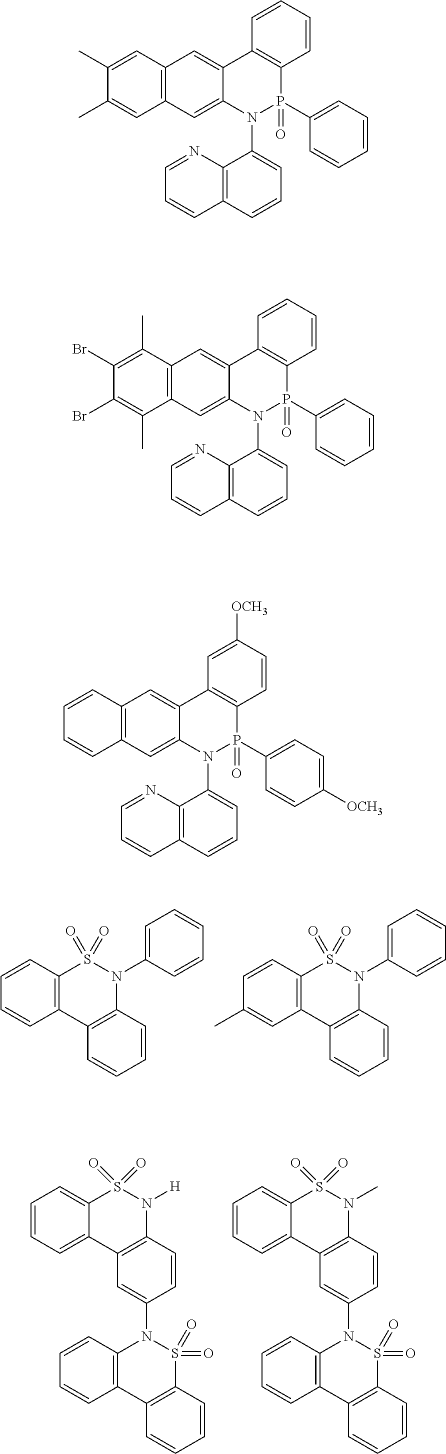

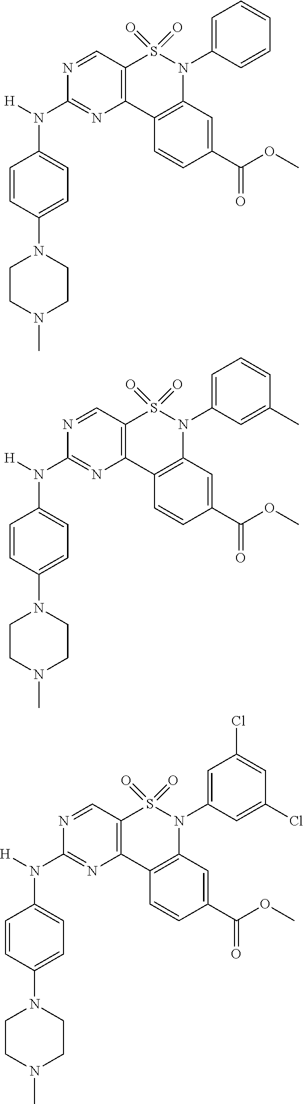 [SMILES: COC(=O)c1ccc2c(c1)N(c1ccccc1)S(=O)(=O)c1cnc(Nc3ccc(N4CCN(C)CC4)cc3)nc1-2 ; COC(=O)c1ccc2c(c1)N(c1cccc(C)c1)S(=O)(=O)c1cnc(Nc3ccc(N4CCN(C)CC4)cc3)nc1-2 ; COC(=O)c1ccc2c(c1)N(c1cc(Cl)cc(Cl)c1)S(=O)(=O)c1cnc(Nc3ccc(N4CCN(C)CC4)cc3)nc1-2]

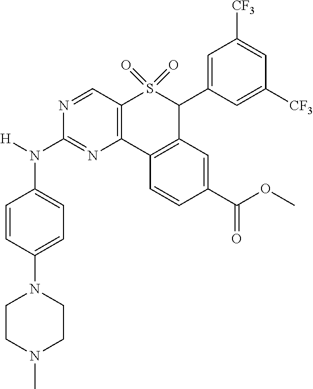 [SMILES: COC(=O)c1ccc2c(c1)C(c1cc(C(F)(F)F)cc(C(F)(F)F)c1)S(=O)(=O)c1cnc(Nc3ccc(N4CCN(C)CC4)cc3)nc1-2]

2. A compound as claimed in claim 1, selected from the compounds of the formulae (1-1) and (5) to (16) 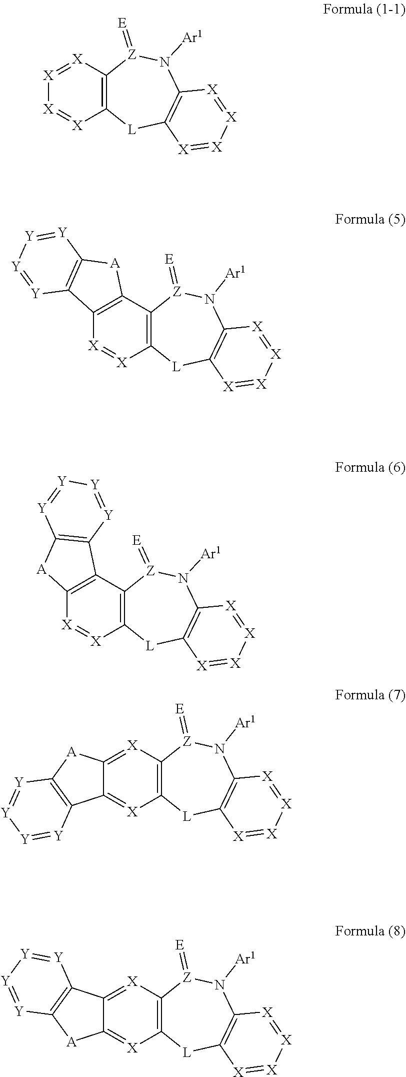 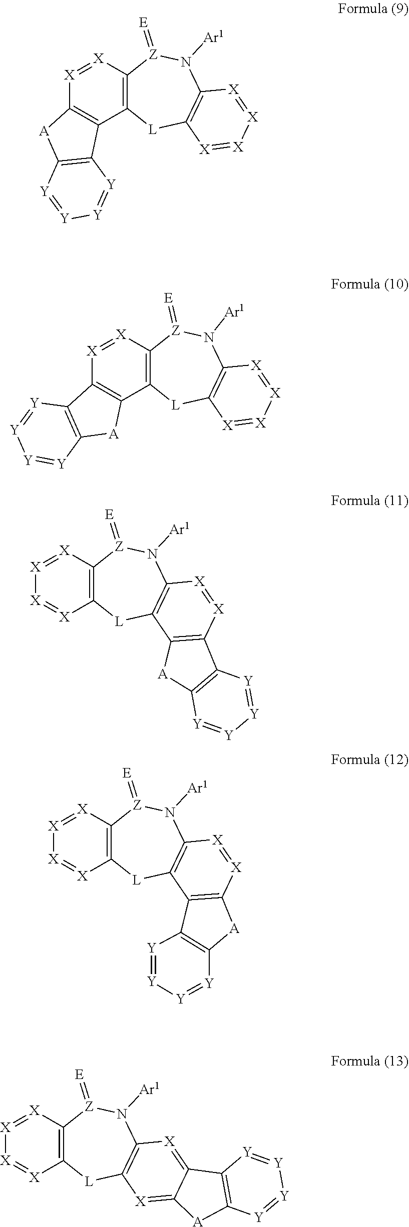 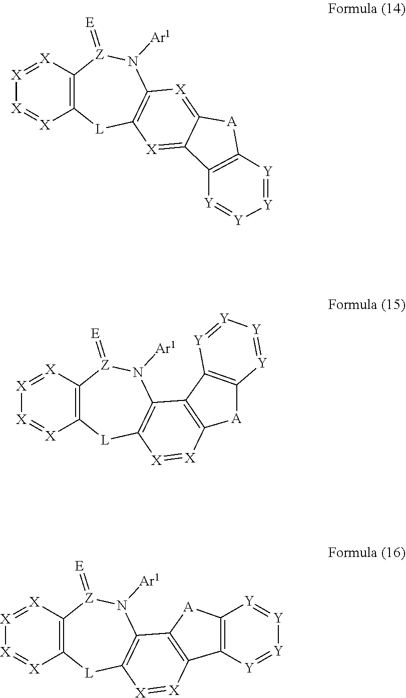
where X is the same or different at each instance and is CR or N, where not more than two X groups per cycle are N, and the further symbols used have the definitions given in claim 1.

3. A compound as claimed in claim 1, characterized in that the group of the formula (2), (3) or (4) is respectively selected from the groups of the formula (2a), (3a) or (4a): 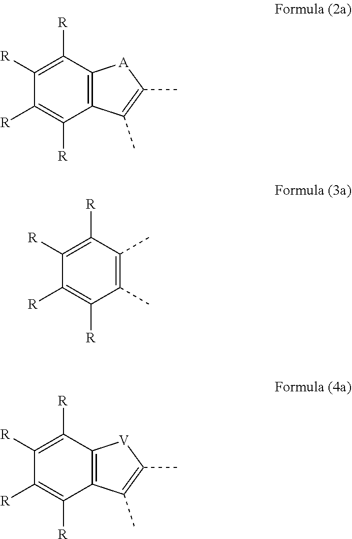
where the symbols used have the definitions given above.

4. A compound as claimed in claim 1, characterized in that the symbols X that are not a group of the formula (2), (3) or (4) are the same or different at each instance and are CR, and in that the symbols Y are the same or different at each instance and are CR.

5. A compound as claimed in claim 1, selected from the structures of the formulae (1a) and (5a) to (16a), 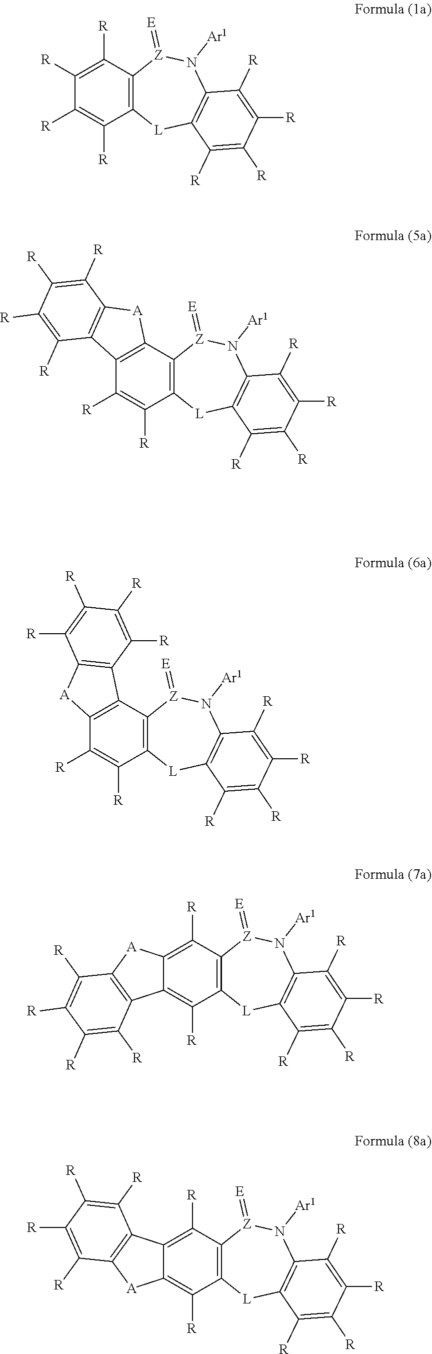 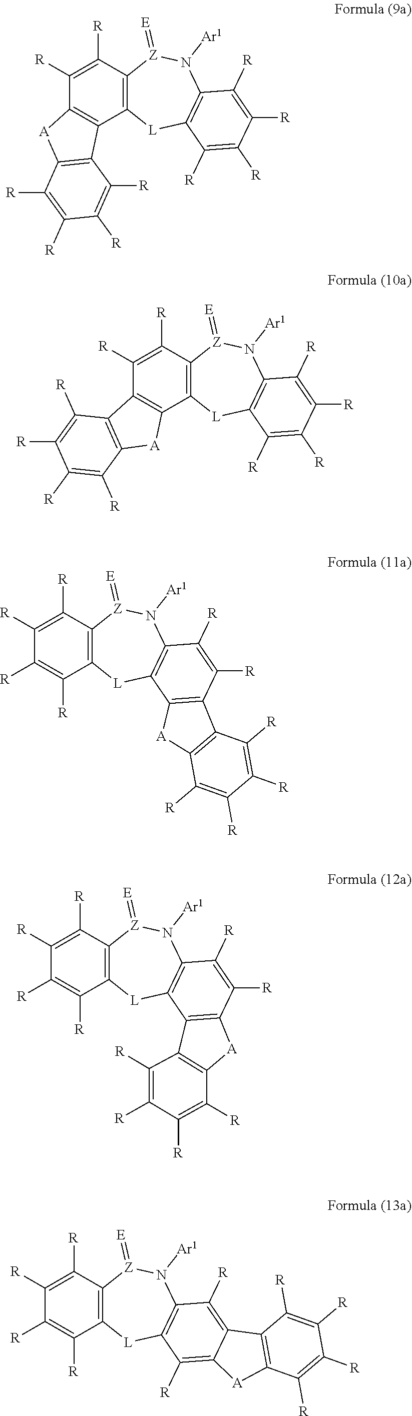 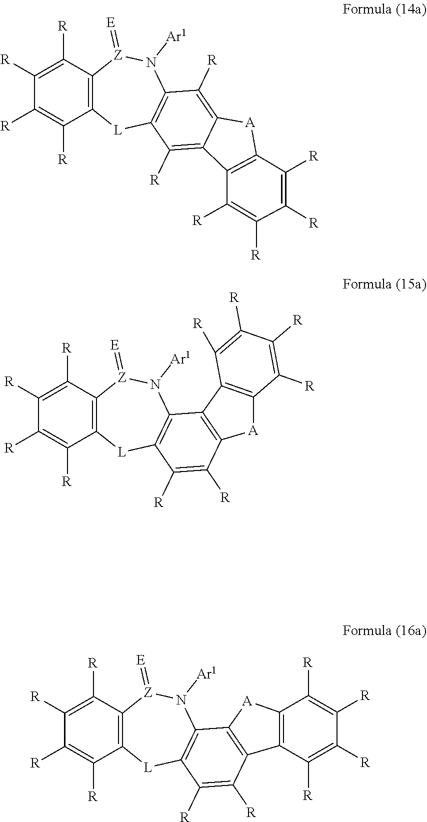
where the symbols have the definitions given in claim 1.

6. A compound as claimed in claim 1, selected from the structures of the formulae (17) and (18): 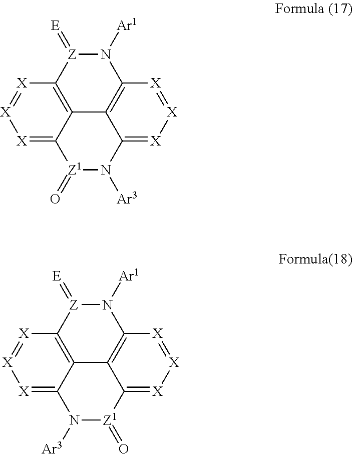
where the symbols used have the definitions given in claim 1 and Z¹ is PR¹ or S(═O).

7. A compound as claimed in claim 1, selected from the structures of the formulae (1b) or (5b) to (16b): 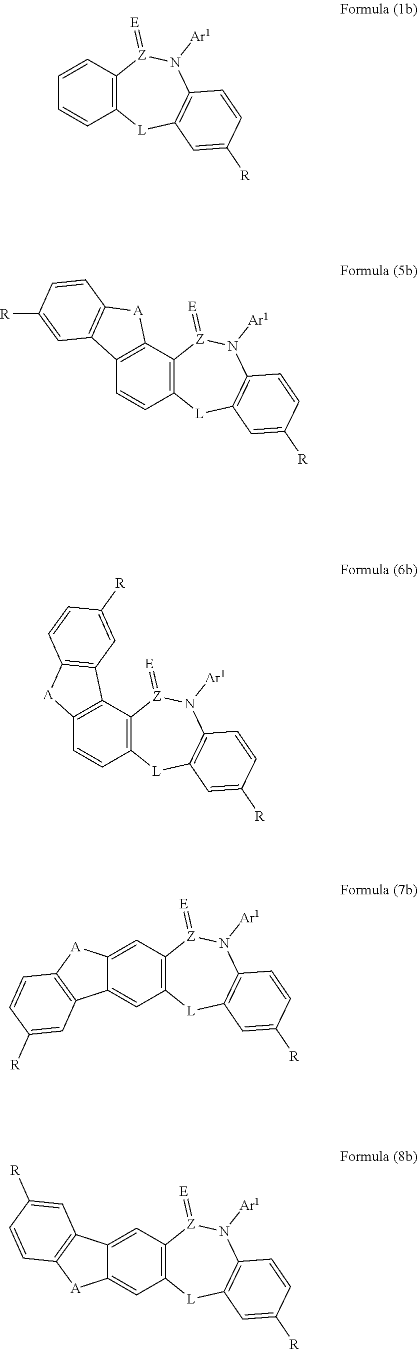 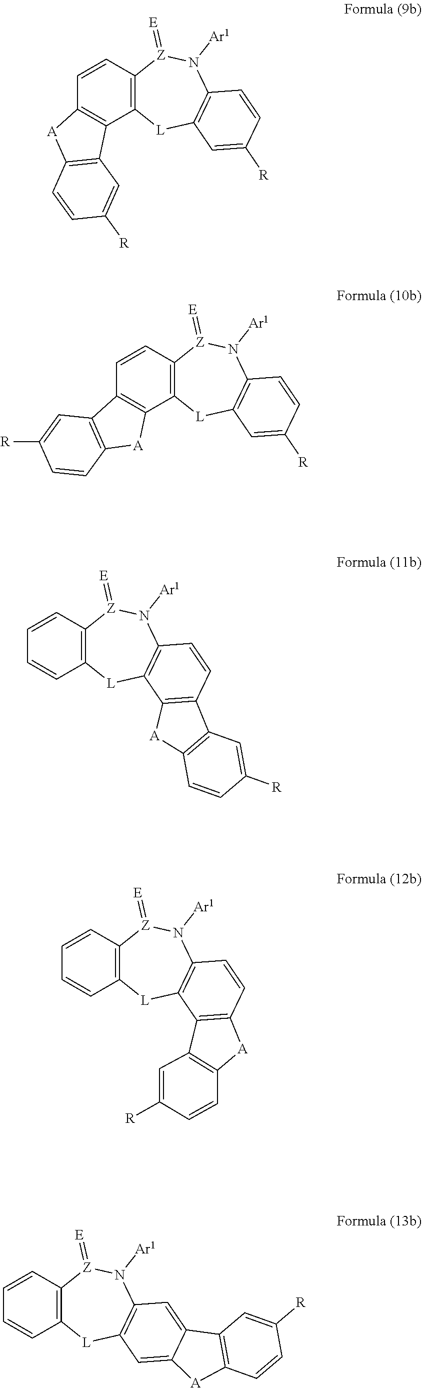 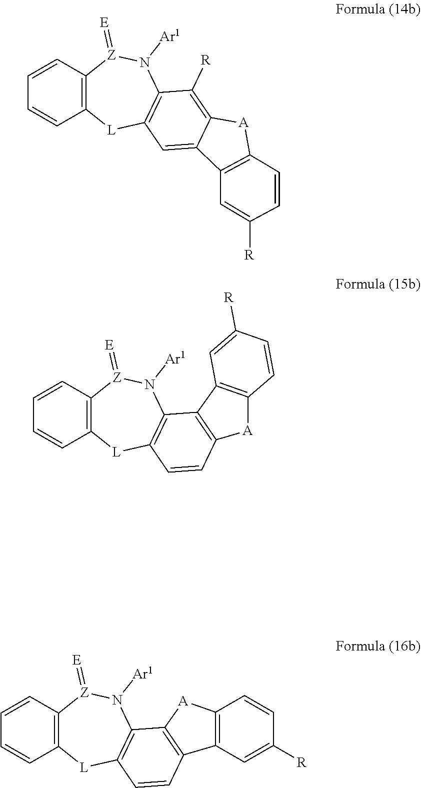
where the symbols used have the definitions given in claim 1.

8. A compound as claimed in claim 1, characterized in that Z is PAr² and E is O and L is a single bond.

9. A compound as claimed in claim 1, characterized in that Ar¹ and Ar² are the same or different at each instance and are an aromatic or heteroaromatic ring system which has 6 to 24 aromatic ring atoms and may be substituted by one or more R radicals, where the R radicals in the case of Ar¹ are not fluorine.

10. A compound as claimed in claim 1, characterized in that R is the same or different at each instance and is selected from the group consisting of H, D, F, N(Ar³)₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R¹ radicals, where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic ring system.

11. A formulation comprising at least one compound as claimed in claim 1 and at least one further compound and/or solvent.

12. The use of a compound as claimed in claim 1 in an electronic device.

13. An electronic device comprising at least one compound as claimed in claim 1.

14. An organic electroluminescent device, characterized in that the compound as claimed in claim 1 present in an emitting layer, especially as matrix material, and/or in an electron transport layer and/or in a hole blocker layer and/or in a hole transport layer and/or in an exciton blocker layer.
